(19) United States
(12) Patent Application Publication
(10) Pub. No.: US 2024/0060135 A1
(43) Pub. Date: Feb. 22, 2024

(54) THERAPEUTIC AND DIAGNOSTIC METHODS FOR CANCER

(71) Applicant: Genentech, Inc., South San Francisco, CA (US)

(72) Inventors: Li-Fen LIU, South San Francisco, CA (US); Sanjeev MARIATHASAN, Millbrae, CA (US); Chi Yung YUEN, Millbrae, CA (US); Mahrukh HUSENI, Union City, CA (US)

(21) Appl. No.: 17/937,102

(22) Filed: Sep. 30, 2022

Related U.S. Application Data
(63) Continuation of application No. PCT/US2021/025511, filed on Apr. 2, 2021.
(60) Provisional application No. 63/005,063, filed on Apr. 3, 2020.

(51) Int. Cl. C12Q 1/6886; A61P 35/00; C07K 16/28; G01N 33/574

(52) U.S. Cl. CPC C12Q 1/6886; A61P 35/00; C07K 16/2827; G01N 33/57484; C07K 2317/24; C12Q 2600/106; C12Q 2600/158; G01N 2333/5421

(57) ABSTRACT

The present invention provides therapeutic and diagnostic methods and compositions for cancer (e.g., bladder cancer (e.g., UC) or kidney cancer (e.g., RCC)). The invention provides methods of identifying an individual having a cancer who is likely to respond to treatment with an anti-cancer therapy comprising a PD-L1 axis binding antagonist, methods for selecting a therapy for an individual having a cancer, methods of identifying an individual having a cancer who is less likely to respond to treatment with an anti-cancer therapy comprising a PD-L1 axis binding antagonist monotherapy, methods of monitoring the response of an individual having a cancer to treatment with an anti-cancer therapy comprising a PD-L1 axis binding antagonist, and methods of treating an individual having cancer, based on expression levels of a biomarker of the invention (e.g., one or more genes set forth in any one of Tables 1-7, e.g., IL8).

### FIELD OF THE INVENTION

Provided herein are therapeutic and diagnostic methods and compositions for pathological conditions, such as cancer. In particular, the invention provides biomarkers for patient identification, selection, and diagnosis; methods of treatment; articles of manufacture; and diagnostic kits.

### BACKGROUND

Cancer remains one of the deadliest threats to human health. Cancers, or malignant tumors, metastasize and grow rapidly in an uncontrolled manner, making timely detection and treatment extremely difficult. In the U.S., cancer affects nearly 1.3 million new patients each year, and is the second leading cause of death after heart disease, accounting for approximately 1 in 4 deaths. Solid tumors are responsible for most of those deaths. As one example, bladder cancer is the fifth-most common malignancy worldwide, with close to 400,000 newly diagnosed cases and approximately 150,000 associated deaths reported per year. In particular, metastatic urothelial carcinoma (UC) is associated with poor outcomes and represents a major unmet medical need with few effective therapies to date. In another example, renal cell carcinoma (RCC) is the most common type of kidney cancer and has multiple histological subtypes.

Programmed death-ligand 1 (PD-L1) is a protein that has been implicated in the suppression of immune system responses during chronic infections, pregnancy, tissue allografts, autoimmune diseases, and cancer. PD-L1 regulates the immune response by binding to an inhibitory receptor, known as programmed death 1 (PD-1), which is expressed on the surface of T-cells, B-cells, and monocytes. PD-L1 negatively regulates T-cell function also through interaction with another receptor, B7-1. Formation of the PD-L1/PD-1 and PD-L1/B7-1 complexes negatively regulates T-cell receptor signaling, resulting in the subsequent downregulation of T-cell activation and suppression of anti-tumor immune activity.

Despite the significant advancement in the treatment of cancer (e.g., bladder cancer (e.g., UC) and kidney cancer (e.g., RCC)), improved therapies and diagnostic methods are still being sought.

### SUMMARY OF THE INVENTION

The present invention provides therapeutic and diagnostic methods and compositions for cancer (e.g., bladder cancer (e.g., UC) and kidney cancer (e.g., RCC)).

In one aspect, the invention features a method of treating an individual having a cancer, the method comprising: (a) determining the expression level of IL8 in a sample from the individual, wherein an expression level of IL8 in the sample that is below a reference level of IL8 identifies the individual as likely to respond to treatment with an anti-cancer therapy comprising a PD-L1 binding antagonist, wherein the response is indicated by overall survival (OS) or overall response rate (ORR); and (b) administering an effective amount of an anti-cancer therapy comprising a PD-L1 binding antagonist to the individual based on the expression level of IL8 determined in step (a).

In another aspect, the invention features a method of treating an individual having a cancer who has been determined to be likely to respond to treatment with an anti-cancer therapy comprising a PD-L1 binding antagonist based on an expression level of IL8 in a sample from the individual that is below a reference level of IL8, the method comprising administering to the individual an effective amount of an anti-cancer therapy comprising a PD-L1 binding antagonist, wherein the response is indicated by OS or ORR.

In another aspect, the invention features a method of identifying an individual having a cancer who is likely to respond to treatment with an anti-cancer therapy comprising a PD-L1 binding antagonist, the method comprising determining the expression level of IL8 in a sample from the individual, wherein an expression level of IL8 in the sample that is below a reference level of IL8 identifies the individual as likely to respond to treatment with an anti-cancer therapy comprising a PD-L1 binding antagonist, wherein the response is indicated by OS or ORR.

In another aspect, the invention features a method for selecting a therapy for an individual having a cancer, the method comprising: (a) determining the expression level of IL8 in a sample from the individual, wherein an expression level of IL8 in the sample that is below a reference level of IL8 identifies the individual as likely to respond to treatment with an anti-cancer therapy comprising a PD-L1 binding antagonist, wherein the response is indicated by OS or ORR; and (b) selecting an anti-cancer therapy comprising a PD-L1 binding antagonist for the individual based on the expression level of IL8 determined in step (a).

In another aspect, the invention features a method of treating an individual having a cancer, the method comprising: (a) determining the expression level of IL8 in a sample from the individual, wherein an expression level of IL8 in the sample that is at or above a reference level of IL8 identifies the individual as less likely to respond to treatment with an anti-cancer therapy comprising a PD-L1 binding antagonist monotherapy, wherein the response is indicated by OS or ORR; and (b) administering an effective amount of an anti-cancer therapy other than or in addition to a PD-L1 binding antagonist to the individual based on the expression level of IL8 determined in step (a).

In another aspect, the invention features a method of treating an individual having a cancer who has been determined to be less likely to respond to treatment with an anti-cancer therapy comprising a PD-L1 binding antagonist monotherapy based on an expression level of IL8 in a sample from the individual that is at or above a reference level of IL8, the method comprising administering to the individual an effective amount of an anti-cancer therapy other than or in addition to a PD-L1 binding antagonist, wherein the response is indicated by OS or ORR.

In another aspect, the invention features a method of identifying an individual having a cancer who is less likely to respond to treatment with an anti-cancer therapy comprising a PD-L1 binding antagonist monotherapy, the method comprising determining the expression level of IL8 in a sample from the individual, wherein an expression level of IL8 in the sample that is at or above a reference level of IL8 identifies the individual as less likely to respond to treatment with an anti-cancer therapy comprising a PD-L1 binding antagonist monotherapy, and wherein the response is indicated by OS or ORR.

In another aspect, the invention features a method for selecting a therapy for an individual having a cancer, the method comprising: (a) determining the expression level of IL8 in a sample from the individual, wherein an expression level of IL8 in the sample that is at or above a reference level of IL8 identifies the individual as less likely to respond to treatment with an anti-cancer therapy comprising a PD-L1 binding antagonist monotherapy, wherein the response is indicated by OS or ORR; and (b) selecting an anti-cancer therapy other than or in addition to a PD-L1 binding antagonist for the individual based on the expression level of IL8 determined in step (a).

In another aspect, the invention features a method of treating an individual having a cancer, the method comprising: (a) administering an effective amount of an anti-cancer therapy comprising a PD-L1 binding antagonist to the individual; (b) determining the expression level of IL8 in a sample obtained from the individual at a time point following administration of the anti-cancer therapy; and (c) continuing to administer the anti-cancer therapy to the individual if the expression level of IL8 in the individual's sample is below the reference level of IL8.

In another aspect, the invention features a method of identifying an individual having a cancer who is likely to respond to treatment with an anti-cancer therapy comprising a PD-L1 binding antagonist, the method comprising determining the expression level of IL8 in a sample obtained from the individual at a time point following administration of the anti-cancer therapy, wherein an expression level of IL8 in the sample that is below a reference level of IL8 identifies the individual as likely to respond to treatment with an anti-cancer therapy comprising a PD-L1 binding antagonist.

In another aspect, the invention features a method of monitoring the response of an individual having a cancer to treatment with an anti-cancer therapy comprising a PD-L1 binding antagonist, the method comprising: (a) determining the expression level of IL8 in a sample obtained from the individual at a time point following administration of the anti-cancer therapy; (b) comparing the expression level of IL8 in the sample with a reference level of IL8; and (c) continuing to administer the anti-cancer therapy to the individual if the expression level of IL8 in the individual's sample is below the reference level.

In another aspect, the invention features a method of treating an individual having a cancer, the method comprising: (a) administering an effective amount of an anti-cancer therapy comprising a PD-L1 binding antagonist to the individual; (b) determining the expression level of IL8 in a sample obtained from the individual at a time point following administration of the anti-cancer therapy comprising a PD-L1 binding antagonist; and (c) administering an anti-cancer therapy other than or in addition to a PD-L1 binding antagonist to the individual if the expression level of IL8 in the individual's sample is at or above the reference level of IL8.

In another aspect, the invention features a PD-L1 binding antagonist for use in treating an individual having a cancer, wherein the individual has been identified as likely to respond to treatment with an anti-cancer therapy comprising a PD-L1 binding antagonist based on an expression level of IL8 in a sample from the individual that is below a reference level of IL8, wherein the response is indicated by OS or ORR.

In another aspect, the invention features an anti-cancer therapy other than or in addition to a PD-L1 binding antagonist for use in treating an individual having a cancer, wherein the individual has been identified as less likely to respond to treatment with an anti-cancer therapy comprising a PD-L1 binding antagonist monotherapy based on an expression level of IL8 in a sample from the individual that is at or above a reference level of IL8, wherein the response is indicated by OS or ORR.

In another aspect, the invention features a PD-L1 binding antagonist for use in a method of treating an individual having a cancer, the method comprising: (a) administering an effective amount of an anti-cancer therapy comprising a PD-L1 binding antagonist to the individual; (b) determining the expression level of IL8 in a sample obtained from the individual at a time point following administration of the anti-cancer therapy; and (c) continuing to administer the anti-cancer therapy to the individual if the expression level of IL8 in the individual's sample is below the reference level of IL8.

In another aspect, the invention features an anti-cancer therapy other than or in addition to a PD-L1 binding antagonist for use in a method of treating an individual having a cancer, the method comprising: (a) administering an effective amount of an anti-cancer therapy comprising a PD-L1 binding antagonist to the individual; (b) determining the expression level of IL8 in a sample obtained from the individual at a time point following administration of the anti-cancer therapy comprising a PD-L1 binding antagonist; and (c) administering the anti-cancer therapy other than or in addition to a PD-L1 binding antagonist to the individual if the expression level of IL8 in the individual's sample is at or above the reference level of IL8.

In another aspect, the invention features a PD-L1 binding antagonist for use in treating an individual having a cancer, wherein the expression level of IL-8 in a sample obtained from the individual at a time point following administration of the PD-L1 binding antagonist has been determined to be below a reference level of IL8.

In another aspect, the invention features an anti-cancer therapy other than or in addition to a PD-L1 binding antagonist for use in treating an individual having a cancer, wherein the expression level of IL-8 in a sample obtained from the individual at a time point following administration of a PD-L1 binding antagonist has been determined to be at or above a reference level of IL8.

In another aspect, the invention features a PD-L1 axis binding antagonist for use in treating an individual having a cancer, wherein the individual has been identified as likely to respond to treatment with an anti-cancer therapy comprising a PD-L1 axis binding antagonist based on an expression level of IL8 in a sample from the individual that is below a reference level of IL8, wherein the sample is obtained from the individual at a time point prior to or concurrently with administration of the anti-cancer therapy comprising a PD-L1 axis binding antagonist, and wherein the response is indicated by OS or ORR.

In another aspect, the invention features an anti-cancer therapy other than or in addition to a PD-L1 axis binding antagonist for use in treating an individual having a cancer, wherein the individual has been identified as less likely to respond to treatment with an anti-cancer therapy comprising a PD-L1 axis binding antagonist monotherapy based on an expression level of IL8 in a sample from the individual that is at or above a reference level of IL8, wherein the sample is obtained from the individual at a time point prior to or concurrently with administration of the anti-cancer therapy comprising a PD-L1 axis binding antagonist, and wherein the response is indicated by OS or ORR.

In another aspect, the invention features a PD-L1 axis binding antagonist for use in a method of treating an individual having a cancer, the method comprising: (a) administering an effective amount of an anti-cancer therapy comprising a PD-L1 axis binding antagonist to the individual; (b) determining the expression level of IL8 in a sample obtained from the individual at a time point following administration of the anti-cancer therapy, wherein the sample is a plasma sample, a tumor tissue sample, or a sample comprising PBMCs; and (c) continuing to administer the anti-cancer therapy to the individual if the expression level of IL8 in the individual's sample is below the reference level of IL8.

In another aspect, the invention features a PD-L1 axis binding antagonist for use in treating an individual having a cancer, wherein the expression level of IL-8 in a sample obtained from the individual at a time point following administration of the PD-L1 axis binding antagonist has been determined to be below a reference level of IL8, wherein the sample is a plasma sample, a tumor tissue sample, or a sample comprising PBMCs.

In another aspect, the invention features an anti-cancer therapy other than or in addition to a PD-L1 axis binding antagonist for use in a method of treating an individual having a cancer, the method comprising: (a) administering an effective amount of an anti-cancer therapy comprising a PD-L1 axis binding antagonist to the individual; (b) determining the expression level of IL8 in a sample obtained from the individual at a time point following administration of an anti-cancer therapy comprising a PD-L1 axis binding antagonist, wherein the sample is a plasma sample, a tumor tissue sample, or a sample comprising PBMCs; and (c) administering the anti-cancer therapy other than or in addition to a PD-L1 axis binding antagonist to the individual if the expression level of IL8 in the individual's sample is at or above the reference level of IL8.

In another aspect, the invention features an anti-cancer therapy other than or in addition to a PD-L1 axis binding antagonist for use in treating an individual having a cancer, wherein the expression level of IL-8 in a sample obtained from the individual at a time point following administration of a PD-L1 axis binding antagonist has been determined to be at or above a reference level of IL8, wherein the sample is a plasma sample, a tumor tissue sample, or a sample comprising PBMCs.

In another aspect, the invention features a PD-L1 axis binding antagonist for use in treating an individual having a cancer, wherein the individual has been identified as likely to respond to treatment with an anti-cancer therapy comprising a PD-L1 axis binding antagonist based on an expression level of IL8 in a plasma sample or a sample comprising PBMCs from the individual that is below a reference level of IL8.

In another aspect, the invention features an anti-cancer therapy other than or in addition to a PD-L1 axis binding antagonist for use in treating an individual having a cancer, wherein the individual has been identified as less likely to respond to treatment with an anti-cancer therapy comprising a PD-L1 axis binding antagonist monotherapy based on an expression level of IL8 in a plasma sample or a sample comprising PBMCs from the individual that is at or above a reference level of IL8.

In another aspect, the invention features a method of identifying an individual having a cancer who is less likely to respond to treatment with an anti-cancer therapy comprising a PD-L1 binding antagonist monotherapy, the method comprising determining the expression level of IL8 in a sample obtained from the individual at a time point following administration of an anti-cancer therapy comprising a PD-L1 binding antagonist, wherein an expression level of IL8 in the sample that is at or above a reference level of IL8 identifies the individual as less likely to respond to treatment with an anti-cancer therapy comprising a PD-L1 binding antagonist monotherapy.

In another aspect, the invention features a method of monitoring the response of an individual having a cancer to treatment with an anti-cancer therapy comprising a PD-L1 binding antagonist, the method comprising: (a) determining the expression level of IL8 in a sample obtained from the individual at a time point following administration of an anti-cancer therapy comprising a PD-L1 binding antagonist; (b) comparing the expression level of IL8 in the sample with a reference level of IL8; and (c) administering an anti-cancer therapy other than or in addition to a PD-L1 binding antagonist to the individual if the expression level of IL8 in the individual's sample is at or above the reference level.

In another aspect, the invention features a method of treating an individual having a cancer, the method comprising: (a) administering an effective amount of an anti-cancer therapy comprising a PD-L1 axis binding antagonist to the individual; (b) determining the expression level of IL8 in a sample obtained from the individual at a time point following administration of the anti-cancer therapy, wherein the sample is a plasma sample, a tumor tissue sample, or a sample comprising peripheral blood mononuclear cells (PBMCs); and (c) continuing to administer the anti-cancer therapy to the individual if the expression level of IL8 in the individual's sample is below the reference level of IL8.

In another aspect, the invention features a method of identifying an individual having a cancer who is likely to respond to treatment with an anti-cancer therapy comprising a PD-L1 axis binding antagonist, the method comprising determining the expression level of IL8 in a sample obtained from the individual at a time point following administration of the anti-cancer therapy, wherein the sample is a plasma sample, a tumor tissue sample, or a sample comprising PBMCs, and wherein an expression level of IL8 in the sample that is below a reference level of IL8 identifies the individual as likely to respond to treatment with an anti-cancer therapy comprising a PD-L1 axis binding antagonist.

In another aspect, the invention features a method of monitoring the response of an individual having a cancer to treatment with an anti-cancer therapy comprising a PD-L1 axis binding antagonist, the method comprising: (a) determining the expression level of IL8 in a sample obtained from the individual at a time point following administration of the anti-cancer therapy, wherein the sample is a plasma sample, a tumor tissue sample, or a sample comprising PBMCs; (b) comparing the expression level of IL8 in the sample with a reference level of IL8; and (c) continuing to administer the anti-cancer therapy to the individual if the expression level of IL8 in the individual's sample is below the reference level.

In another aspect, the invention features a method of treating an individual having a cancer, the method comprising: (a) administering an effective amount of an anti-cancer therapy comprising a PD-L1 axis binding antagonist to the individual; (b) determining the expression level of IL8 in a sample obtained from the individual at a time point following administration of the anti-cancer therapy comprising a PD-L1 binding antagonist, wherein the sample is a plasma sample, a tumor tissue sample, or a sample comprising PBMCs; and (c) administering an anti-cancer therapy other than or in addition to a PD-L1 axis binding antagonist to the individual if the expression level of IL8 in the individual's sample is at or above the reference level of IL8.

In another aspect, the invention features a method of identifying an individual having a cancer who is less likely to respond to treatment with an anti-cancer therapy comprising a PD-L1 axis binding antagonist monotherapy, the method comprising determining the expression level of IL8 in a sample obtained from the individual at a time point following administration of an anti-cancer therapy comprising a PD-L1 axis binding antagonist, wherein the sample is a plasma sample, a tumor tissue sample, or a sample comprising PBMCs, and wherein an expression level of IL8 in the sample that is at or above a reference level of IL8 identifies the individual as less likely to respond to treatment with an anti-cancer therapy comprising a PD-L1 axis binding antagonist monotherapy.

In another aspect, the invention features a method of monitoring the response of an individual having a cancer to treatment with an anti-cancer therapy comprising a PD-L1 axis binding antagonist, the method comprising: (a) determining the expression level of IL8 in a sample obtained from the individual at a time point following administration of an anti-cancer therapy comprising a PD-L1 axis binding antagonist, wherein the sample is a plasma sample, a tumor tissue sample, or a sample comprising PBMCs; (b) comparing the expression level of IL8 in the sample with a reference level of IL8; and (c) administering an anti-cancer therapy other than or in addition to a PD-L1 axis binding antagonist to the individual if the expression level of IL8 in the individual's sample is at or above the reference level.

In some aspects, the time point following administration of the anti-cancer therapy is about 4 to about 8 weeks following administration of the anti-cancer therapy.

In some aspects, the time point following administration of the anti-cancer therapy is about 6 weeks following administration of the anti-cancer therapy.

In another aspect, the invention features a method of treating an individual having a cancer, the method comprising: (a) determining the expression level of IL8 in a sample from the individual that has been obtained at a time point prior to or concurrently with administration of an anti-cancer therapy comprising a PD-L1 axis binding antagonist, wherein an expression level of IL8 in the sample that is below a reference level of IL8 identifies the individual as likely to respond to treatment with an anti-cancer therapy comprising a PD-L1 binding antagonist, wherein the response is indicated by OS or ORR; and (b) administering an effective amount of an anti-cancer therapy comprising a PD-L1 axis binding antagonist to the individual based on the expression level of IL8 determined in step (a).

In another aspect, the invention features a method of treating an individual having a cancer who has been determined to be likely to respond to treatment with an anti-cancer therapy comprising a PD-L1 axis binding antagonist based on an expression level of IL8 in a sample from the individual that is below a reference level of IL8, the method comprising administering to the individual an effective amount of an anti-cancer therapy comprising a PD-L1 axis binding antagonist, wherein the response is indicated by OS or ORR, and wherein the sample has been obtained at a time point prior to or concurrently with administration of the anti-cancer therapy comprising a PD-L1 axis binding antagonist.

In another aspect, the invention features a method of identifying an individual having a cancer who is likely to respond to treatment with an anti-cancer therapy comprising a PD-L1 axis binding antagonist, the method comprising determining the expression level of IL8 in a sample from the individual that has been obtained at a time point prior to or concurrently with administration of an anti-cancer therapy comprising a PD-L1 axis binding antagonist, wherein an expression level of IL8 in the sample that is below a reference level of IL8 identifies the individual as likely to respond to treatment with an anti-cancer therapy comprising a PD-L1 axis binding antagonist, wherein the response is indicated by OS or ORR.

In another aspect, the invention features a method for selecting a therapy for an individual having a cancer, the method comprising: (a) determining the expression level of IL8 in a sample from the individual that has been obtained at a time point prior to or concurrently with administration of an anti-cancer therapy comprising a PD-L1 axis binding antagonist, wherein an expression level of IL8 in the sample that is below a reference level of IL8 identifies the individual as likely to respond to treatment with an anti-cancer therapy comprising a PD-L1 axis binding antagonist, wherein the response is indicated by OS or ORR; and (b) selecting an anti-cancer therapy comprising a PD-L1 axis binding antagonist for the individual based on the expression level of IL8 determined in step (a).

In another aspect, the invention features a method of treating an individual having a cancer, the method comprising: (a) determining the expression level of IL8 in a sample from the individual that has been obtained at a time point prior to or concurrently with administration of an anti-cancer therapy comprising a PD-L1 axis binding antagonist, wherein an expression level of IL8 in the sample that is at or above a reference level of IL8 identifies the individual as less likely to respond to treatment with an anti-cancer therapy comprising a PD-L1 axis binding antagonist monotherapy, wherein the response is indicated by OS or ORR; and (b) administering an effective amount of an anti-cancer therapy other than or in addition to a PD-L1 axis binding antagonist to the individual based on the expression level of IL8 determined in step (a).

In another aspect, the invention features a method of treating an individual having a cancer who has been determined to be less likely to respond to treatment with an anti-cancer therapy comprising a PD-L1 axis binding antagonist monotherapy based on an expression level of IL8 in a sample from the individual that is at or above a reference level of IL8, the method comprising administering to the individual an effective amount of an anti-cancer therapy other than or in addition to a PD-L1 axis binding antagonist, wherein the response is indicated by OS or ORR, and wherein the sample has been obtained at a time point prior to or concurrently with administration of an anti-cancer therapy comprising a PD-L1 axis binding antagonist.

In another aspect, the invention features a method of identifying an individual having a cancer who is less likely to respond to treatment with an anti-cancer therapy comprising a PD-L1 axis binding antagonist monotherapy, the method comprising determining the expression level of IL8 in a sample from the individual that has been obtained at a time point prior to or concurrently with administration of an anti-cancer therapy comprising a PD-L1 axis binding antagonist, wherein an expression level of IL8 in the sample that is at or above a reference level of IL8 identifies the individual as less likely to respond to treatment with an anti-cancer therapy comprising a PD-L1 axis binding antagonist monotherapy, and wherein the response is indicated by OS or ORR.

In another aspect, the invention features a method for selecting a therapy for an individual having a cancer, the method comprising: (a) determining the expression level of IL8 in a sample from the individual that has been obtained at a time point prior to or concurrently with administration of an anti-cancer therapy comprising a PD-L1 axis binding antagonist, wherein an expression level of IL8 in the sample that is at or above a reference level of IL8 identifies the individual as less likely to respond to treatment with an anti-cancer therapy comprising a PD-L1 axis binding antagonist monotherapy, wherein the response is indicated by OS or ORR; and (b) selecting an anti-cancer therapy other than or in addition to a PD-L1 axis binding antagonist for the individual based on the expression level of IL8 determined in step (a).

In some aspects, the sample is a plasma sample, a tissue sample, a cell sample, a whole blood sample, a serum sample, or a combination thereof.

In some aspects, the sample is a plasma sample.

In some aspects, the tissue sample is a tumor tissue sample.

In some aspects, the tumor tissue sample comprises tumor cells, tumor-infiltrating immune cells, stromal cells, or a combination thereof.

In some aspects, the tumor-infiltrating immune cells comprise tumor-infiltrating myeloid cells.

In some aspects, the tumor tissue sample is a formalin-fixed and paraffin-embedded (FFPE) sample, an archival sample, a fresh sample, or a frozen sample.

In some aspects, the cell sample comprises peripheral blood mononuclear cells (PBMCs).

In another aspect, the invention features a method of treating an individual having a cancer, the method comprising: (a) determining the expression level of IL8 in a plasma sample or a sample comprising PBMCs from the individual, wherein an expression level of IL8 in the plasma sample or in the sample comprising PBMCs that is below a reference level of IL8 identifies the individual as likely to respond to treatment with an anti-cancer therapy comprising a PD-L1 axis binding antagonist; and (b) administering an effective amount of an anti-cancer therapy comprising a PD-L1 axis binding antagonist to the individual based on the expression level of IL8 determined in the plasma sample or in the sample comprising PBMCs in step (a).

In another aspect, the invention features a method of treating an individual having a cancer who has been determined to be likely to respond to treatment with an anti-cancer therapy comprising a PD-L1 axis binding antagonist based on an expression level of IL8 in a plasma sample or a sample comprising PBMCs from the individual that is below a reference level of IL8, the method comprising administering to the individual an effective amount of an anti-cancer therapy comprising a PD-L1 axis binding antagonist.

In another aspect, the invention features a method of identifying an individual having a cancer who is likely to respond to treatment with an anti-cancer therapy comprising a PD-L1 axis binding antagonist, the method comprising determining the expression level of IL8 in a plasma sample or a sample comprising PBMCs from the individual, wherein an expression level of IL8 in the plasma sample or the sample comprising PBMCs that is below a reference level of IL8 identifies the individual as likely to respond to treatment with an anti-cancer therapy comprising a PD-L1 axis binding antagonist.

In another aspect, the invention features a method for selecting a therapy for an individual having a cancer, the method comprising: (a) determining the expression level of IL8 in a plasma sample or a sample comprising PBMCs from the individual, wherein an expression level of IL8 in the plasma sample or the sample comprising PBMCs that is below a reference level of IL8 identifies the individual as likely to respond to treatment with an anti-cancer therapy comprising a PD-L1 axis binding antagonist; and (b) selecting an anti-cancer therapy comprising a PD-L1 axis binding antagonist for the individual based on the expression level of IL8 in the plasma sample or in the sample comprising PBMCs determined in step (a).

In another aspect, the invention features a method of treating an individual having a cancer, the method comprising: (a) determining the expression level of IL8 in a plasma sample or a sample comprising PBMCs from the individual, wherein an expression level of IL8 in the plasma sample or in the sample comprising PBMCs that is at or above a reference level of IL8 identifies the individual as less likely to respond to treatment with an anti-cancer therapy comprising a PD-L1 axis binding antagonist monotherapy; and (b) administering an effective amount of an anti-cancer therapy other than or in addition to a PD-L1 axis binding antagonist to the individual based on the expression level of IL8 in the plasma sample or in the sample comprising PBMCs determined in step (a).

In another aspect, the invention features a method of treating an individual having a cancer who has been determined to be less likely to respond to treatment with an anti-cancer therapy comprising a PD-L1 axis binding antagonist monotherapy based on an expression level of IL8 in a plasma sample or a sample comprising PBMCs from the individual that is at or above a reference level of IL8, the method comprising administering to the individual an effective amount of an anti-cancer therapy other than or in addition to a PD-L1 axis binding antagonist.

In another aspect, the invention features a method of identifying an individual having a cancer who is less likely to respond to treatment with an anti-cancer therapy comprising a PD-L1 axis binding antagonist monotherapy, the method comprising determining the expression level of IL8 in a plasma sample or a sample comprising PBMCs from the individual, wherein an expression level of IL8 in the plasma sample or the sample comprising PBMCs that is at or above a reference level of IL8 identifies the individual as less likely to respond to treatment with an anti-cancer therapy comprising a PD-L1 axis binding antagonist monotherapy.

In another aspect, the invention features a method for selecting a therapy for an individual having a cancer, the method comprising: (a) determining the expression level of IL8 in a plasma sample or a sample comprising PBMCs from the individual, wherein an expression level of IL8 in the plasma sample or the sample comprising PBMCs that is at or above a reference level of IL8 identifies the individual as less likely to respond to treatment with an anti-cancer therapy comprising a PD-L1 axis binding antagonist monotherapy; and (b) selecting an anti-cancer therapy other than or in addition to a PD-L1 axis binding antagonist for the individual based on the expression level of IL8 in the plasma sample or in the sample comprising PBMCs determined in step (a).

In some aspects, the response is indicated by OS or ORR.

In some aspects, the response is indicated by OS.

In some aspects, the individual is likely to have an extended OS from treatment with the anti-cancer therapy comprising the PD-L1 binding antagonist compared to treatment with an anti-cancer therapy that does not comprise the PD-L1 binding antagonist.

In some aspects, the response is indicated by OS or ORR.

In some aspects, the response is indicated by OS.

In some aspects, the individual is likely to have an extended OS from treatment with the anti-cancer therapy comprising the PD-L1 axis binding antagonist compared to treatment with an anti-cancer therapy that does not comprise the PD-L1 axis binding antagonist.

In some aspects, the response is indicated by OS or ORR.

In some aspects, the response is indicated by OS.

In some aspects, the individual is likely to have a reduced OS from treatment with the anti-cancer therapy comprising the PD-L1 binding antagonist monotherapy compared to treatment with an anti-cancer therapy other than or in addition to a PD-L1 binding antagonist.

In some aspects, the response is indicated by OS or ORR.

In some aspects, the response is indicated by OS.

In some aspects, the individual is likely to have a reduced OS from treatment with the anti-cancer therapy comprising the PD-L1 axis binding antagonist monotherapy compared to treatment with an anti-cancer therapy other than or in addition to a PD-L1 axis binding antagonist.

In some aspects, the reference level of IL8 is determined from a population of individuals having a cancer.

In some aspects, the reference level of IL8 is a median expression level, a tertile expression level, or a maximally-selected log-rank reference level determined in a population of patients having a cancer.

In some aspects, the reference level of IL8 is a median expression level determined in a population of patients having a cancer.

In some aspects, the maximally-selected log-rank reference level is 12 pg/mL of IL8.

In some aspects, the reference level is the level of IL8 in a sample obtained from the individual at a time point prior to or concurrently with administration of the anti-cancer therapy comprising a PD-L1 binding antagonist.

In some aspects, the reference level is the level of IL8 in a sample obtained from the individual at a time point prior to or concurrently with administration of the anti-cancer therapy comprising a PD-L1 axis binding antagonist.

In some aspects, the expression level of IL8 is a nucleic acid expression level.

In some aspects, the nucleic acid expression level is an mRNA expression level.

In some aspects, the mRNA expression level is determined by RNA sequencing (RNA-seq), real-time-quantitative polymerase chain reaction (RT-qPCR), quantitative PCR (qPCR), multiplex qPCR or RT-qPCR, microarray analysis, serial analysis of gene expression (SAGE), MassARRAY technique, in situ hybridization (ISH), or a combination thereof.

In some aspects, the RNA-seq is single-cell RNA sequencing (scRNA-seq).

In some aspects, the expression level of IL8 is a protein expression level.

In some aspects, the protein expression level is determined by immunohistochemistry (IHC), Western blot, enzyme-linked immunosorbent assay (ELISA), immunoprecipitation, immunofluorescence, radioimmunoassay, or mass spectrometry.

In some aspects, the individual has an expression level of a T effector (T_{eff}) signature in a tumor sample that is at or above a reference level for the T_{eff} signature.

In some aspects, the T_{eff} signature comprises one or more genes selected from CD8A, GZMA, GZMB, and PRF1.

In some aspects, the T_{eff} signature comprises two or more genes selected from CD8A, GZMA, GZMB, and PRF1.

In some aspects, the T_{eff} signature comprises three or more genes selected from CD8A, GZMA, GZMB, and PRF1.

In some aspects, the T_{eff} signature comprises CD8A, GZMA, GZMB, and PRF1.

In some aspects, the individual has not been previously treated for the cancer.

In some aspects, the individual has previously been treated for the cancer.

In some aspects, the cancer is a bladder cancer, a kidney cancer, a breast cancer, a colorectal cancer, a lung cancer, a lymphoma, a prostate cancer, a liver cancer, a head and neck cancer, a melanoma, an ovarian cancer, a mesothelioma, or a myeloma.

In some aspects, the bladder cancer is urothelial carcinoma (UC).

In some aspects, the UC is locally advanced or metastatic UC.

In some aspects, the individual has received a prior platinum-based chemotherapy.

In some aspects, the individual has progressed after the prior platinum-based chemotherapy.

In some aspects, the individual is previously untreated for the locally advanced or metastatic UC.

In some aspects, the individual is ineligible for platinum-based chemotherapy.

In some aspects, the individual is cisplatin-ineligible.

In some aspects, the kidney cancer is renal cell carcinoma (RCC).

In some aspects, the RCC is metastatic RCC (mRCC).

In some aspects, the individual is previously untreated for the mRCC.

In some aspects, the lung cancer is non-small cell lung cancer or small-cell lung cancer.

In some aspects, the breast cancer is triple-negative breast cancer (TNBC) or HER2-positive breast cancer.

In some aspects, the breast cancer is TNBC.

In some aspects, the method further comprises administering an anti-cancer therapy comprising a PD-L1 binding antagonist to the individual.

In some aspects, the method further comprises administering an anti-cancer therapy comprising a PD-L1 axis binding antagonist to the individual.

In some aspects, the method further comprises administering an anti-cancer therapy other than or in addition to a PD-L1 binding antagonist to the individual.

In some aspects, the method further comprises administering an anti-cancer therapy other than or in addition to a PD-L1 axis binding antagonist to the individual.

In some aspects, the anti-cancer therapy other than or in addition to a PD-L1 binding antagonist comprises a VEGF antagonist, an IL8 antagonist, an IL1B antagonist, an IL1R antagonist, or a combination thereof.

In some aspects, the anti-cancer therapy other than or in addition to a PD-L1 axis binding antagonist comprises a VEGF antagonist, an IL1B antagonist, an IL1R antagonist, or a combination thereof.

In some aspects, the anti-cancer therapy comprises a VEGF antagonist and a PD-L1 binding antagonist.

In some aspects, the VEGF antagonist is an anti-VEGF antibody or a VEGF receptor (VEGFR) inhibitor.

In some aspects, the VEGF antagonist is an anti-VEGF antibody.

In some aspects, the anti-VEGF antibody is bevacizumab.

In some aspects, the IL8 antagonist is an anti-IL8 antibody or a small molecule IL8 inhibitor.

In some aspects, the IL1B antagonist is an anti-IL1B antibody or a small molecule IL1B inhibitor.

In some aspects, the IL1R antagonist is an anti-IL1R antibody or a small molecule IL1R inhibitor.

In some aspects, the PD-L1 axis binding antagonist is selected from the group consisting of a PD-L1 binding antagonist, a PD-1 binding antagonist, and a PD-L2 binding antagonist.

In some aspects, the PD-L1 axis binding antagonist is a PD-L1 binding antagonist.

In some aspects, the PD-L1 binding antagonist inhibits the binding of PD-L1 to one or more of its ligand binding partners.

In some aspects, the PD-L1 binding antagonist inhibits the binding of PD-L1 to PD-1.

In some aspects, the PD-L1 binding antagonist inhibits the binding of PD-L1 to B7-1.

In some aspects, the PD-L1 binding antagonist inhibits the binding of PD-L1 to both PD-1 and B-1.

In some aspects, the PD-L1 binding antagonist is an anti-PD-L1 antibody.

In some aspects, the anti-PD-L1 antibody is selected from the group consisting of: atezolizumab, MDX-1105, durvalumab, and avelumab.

In some aspects, the anti-PD-L1 antibody comprises the following hypervariable regions (HVRs): (a) an HVR-H1 sequence of GFTFSDSWIH (SEQ ID NO: 19); (b) an HVR-H2 sequence of AWISPYGGSTYYADSVKG (SEQ ID NO: 20); (c) an HVR-H3 sequence of RHWPGGFDY (SEQ ID NO: 21); (d) an HVR-L1 sequence of RASQDVSTAVA (SEQ ID NO: 22); (e) an HVR-L2 sequence of SASFLYS (SEQ ID NO: 23); and (f) an HVR-L3 sequence of QQYLYHPAT (SEQ ID NO: 24).

In some aspects, the anti-PD-L1 antibody comprises: (a) a heavy chain variable (VH) domain comprising an amino acid sequence having at least 90% sequence identity to the amino acid sequence of EVQLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSS (SEQ ID NO: 25); (b) a light chain variable (VL) domain comprising an amino acid sequence having at least 90% sequence identity to the amino acid sequence of DIQMTQSPSSLSASVGDRVTITCRASQDVSTAVAWYQQKPGKAPKLLIYSASFLYSGVPSRFSGSGSGTD FTLTISSLQPEDFATYYCQQYLYHPATFGQGTKVEIKR (SEQ ID NO: 4); or (c) a VH domain as in (a) and a VL domain as in (b).

In some aspects, the anti-PD-L1 antibody comprises: (a) a VH domain comprising an amino acid sequence having at least 95% sequence identity to the amino acid sequence of SEQ ID NO: 25; (b) a VL domain comprising an amino acid sequence having at least 95% sequence identity to the amino acid sequence of SEQ ID NO: 4; or (c) a VH domain as in (a) and a VL domain as in (b).

In some aspects, the anti-PD-L1 antibody comprises: (a) a VH domain comprising an amino acid sequence having at least 96% sequence identity to the amino acid sequence of SEQ ID NO: 25; (b) a VL domain comprising an amino acid sequence having at least 96% sequence identity to the amino acid sequence of SEQ ID NO: 4; or (c) a VH domain as in (a) and a VL domain as in (b).

In some aspects, the anti-PD-L1 antibody comprises: (a) a VH domain comprising an amino acid sequence having at least 97% sequence identity to the amino acid sequence of SEQ ID NO: 25; (b) a VL domain comprising an amino acid sequence having at least 97% sequence identity to the amino acid sequence of SEQ ID NO: 4; or (c) a VH domain as in (a) and a VL domain as in (b).

In some aspects, the anti-PD-L1 antibody comprises: (a) a VH domain comprising an amino acid sequence having at least 98% sequence identity to the amino acid sequence of SEQ ID NO: 25; (b) a VL domain comprising an amino acid sequence having at least 98% sequence identity to the amino acid sequence of SEQ ID NO: 4; or (c) a VH domain as in (a) and a VL domain as in (b).

In some aspects, the anti-PD-L1 antibody comprises: (a) a VH domain comprising an amino acid sequence having at least 99% sequence identity to the amino acid sequence of SEQ ID NO: 25; (b) a VL domain comprising an amino acid sequence having at least 99% sequence identity to the amino acid sequence of SEQ ID NO: 4; or (c) a VH domain as in (a) and a VL domain as in (b).

In some aspects, the anti-PD-L1 antibody comprises: (a) a VH domain comprising the amino acid sequence of SEQ ID NO: 25; (b) a VL domain comprising the amino acid sequence of SEQ ID NO: 4; or (c) a VH domain as in (a) and a VL domain as in (b).

In some aspects, the anti-PD-L1 antibody comprises: (a) a VH domain comprising the amino acid sequence of SEQ ID NO: 25; and (b) a VL domain comprising the amino acid sequence of SEQ ID NO: 4.

In some aspects, the anti-PD-L1 antibody is atezolizumab.

In some aspects, the method further comprises administering an additional therapeutic agent to the individual.

In some aspects, the additional therapeutic agent is selected from the group consisting of an immunotherapy agent, a cytotoxic agent, a growth inhibitory agent, a radiation therapy agent, an anti-angiogenic agent, and combinations thereof.

In some aspects, the individual is a human.

In another aspect, the invention features a kit for identifying an individual having a cancer who may benefit from treatment with an anti-cancer therapy comprising a PD-L1 binding antagonist, the kit comprising: (a) reagents for determining the expression level of IL8; and, optionally, (b) instructions for using the reagents to identify an individual having a cancer who is likely to respond to treatment with an anti-cancer therapy comprising a PD-L1 binding antagonist in accordance with any one of the methods described herein.

In another aspect, the invention features a kit for identifying an individual having a cancer who may benefit from treatment with an anti-cancer therapy other than or in addition to a PD-L1 binding antagonist, the kit comprising: (a) reagents for determining the expression level of IL8; and, optionally, (b) instructions for using the reagents to identify an individual having a cancer who is less likely to respond to treatment with an anti-cancer therapy comprising a PD-L1 binding antagonist monotherapy in accordance with any one of the methods described herein, thereby identifying the individual as one who may benefit from treatment with an anti-cancer therapy other than or in addition to a PD-L1 binding antagonist.

In another aspect, the invention features a kit for treating an individual having a cancer, the kit comprising: (a) a PD-L1 binding antagonist; and, optionally, (b) instructions to administer the PD-L1 binding antagonist to an individual having a cancer who has been identified as likely to respond to an anti-cancer therapy comprising a PD-L1 binding antagonist in accordance with any one of the methods described herein.

In another aspect, the invention features a kit for treating an individual having a cancer, the kit comprising: (a) an anti-cancer therapy other than or in addition to a PD-L1 binding antagonist; and, optionally, (b) instructions to administer the anti-cancer therapy other than or in addition to a PD-L1 binding antagonist to an individual having a cancer who has been identified as less likely to respond to an anti-cancer therapy comprising a PD-L1 binding antagonist monotherapy in accordance with any one of the methods described herein.

In another aspect, the invention features a kit for identifying an individual having a cancer who may benefit from treatment with an anti-cancer therapy comprising a PD-L1 axis binding antagonist, the kit comprising: (a) reagents for determining the expression level of IL8; and, optionally, (b) instructions for using the reagents to identify an individual having a cancer who is likely to respond to treatment with an anti-cancer therapy comprising a PD-L1 axis binding antagonist in accordance with any one of the methods described herein.

In another aspect, the invention features a kit for identifying an individual having a cancer who may benefit from treatment with an anti-cancer therapy other than or in addition to a PD-L1 axis binding antagonist, the kit comprising: (a) reagents for determining the expression level of IL8; and, optionally, (b) instructions for using the reagents to identify an individual having a cancer who is less likely to respond to treatment with an anti-cancer therapy comprising a PD-L1 axis binding antagonist monotherapy in accordance with any one of the methods described herein, thereby identifying the individual as one who may benefit from treatment with an anti-cancer therapy other than or in addition to a PD-L1 axis binding antagonist.

In another aspect, the invention features a kit for treating an individual having a cancer, the kit comprising: (a) a PD-L1 axis binding antagonist; and, optionally, (b) instructions to administer the PD-L1 axis binding antagonist to an individual having a cancer who has been identified as likely to respond to an anti-cancer therapy comprising a PD-L1 axis binding antagonist in accordance with any one of the methods described herein.

In another aspect, the invention features a kit for treating an individual having a cancer, the kit comprising: (a) an anti-cancer therapy other than or in addition to a PD-L1 axis binding antagonist; and, optionally, (b) instructions to administer the anti-cancer therapy other than or in addition to a PD-L1 axis binding antagonist to an individual having a cancer who has been identified as less likely to respond to an anti-cancer therapy comprising a PD-L1 axis binding antagonist monotherapy in accordance with any one of the methods described herein.

### BRIEF DESCRIPTION OF THE DRAWINGS

FIG. 1 is a schematic diagram summarizing the clinical trials in which the association of IL8 expression with clinical outcome was evaluated. Atezo, atezolizumab; BEP, biomarker-evaluable population.

FIG. 2 is a table showing demographics and baseline characteristics of biomarker-evaluable patients as well as the IL8 low and IL8 high subgroups in the IMvigor210 (including cohorts 1 and 2) and IMvigor211 (including atezolizumab- and chemotherapy-treated patients) clinical trials.

FIG. 3 is a table showing demographics and baseline characteristics of biomarker-evaluable patients as well as the IL8 low and IL8 high subgroups in the IMmotion150 clinical trial.

FIGS. 4A-4E are a series of graphs showing that high plasma IL8 is associated with poor outcome in metastatic UC (mUC) and metastatic RCC (mRCC). In FIG. 4A, high baseline plasma IL8 (pIL8) levels (median cutoff: 15 pg/mL) were significantly associated with worse overall survival (OS) in cohort 2 of IMvigor210 (HR=1.90, 95% CI: 1.31, 2.74, P<0.0001). In FIG. 4B, high baseline IL8 plasma levels were significantly associated with higher number of non-responders (SD and PD) (P=0.01, two-sided Fisher's exact test) by Response Evaluation Criteria in Solid Tumors (RECIST) 2.1. CR, complete response; PR, partial response; SD, stable disease; PD, progressive disease. In FIG. 4C, high baseline plasma IL8 was associated with poor OS even in tumors with T_{eff} signature expression in mUC patients in cohort 2 of IMvigor210. Elevated plasma IL8 expression was associated with worse OS compared to low plasma IL8 expression, even in T_{eff} high tumors (HR: 1.71; 95% CI: 1.12, 3.26, P=0.017). In FIG. 4D, in a randomized mUC Phase 3 trial (IMvigor211), high baseline plasma IL8 levels were significantly associated with worse OS in both the atezolizumab (HR: 1.83; 95% CI: 1.48, 2.26, P<0.0001) and chemotherapy (HR: 1.67; 95% CI: 1.38, 2.03, P<0.0001) arms. However, low baseline plasma IL8 significantly improved OS in the atezolizumab arm compared to the chemotherapy arm (HR: 0.76; 95% CI: 0.61, 0.94, P=0.01). FIG. 4E shows high baseline plasma IL8 was significantly associated with worse progression free survival (PFS) in the atezolizumab (HR 1.98; 95% CI: 1.12, 3.5, P=0.018) and the atezolizumab+bevacizumab (HR 1.89, 95% CI: 1.08, 3.3, P=0.025) arms, but not in the sunitinib arm (HR 1.29, 95% CI: 0.73, 2.3, P=0.377) in a randomized mRCC Phase 2 trial (IMmotion150).

FIGS. 5A and 5B are a series of graphs showing an association of high plasma IL8 expression with worse OS (FIG. 5A) and ORR (FIG. 5B) in mUC patients in IMvigor 210 cohort 1.

FIGS. 6A and 6B are a series of graphs showing that plasma IL8 expression had a moderate correlation with the neutrophil to lymphoid ratio (NLR) in the IMvigor210 (FIG. 6A) and IMmotion150 (FIG. 6B) clinical trials.

FIGS. 7A and 7B are a series of graphs showing that plasma IL8 levels did not correlate with markers of high tumoral immune presence such as tumor T-effector (T_{eff}) gene expression (FIG. 7A) or tumor mutation burden (FIG. 7B).

FIGS. 8A and 8B are a series of graphs showing that an on-treatment increase in plasma IL8 expression was associated with worse OS in mUC in the atezolizumab arm. In FIG. 8A, on-treatment changes of plasma IL8 are referred to as ratios of plasma IL8 levels between on-treatment cycle 3 day 1 (C3D1) and baseline cycle 1 day 1 (C1D1). A high on-treatment increase of plasma IL8 levels (median cutoff: 1.09 pg/mL) was significantly associated with worse OS in cohort 2 of IMvigor210 (HR: 0.48; 95% CI: 0.31, 0.76, P<0.01). As shown in FIG. 8B, a similar trend was observed in the IMvigor211 clinical trial in which a high on-treatment increase of plasma IL8 levels was significantly associated with worse OS only in the atezolizumab arm (HR: 0.49, 95% CI: 0.36; 0.66, P<0.001), but not in the chemotherapy arm (HR: 0.83, 95% CI: 0.63; 1.1, P=0.19).

FIGS. 9A-9E are a series of graphs showing that an on-treatment increase in plasma IL8 expression was associated with poor outcome in terms of ORR and OS in mUC. FIGS. 9A and 9B show that a high on-treatment increase of plasma IL8 levels was associated with worse ORR in cohort 1 and cohort 2, respectively, of IMvigor210. FIG. 9C shows that a high on-treatment increase of plasma IL8 levels was associated with worse ORR in the atezolizumab arm but not the chemotherapy arm in IMvigor211. FIG. 9D shows that a high on-treatment increase of plasma IL8 levels was significantly associated with worse OS in cohort 1 of IMvigor210. FIG. 9E shows that there was no significant difference in absolute lymphoid and myeloid counts in patients treated with atezolizumab, whereas there was an increase in absolute lymphoid and myeloid counts in patients treated with chemotherapy in IMvigor211. The poor response associated with chemotherapy in low plasma IL8 was therefore not due to a reduction of lymphoid and myeloid cells.

FIGS. 10A-10H are a series of graphs showing that poor clinical outcome associated with high IL8 gene expression in subsets of myeloid cells was correlated with downregulation of genes including antigen presentation genes. In FIG. 10A, subsets of distinct myeloid and lymphoid clusters were revealed by single-cell RNA-seq (scRNA-seq) of peripheral blood mononuclear cells (PBMCs) isolated from five responders and five non-responders in the mUC IMvigor210 study. The clusters were identified by the expression of lymphocyte (upper panel) and myeloid (lower panel) cell-specific markers shown in FIG. 10B. FIG. 10C shows high IL8 gene expression for subsets of myeloid clusters. In FIG. 10D, different gene expression analysis between IL8 high and low myeloid cells showed a significant enrichment of myeloid inflammatory response genes (light grey) in IL8 high myeloid cells whereas a significant enrichment of antigen presentation machinery genes (dark grey) was observed in IL8 low myeloid cells.

FIG. 10E shows an increase in IL8 gene expression in myeloid clusters of non-responders compared to responders, in which subsets of myeloid clusters had significantly higher IL8 expression in non-responders, as shown in FIG. 10F (***false discovery rate (FDR)<0.001; **FDR<0.01). In FIG. 10G, high IL8 gene expression in PBMCs was significantly associated with worse OS in the mUC IMvigor210 trial (HR: 1.36, 95% CI: 1.07, 1.73, P=0.013). In FIG. 10H, high IL8 gene expression in PBMCs was significantly associated with worse PFS only in the atezolizumab arm (HR 2.52; 95% CI: 1.29, 4.9, P=0.007) of the RCC IMmotion150 trial but not in the atezolizumab+bevacizumab (HR 1.11; 95% CI: 0.64, 1.9, P=0.708) or sunitinib (HR 0.81; 95% CI: 0.47, 1.4, P=0.436) arms.

FIGS. 11A-11F are a series of graphs showing results from scRNA-seq of PBMCs from five responders and five non-responders from the IMvigor210 study. FIG. 11A shows visualization of scRNA-seq showing the distribution of cells (each dot represents one cell) between responders and non-responders as assessed by Uniform Manifold Approximation and Projection. FIG. 11B shows visualization of scRNA-seq showing the distribution of cells (each dot represents one cell) for the indicated groups as assessed by UMAP. R1-5 represent Responders 1-5 and NR1-5 represent Non-Responders 1-5. FIG. 11C shows the number of RNA transcripts detected in each cell (color-coded based on a log 10 scale). FIG. 11D shows the fraction of cells belonging to the indicated clusters for responders and non-responders. FIG. 11E shows the fraction of cells belonging to the indicated groups for the indicated clusters. FIG. 11F shows the number of cells for the indicated clusters.

FIG. 12 is a graph showing gene set enrichment pathways analysis between IL8 high versus IL8 low cells (median cutoff) in all myeloid clusters.

FIGS. 13A and 13B are a series of graphs showing differential gene expression analysis between responders and non-responders in monocyte clusters. There is a significant enrichment of myeloid inflammatory response genes in non-responders whereas a significant enrichment of antigen presentation machinery genes, such as human leukocyte antigen (HLA) genes and interferon gamma (IFNg)-induced genes in responders.

FIGS. 14A-14H are a series of graphs showing evaluation of IL8 gene expression in immune subsets from tumor and blood by scRNA-seq analysis in RCC and association of IL8 gene expression with clinical outcome in RCC and mUC. In FIG. 14A, scRNA-seq identified immune subsets in leukocytes in matched blood and tumor. In FIG. 14B, expression of select lymphoid and myeloid associated genes in immune subsets from tumor and blood from RCC is shown. FIG. 14C shows a heat map reporting scaled expression (log expression count values) of selected gene sets. The gene expression shading scheme is based on scale log count expression distribution, from −2.0 to 2.0. Color bars in the right margin highlight exemplary cell subsets of interest. FIG. 14D presents a differential gene expression analysis between IL8 high and IL8 low tumor myeloid cells and shows a significant enrichment of myeloid inflammatory response genes (dark grey) in IL8 high tumor myeloid cells whereas a significant enrichment of antigen presentation machinery genes (light grey) was observed in IL8 low myeloid cells in tumor samples. FIG. 14E shows that high tumor IL8 gene expression was associated with reduced OS (HR: 3.97, 95% CI: 1.83, 8.6, P<0.001) compared to low tumor IL8 gene expression in the atezolizumab monotherapy arm, but not in the atezolizumab+bevacizumab arm (HR: 1.78, 95% CI: 0.92, 3.4, P=0.088) and sunitinib arm (HR: 1.75, 95% CI: 0.83, 3.7, P=0.144). FIG. 14F shows that high tumor IL8 gene expression was associated with worse OS compared to low tumor IL8 gene expression in the atezolizumab monotherapy arm in the IMvigor210 trial (HR: 1.34, 95% CI: 1.03, 1.74, P=0.026). FIG. 14G shows that high tumor IL8 gene expression was associated with reduced OS in T-effector (T_{eff}) high patients (HR: 15.6, 95% CI: 3.15, 77.6, P<0.001) compared to low tumor IL8 expression in the atezolizumab monotherapy arm, but not in the atezolizumab+bevacizumab arm (HR: 0.96, 95% CI: 0.29, 3.2, P=0.945), and sunitinib arm (HR: 1.94, 95% CI: 0.67, 5.6, P=0.225). FIG. 14H shows that high tumor IL8 gene expression was associated with worse OS in T_{eff} high patients in the atezolizumab monotherapy-treated patients in IMvigor210 (HR: 1.67, 95% CI: 1.12, 249, P=0.012).

FIG. 15 is a schematic diagram outlining an exemplary RCC scRNA data analysis workflow using Seurat. Briefly, Seurat (version 3.0) was used to perform basic quality control on the raw 50 gene expression (GEX) matrices output from Cell Ranger 2.2.1, followed by sample de-multiplexing, alignment, filtering, and UMI (universal molecular identifier) counting. RCC blood and tumor single cell data was integrated together for normalization. The first 20 principal components were used for clustering (resolution=0.6) and for UMAP visualization. Clusters were identified based on genes that are enriched in a specific cluster.

FIGS. 16A and 16B are a series of graphs showing results from UMAP visualization of lymphoid and myeloid cells. FIG. 16A shows the fraction of cells from blood or tumor (see FIG. 15) plotted for the indicated clusters. FIG. 16B shows the proportion of cells for the indicated blood or tumor groups in the indicated clusters.

FIG. 17 is a graph showing scaled average gene expression of cell type specific markers in single cell RNAseq of mRCC tumor. Tcm, central memory T cell; Tem, effector memory; M1-like, M1-like macrophages; M2-like, M2-like macrophages.

FIG. 18 is a graph showing differential gene expression analysis of IL8 high versus IL8 low intratumoral myeloid cells (cluster 7) revealing higher expression of inflammatory genes (e.g., IL1B, PTGS2, IL1RN, andNLRP3) and, among other genes, reduced expression of genes involved in antigen processing and presentation (e.g., HLA-DR5, HLA-DRB6, HLA-C, and B2M) in IL8 high cells.

FIG. 19 is a graph showing gene set enrichment pathways analysis between IL8-high versus IL8-low single cells in peripheral blood leukocytes in mRCC. Pathways that are enriched in IL8-low cells are shown in dark grey (negative enrichment score) whereas pathways that are enriched in IL8-high are shown in light grey (positive enrichment score).

FIG. 20 is a series of graphs showing that elevated IL8 gene expression was correlated with higher neutrophil presence by histological assessment.

### DETAILED DESCRIPTION OF THE INVENTION

### I. Introduction

The present invention provides therapeutic and diagnostic methods and compositions for cancer, for example, bladder cancer (e.g., UC) and kidney cancer (e.g., RCC). The invention is based, at least in part, on the discovery that determination of expression levels of biomarkers of the invention, for example, any biomarker set forth in any one of Tables 1-7, e.g., IL8, in samples obtained from an individual is useful in treatment of an individual having cancer, for diagnosing an individual having cancer, for determining whether an individual having is likely to respond to treatment with an anti-cancer therapy that includes a PD-L1 axis binding antagonist (e.g., an anti-PD-L1 antibody, e.g., atezolizumab), for optimizing therapeutic efficacy of an anti-cancer therapy that includes a PD-L1 axis binding antagonist (e.g., an anti-PD-L1 antibody, e.g., atezolizumab), and/or for patient selection for an anti-cancer therapy comprising a PD-L1 axis binding antagonist (e.g., an anti-PD-L1 antibody, e.g., atezolizumab).

For example, the data provided herein demonstrate that peripheral and intratumoral IL8 and/or associated myeloid inflammation confers resistance to checkpoint blockade. Further, expression of IL8 can be used as a predictive biomarker for treatment response to checkpoint inhibitor therapy, including with an anti-cancer therapy that includes a PD-L1 axis binding antagonist (e.g., an anti-PD-L1 antibody such as atezolizumab). In particular, individuals with low IL8 expression, or on-treatment decreases in IL8 expression, are likely to respond to treatment with an anti-cancer therapy that includes a PD-L1 axis binding antagonist. In contrast, patients with high IL8 expression, or on-treatment increases in IL8 expression, may benefit from treatment with an anti-cancer therapy other than or in addition to a PD-L1 axis binding antagonist (e.g., a combination therapy that includes a PD-L1 axis binding antagonist and one or more additional anti-cancer therapeutic agents, or an anti-cancer therapy that does not include a PD-L1 axis binding antagonist). The data provided herein also provide genes that are upregulated or downregulated in the context of IL8 high cancer, which can be used as proxies for IL8 expression. See, e.g., Tables 2-4 (upregulated genes) and Tables 5-7 (downregulated genes).

### II. Definitions

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.”

The terms “interleukin 8” and “IL8,” as used interchangeably herein, refer to any native IL8 (also known as CXCL8 (chemokine (C-X-C motif) ligand 8)) from any vertebrate source, including mammals such as primates (e.g., humans) and rodents (e.g., mice and rats), unless otherwise indicated. The term encompasses “full-length,” unprocessed IL8 as well as any form of IL8 that results from processing in the cell. The term also encompasses naturally occurring variants of IL8, e.g., splice variants or allelic variants. The nucleic acid sequence of an exemplary human IL8 is shown under NCBI Entrez Gene ID No. 3576. The amino acid sequence of an exemplary protein encoded by human IL8 is shown under UniProt accession number P10145.

The term “CD8A” as used herein, refers to any native CD8A from any vertebrate source, including mammals such as primates (e.g., humans) and rodents (e.g., mice and rats), unless otherwise indicated. The term encompasses “full-length,” unprocessed CD8A as well as any form of CD8A that results from processing in the cell. The term also encompasses naturally occurring variants of CD8A, e.g., splice variants or allelic variants. The nucleic acid sequence of an exemplary human CD8A is set forth in UniProt Accession No. M12824. The amino acid sequence of an exemplary protein encoded by human CD8A is set forth in UniProt Accession No. P01732-1.

The term “GZMA” as used herein, refers to any native GZMA (Granzyme A) from any vertebrate source, including mammals such as primates (e.g., humans) and rodents (e.g., mice and rats), unless otherwise indicated. The term encompasses “full-length,” unprocessed GZMA as well as any form of GZMA that results from processing in the cell. The term also encompasses naturally occurring variants of GZMA, e.g., splice variants or allelic variants. The nucleic acid sequence of an exemplary human GZMA is set forth in UniProt Accession No. M18737. The amino acid sequence of an exemplary protein encoded by human GZMA is set forth in UniProt Accession No. P12544-1.

The term “GZMB” as used herein, refers to any native GZMB (Granzyme B) from any vertebrate source, including mammals such as primates (e.g., humans) and rodents (e.g., mice and rats), unless otherwise indicated. The term encompasses “full-length,” unprocessed GZMB as well as any form of GZMB that results from processing in the cell. The term also encompasses naturally occurring variants of GZMB, e.g., splice variants or allelic variants. The nucleic acid sequence of an exemplary human GZMB is set forth in UniProt Accession No. M17016. The amino acid sequence of an exemplary protein encoded by human GZMB is set forth in UniProt Accession No. P10144-1.

The term “PRF1” as used herein, refers to any native PRF1 (Perforin 1; also known as Pore Forming Protein) from any vertebrate source, including mammals such as primates (e.g., humans) and rodents (e.g., mice and rats), unless otherwise indicated. The term encompasses “full-length,” unprocessed PRF1 as well as any form of PRF1 that results from processing in the cell. The term also encompasses naturally occurring variants of PRF1, e.g., splice variants or allelic variants. Exemplary nucleic acid sequences of human PRF1 are set forth in UniProt Accession Nos. X13224 and X12940. The amino acid sequence of an exemplary protein encoded by human PRF1 is set forth in UniProt Accession No. P14222-1.

The term “detection” includes any means of detecting, including direct and indirect detection.

The term “biomarker” as used herein refers to an indicator, e.g., predictive, diagnostic, and/or prognostic, which can be detected in a sample, for example, any gene set forth in any one of Tables 1-7, e.g., IL8. The biomarker may serve as an indicator of the likelihood of an individual to respond to or benefit from a therapy (e.g., an anti-cancer therapy that includes a PD-L1 axis binding antagonist) and/or as an indicator of a particular subtype of a disease or disorder (e.g., cancer (e.g., bladder cancer (e.g., UC) and kidney cancer (e.g., RCC))) characterized by certain, molecular, pathological, histological, and/or clinical features. In some embodiments, a biomarker is a gene. Biomarkers include, but are not limited to, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The cell sample may include, for example, PBMCs. The tissue sample may also be primary or cultured cells or cell lines. Optionally, the tissue or cell sample is obtained from a disease tissue/organ. For instance, a “tumor sample” is a tissue sample obtained from a tumor or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like.

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, T lymphocytes (such as CD8+ T lymphocytes and/or CD4+ T lymphocytes), B lymphocytes, or other bone marrow-lineage cells, including granulocytes (e.g., neutrophils, eosinophils, and basophils), monocytes, macrophages, dendritic cells (e.g., interdigitating dendritic cells), histiocytes, and natural killer cells.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

For the purposes herein a “section” of a tissue sample is meant a single part or piece of a tissue sample, for example, a thin slice of tissue or cells cut from a tissue sample (e.g., a tumor sample). It is to be understood that multiple sections of tissue samples may be taken and subjected to analysis, provided that it is understood that the same section of tissue sample may be analyzed at both morphological and molecular levels, or analyzed with respect to polypeptides (e.g., by immunohistochemistry) and/or polynucleotides (e.g., by in situ hybridization).

The “amount” or “level” of a biomarker associated with an increased clinical benefit to an individual is a detectable level in a biological sample. These can be measured by methods known to one skilled in the art and also disclosed herein. The expression level or amount of biomarker assessed can be used to determine the response to the treatment.

The terms “level of expression” or “expression level” in general are used interchangeably and generally refer to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

As used herein, the terms “reference expression level” and “reference level” refer interchangeably to an expression level against which another expression level, e.g., the expression level of one or more genes described herein (e.g., one or more genes set forth in any one of Tables 1-7, e.g., IL8) in a sample from an individual is compared, e.g., to make a predictive, diagnostic, prognostic, and/or therapeutic determination. For example, the reference expression level may be derived from expression levels in a reference population (e.g., the median expression level, the tertile expression level, or a maximally-selected log-rank reference level in a reference population, e.g., a population of patients having a cancer), a reference sample, and/or a pre-assigned value (e.g., a cut-off value which was previously determined to significantly (e.g., statistically significantly) separate a first subset of individuals who have been treated with an anti-cancer therapy (e.g., an anti-cancer therapy including a PD-L1 axis binding antagonist (e.g., a PD-L1 binding antagonist (e.g., anti-PD-L1 antibody, e.g., atezolizumab) or a PD-1 binding antagonist (e.g., anti-PD-1 antibody))) in a reference population and a second subset of individuals who have been treated with a different anti-cancer therapy (or who have not been treated with the anti-cancer therapy) in the same reference population based on a significant difference between an individual's responsiveness to treatment with the anti-cancer therapy and an individual's responsiveness to treatment with the different anti-cancer therapy above the cut-off value and/or below the cut-off value). In some embodiments, the maximally-selected log-rank reference level is 12 pg/mL of IL8. In some instances, a reference level may be a reference level in a sample obtained from the individual at a time point prior to, concurrently with, or following administration of a therapy (e.g., an anti-cancer therapy that includes a PD-L1 axis binding antagonist) to the individual. In some embodiments, the cut-off value may be the median or mean expression level in the reference population. In other embodiments, the reference level may be the top 40%, the top 30%, the top 20%, the top 10%, the top 5%, or the top 1% of the expression level in the reference population. In particular embodiments, the cut-off value may be the median expression level in the reference population. It will be appreciated by one skilled in the art that the numerical value for the reference expression level may vary depending on the indication (e.g., a cancer (e.g., bladder cancer (e.g., UC) and kidney cancer (e.g., RCC)), the methodology used to detect expression levels (e.g., RNA-seq (e.g., scRNA-seq) or RT-qPCR), and/or the specific combinations of genes examined.

Expression “above” a level (e.g., above a reference level), “increased expression,” “increased expression level,” “increased levels,” “elevated expression,” “elevated expression levels,” or “elevated levels” refers to an increased expression or increased levels of a biomarker (e.g., one or more genes set forth in any one of Tables 1-7, e.g., IL8) in an individual relative to the expression level of the biomarker in a control (e.g., an individual or individuals who are not suffering from the disease or disorder (e.g., cancer), an internal control (e.g., a housekeeping biomarker), or the level of a biomarker in a sample obtained prior to administration of a therapy (e.g., an anti-cancer therapy that includes a PD-L1 axis binding antagonist)), or relative to a reference level (e.g., the median expression level of the biomarker in samples from a group/population of patients, e.g., patients having cancer who are being tested for responsiveness to a PD-L1 axis binding antagonist; the median expression level of the biomarker in samples from a group/population of patients, e.g., patients having cancer who have been identified as not responding to a PD-L1 axis binding antagonist; or the level in a sample previously obtained from the individual at a prior time).

Expression “below” a level (e.g., below a reference level), “decreased expression,” “decreased expression level,” “decreased levels,” “reduced expression,” “reduced expression levels,” or “reduced levels” refers to a decrease expression or decreased levels of a biomarker (e.g., one or more genes set forth in any one of Tables 1-7, e.g., IL8) in an individual relative to the expression level of the biomarker in a control (e.g., an individual or individuals who are not suffering from the disease or disorder (e.g., cancer), an internal control (e.g., a housekeeping biomarker), or the level of a biomarker in a sample obtained prior to administration of a therapy (e.g., an anti-cancer therapy that includes a PD-L1 axis binding antagonist)), or relative to a reference level (e.g., the median expression level of the biomarker in samples from a group/population of patients, e.g., patients having cancer who are being tested for responsiveness to a PD-L1 axis binding antagonist; the median expression level of the biomarker in samples from a group/population of patients, e.g., patients having cancer who have been identified as not responding to a PD-L1 axis binding antagonist; or the level in a sample previously obtained from the individual at a prior time). In some embodiments, reduced expression is little or no expression.

A “reference sample,” “reference cell,” “reference tissue,” “control sample,” “control cell,” or “control tissue,” as used herein, refers to a sample, cell, tissue, or standard that is used for comparison purposes. In one embodiment, a reference sample, reference cell, reference tissue, control sample, control cell, or control tissue is obtained from a healthy and/or non-diseased part of the body (e.g., tissue or cells) of the same patient or individual. For example, a reference sample, reference cell, reference tissue, control sample, control cell, or control tissue may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same patient or individual. In yet another embodiment, a reference sample, reference cell, reference tissue, control sample, control cell, or control tissue is obtained from a healthy and/or non-diseased part of the body (e.g., tissues or cells) of an individual who is not the patient or individual. In even another embodiment, a reference sample, reference cell, reference tissue, control sample, control cell, or control tissue is obtained from an untreated tissue and/or cell of the body of an individual who is not the patient or individual. In another embodiment, a reference sample, reference cell, reference tissue, control sample, control cell, or control tissue is obtained from a patient prior to administration of a therapy (e.g., an anti-cancer therapy that includes a PD-L1 axis binding antagonist).

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The term “multiplex-PCR” refers to a single PCR reaction carried out on nucleic acid obtained from a single source (e.g., an individual) using more than one primer set for the purpose of amplifying two or more DNA sequences in a single reaction.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology, (Stockton Press, N Y, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

“Quantitative real-time polymerase chain reaction” or “qRT-PCR” refers to a form of PCR wherein the amount of PCR product is measured at each step in a PCR reaction. This technique has been described in various publications including, for example, Cronin et al., Am. J. Pathol. 164(1):35-42 (2004) and Ma et al., Cancer Cell 5:607-616 (2004).

The term “microarray” refers to an ordered arrangement of hybridizable array elements, preferably polynucleotide probes, on a substrate.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain biomarkers (e.g., one or more genes set forth in any one of Tables 1-7, e.g., IL8) in a biological sample from an individual.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Response to a treatment,” “responsiveness to treatment,” or “benefit from a treatment” can be assessed using any endpoint indicating a benefit to the individual, including, without limitation, (1) inhibition, to some extent, of disease progression (e.g., cancer progression), including slowing down and complete arrest; (2) a reduction in tumor size (e.g., an objective response, including a complete response or a partial response, or the rate or likelihood of such responses); (3) inhibition (i.e., reduction, slowing down or complete stopping) of cancer cell infiltration into adjacent peripheral organs and/or tissues; (4) inhibition (i.e., reduction, slowing down or complete stopping) of metastasis; (5) relief, to some extent, of one or more symptoms associated with the disease or disorder (e.g., cancer); (6) an increase or extension in the length of survival, including overall survival (e.g., OS hazard ratio (HR)<1) and progression free survival (e.g., PFS HR<1); and/or (9) decreased mortality at a given point of time following treatment (e.g., treatment with an anti-cancer therapy that includes a PD-L1 axis binding antagonist, or treatment with an anti-cancer therapy other than or in addition to a PD-L1 axis binding antagonist). In one embodiment, a biomarker (e.g., one or more genes set forth in any one of Tables 1-7, e.g., IL8) is used to identify an individual who is predicted to have an increased likelihood of being responsive to treatment with a medicament (e.g., treatment with an anti-cancer therapy that includes a PD-L1 axis binding antagonist, or treatment with an anti-cancer therapy other than or in addition to a PD-L1 axis binding antagonist), relative to a patient who does not express the biomarker.

An “objective response” refers to a measurable response, including complete response (CR) or partial response (PR). In some embodiments, the “objective response rate (ORR)” refers to the sum of complete response (CR) rate and partial response (PR) rate.

By “complete response” or “CR” is intended the disappearance of all signs of cancer (e.g., disappearance of all target lesions) in response to treatment. This does not always mean the cancer has been cured.

“Sustained response” refers to the sustained effect on reducing tumor growth after cessation of a treatment. For example, the tumor size may be the same size or smaller as compared to the size at the beginning of the medicament administration phase. In some embodiments, the sustained response has a duration at least the same as the treatment duration, at least 1.5×, 2.0×, 2.5×, or 3.0× length of the treatment duration, or longer.

As used herein, “reducing or inhibiting cancer relapse” means to reduce or inhibit tumor or cancer relapse or tumor or cancer progression. As disclosed herein, cancer relapse and/or cancer progression include, without limitation, cancer metastasis.

As used herein, “partial response” or “PR” refers to a decrease in the size of one or more tumors or lesions, or in the extent of cancer in the body, in response to treatment. For example, in some embodiments, PR refers to at least a 30% decrease in the sum of the longest diameters (SLD) of target lesions, taking as reference the baseline SLD.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

The term “survival” refers to the patient remaining alive, and includes overall survival as well as progression-free survival

As used herein, “progression-free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall survival” (OS) refers to the percentage of individuals in a group who are likely to be alive after a particular duration of time.

By “extending survival” is meant increasing overall or progression-free survival in a treated patient relative to an untreated patient (i.e. relative to a patient not treated with the medicament), or relative to a patient who does not express a biomarker at the designated level, and/or relative to a patient treated with an anti-tumor agent.

The term “dysfunction,” in the context of immune dysfunction, refers to a state of reduced immune responsiveness to antigenic stimulation. The term includes the common elements of both “exhaustion” and/or “anergy” in which antigen recognition may occur, but the ensuing immune response is ineffective to control infection or tumor growth.

The term “dysfunctional,” as used herein, also includes refractory or unresponsive to antigen recognition, specifically, impaired capacity to translate antigen recognition into down-stream T-cell effector functions, such as proliferation, cytokine production (e.g., IL-2) and/or target cell killing.

The term “anergy” refers to the state of unresponsiveness to antigen stimulation resulting from incomplete or insufficient signals delivered through the T-cell receptor (e.g., increase in intracellular Ca²⁺ in the absence of Ras activation). T-cell anergy can also result upon stimulation with antigen in the absence of co-stimulation, resulting in the cell becoming refractory to subsequent activation by the antigen even in the context of co-stimulation. The unresponsive state can often be overridden by the presence of interleukin-2. Anergic T-cells do not undergo clonal expansion and/or acquire effector functions.

The term “exhaustion” refers to T-cell exhaustion as a state of T-cell dysfunction that arises from sustained TCR signaling that occurs during many chronic infections and cancer. It is distinguished from anergy in that it arises not through incomplete or deficient signaling, but from sustained signaling. It is defined by poor effector function, sustained expression of inhibitory receptors and a transcriptional state distinct from that of functional effector or memory T-cells. Exhaustion prevents optimal control of infection and tumors. Exhaustion can result from both extrinsic negative regulatory pathways (e.g., immunoregulatory cytokines) as well as cell-intrinsic negative regulatory (co-stimulatory) pathways (PD-1, B7-H3, B7-H4, etc.).

“Enhancing T-cell function” means to induce, cause or stimulate a T-cell to have a sustained or amplified biological function, or renew or reactivate exhausted or inactive T-cells. Examples of enhancing T-cell function include: increased secretion of γ-interferon from CD8+ T-cells, increased proliferation, increased antigen responsiveness (e.g., viral, pathogen, or tumor clearance) relative to such levels before the intervention. In one embodiment, the level of enhancement is at least 50%, alternatively 60%, 70%, 80%, 90%, 100%, 120%, 150%, or 200% enhancement. The manner of measuring this enhancement is known to one of ordinary skill in the art.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

“Immunogenicity” refers to the ability of a particular substance to provoke an immune response. Tumors are immunogenic and enhancing tumor immunogenicity aids in the clearance of the tumor cells by the immune response. Examples of enhancing tumor immunogenicity include treatment with a PD-L1 axis binding antagonist.

The terms “Programmed Death Ligand 1” and “PD-L1” refer herein to a native sequence PD-L1 polypeptide, polypeptide variants, and fragments of a native sequence polypeptide and polypeptide variants (which are further defined herein). The PD-L1 polypeptide described herein may be that which is isolated from a variety of sources, such as from human tissue types or from another source, or prepared by recombinant or synthetic methods.

A “native sequence PD-L1 polypeptide” comprises a polypeptide having the same amino acid sequence as the corresponding PD-L1 polypeptide derived from nature.

A “PD-L1 polypeptide variant,” or variations thereof, means a PD-L1 polypeptide, generally an active PD-L1 polypeptide, as defined herein having at least about 80% amino acid sequence identity with any of the native sequence PD-L1 polypeptide sequences as disclosed herein. Such PD-L1 polypeptide variants include, for instance, PD-L1 polypeptides wherein one or more amino acid residues are added, or deleted, at the N- or C-terminus of a native amino acid sequence. Ordinarily, a PD-L1 polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a native sequence PD-L1 polypeptide sequence as disclosed herein.

Ordinarily, PD-L1 variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 281, 282, 283, 284, 285, 286, 287, 288, or 289 amino acids in length, or more. Optionally, PD-L1 variant polypeptides will have no more than one conservative amino acid substitution as compared to a native PD-L1 polypeptide sequence, alternatively no more than 2, 3, 4, 5, 6, 7, 8, 9, or 10 conservative amino acid substitutions as compared to a native PD-L1 polypeptide sequence.

The term “vascular endothelial growth factor” or “VEGF” refers to vascular endothelial growth factor protein A (VEGFA), as exemplified by Swiss Prot Accession Number P15692, Gene ID (NCBI): 7422. The term “VEGF” encompasses the protein having the amino acid sequence of Swiss Prot Accession Number P15692, Gene ID (NCBI): 7422 as well as homologues and isoforms thereof. The term “VEGF” also encompasses the known isoforms, e.g., splice isoforms, of VEGF, e.g., VEGF₁₁₁, VEGF₁₂₁, VEGF₁₄₅, VEGF₁₆₅, VEGF₁₈₉, and VEGF₂₀₆, together with the naturally-occurring allelic and processed forms thereof, including the 110 amino acid human vascular endothelial cell growth factor generated by plasmin cleavage of VEGF₁₆₅ as described in Ferrara Mol. Biol. Cell. 21:687, 2010; Leung et al., Science, 246:1306. 1989; and Houck et al., Mol. Endocrin., 5:1806, 1991. The term “VEGF” also refers to VEGFs from non-human species such as mouse, rat or primate. Sometimes the VEGF from a specific species are indicated by terms such as hVEGF for human VEGF, mVEGF for murine VEGF, and the like. The term “VEGF” is also used to refer to truncated forms of the polypeptide comprising amino acids 8 to 109 or 1 to 109 of the 165-amino acid human vascular endothelial cell growth factor. Reference to any such forms of VEGF may be identified in the present application, e.g., by “VEGF₁₀₉” “VEGF (8-109),” “VEGF (1-109)” or “VEGF₁₆₅.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF. The term “VEGF variant” as used herein refers to a VEGF polypeptide which includes one or more amino acid mutations in the native VEGF sequence. Optionally, the one or more amino acid mutations include amino acid substitution(s). For purposes of shorthand designation of VEGF variants described herein, it is noted that numbers refer to the amino acid residue position along the amino acid sequence of the putative native VEGF (provided in Leung et al., supra and Houck et al., supra). Unless specified otherwise, the term “VEGF” as used herein indicates VEGF-A.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “primer” refers to a single-stranded polynucleotide that is capable of hybridizing to a nucleic acid and allowing polymerization of a complementary nucleic acid, generally by providing a free 3′—OH group.

The term “small molecule” refers to any molecule with a molecular weight of about 2000 daltons or less, preferably of about 500 daltons or less.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

A “disorder” is any condition that would benefit from treatment including, but not limited to, chronic and acute disorders or diseases including those pathological conditions which predispose the mammal to the disorder in question.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia or lymphoid malignancies. More particular examples of such cancers include, but are not limited to, kidney or renal cancer (e.g., renal cell carcinoma (RCC)); lung cancer, including small-cell lung cancer, non-small cell lung cancer, adenocarcinoma of the lung, and squamous carcinoma of the lung; bladder cancer (e.g., urothelial carcinoma (UC), muscle invasive bladder cancer (MIBC), and BCG-refractory non-muscle invasive bladder cancer (NMIBC)); cancer of the urinary tract; breast cancer (e.g., HER2+ breast cancer and triple-negative breast cancer (TNBC), which are estrogen receptors (ER−), progesterone receptors (PR−), and HER2 (HER2−) negative); prostate cancer, such as castration-resistant prostate cancer (CRPC); cancer of the peritoneum; hepatocellular cancer; gastric or stomach cancer, including gastrointestinal cancer and gastrointestinal stromal cancer; pancreatic cancer; glioblastoma; cervical cancer; ovarian cancer; liver cancer (e.g., hepatocellular carcinoma (HCC)); hepatoma; colon cancer; rectal cancer; colorectal cancer; endometrial or uterine carcinoma; salivary gland carcinoma; prostate cancer; vulval cancer; thyroid cancer; hepatic carcinoma; anal carcinoma; penile carcinoma; melanoma, including superficial spreading melanoma, lentigo maligna melanoma, acral lentiginous melanomas, and nodular melanomas; multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases. In some embodiments, the cancer is bladder cancer. In particular embodiments, the bladder cancer is UC (e.g., advanced UC or metastatic UC (mUC)). In some embodiments, the cancer is kidney cancer. In particular embodiments, the kidney cancer is RCC (e.g., advanced RCC or metastatic RCC (mRCC), including previously untreated RCC).

By “early stage cancer” or “early stage tumor” is meant a cancer that is not invasive or metastatic or is classified as a Stage 0, I, or II cancer.

An “advanced” cancer is one which has spread outside the site or organ of origin, either by local invasion or metastasis.

A “refractory” cancer is one which progresses even though an anti-tumor agent, such as a chemotherapeutic agent, is being administered to the cancer patient. An example of a refractory cancer is one which is platinum refractory.

A “recurrent” cancer is one which has regrown, either at the initial site or at a distant site, after a response to initial therapy.

The terms “cell proliferative disorder” and “proliferative disorder” refer to disorders that are associated with some degree of abnormal cell proliferation. In one embodiment, the cell proliferative disorder is cancer.

The term “tumor,” as used herein, refers to all neoplastic cell growth and proliferation, whether malignant or benign, and all pre-cancerous and cancerous cells and tissues.

The terms “cancer,” “cancerous,” “cell proliferative disorder,” “proliferative disorder,” and “tumor” are not mutually exclusive as referred to herein.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies (e.g., anti-PD-L1 antibodies and/or anti-PD-1 antibodies) are used to delay development of a disease or to slow the progression of a disease.

The term “anti-cancer therapy” refers to a therapy useful in treating cancer. Examples of anti-cancer therapeutic agents include, but are limited to, checkpoint inhibitors (e.g., PD-L1 axis binding antagonists (e.g., PD-L1 antagonists (e.g., anti-PD-L1 antibodies such as atezolizumab)) or PD-1 antagonists (e.g., anti-PD-1 antibodies)), cytotoxic agents, chemotherapeutic agents, growth inhibitory agents, agents used in radiation therapy, anti-angiogenesis agents (e.g., VEGF antagonists (e.g., anti-VEGF antibodies (e.g., bevacizumab)), apoptotic agents, anti-tubulin agents, and other agents to treat cancer, for example, anti-CD20 antibodies, platelet derived growth factor inhibitors (e.g., GLEEVEC™ (imatinib mesylate)), a COX-2 inhibitor (e.g., celecoxib), interferons, cytokines, antagonists (e.g., neutralizing antibodies) that bind to one or more of the following targets PDGFR-β, BlyS, APRIL, BCMA receptor(s), TRAIL/Apo2, other bioactive and organic chemical agents, and the like. Combinations thereof are also included in the invention.

A “monotherapy” refers to a therapy (e.g., an anti-cancer therapy) that uses one type of treatment (e.g., therapeutic agent or other treatment modality (e.g., radiation)) to treat a disorder (e.g., cancer)). For example, a monotherapy may refer to use of a single therapeutic agent to treat a disorder. For example, a PD-L1 axis binding antagonist monotherapy is a therapy that involves administration of a PD-L1 axis binding antagonist without any additional anti-cancer therapeutic agents. In another example, a PD-L1 binding antagonist monotherapy is a therapy that involves administration of a PD-L1 binding antagonist without any additional anti-cancer therapeutic agents.

The term “PD-L1 axis binding antagonist” refers to a molecule that inhibits the interaction of a PD-L1 axis binding partner with one or more of its binding partners, so as to remove T-cell dysfunction resulting from signaling on the PD-1 signaling axis, with a result being restored or enhanced T-cell function. As used herein, a PD-L1 axis binding antagonist includes a PD-L1 binding antagonist and a PD-1 binding antagonist as well as molecules that interfere with the interaction between PD-L1 and PD-1 (e.g., a PD-L2-Fc fusion).

As used herein, a “PD-L1 binding antagonist” is a molecule that decreases, blocks, inhibits, abrogates or interferes with signal transduction resulting from the interaction of PD-L1 with either one or more of its binding partners, such as PD-1 and/or B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, PD-L1 binding antagonists include anti-PD-L1 antibodies and antigen-binding fragments thereof, immunoadhesins, fusion proteins, oligopeptides, small molecule antagonists, polynucleotide antagonists, and other molecules that decrease, block, inhibit, abrogate or interfere with signal transduction resulting from the interaction of PD-L1 with one or more of its binding partners, such as PD-1 and/or B7-1. In one embodiment, a PD-L1 binding antagonist reduces the negative signal mediated by or through cell surface proteins expressed on T lymphocytes and other cells through PD-L1 or PD-1 so as to render a dysfunctional T-cell less dysfunctional. In some embodiments, a PD-L1 binding antagonist is an anti-PD-L1 antibody. In a specific aspect, an anti-PD-L1 antibody is YW243.55.S70 described herein. In another specific aspect, an anti-PD-L1 antibody is MDX-1105 described herein. In still another specific aspect, an anti-PD-L1 antibody is atezolizumab described herein. In still another specific aspect, an anti-PD-L1 antibody is MEDI4736 (durvalumab) described herein. In still another specific aspect, an anti-PD-L1 antibody is MSB0010718C (avelumab) described herein.

As used herein, a “PD-1 binding antagonist” is a molecule that decreases, blocks, inhibits, abrogates or interferes with signal transduction resulting from the interaction of PD-1 with one or more of its binding partners, such as PD-L1 and/or PD-L2. In some embodiments, the PD-1 binding antagonist is a molecule that inhibits the binding of PD-1 to its binding partners. In a specific aspect, the PD-1 binding antagonist inhibits the binding of PD-1 to PD-L1 and/or PD-L2. For example, PD-1 binding antagonists include anti-PD-1 antibodies and antigen-binding fragments thereof, immunoadhesins, fusion proteins, oligopeptides, small molecule antagonists, polynucleotide antagonists, and other molecules that decrease, block, inhibit, abrogate or interfere with signal transduction resulting from the interaction of PD-1 with PD-L1 and/or PD-L2. In one embodiment, a PD-1 binding antagonist reduces the negative signal mediated by or through cell surface proteins expressed on T lymphocytes and other cells through PD-1 or PD-L1 so as to render a dysfunctional T-cell less dysfunctional. In some embodiments, the PD-1 binding antagonist is an anti-PD-1 antibody. In a specific aspect, a PD-1 binding antagonist is MDX-1106 (nivolumab) described herein. In another specific aspect, a PD-1 binding antagonist is MK-3475 (pembrolizumab) described herein. In another specific aspect, a PD-1 binding antagonist is MEDI-0680 (AMP-514) described herein. In another specific aspect, a PD-1 binding antagonist is PDR001 described herein. In another specific aspect, a PD-1 binding antagonist is REGN2810 described herein. In another specific aspect, a PD-1 binding antagonist is BGB-108 described herein. In another specific aspect, a PD-1 binding antagonist is AMP-224 described herein.

A “VEGF antagonist” or “VEGF-specific antagonist” refers to a molecule capable of binding to VEGF, reducing VEGF expression levels, or neutralizing, blocking, inhibiting, abrogating, reducing, or interfering with VEGF biological activities, including, but not limited to, VEGF binding to one or more VEGF receptors, VEGF signaling, and VEGF mediated angiogenesis and endothelial cell survival or proliferation. For example, a molecule capable of neutralizing, blocking, inhibiting, abrogating, reducing, or interfering with VEGF biological activities can exert its effects by binding to one or more VEGF receptor (VEGFR) (e.g., VEGFR1, VEGFR2, VEGFR3, membrane-bound VEGF receptor (mbVEGFR), or soluble VEGF receptor (sVEGFR)). Such antagonists are also referred to herein as “VEGFR inhibitors.” Included as VEGF-specific antagonists useful in the methods of the invention are polypeptides that specifically bind to VEGF, anti-VEGF antibodies and antigen-binding fragments thereof, receptor molecules and derivatives which bind specifically to VEGF thereby sequestering its binding to one or more receptors, fusions proteins (e.g., VEGF-Trap (Regeneron)), and VEGF₁₂₁-gelonin (Peregrine). VEGF-specific antagonists also include antagonist variants of VEGF polypeptides, antisense nucleobase oligomers complementary to at least a fragment of a nucleic acid molecule encoding a VEGF polypeptide; small RNAs complementary to at least a fragment of a nucleic acid molecule encoding a VEGF polypeptide; ribozymes that target VEGF; peptibodies to VEGF; and VEGF aptamers. VEGF antagonists also include polypeptides that bind to VEGFR, anti-VEGFR antibodies, and antigen-binding fragments thereof, and derivatives which bind to VEGFR thereby blocking, inhibiting, abrogating, reducing, or interfering with VEGF biological activities (e.g., VEGF signaling), or fusions proteins. VEGF-specific antagonists also include nonpeptide small molecules that bind to VEGF or VEGFR and are capable of blocking, inhibiting, abrogating, reducing, or interfering with VEGF biological activities. Thus, the term “VEGF activities” specifically includes VEGF mediated biological activities of VEGF. In certain embodiments, the VEGF antagonist reduces or inhibits, by at least 10%, 20%, 30%, 40%, 50%, 60%, 70%, 80%, 90% or more, the expression level or biological activity of VEGF. In some embodiments, the VEGF inhibited by the VEGF-specific antagonist is VEGF (8-109), VEGF (1-109), or VEGF₁₆₅.

As used herein VEGF antagonists can include, but are not limited to, anti-VEGFR2 antibodies and related molecules (e.g., ramucirumab, tanibirumab, aflibercept), anti-VEGFR1 antibodies and related molecules (e.g., icrucumab, aflibercept (VEGF Trap-Eye; EYLEA®), and ziv-aflibercept (VEGF Trap; ZALTRAP®)), bispecific VEGF antibodies (e.g., MP-0250, vanucizumab (VEGF-ANG2), and bispecific antibodies disclosed in US 2001/0236388), bispecific antibodies including combinations of two of anti-VEGF, anti-VEGFR1, and anti-VEGFR2 arms, anti-VEGFA antibodies (e.g., bevacizumab, sevacizumab), anti-VEGFB antibodies, anti-VEGFC antibodies (e.g., VGX-100), anti-VEGFD antibodies, and nonpeptide small molecule VEGF antagonists (e.g., pazopanib, axitinib, vandetanib, stivarga, cabozantinib, lenvatinib, nintedanib, orantinib, telatinib, dovitinig, cediranib, motesanib, sulfatinib, apatinib, foretinib, famitinib, and tivozanib).

An “anti-VEGF antibody” is an antibody that binds to VEGF with sufficient affinity and specificity. In certain embodiments, the antibody will have a sufficiently high binding affinity for VEGF, for example, the antibody may bind hVEGF with a Kd value of between 100 nM-1 pM. Antibody affinities may be determined, e.g., by a surface plasmon resonance-based assay (such as the BIAcore® assay as described in PCT Application Publication No. WO2005/012359); enzyme-linked immunoabsorbent assay (ELISA); and competition assays (e.g. radioimmunoassays (RIAs)).

In certain embodiments, the anti-VEGF antibody can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as PIGF, PDGF, or bFGF. In one embodiment, anti-VEGF antibody is a monoclonal antibody that binds to the same epitope as the monoclonal anti-VEGF antibody A4.6.1 produced by hybridoma ATCC HB 10709. In another embodiment, the anti-VEGF antibody is a recombinant humanized anti-VEGF monoclonal antibody generated according to Presta et al. (Cancer Res. 57:4593-4599, 1997), including but not limited to the antibody known as bevacizumab (BV; AVASTIN®).

The anti-VEGF antibody “Bevacizumab (BV),” also known as “rhuMAb VEGF” or “AVASTIN®,” is a recombinant humanized anti-VEGF monoclonal antibody generated according to Presta et al. (Cancer Res. 57:4593-4599, 1997). It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879 issued Feb. 26, 2005, the entire disclosure of which is expressly incorporated herein by reference. Additional preferred antibodies include the G6 or B20 series antibodies (e.g., G6-31, B20-4.1), as described in PCT Application Publication No. WO 2005/012359. For additional preferred antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al., (Journal of Immunological Methods 288:149-164, 2004). Other preferred antibodies include those that bind to a functional epitope on human VEGF comprising of residues F17, M18, D19, Y21, Y25, Q89, 191, K101, E103, and C104 or, alternatively, comprising residues F17, Y21, Q22, Y25, D63, 183, and Q89.

An “IL8 antagonist” refers to a molecule capable of binding to IL8, reducing IL8 expression levels, or neutralizing, blocking, inhibiting, abrogating, reducing, or interfering with IL8 biological activities, including, but not limited to, IL8 binding to one or more IL8 receptors, and IL8 signaling. For example, a molecule capable of neutralizing, blocking, inhibiting, abrogating, reducing, or interfering with IL8 biological activities can exert its effects by binding to one or more IL8 receptors (e.g., IL8RA and/or IL8RB). In some instances, the IL8 antagonist is an anti-IL8 antibody. Exemplary, non-limiting anti-IL8 antibodies include HuMax-IL8 (also known as BMS-986253). In other instances, the IL8 antagonist is a small molecule IL8 inhibitor. Exemplary, non-limiting small molecule IL8 inhibitors include reparixin (see, e.g., Leitner et al. Int. J. Immunopathol. Pharmacol. 20(1):25-36, 2007).

An “anti-IL8 antibody” is an antibody that binds to IL8 with sufficient affinity and specificity. In certain embodiments, the antibody will have a sufficiently high binding affinity for IL8, for example, the antibody may bind IL8 with a Kd value of between 100 nM-1 pM. Antibody affinities may be determined, e.g., by a surface plasmon resonance-based assay (such as the BIAcore® assay as described in PCT Application Publication No. WO2005/012359); enzyme-linked immunoabsorbent assay (ELISA); and competition assays (e.g. radioimmunoassays (RIAs)). The anti-IL8 antibody may be a neutralizing anti-IL8 antibody. Exemplary anti-IL8 antibodies include HuMax-IL8 (also known as BMS-986253).

An “IL1B antagonist” refers to a molecule capable of binding to IL1B, reducing IL1B expression levels, or neutralizing, blocking, inhibiting, abrogating, reducing, or interfering with IL1B biological activities, including, but not limited to, IL1B binding to one or more IL1B receptors, and IL1B signaling. For example, a molecule capable of neutralizing, blocking, inhibiting, abrogating, reducing, or interfering with IL1B biological activities can exert its effects by binding to one or more IL1B receptors (e.g., IL1R1, IL1R2, and/or IL-1 RAcP), also referred to as an “IL1R antagonist” (see below). In some instances, the IL1B antagonist is an anti-IL1B antibody. Exemplary, non-limiting anti-IL1B antibodies include canakinumab, gevokizumab, LY2189102, XOMA 052, and 2H (see, e.g., Goh et al. MAbs 6(3):764-772, 2014). In other instances, the IL1B antagonist is a small molecule IL1B inhibitor (e.g., a small molecule that inhibits IL1B release, e.g., a caspase 1 inhibitor (e.g., pralnacasan (VX-740) and VX-765)). Exemplary IL1B antagonists are described, e.g., in Dinarello et al. Nat. Rev. Drug Discov. 11(8):633-652, 2012

An “anti-IL1B antibody” is an antibody that binds to IL1B with sufficient affinity and specificity. In certain embodiments, the antibody will have a sufficiently high binding affinity for IL1B, for example, the antibody may bind IL1B with a Kd value of between 100 nM-1 pM. Antibody affinities may be determined, e.g., by a surface plasmon resonance-based assay (such as the BIAcore® assay as described in PCT Application Publication No. WO2005/012359); enzyme-linked immunoabsorbent assay (ELISA); and competition assays (e.g. radioimmunoassays (RIAs)). The anti-IL1B antibody may be a neutralizing anti-IL1B antibody. Exemplary, non-limiting anti-IL1B antibodies include canakinumab, gevokizumab, LY2189102, XOMA 052, and 2H (see, e.g., Goh et al. MAbs 6(3):764-772, 2014).

An “IL1R antagonist” refers to a molecule capable of binding to IL1R, reducing IL1R expression levels, or neutralizing, blocking, inhibiting, abrogating, reducing, or interfering with IL1R biological activities, including, but not limited to, IL1R binding to one or more ligands. In some instances, the IL1R antagonist is an anti-IL1R antibody. Exemplary, non-limiting anti-IL1R antibodies include AMG108. In other instances, the IL1R antagonist is a recombinant IL-1 RA protein or an engineered version thereof, e.g., anakinra (KINERET®). In yet other instances, the IL1R antagonist is a soluble decoy receptor, e.g., rilonacept (ARCALYST®), which is a dimeric fusion protein that includes the ligand-binding domains of the extracellular portions of the human IL1R (IL1R1) and an IL1 receptor accessory protein (IL-1 RAcP) linked in-line to the Fc region of human IgG1. In other instances, the IL1B antagonist is a small molecule IL1B inhibitor. Exemplary IL1R antagonists are described, e.g., in Dinarello et al. Nat. Rev. Drug Discov. 11(8):633-652, 2012.

An “anti-IL1R antibody” is an antibody that binds to IL1R with sufficient affinity and specificity. In certain embodiments, the antibody will have a sufficiently high binding affinity for IL1R, for example, the antibody may bind IL1R with a Kd value of between 100 nM-1 pM. Antibody affinities may be determined, e.g., by a surface plasmon resonance-based assay (such as the BIAcore® assay as described in PCT Application Publication No. WO2005/012359); enzyme-linked immunoabsorbent assay (ELISA); and competition assays (e.g. radioimmunoassays (RIAs)). The anti-IL1R antibody may be a neutralizing anti-IL1R antibody. Exemplary, non-limiting anti-IL1R antibodies include AMG108.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², and radioactive isotopes of Lu), chemotherapeutic agents, e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (see, e.g., Nicolaou et al., Angew. Chem Intl. Ed. Engl., 33: 183-186 (1994)); dynemicin, including dynemicin A; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycin, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, for example taxanes including TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Illinois), and TAXOTERE® docetaxel (Rhône-Poulenc Rorer, Antony, France); chloranbucil; gemcitabine (GEMZAR®); 6-thioguanine; mercaptopurine; methotrexate; platinum or platinum-based chemotherapy agents and platinum analogs, such as cisplatin, carboplatin, oxaliplatin (ELOXATIN™), satraplatin, picoplatin, nedaplatin, triplatin, and lipoplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin. Additional chemotherapeutic agents include the cytotoxic agents useful as antibody drug conjugates, such as maytansinoids (DM1, for example) and the auristatins MMAE and MMAF, for example.

“Chemotherapeutic agents” also include “anti-hormonal agents” or “endocrine therapeutics” that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone, and FARESTON® toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGFR); vaccines such as THERATOPE® vaccine and gene therapy vaccines, for example, ALLOVECTIN® vaccine, LEUVECTIN® vaccine, and VAXID® vaccine; LURTOTECAN® topoisomerase 1 inhibitor; ABARELIX® rmRH; lapatinib ditosylate (an ErbB-2 and EGFR dual tyrosine kinase small-molecule inhibitor also known as GW572016); and pharmaceutically acceptable salts, acids or derivatives of any of the above.

Chemotherapeutic agents also include antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1λ antibody genetically modified to recognize interleukin-12 p40 protein.

Chemotherapeutic agents also include “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3, and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP 659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO 98/14451, WO 98/50038, WO 99/09016, and WO 99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitors such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1 C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (ENBREL®), infliximab (REMICADE®), adalimumab (HUMIRA®), certolizumab pegol (CIMZIA®), golimumab (SIMPONI®), Interleukin 1 (IL-1) blockers such as anakinra (KINERET®), T-cell co-stimulation blockers such as abatacept (ORENCIA®), Interleukin 6 (IL-6) blockers such as tocilizumab (ACTEMERA®); Interleukin 13 (IL-13) blockers such as lebrikizumab; Interferon alpha (IFN) blockers such as rontalizumab; beta 7 integrin blockers such as rhuMAb Beta7; IgE pathway blockers such as anti-M1 prime; Secreted homotrimeric LTa3 and membrane bound heterotrimer LTa1/β2 blockers such as anti-lymphotoxin alpha (LTa); miscellaneous investigational agents such as thioplatin, PS-341, phenylbutyrate, ET-18-OCH3, and farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g., celecoxib or etoricoxib), proteosome inhibitor (e.g., PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above.

The term “prodrug” as used herein refers to a precursor or derivative form of a pharmaceutically active substance that is less cytotoxic to tumor cells compared to the parent drug and is capable of being enzymatically activated or converted into the more active parent form. See, for example, Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

A “growth inhibitory agent” when used herein refers to a compound or composition which inhibits growth and/or proliferation of a cell (e.g., a cell whose growth is dependent on PD-L1 expression) either in vitro or in vivo. Thus, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as the anthracycline antibiotic doxorubicin ((8S-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-naphthacenedione), epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in “The Molecular Basis of Cancer,” Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

As used herein, the terms “individual,” “patient,” and “subject” are used interchangeably and refer to any single animal, more preferably a mammal (including such non-human animals as, for example, dogs, cats, horses, rabbits, zoo animals, cows, pigs, sheep, and non-human primates) for which treatment is desired. In particular embodiments, the individual herein is a human.

As used herein, “administering” is meant a method of giving a dosage of a compound (e.g., a PD-L1 axis binding antagonist, a VEGF antagonist, or any other anti-cancer therapeutic agent) or a pharmaceutical composition (e.g., a pharmaceutical composition including an PD-L1 axis binding antagonist, a VEGF antagonist, or any other anti-cancer therapeutic agent) to an individual (e.g., a patient). The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subcutaneously, subconjunctivally, intravesicularly, mucosally, intrapericardially, intraumbilically, intraocularly, intraorbitally, intravitreally (e.g., by intravitreal injection), by eye drop, orally, topically, transdermally, parenterally, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally.

The method of administration can vary depending on various factors (e.g., the compound or composition being administered and the severity of the condition, disease, or disorder being treated). Various dosing schedules including but not limited to single or multiple administrations over various time-points, bolus administration, and pulse infusion are contemplated herein.

A “therapeutically effective amount” or an “effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., ORR, CR, and/or PR), duration of response, and/or quality of life.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 20%, 30%, 40%, 50%, 60%, 70%, 75%, 80%, 85%, 90%, 95%, or greater. Reduce or inhibit can refer, for example, to the symptoms of the disorder being treated, the presence or size of metastases, or the size of the primary tumor.

The term “package insert” is used to refer to instructions customarily included in commercial packages of therapeutic products, that contain information about the indications, usage, dosage, administration, combination therapy, contraindications, and/or warnings concerning the use of such therapeutic products.

A “sterile” formulation is aseptic or free from all living microorganisms and their spores.

An “article of manufacture” is any manufacture (e.g., a package or container) or kit comprising at least one reagent, e.g., a medicament for treatment of a disease or disorder (e.g., cancer), or a probe for specifically detecting a biomarker (e.g., IL8) described herein. In certain embodiments, the manufacture or kit is promoted, distributed, or sold as a unit for performing the methods described herein.

The phrase “based on” when used herein means that the information about one or more biomarkers is used to inform a treatment decision, information provided on a package insert, or marketing/promotional guidance, etc.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “isolated” antibody is one which has been identified and separated and/or recovered from a component of its natural environment. Contaminant components of its natural environment are materials which would interfere with research, diagnostic, and/or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. An isolated antibody includes the antibody in situ within recombinant cells since at least one component of the antibody's natural environment will not be present. Ordinarily, however, an isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The “light chains” of antibodies (immunoglobulins) from any mammalian species can be assigned to one of two clearly distinct types, called kappa (“κ”) and lambda (“λ”), based on the amino acid sequences of their constant domains.

The term “constant domain” refers to the portion of an immunoglobulin molecule having a more conserved amino acid sequence relative to the other portion of the immunoglobulin, the variable domain, which contains the antigen binding site. The constant domain contains the CH₁, CH₂, and CH₃ domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The term “hypervariable region,” “HVR,” or “HV,” as used herein, refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, for example, Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, for example, Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35b | H26-H35b | H26-H32 | H30-H35b |
| | | | | (Kabat Numbering) |
| H1 | H31-H35 | H26-H35 | H26-H32 | H30-H35 |
| | | | | (Chothia Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody, preferably comprising the antigen-binding region thereof. In some embodiments, the antibody fragment described herein is an antigen-binding fragment. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH₁) of the heavy chain. Fab′ fragments differ from Fab fragments by the addition of a few residues at the carboxy terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthun, in The Pharmacology of Monoclonal Antibodies, vol. 113, Rosenburg and Moore eds., (Springer-Verlag, New York, 1994), pp. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of antibodies are called α, δ, ε, γ, and μ, respectively.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target-binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target-binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target-binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual(Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg et al., Intern. Rev. Immunol. 13: 65-93 (1995)).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The terms “anti-PD-L1 antibody” and “an antibody that binds to PD-L1” refer to an antibody that is capable of binding PD-L1 with sufficient affinity such that the antibody is useful as a diagnostic and/or therapeutic agent in targeting PD-L1. In one embodiment, the extent of binding of an anti-PD-L1 antibody to an unrelated, non-PD-L1 protein is less than about 10% of the binding of the antibody to PD-L1 as measured, for example, by a radioimmunoassay (RIA). In certain embodiments, an anti-PD-L1 antibody binds to an epitope of PD-L1 that is conserved among PD-L1 from different species.

The terms “anti-PD-1 antibody” and “an antibody that binds to PD-1” refer to an antibody that is capable of binding PD-1 with sufficient affinity such that the antibody is useful as a diagnostic and/or therapeutic agent in targeting PD-1. In one embodiment, the extent of binding of an anti-PD-1 antibody to an unrelated, non-PD-1 protein is less than about 10% of the binding of the antibody to PD-1 as measured, for example, by a radioimmunoassay (RIA). In certain embodiments, an anti-PD-1 antibody binds to an epitope of PD-1 that is conserved among PD-1 from different species.

A “blocking” antibody or an “antagonist” antibody is one which inhibits or reduces biological activity of the antigen it binds. Preferred blocking antibodies or antagonist antibodies substantially or completely inhibit the biological activity of the antigen.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

An “immunoconjugate” is an antibody conjugated to one or more heterologous molecule(s), including but not limited to a cytotoxic agent.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG1, IgG2 (including IgG2A and IgG2B), IgG3, or IgG4 subtypes, IgA (including IgA1 and IgA2), IgE, IgD or IgM. The Ig fusions preferably include the substitution of a domain of a polypeptide or antibody described herein in the place of at least one variable region within an Ig molecule. In a particularly preferred embodiment, the immunoglobulin fusion includes the hinge, CH₂ and CH₃, or the hinge, CH₁, CH₂ and CH₃ regions of an IgG1 molecule. For the production of immunoglobulin fusions see also U.S. Pat. No. 5,428,130. For example, useful immunoadhesins as medicaments useful for therapy herein include polypeptides that comprise the extracellular domain (ECD) or PD-1-binding portions of PD-L1 or PD-L2, or the extracellular or PD-L1- or PD-L2-binding portions of PD-1, fused to a constant domain of an immunoglobulin sequence, such as a PD-L1 ECD-Fc, a PD-L2 ECD-Fc, and a PD-1 ECD-Fc, respectively. Immunoadhesin combinations of Ig Fc and ECD of cell surface receptors are sometimes termed soluble receptors.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target molecule, catalysis of a reaction, and the like. The two portions may be linked directly by a single peptide bond or through a peptide linker but are in reading frame with each other.

“Percent (%) amino acid sequence identity” with respect to the polypeptide sequences identified herein is defined as the percentage of amino acid residues in a candidate sequence that are identical with the amino acid residues in the polypeptide being compared, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, California. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “substantially the same,” as used herein, denotes a sufficiently high degree of similarity between two numeric values, such that one of skill in the art would consider the difference between the two values to be of little or no biological and/or statistical significance within the context of the biological characteristic measured by said values (e.g., Kd values or expression levels). The difference between said two values is, for example, less than about 50%, less than about 40%, less than about 30%, less than about 20%, and/or less than about 10%, as a function of the reference/comparator value.

The phrase “substantially different,” as used herein, denotes a sufficiently high degree of difference between two numeric values such that one of skill in the art would consider the difference between the two values to be of statistical significance within the context of the biological characteristic measured by said values (e.g., Kd values or expression levels). The difference between said two values is, for example, greater than about 10%, greater than about 20%, greater than about 30%, greater than about 40%, and/or greater than about 50%, as a function of the value for the reference/comparator molecule.

The word “label” when used herein refers to a compound or composition that is conjugated or fused directly or indirectly to a reagent such as a polynucleotide probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable. The term is intended to encompass direct labeling of a probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin.

### III. Methods

A. Methods of Diagnosis, Identification, Prediction, and Selection

The present disclosure provides, inter alia, methods of identifying an individual having a cancer (e.g., bladder cancer (e.g., UC) or kidney cancer (e.g., RCC)) who is likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), methods for selecting a therapy for an individual having a cancer, and methods of identifying an individual having a cancer who is less likely to respond to treatment with an anti-cancer therapy comprising a PD-L1 axis binding antagonist monotherapy. Any of the preceding methods may be based on the expression level of a biomarker provided herein, for example, any gene set forth in any one of Tables 1-7, for example, IL8 expression, in a sample obtained from the individual. As described herein, e.g., in Example 1, an expression level of IL8 in a sample obtained from an individual that is below a reference level of IL8, or an on-treatment decrease in the expression level of IL8 relative to a reference level of IL8, is associated with improved treatment response from anti-cancer therapy that includes a PD-L1 axis binding antagonist. In contrast, as is also described herein, e.g., in Example 1, an expression level of IL8 in a sample obtained from an individual that is at or above a reference level of IL8, or an on-treatment increase in the expression level of IL8 relative to a reference level of IL8, is associated with reduced treatment response from anti-cancer therapy that includes a PD-L1 axis binding antagonist monotherapy, which can identify the individual as one who may benefit from anti-cancer therapy other than or in addition to a PD-L1 axis binding antagonist. As is also described herein, the invention also provides genes that are upregulated or downregulated in the context of IL8 high cancer, e.g., in tumor samples and/or in PBMCs. See, e.g., Example 1. The expression level of one or more genes that are upregulated or downregulated in the context of IL8 high cancer, e.g., one or more genes set forth in any one of Tables 2-7, can be used as proxies for IL8 expression, e.g., in methods of identifying an individual having a cancer (e.g., bladder cancer (e.g., UC) or kidney cancer (e.g., RCC)) who is likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), methods for selecting a therapy for an individual having a cancer, and methods of identifying an individual having a cancer who is less likely to respond to treatment with an anti-cancer therapy comprising a PD-L1 axis binding antagonist monotherapy. Any of the methods may further include administering to the patient an anti-cancer therapy to the individual, for example, an anti-cancer therapy that includes a PD-L1 axis binding antagonist (for example, as described in Section IV, below) or an anti-cancer therapy other than or in addition to a PD-L1 axis binding antagonist. Any of the methods may further include administering an effective amount of one or more additional therapeutic agents to the individual.

TABLE 1
Exemplary Biomarkers of the Invention

| | | | | | |
|---|---|---|---|---|---|
| IL8 | DONSON | MIR155HG | ZNF331 | FGR | NPM1 |
| ALOX5AP | DUSP2 | NAMPT | ATP5E | FKBP1A | PF4 |
| AMFR | DUSP6 | NCF1C | AIF1 | GLRX | PFN1 |
| ANKRD28 | EGR1 | NFKBIA | ANXA2 | HES4 | PLAC8 |
| AREG | ELL2 | NFKBIZ | ANXA5 | HLA-DMA | POU2F2 |
| ARL5B | EREG | NLRP3 | ARHGDIB | HLA-DMB | PPA1 |
| ATF3 | FOS | NR4A2 | ARPC1B | HLA-DQB1 | PPBP |
| B4GALT1 | FPR1 | NR4A3 | ARPC3 | HLA-DRB1 | PPIA |
| B4GALT5 | FTH1 | NRG1 | ATP5G3 | HLA-DRB5 | PRELID1 |
| BCL2A1 | FTH1P3 | OSM | ATP6 | HLA-DRB6 | PRKCB |
| BID | FTL | PHLDA1 | BIN2 | HLA-L | PSMB10 |
| C15orf48 | G0S2 | PLAUR | C1orf162 | HN1 | PSMB9 |
| C1orf56 | GLUL | PLIN2 | C1QA | HNRNPA1 | PYCARD |
| C5AR1 | GNLY | PLSCR1 | C4orf48 | HSPA8 | RAC2 |
| CCL20 | GPR183 | PPP1R15A | CAPNS1 | ID2 | RETN |
| CCL3 | GPX1 | PTGES | CCR1 | IER5 | RGS10 |
| CCL3L1 | GYPC | PTGS2 | CD36 | IFITM1 | RHOC |
| CCL4 | H1F0 | RASGEF1B | CD52 | IFITM2 | RNR2 |
| CCL4L1 | H1FX | RBP7 | CD68 | IFITM3 | RNVU1-19 |
| CCL4L2 | H3F3A | RGCC | CD74 | ITGAL | RPL7A |
| CD14 | HBB | RGS2 | CDA | JUNB | RPS24 |
| CD69 | HBEGF | RPS4Y1 | CFL1 | LGALS9 | RPS26 |
| CD83 | HCAR3 | S100A12 | CFP | LILRB2 | RPS4X |
| CD93 | HILPDA | S100A8 | CLEC12A | LINC01272 | RPSA |
| CDA | HIST1H1C | S100A9 | CLU | LOC100509457 | RPSAP58 |
| CDC42SE1 | HSPA5 | SBDS | CORO1A | LOC101929876 | S100A12 |
| CDKN1A | ICAM1 | SEMA6B | COTL1 | LOC606724 | S100A6 |
| CEBPD | IDS | SERPINB2 | CRIP1 | LST1 | S100A8 |
| CH25H | IER3 | SGK1 | CST3 | LY6E | S100A9 |
| CLDND1 | IL10 | SLC25A37 | CSTB | MPEG1 | SLC25A5 |
| CLEC7A | IL1B | SOD2 | CTSC | MS4A7 | SLC7A7 |
| CSF3R | INSIG1 | SPI1 | CXCL13 | MT2A | SNHG5 |
| CSRNP1 | IRF2BP2 | SRGN | CYTB | NAP1L1 | TCF7L2 |
| CTNNB1 | LGALS1 | THBD | DUSP6 | NCF2 | TPT1 |
| CTSD | LGALS2 | THBS1 | EEF1A1 | ND1 | TSC22D3 |
| CXCL1 | LINC00936 | TIMP1 | EEF1B2 | ND2 | TUBA1B |
| CXCL16 | LOC101930114 | TNF | EEF1G | ND3 | UCP2 |
| CXCL2 | LYZ | TNFAIP3 | FAM110A | ND4 | UQCRB |
| CXCL3 | MAFB | TRAF1 | FAM26F | ND5 | VCAN |
| CXCL8 | MARCKS | TSPYL2 | FAM65B | NDUFB1 | WFDC2 |
| CYP1B1 | MCEMP1 | VNN2 | FCGR3A | NDUFB5 | YBX1 |

1. IL8

In several aspects of the present disclosure, decreased or low IL8 levels can be used to identify and/or select individuals who are likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)). For example, decreased or low baseline or on-treatment IL8 expression levels (e.g., relative to a reference level of IL8) can be used to identify individuals who are likely to respond to treatment with an anti-cancer therapy including a PD-L1 axis binding antagonist.

For example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), the method including determining the expression level of IL8 in a sample from the individual, wherein an expression level of IL8 in the sample that is below a reference level of IL8 identifies the individual as likely to respond to treatment with an anti-cancer therapy including a PD-L1 axis binding antagonist.

In another example, provided herein is a method for selecting a therapy for an individual having a cancer (e.g., bladder cancer (e.g., UC) or kidney cancer (e.g., RCC)), the method including: (a) determining the expression level of IL8 in a sample from the individual, wherein an expression level of IL8 in the sample that is below a reference level of IL8 identifies the individual as likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)); and (b) selecting an anti-cancer therapy including a PD-L1 axis binding antagonist for the individual based on the expression level of IL8 determined in step (a).

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of IL8 in a sample from the individual, wherein an expression level of IL8 in the sample that is below a reference level of IL8 identifies the individual as likely to respond to treatment with an anti-cancer therapy including a PD-L1 axis binding antagonist, wherein the response is indicated by OS or ORR.

In another example, provided herein is a method for selecting a therapy for an individual having a cancer (e.g., bladder cancer (e.g., UC) or kidney cancer (e.g., RCC)), the method including: (a) determining the expression level of IL8 in a sample from the individual, wherein an expression level of IL8 in the sample that is below a reference level of IL8 identifies the individual as likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), wherein the response is indicated by OS or ORR; and (b) selecting an anti-cancer therapy including a PD-L1 axis binding antagonist for the individual based on the expression level of IL8 determined in step (a).

In yet another example, provided herein is a method of identifying an individual having a cancer (e.g., bladder cancer (e.g., UC) or kidney cancer (e.g., RCC)) who is likely to respond to treatment with an anti-cancer therapy including a PD-L1 binding antagonist (e.g., an anti-PD-L1 antibody such as atezolizumab), the method including determining the expression level of IL8 in a sample from the individual, wherein an expression level of IL8 in the sample that is below a reference level of IL8 identifies the individual as likely to respond to treatment with an anti-cancer therapy including a PD-L1 binding antagonist, wherein the response is indicated by OS or ORR.

In a further example, provided herein is a method for selecting a therapy for an individual having a cancer (e.g., bladder cancer (e.g., UC) or kidney cancer (e.g., RCC)), the method including: (a) determining the expression level of IL8 in a sample from the individual, wherein an expression level of IL8 in the sample that is below a reference level of IL8 identifies the individual as likely to respond to treatment with an anti-cancer therapy including a PD-L1 binding antagonist (e.g., an anti-PD-L1 antibody such as atezolizumab), wherein the response is indicated by OS or ORR; and (b) selecting an anti-cancer therapy including a PD-L1 binding antagonist for the individual based on the expression level of IL8 determined in step (a).

In another example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of IL8 in a plasma sample or a sample including PBMCs from the individual, wherein an expression level of IL8 in the plasma sample or the sample including PBMCs that is below a reference level of IL8 identifies the individual as likely to respond to treatment with an anti-cancer therapy including a PD-L1 axis binding antagonist. In some instances, the sample is a plasma sample. In other instances, the sample includes PBMCs.

In yet another example, provided herein is a method for selecting a therapy for an individual having a cancer (e.g., bladder cancer (e.g., UC) or kidney cancer (e.g., RCC)), the method including: (a) determining the expression level of IL8 in a plasma sample or a sample including PBMCs from the individual, wherein an expression level of IL8 in the plasma sample or the sample including PBMCs that is below a reference level of IL8 identifies the individual as likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)); and (b) selecting a therapy including a PD-L1 axis binding antagonist for the individual based on the expression level of IL8 in the plasma sample or in the sample including PBMCs determined in step (a). In some instances, the sample is a plasma sample. In other instances, the sample includes PBMCs.

In another example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of IL8 in a sample obtained from the individual at a time point following administration of the anti-cancer therapy, wherein an expression level of IL8 in the sample that is below a reference level of IL8 identifies the individual as likely to respond to treatment with an anti-cancer therapy including a PD-L1 axis binding antagonist.

In yet another example, provided herein is a method for selecting a therapy for an individual having a cancer (e.g., bladder cancer (e.g., UC) or kidney cancer (e.g., RCC)), the method including: (a) determining the expression level of IL8 in a sample obtained from the individual at a time point following administration of the anti-cancer therapy, wherein an expression level of IL8 in the sample that is below a reference level of IL8 identifies the individual as likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)); and (b) selecting an anti-cancer therapy including a PD-L1 axis binding antagonist for the individual based on the expression level of IL8 determined in step (a).

In another example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of IL8 in a sample obtained from the individual at a time point following administration of the anti-cancer therapy, wherein the sample is a plasma sample, a tumor tissue sample, or a sample including PBMCs, and wherein an expression level of IL8 in the sample that is below a reference level of IL8 identifies the individual as likely to respond to treatment with an anti-cancer therapy including a PD-L1 axis binding antagonist.

In yet another example, provided herein is a method for selecting a therapy for an individual having a cancer (e.g., bladder cancer (e.g., UC) or kidney cancer (e.g., RCC)), the method including: (a) determining the expression level of IL8 in a sample obtained from the individual at a time point following administration of the anti-cancer therapy, wherein the sample is a plasma sample, a tumor tissue sample, or a sample including PBMCs, and wherein an expression level of IL8 in the sample that is below a reference level of IL8 identifies the individual as likely to respond to treatment with an anti-cancer therapy including a PD-L1 axis binding antagonist; and (b) selecting an anti-cancer therapy including a PD-L1 axis binding antagonist for the individual based on the expression level of IL8 determined in step (a).

Any suitable time point following administration of the anti-cancer therapy may be used. For example, the time point following administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) following administration of the anti-cancer therapy. In some instances, the time point following administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks following administration of the anti-cancer therapy. In some instances, the time point following administration of the anti-cancer therapy is about 4 to about 8 weeks following administration of the anti-cancer therapy. For example, in some aspects, the time point following administration of the anti-cancer therapy is about 6 weeks following administration of the anti-cancer therapy.

In another example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of IL8 in a sample from the individual that has been obtained at a time point prior to or concurrently with administration of an anti-cancer therapy including a PD-L1 axis binding antagonist, wherein an expression level of IL8 in the sample that is below a reference level of IL8 identifies the individual as likely to respond to treatment with an anti-cancer therapy including a PD-L1 axis binding antagonist.

In yet another example, provided herein is a method for selecting a therapy for an individual having a cancer (e.g., bladder cancer (e.g., UC) or kidney cancer (e.g., RCC)), the method including: (a) determining the expression level of IL8 in a sample from the individual that has been obtained at a time point prior to or concurrently with administration of an anti-cancer therapy including a PD-L1 axis binding antagonist (e.g., an anti-PD-L1 antibody such as atezolizumab) or a PD-1 binding antagonist (e.g., an anti-PD-1 antibody)), wherein an expression level of IL8 in the sample that is below a reference level of IL8 identifies the individual as likely to respond to treatment with an anti-cancer therapy including a PD-L1 axis binding antagonist; and (b) selecting an anti-cancer therapy including a PD-L1 axis binding antagonist for the individual based on the expression level of IL8 determined in step (a).

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of IL8 in a sample from the individual that has been obtained at a time point prior to or concurrently with administration of an anti-cancer therapy including a PD-L1 axis binding antagonist, wherein an expression level of IL8 in the sample that is below a reference level of IL8 identifies the individual as likely to respond to treatment with an anti-cancer therapy including a PD-L1 axis binding antagonist, wherein the response is indicated by OS or ORR.

In another example, provided herein is a method for selecting a therapy for an individual having a cancer (e.g., bladder cancer (e.g., UC) or kidney cancer (e.g., RCC)), the method including: (a) determining the expression level of IL8 in a sample from the individual that has been obtained at a time point prior to or concurrently with administration of an anti-cancer therapy including a PD-L1 axis binding antagonist (e.g., an anti-PD-L1 antibody such as atezolizumab) or a PD-1 binding antagonist (e.g., an anti-PD-1 antibody)), wherein an expression level of IL8 in the sample that is below a reference level of IL8 identifies the individual as likely to respond to treatment with an anti-cancer therapy including a PD-L1 axis binding antagonist, wherein the response is indicated by OS or ORR; and (b) selecting an anti-cancer therapy including a PD-L1 axis binding antagonist for the individual based on the expression level of IL8 determined in step (a).

Any suitable time point prior to administration of the anti-cancer therapy may be used. For example, the time point prior to administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) prior to administration of the anti-cancer therapy. In some instances, the time point prior to administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks prior to administration of the anti-cancer therapy.

In any of the preceding methods, the individual may be likely to have improved response from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). For example, the individual may be likely to have an improved ORR, improved CR rate, improved PR rate, extended PFS, and/or extended OS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In some particular instances, the individual may be likely to have an improved ORR from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In other particular instances, the individual may be likely to have an extended PFS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In yet other particular instances, the individual may be likely to have an extended OS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist).

In other aspects of the present disclosure, elevated or high IL8 levels can be used to identify and/or select individuals who are less likely to respond to treatment with an anti-cancer therapy including a PD-L1 binding antagonist monotherapy. For example, elevated or high baseline or on-treatment IL8 expression levels (e.g., relative to a reference level of IL8) can be used to identify individuals who are less likely to respond to treatment with an anti-cancer therapy including a PD-L1 binding antagonist monotherapy. Such patients may be candidates for treatment with an anti-cancer therapy other than or in addition to a PD-L1 axis binding antagonist (e.g., combination therapy with a PD-L1 axis binding antagonist and one or more additional therapeutic agents (e.g., VEGF antagonists (e.g., anti-VEGF antibodies such as bevacizumab), or an anti-cancer therapy that does not include a PD-L1 axis binding antagonist).

For example, provided herein is a method of identifying an individual having a cancer (e.g., bladder cancer (e.g., UC) or kidney cancer (e.g., RCC)) who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of IL8 in a sample from the individual, wherein an expression level of IL8 in the sample that is at or above a reference level of IL8 identifies the individual as less likely to respond to treatment with an anti-cancer therapy including a PD-L1 axis binding antagonist monotherapy.

In another example, provided herein is a method for selecting a therapy for an individual having a cancer (e.g., bladder cancer (e.g., UC) or kidney cancer (e.g., RCC)), the method including: (a) determining the expression level of IL8 in a sample from the individual, wherein an expression level of IL8 in the sample that is at or above a reference level of IL8 identifies the individual as less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy; and (b) selecting an anti-cancer therapy other than or in addition to a PD-L1 axis binding antagonist for the individual based on the expression level of IL8 determined in step (a).

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of identifying an individual having a cancer (e.g., bladder cancer (e.g., UC) or kidney cancer (e.g., RCC)) who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of IL8 in a sample from the individual, wherein an expression level of IL8 in the sample that is at or above a reference level of IL8 identifies the individual as less likely to respond to treatment with an anti-cancer therapy including a PD-L1 axis binding antagonist monotherapy, and wherein the response is indicated by OS or ORR.

In another example, provided herein is a method for selecting a therapy for an individual having a cancer (e.g., bladder cancer (e.g., UC) or kidney cancer (e.g., RCC)), the method including: (a) determining the expression level of IL8 in a sample from the individual, wherein an expression level of IL8 in the sample that is at or above a reference level of IL8 identifies the individual as less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, wherein the response is indicated by OS or ORR; and (b) selecting an anti-cancer therapy other than or in addition to a PD-L1 axis binding antagonist for the individual based on the expression level of IL8 determined in step (a).

For example, provided herein is a method of identifying an individual having a cancer (e.g., bladder cancer (e.g., UC) or kidney cancer (e.g., RCC)) who is less likely to respond to treatment with an anti-cancer therapy including a PD-L1 binding antagonist (e.g., an anti-PD-L1 antibody such as atezolizumab), the method including determining the expression level of IL8 in a sample from the individual, wherein an expression level of IL8 in the sample that is at or above a reference level of IL8 identifies the individual as less likely to respond to treatment with an anti-cancer therapy including a PD-L1 binding antagonist monotherapy, and wherein the response is indicated by OS or ORR.

In another example, provided herein is a method for selecting a therapy for an individual having a cancer (e.g., bladder cancer (e.g., UC) or kidney cancer (e.g., RCC)), the method including: (a) determining the expression level of IL8 in a sample from the individual, wherein an expression level of IL8 in the sample that is at or above a reference level of IL8 identifies the individual as less likely to respond to treatment with an anti-cancer therapy including a PD-L1 binding antagonist (e.g., an anti-PD-L1 antibody such as atezolizumab) monotherapy, wherein the response is indicated by OS or ORR; and (b) selecting an anti-cancer therapy other than or in addition to a PD-L1 binding antagonist for the individual based on the expression level of IL8 determined in step (a).

In another example, provided herein is a method of identifying an individual having a cancer (e.g., bladder cancer (e.g., UC) or kidney cancer (e.g., RCC)) who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of IL8 in a plasma sample or a sample including PBMCs from the individual, wherein an expression level of IL8 in the plasma sample or the sample including PBMCs that is at or above a reference level of IL8 identifies the individual as less likely to respond to treatment with an anti-cancer therapy including a PD-L1 axis binding antagonist monotherapy.

In yet another example, provided herein is a method for selecting a therapy for an individual having a cancer (e.g., bladder cancer (e.g., UC) or kidney cancer (e.g., RCC)), the method including: (a) determining the expression level of IL8 in a plasma sample or a sample including PBMCs from the individual, wherein an expression level of IL8 in the plasma sample or the sample including PBMCs that is at or above a reference level of IL8 identifies the individual as less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy; and (b) selecting a therapy other than or in addition to a PD-L1 axis binding antagonist for the individual based on the expression level of IL8 in the plasma sample or in the sample including PBMCs determined in step (a).

In another example, provided herein is a method of identifying an individual having a cancer who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of IL8 in a sample obtained from the individual at a time point following administration of an anti-cancer therapy including a PD-L1 axis binding antagonist, wherein an expression level of IL8 in the sample that is at or above a reference level of IL8 identifies the individual as less likely to respond to treatment with an anti-cancer therapy including a PD-L1 axis binding antagonist monotherapy.

In another example, provided herein is a method for selecting a therapy for an individual having a cancer (e.g., bladder cancer (e.g., UC) or kidney cancer (e.g., RCC)), the method including: (a) determining the expression level of IL8 in a sample obtained from the individual at a time point following administration of an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein an expression level of IL8 in the sample that is at or above a reference level of IL8 identifies the individual as less likely to respond to treatment with an anti-cancer therapy including a PD-L1 axis binding antagonist monotherapy; and (b) selecting an anti-cancer therapy other than or in addition to a PD-L1 axis binding antagonist for the individual based on the expression level of IL8 determined in step (a).

In another example, provided herein is a method of identifying an individual having a cancer who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of IL8 in a sample obtained from the individual at a time point following administration of an anti-cancer therapy including a PD-L1 axis binding antagonist, wherein the sample is a plasma sample, a tumor tissue sample, or a sample including PBMCs, and wherein an expression level of IL8 in the sample that is at or above a reference level of IL8 identifies the individual as less likely to respond to treatment with an anti-cancer therapy including a PD-L1 axis binding antagonist monotherapy.

In yet another example, provided herein is a method for selecting a therapy for an individual having a cancer (e.g., bladder cancer (e.g., UC) or kidney cancer (e.g., RCC)), the method including: (a) determining the expression level of IL8 in a sample obtained from the individual at a time point following administration of an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein the sample is a plasma sample, a tumor tissue sample, or a sample including PBMCs, and wherein an expression level of IL8 in the sample that is at or above a reference level of IL8 identifies the individual as less likely to respond to treatment with an anti-cancer therapy including a PD-L1 axis binding antagonist monotherapy; and (b) selecting an anti-cancer therapy other than or in addition to a PD-L1 axis binding antagonist for the individual based on the expression level of IL8 determined in step (a).

Any suitable time point following administration of the anti-cancer therapy may be used. For example, the time point following administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) following administration of the anti-cancer therapy. In some instances, the time point following administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks following administration of the anti-cancer therapy. In some instances, the time point following administration of the anti-cancer therapy is about 4 to about 8 weeks following administration of the anti-cancer therapy. For example, in some aspects, the time point following administration of the anti-cancer therapy is about 6 weeks following administration of the anti-cancer therapy.

In another example, provided herein is a method of identifying an individual having a cancer (e.g., bladder cancer (e.g., UC) or kidney cancer (e.g., RCC)) who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of IL8 in a sample from the individual that has been obtained at a time point prior to or concurrently with administration of an anti-cancer therapy including a PD-L1 axis binding antagonist, wherein an expression level of IL8 in the sample that is at or above a reference level of IL8 identifies the individual as less likely to respond to treatment with an anti-cancer therapy including a PD-L1 axis binding antagonist monotherapy.

In yet another example, provided herein is a method for selecting a therapy for an individual having a cancer (e.g., bladder cancer (e.g., UC) or kidney cancer (e.g., RCC)), the method including: (a) determining the expression level of IL8 in a sample from the individual that has been obtained at a time point prior to or concurrently with administration of an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein an expression level of IL8 in the sample that is at or above a reference level of IL8 identifies the individual as less likely to respond to treatment with an anti-cancer therapy including a PD-L1 axis binding antagonist monotherapy; and (b) selecting an anti-cancer therapy other than or in addition to a PD-L1 axis binding antagonist for the individual based on the expression level of IL8 determined in step (a).

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of identifying an individual having a cancer (e.g., bladder cancer (e.g., UC) or kidney cancer (e.g., RCC)) who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of IL8 in a sample from the individual that has been obtained at a time point prior to or concurrently with administration of an anti-cancer therapy including a PD-L1 axis binding antagonist, wherein an expression level of IL8 in the sample that is at or above a reference level of IL8 identifies the individual as less likely to respond to treatment with an anti-cancer therapy including a PD-L1 axis binding antagonist monotherapy, and wherein the response is indicated by OS or ORR.

In another example, provided herein is a method for selecting a therapy for an individual having a cancer (e.g., bladder cancer (e.g., UC) or kidney cancer (e.g., RCC)), the method including: (a) determining the expression level of IL8 in a sample from the individual that has been obtained at a time point prior to or concurrently with administration of an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein an expression level of IL8 in the sample that is at or above a reference level of IL8 identifies the individual as less likely to respond to treatment with an anti-cancer therapy including a PD-L1 axis binding antagonist monotherapy, wherein the response is indicated by OS or ORR; and (b) selecting an anti-cancer therapy other than or in addition to a PD-L1 axis binding antagonist for the individual based on the expression level of IL8 determined in step (a).

Any suitable time point prior to administration of the anti-cancer therapy may be used. For example, the time point prior to administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) prior to administration of the anti-cancer therapy. In some instances, the time point prior to administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks prior to administration of the anti-cancer therapy.

In any of the preceding methods, the individual may be likely to have a reduced response from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). For example, the individual may be likely to have a reduced ORR, reduced CR rate, reduced PR rate, shortened PFS, or shortened OS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In some particular instances, the individual may be likely to have a reduced ORR from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In other particular instances, the individual may be likely to have a shortened PFS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In other particular instances, the individual may be likely to have a shortened OS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist).

2. Genes Upregulated in IL8 High Cancer

In several aspects of the present disclosure, decreased or low levels of one or more genes that are upregulated in IL8 high cancer can be used to identify and/or select individuals who are likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)). For example, decreased or low baseline or on-treatment expression levels of one or more genes set forth in any one of Tables 2-4 (e.g., relative to a reference level of one or more genes set forth in any one of Tables 2-4) can be used to identify individuals who are likely to respond to treatment with an anti-cancer therapy including a PD-L1 axis binding antagonist.

TABLE 2
Genes Upregulated in IL8-High Cancer

| | | | | | | |
|---|---|---|---|---|---|---|
| ALOX5AP | CCL4L2 | CXCL2 | GPR183 | IRF2BP2 | OSM | SERPINB2 |
| AMFR | CD14 | CXCL3 | GPX1 | LGALS1 | PHLDA1 | SGK1 |
| ANKRD28 | CD69 | CXCL8 | GYPC | LGALS2 | PLAUR | SLC25A37 |
| AREG | CD83 | CYP1B1 | H1F0 | LINC00936 | PLIN2 | SOD2 |
| ARL5B | CD93 | DONSON | H1FX | LOC101930114 | PLSCR1 | SPI1 |
| ATF3 | CDA | DUSP2 | H3F3A | LYZ | PPP1R15A | SRGN |
| B4GALT1 | CDC42SE1 | DUSP6 | HBB | MAFB | PTGES | THBD |
| B4GALT5 | CDKN1A | EGR1 | HBEGF | MARCKS | PTGS2 | THBS1 |
| BCL2A1 | CEBPD | ELL2 | HCAR3 | MCEMP1 | RASGEF1B | TIMP1 |
| BID | CH25H | EREG | HILPDA | MIR155HG | RBP7 | TNF |
| C15orf48 | CLDND1 | FOS | HIST1H1C | NAMPT | RGCC | TNFAIP3 |
| C1orf56 | CLEC7A | FPR1 | HSPA5 | NCF1C | RGS2 | TRAF1 |
| C5AR1 | CSF3R | FTH1 | ICAM1 | NFKBIA | RPS4Y1 | TSPYL2 |
| CCL20 | CSRNP1 | FTH1P3 | IDS | NFKBIZ | S100A12 | VNN2 |
| CCL3 | CTNNB1 | FTL | IER3 | NLRP3 | S100A8 | ZNF331 |
| CCL3L1 | CTSD | G0S2 | IL10 | NR4A2 | S100A9 | |
| CCL4 | CXCL1 | GLUL | IL1B | NR4A3 | SBDS | |
| CCL4L1 | CXCL16 | GNLY | INSIG1 | NRG1 | SEMA6B | |

TABLE 3
Genes Upregulated in IL8-High RCC Tumors

| | | | | | |
|---|---|---|---|---|---|
| AREG | CCL4 | CXCL2 | ICAM1 | NLRP3 | RASGEF1B |
| ARL5B | CCL4L1 | CXCL3 | IER3 | NR4A2 | SOD2 |
| ATF3 | CCL4L2 | CXCL8 | IL10 | NR4A3 | THBS1 |
| BCL2A1 | CD83 | DUSP2 | IL1B | PHLDA1 | TNF |
| C15orf48 | CDKN1A | ELL2 | INSIG1 | PLAUR | TNFAIP3 |
| CCL20 | CH25H | EREG | MIR155HG | PLIN2 | TRAF1 |
| CCL3 | CSRNP1 | G0S2 | NFKBIA | PPP1R15A | ZNF331 |
| CCL3L1 | CXCL1 | GPR183 | NFKBIZ | PTGS2 | |

TABLE 4
Genes Upregulated in IL8-High Bladder Cancer PBMCs

| | | | | | |
|---|---|---|---|---|---|
| ALOX5AP | CEBPD | FPR1 | HILPDA | NCF1C | SEMA6B |
| AMFR | CLDND1 | FTH1 | HIST1H1C | NRG1 | SERPINB2 |
| ANKRD28 | CLEC7A | FTH1P3 | HSPA5 | OSM | SGK1 |
| B4GALT1 | CSF3R | FTL | IDS | PHLDA1 | SLC25A37 |
| B4GALT5 | CTNNB1 | G0S2 | IER3 | PLAUR | SOD2 |
| BCL2A1 | CTSD | GLUL | IL1B | PLIN2 | SPI1 |
| BID | CXCL16 | GNLY | IRF2BP2 | PLSCR1 | SRGN |
| C15orf48 | CXCL2 | GPR183 | LGALS1 | PTGES | THBD |
| C1orf56 | CXCL3 | GPX1 | LGALS2 | RBP7 | THBS1 |
| C5AR1 | CXCL8 | GYPC | LINC00936 | RGCC | TIMP1 |
| CCL3 | CYP1B1 | H1F0 | LOC101930114 | RGS2 | TSPYL2 |
| CD14 | DONSON | H1FX | LYZ | RPS4Y1 | VNN2 |
| CD69 | DUSP6 | H3F3A | MAFB | S100A12 | |
| CD93 | EGR1 | HBB | MARCKS | S100A8 | |
| CDA | EREG | HBEGF | MCEMP1 | S100A9 | |
| CDC42SE1 | FOS | HCAR3 | NAMPT | SBDS | |

For example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), the method including determining the expression level of one or more genes set forth in any one of Tables 2-4 in a sample from the individual, wherein an expression level of the one or more genes set forth in any one of Tables 2-4 in the sample that is below a reference level of the one or more genes set forth in any one of Tables 2-4 identifies the individual as likely to respond to treatment with an anti-cancer therapy including a PD-L1 axis binding antagonist.

In another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 2-4 in a sample from the individual, wherein an expression level of the one or more genes set forth in any one of Tables 2-4 in the sample that is below a reference level of the one or more genes set forth in any one of Tables 2-4 identifies the individual as likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)); and (b) selecting an anti-cancer therapy including a PD-L1 axis binding antagonist for the individual based on the expression level of the one or more genes set forth in any one of Tables 2-4 determined in step (a).

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of one or more genes set forth in any one of Tables 2-4 in a sample from the individual, wherein an expression level of the one or more genes set forth in any one of Tables 2-4 in the sample that is below a reference level of the one or more genes set forth in any one of Tables 2-4 identifies the individual as likely to respond to treatment with an anti-cancer therapy including a PD-L1 axis binding antagonist, wherein the response is indicated by OS or ORR.

In another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 2-4 in a sample from the individual, wherein an expression level of the one or more genes set forth in any one of Tables 2-4 in the sample that is below a reference level of the one or more genes set forth in any one of Tables 2-4 identifies the individual as likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), wherein the response is indicated by OS or ORR; and (b) selecting an anti-cancer therapy including a PD-L1 axis binding antagonist for the individual based on the expression level of the one or more genes set forth in any one of Tables 2-4 determined in step (a).

In yet another example, provided herein is a method of identifying an individual having a cancer (e.g., bladder cancer (e.g., UC) or kidney cancer (e.g., RCC)) who is likely to respond to treatment with an anti-cancer therapy including a PD-L1 binding antagonist (e.g., an anti-PD-L1 antibody such as atezolizumab), the method including determining the expression level of one or more genes set forth in any one of Tables 2-4 in a sample from the individual, wherein an expression level of the one or more genes set forth in any one of Tables 2-4 in the sample that is below a reference level of the one or more genes set forth in any one of Tables 2-4 identifies the individual as likely to respond to treatment with an anti-cancer therapy including a PD-L1 binding antagonist, wherein the response is indicated by OS or ORR.

In a further example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 2-4 in a sample from the individual, wherein an expression level of the one or more genes set forth in any one of Tables 2-4 in the sample that is below a reference level of the one or more genes set forth in any one of Tables 2-4 identifies the individual as likely to respond to treatment with an anti-cancer therapy including a PD-L1 binding antagonist (e.g., an anti-PD-L1 antibody such as atezolizumab), wherein the response is indicated by OS or ORR; and (b) selecting an anti-cancer therapy including a PD-L1 binding antagonist for the individual based on the expression level of the one or more genes set forth in any one of Tables 2-4 determined in step (a).

In another example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of one or more genes set forth in any one of Tables 2-4 in a plasma sample or a sample including PBMCs from the individual, wherein an expression level of the one or more genes set forth in any one of Tables 2-4 in the plasma sample or the sample including PBMCs that is below a reference level of the one or more genes set forth in any one of Tables 2-4 identifies the individual as likely to respond to treatment with an anti-cancer therapy including a PD-L1 axis binding antagonist. In some instances, the sample is a plasma sample. In other instances, the sample includes PBMCs.

In yet another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 2-4 in a plasma sample or a sample including PBMCs from the individual, wherein an expression level of the one or more genes set forth in any one of Tables 2-4 in the plasma sample or the sample including PBMCs that is below a reference level of the one or more genes set forth in any one of Tables 2-4 identifies the individual as likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)); and (b) selecting a therapy including a PD-L1 axis binding antagonist for the individual based on the expression level of the one or more genes set forth in any one of Tables 2-4 in the plasma sample or in the sample including PBMCs determined in step (a). In some instances, the sample is a plasma sample. In other instances, the sample includes PBMCs.

In another example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of one or more genes set forth in any one of Tables 2-4 in a sample obtained from the individual at a time point following administration of the anti-cancer therapy, wherein an expression level of the one or more genes set forth in any one of Tables 2-4 in the sample that is below a reference level of the one or more genes set forth in any one of Tables 2-4 identifies the individual as likely to respond to treatment with an anti-cancer therapy including a PD-L1 axis binding antagonist.

In yet another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 2-4 in a sample obtained from the individual at a time point following administration of the anti-cancer therapy, wherein an expression level of the one or more genes set forth in any one of Tables 2-4 in the sample that is below a reference level of the one or more genes set forth in any one of Tables 2-4 identifies the individual as likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)); and (b) selecting an anti-cancer therapy including a PD-L1 axis binding antagonist for the individual based on the expression level of the one or more genes set forth in any one of Tables 2-4 determined in step (a).

In another example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of one or more genes set forth in any one of Tables 2-4 in a sample obtained from the individual at a time point following administration of the anti-cancer therapy, wherein the sample is a plasma sample, a tumor tissue sample, or a sample including PBMCs, and wherein an expression level of the one or more genes set forth in any one of Tables 2-4 in the sample that is below a reference level of the one or more genes set forth in any one of Tables 2-4 identifies the individual as likely to respond to treatment with an anti-cancer therapy including a PD-L1 axis binding antagonist.

In yet another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 2-4 in a sample obtained from the individual at a time point following administration of the anti-cancer therapy, wherein the sample is a plasma sample, a tumor tissue sample, or a sample including PBMCs, and wherein an expression level of the one or more genes set forth in any one of Tables 2-4 in the sample that is below a reference level of the one or more genes set forth in any one of Tables 2-4 identifies the individual as likely to respond to treatment with an anti-cancer therapy including a PD-L1 axis binding antagonist; and (b) selecting an anti-cancer therapy including a PD-L1 axis binding antagonist for the individual based on the expression level of the one or more genes set forth in any one of Tables 2-4 determined in step (a).

Any suitable time point following administration of the anti-cancer therapy may be used. For example, the time point following administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) following administration of the anti-cancer therapy. In some instances, the time point following administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks following administration of the anti-cancer therapy. In some instances, the time point following administration of the anti-cancer therapy is about 4 to about 8 weeks following administration of the anti-cancer therapy. For example, in some aspects, the time point following administration of the anti-cancer therapy is about 6 weeks following administration of the anti-cancer therapy.

In another example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of one or more genes set forth in any one of Tables 2-4 in a sample from the individual that has been obtained at a time point prior to or concurrently with administration of an anti-cancer therapy including a PD-L1 axis binding antagonist, wherein an expression level of the one or more genes set forth in any one of Tables 2-4 in the sample that is below a reference level of the one or more genes set forth in any one of Tables 2-4 identifies the individual as likely to respond to treatment with an anti-cancer therapy including a PD-L1 axis binding antagonist.

In yet another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 2-4 in a sample from the individual that has been obtained at a time point prior to or concurrently with administration of an anti-cancer therapy including a PD-L1 axis binding antagonist (e.g., an anti-PD-L1 antibody such as atezolizumab) or a PD-1 binding antagonist (e.g., an anti-PD-1 antibody)), wherein an expression level of the one or more genes set forth in any one of Tables 2-4 in the sample that is below a reference level of the one or more genes set forth in any one of Tables 2-4 identifies the individual as likely to respond to treatment with an anti-cancer therapy including a PD-L1 axis binding antagonist; and (b) selecting an anti-cancer therapy including a PD-L1 axis binding antagonist for the individual based on the expression level of the one or more genes set forth in any one of Tables 2-4 determined in step (a).

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of one or more genes set forth in any one of Tables 2-4 in a sample from the individual that has been obtained at a time point prior to or concurrently with administration of an anti-cancer therapy including a PD-L1 axis binding antagonist, wherein an expression level of the one or more genes set forth in any one of Tables 2-4 in the sample that is below a reference level of the one or more genes set forth in any one of Tables 2-4 identifies the individual as likely to respond to treatment with an anti-cancer therapy including a PD-L1 axis binding antagonist, wherein the response is indicated by OS or ORR.

In another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 2-4 in a sample from the individual that has been obtained at a time point prior to or concurrently with administration of an anti-cancer therapy including a PD-L1 axis binding antagonist (e.g., an anti-PD-L1 antibody such as atezolizumab) or a PD-1 binding antagonist (e.g., an anti-PD-1 antibody)), wherein an expression level of the one or more genes set forth in any one of Tables 2-4 in the sample that is below a reference level of the one or more genes set forth in any one of Tables 2-4 identifies the individual as likely to respond to treatment with an anti-cancer therapy including a PD-L1 axis binding antagonist, wherein the response is indicated by OS or ORR; and (b) selecting an anti-cancer therapy including a PD-L1 axis binding antagonist for the individual based on the expression level of the one or more genes set forth in any one of Tables 2-4 determined in step (a).

Any suitable time point prior to administration of the anti-cancer therapy may be used. For example, the time point prior to administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) prior to administration of the anti-cancer therapy. In some instances, the time point prior to administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks prior to administration of the anti-cancer therapy.

In any of the preceding methods, the individual may be likely to have improved response from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). For example, the individual may be likely to have an improved ORR, improved CR rate, improved PR rate, extended PFS, and/or extended OS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In some particular instances, the individual may be likely to have an improved ORR from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In other particular instances, the individual may be likely to have an extended PFS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In yet other particular instances, the individual may be likely to have an extended OS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist).

In other aspects of the present disclosure, elevated or high levels of one or more genes set forth in any one of Tables 2-4 can be used to identify and/or select individuals who are less likely to respond to treatment with an anti-cancer therapy including a PD-L1 binding antagonist monotherapy. For example, elevated or high baseline or on-treatment expression levels of one or more genes set forth in any one of Tables 2-4 (e.g., relative to a reference level of the one or more genes set forth in any one of Tables 2-4) can be used to identify individuals who are less likely to respond to treatment with an anti-cancer therapy including a PD-L1 binding antagonist monotherapy. Such patients may be candidates for treatment with an anti-cancer therapy other than or in addition to a PD-L1 axis binding antagonist (e.g., combination therapy with a PD-L1 axis binding antagonist and one or more additional therapeutic agents (e.g., VEGF antagonists (e.g., anti-VEGF antibodies such as bevacizumab), or an anti-cancer therapy that does not include a PD-L1 axis binding antagonist).

For example, provided herein is a method of identifying an individual having a cancer (e.g., bladder cancer (e.g., UC) or kidney cancer (e.g., RCC)) who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of one or more genes set forth in any one of Tables 2-4 in a sample from the individual, wherein an expression level of the one or more genes set forth in any one of Tables 2-4 in the sample that is at or above a reference level of IL8 identifies the individual as less likely to respond to treatment with an anti-cancer therapy including a PD-L1 axis binding antagonist monotherapy.

In another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 2-4 in a sample from the individual, wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy; and (b) selecting an anti-cancer therapy other than or in addition to a PD-L1 axis binding antagonist for the individual based on the expression level of the one or more genes set forth in any one of Tables 2-4 determined in step (a).

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of identifying an individual having a cancer (e.g., bladder cancer (e.g., UC) or kidney cancer (e.g., RCC)) who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of one or more genes set forth in any one of Tables 2-4 in a sample from the individual, wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy including a PD-L1 axis binding antagonist monotherapy, and wherein the response is indicated by OS or ORR.

In another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 2-4 in a sample from the individual, wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, wherein the response is indicated by OS or ORR; and (b) selecting an anti-cancer therapy other than or in addition to a PD-L1 axis binding antagonist for the individual based on the expression level of the one or more genes set forth in any one of Tables 2-4 determined in step (a).

For example, provided herein is a method of identifying an individual having a cancer (e.g., bladder cancer (e.g., UC) or kidney cancer (e.g., RCC)) who is less likely to respond to treatment with an anti-cancer therapy including a PD-L1 binding antagonist (e.g., an anti-PD-L1 antibody such as atezolizumab), the method including determining the expression level of one or more genes set forth in any one of Tables 2-4 in a sample from the individual, wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy including a PD-L1 binding antagonist monotherapy, and wherein the response is indicated by OS or ORR.

In another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 2-4 in a sample from the individual, wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy including a PD-L1 binding antagonist (e.g., an anti-PD-L1 antibody such as atezolizumab) monotherapy, wherein the response is indicated by OS or ORR; and (b) selecting an anti-cancer therapy other than or in addition to a PD-L1 binding antagonist for the individual based on the expression level of IL8 determined in step (a).

In another example, provided herein is a method of identifying an individual having a cancer (e.g., bladder cancer (e.g., UC) or kidney cancer (e.g., RCC)) who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of one or more genes set forth in any one of Tables 2-4 in a plasma sample or a sample including PBMCs from the individual, wherein an expression level of the one or more genes set forth in any one of Tables 2-4 in the plasma sample or the sample including PBMCs that is at or above a reference level of the one or more genes set forth in any one of Tables 2-4 identifies the individual as less likely to respond to treatment with an anti-cancer therapy including a PD-L1 axis binding antagonist monotherapy.

In yet another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 2-4 in a plasma sample or a sample including PBMCs from the individual, wherein an expression level of the one or more genes set forth in any one of Tables 2-4 in the plasma sample or the sample including PBMCs that is at or above a reference level of the one or more genes set forth in any one of Tables 2-4 identifies the individual as less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy; and (b) selecting a therapy other than or in addition to a PD-L1 axis binding antagonist for the individual based on the expression level of the one or more genes set forth in any one of Tables 2-4 in the plasma sample or in the sample including PBMCs determined in step (a).

In another example, provided herein is a method of identifying an individual having a cancer who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of one or more genes set forth in any one of Tables 2-4 in a sample obtained from the individual at a time point following administration of an anti-cancer therapy including a PD-L1 axis binding antagonist, wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy including a PD-L1 axis binding antagonist monotherapy.

In another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 2-4 in a sample obtained from the individual at a time point following administration of an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy including a PD-L1 axis binding antagonist monotherapy; and (b) selecting an anti-cancer therapy other than or in addition to a PD-L1 axis binding antagonist for the individual based on the expression level of the one or more genes set forth in any one of Tables 2-4 determined in step (a).

In another example, provided herein is a method of identifying an individual having a cancer who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of one or more genes set forth in any one of Tables 2-4 in a sample obtained from the individual at a time point following administration of an anti-cancer therapy including a PD-L1 axis binding antagonist, wherein the sample is a plasma sample, a tumor tissue sample, or a sample including PBMCs, and wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy including a PD-L1 axis binding antagonist monotherapy.

In yet another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 2-4 in a sample obtained from the individual at a time point following administration of an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein the sample is a plasma sample, a tumor tissue sample, or a sample including PBMCs, and wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy including a PD-L1 axis binding antagonist monotherapy; and (b) selecting an anti-cancer therapy other than or in addition to a PD-L1 axis binding antagonist for the individual based on the expression level of the one or more genes set forth in any one of Tables 2-4 determined in step (a).

Any suitable time point following administration of the anti-cancer therapy may be used. For example, the time point following administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) following administration of the anti-cancer therapy. In some instances, the time point following administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks following administration of the anti-cancer therapy. In some instances, the time point following administration of the anti-cancer therapy is about 4 to about 8 weeks following administration of the anti-cancer therapy. For example, in some aspects, the time point following administration of the anti-cancer therapy is about 6 weeks following administration of the anti-cancer therapy.

In another example, provided herein is a method of identifying an individual having a cancer (e.g., bladder cancer (e.g., UC) or kidney cancer (e.g., RCC)) who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of one or more genes set forth in any one of Tables 2-4 in a sample from the individual that has been obtained at a time point prior to or concurrently with administration of an anti-cancer therapy including a PD-L1 axis binding antagonist, wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy including a PD-L1 axis binding antagonist monotherapy.

In yet another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 2-4 in a sample from the individual that has been obtained at a time point prior to or concurrently with administration of an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy including a PD-L1 axis binding antagonist monotherapy; and (b) selecting an anti-cancer therapy other than or in addition to a PD-L1 axis binding antagonist for the individual based on the expression level of IL8 determined in step (a).

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of identifying an individual having a cancer (e.g., bladder cancer (e.g., UC) or kidney cancer (e.g., RCC)) who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of one or more genes set forth in any one of Tables 2-4 in a sample from the individual that has been obtained at a time point prior to or concurrently with administration of an anti-cancer therapy including a PD-L1 axis binding antagonist, wherein an expression level of IL8 in the sample that is at or above a reference level of IL8 identifies the individual as less likely to respond to treatment with an anti-cancer therapy including a PD-L1 axis binding antagonist monotherapy, and wherein the response is indicated by OS or ORR.

In another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 2-4 in a sample from the individual that has been obtained at a time point prior to or concurrently with administration of an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy including a PD-L1 axis binding antagonist monotherapy, wherein the response is indicated by OS or ORR; and (b) selecting an anti-cancer therapy other than or in addition to a PD-L1 axis binding antagonist for the individual based on the expression level of the one or more genes set forth in any one of Tables 2-4 determined in step (a).

Any suitable time point prior to administration of the anti-cancer therapy may be used. For example, the time point prior to administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) prior to administration of the anti-cancer therapy. In some instances, the time point prior to administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks prior to administration of the anti-cancer therapy.

In any of the preceding methods, the individual may be likely to have a reduced response from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). For example, the individual may be likely to have a reduced ORR, reduced CR rate, reduced PR rate, shortened PFS, or shortened OS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In some particular instances, the individual may be likely to have a reduced ORR from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In other particular instances, the individual may be likely to have a shortened PFS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In other particular instances, the individual may be likely to have a shortened OS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist).

3. Genes Downregulated in IL8 High Cancer

In several aspects of the present disclosure, increased or high levels of one or more genes that are downregulated in IL8-high cancer can be used to identify and/or select individuals who are likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)). For example, increased or high baseline or on-treatment expression levels of one or more genes set forth in any one of Tables 5-7 (e.g., relative to a reference level of one or more genes set forth in any one of Tables 5-7) can be used to identify individuals who are likely to respond to treatment with an anti-cancer therapy including a PD-L1 axis binding antagonist.

TABLE 5
Genes Downregulated in IL8 High Cancer

| | | | | | | |
|---|---|---|---|---|---|---|
| ATP5E | CD74 | FAM110A | ID2 | NAP1L1 | PRKCB | S100A8 |
| AIF1 | CDA | FAM26F | IER5 | NCF2 | PSMB10 | S100A9 |
| ANXA2 | CFL1 | FAM65B | IFITM1 | ND1 | PSMB9 | SLC25A5 |
| ANXA5 | CFP | FCGR3A | IFITM2 | ND2 | PYCARD | SLC7A7 |
| ARHGDIB | CLEC12A | FGR | IFITM3 | ND3 | RAC2 | SNHG5 |
| ARPC1B | CLU | FKBP1A | ITGAL | ND4 | RETN | TCF7L2 |
| ARPC3 | CORO1A | GLRX | JUNB | ND5 | RGS10 | TPT1 |
| ATP5G3 | COTL1 | HES4 | LGALS9 | NDUFB1 | RHOC | TSC22D3 |
| ATP6 | CRIP1 | HLA-DMA | LILRB2 | NDUFB5 | RNR2 | TUBA1B |
| BIN2 | CST3 | HLA-DMB | LINC01272 | NPM1 | RNVU1- | UCP2 |
| | | | | | 19 | |
| C1orf162 | CSTB | HLA- | LOC100509457 | PF4 | RPL7A | UQCRB |
| | | DQB1 | | | | |
| C1QA | CTSC | HLA- | LOC101929876 | PFN1 | RPS24 | VCAN |
| | | DRB1 | | | | |
| C4orf48 | CXCL13 | HLA- | LOC606724 | PLAC8 | RPS26 | WFDC2 |
| | | DRB5 | | | | |
| CAPNS1 | CYTB | HLA- | LST1 | POU2F2 | RPS4X | YBX1 |
| | | DRB6 | | | | |
| CCR1 | DUSP6 | HLA-L | LY6E | PPA1 | RPSA | |
| CD36 | EEF1A1 | HN1 | MPEG1 | PPBP | RPSAP58 | |
| CD52 | EEF1B2 | HNRNPA1 | MS4A7 | PPIA | S100A12 | |
| CD68 | EEF1G | HSPA8 | MT2A | PRELID1 | S100A6 | |

TABLE 6
Genes Downregulated in IL8 High RCC Tumors

| | | | | | |
|---|---|---|---|---|---|
| ATP5E | CDA | CXCL13 | ITGAL | PLAC8 | S100A6 |
| C4orf48 | CFP | DUSP6 | LINC01272 | POU2F2 | S100A8 |
| CAPNS1 | CLEC12A | FAM110A | LOC101929876 | PRKCB | S100A9 |
| CCR1 | CLU | FAM65B | LST1 | RETN | TCF7L2 |
| CD36 | COTL1 | HLA-DRB6 | MPEG1 | RPS26 | VCAN |
| CD52 | CRIP1 | HLA-L | NDUFB1 | S100A12 | WFDC2 |

TABLE 7
Genes Downregulated in IL8 High Bladder Cancer PBMCs

| | | | | | | |
|---|---|---|---|---|---|---|
| AIF1 | CD74 | FCGR3A | ID2 | MT2A | PPBP | RPS26 |
| ANXA2 | CFL1 | FGR | IER5 | NAP1L1 | PPIA | RPS4X |
| ANXA5 | CFP | FKBP1A | IFITM1 | NCF2 | PRELID1 | RPSA |
| ARHGDIB | CORO1A | GLRX | IFITM2 | ND1 | PSMB10 | RPSAP58 |
| ARPC1B | COTL1 | HES4 | IFITM3 | ND2 | PSMB9 | SLC25A5 |
| ARPC3 | CST3 | HLA- | JUNB | ND3 | PYCARD | SLC7A7 |
| | | DQB1 | | | | |
| ATP5G3 | CSTB | HLA- | LGALS9 | ND4 | RAC2 | SNHG5 |
| | | DRB1 | | | | |
| ATP6 | CTSC | HLA- | LILRB2 | ND5 | RGS10 | TPT1 |
| | | DRB5 | | | | |
| BIN2 | CYTB | HLA-DMB | LOC100509457 | NDUFB5 | RHOC | TSC22D3 |
| C1orf162 | EEF1A1 | HLA-DMA | LOC606724 | NPM1 | RNR2 | TUBA1B |
| C1QA | EEF1B2 | HN1 | LST1 | PF4 | RNVU1- | UCP2 |
| | | | | | 19 | |
| CD52 | EEF1G | HNRNPA1 | LY6E | PFN1 | RPL7A | UQCRB |
| CD68 | FAM26F | HSPA8 | MS4A7 | PPA1 | RPS24 | YBX1 |

For example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), the method including determining the expression level of one or more genes set forth in any one of Tables 5-7 in a sample from the individual, wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy including a PD-L1 axis binding antagonist.

In another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 5-7 in a sample from the individual, wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)); and (b) selecting an anti-cancer therapy including a PD-L1 axis binding antagonist for the individual based on the expression level of the one or more genes set forth in any one of Tables 5-7 determined in step (a).

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of one or more genes set forth in any one of Tables 5-7 in a sample from the individual, wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy including a PD-L1 axis binding antagonist, wherein the response is indicated by OS or ORR.

In another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 5-7 in a sample from the individual, wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), wherein the response is indicated by OS or ORR; and (b) selecting an anti-cancer therapy including a PD-L1 axis binding antagonist for the individual based on the expression level of the one or more genes set forth in any one of Tables 5-7 determined in step (a).

In yet another example, provided herein is a method of identifying an individual having a cancer (e.g., bladder cancer (e.g., UC) or kidney cancer (e.g., RCC)) who is likely to respond to treatment with an anti-cancer therapy including a PD-L1 binding antagonist (e.g., an anti-PD-L1 antibody such as atezolizumab), the method including determining the expression level of one or more genes set forth in any one of Tables 5-7 in a sample from the individual, wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy including a PD-L1 binding antagonist, wherein the response is indicated by OS or ORR.

In a further example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 5-7 in a sample from the individual, wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy including a PD-L1 binding antagonist (e.g., an anti-PD-L1 antibody such as atezolizumab), wherein the response is indicated by OS or ORR; and (b) selecting an anti-cancer therapy including a PD-L1 binding antagonist for the individual based on the expression level of the one or more genes set forth in any one of Tables 5-7 determined in step (a).

In another example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of one or more genes set forth in any one of Tables 5-7 in a plasma sample or a sample including PBMCs from the individual, wherein an expression level of the one or more genes set forth in any one of Tables 5-7 in the plasma sample or the sample including PBMCs that is at or above a reference level of the one or more genes set forth in any one of Tables 5-7 identifies the individual as likely to respond to treatment with an anti-cancer therapy including a PD-L1 axis binding antagonist. In some instances, the sample is a plasma sample. In other instances, the sample includes PBMCs.

In yet another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 5-7 in a plasma sample or a sample including PBMCs from the individual, wherein an expression level of the one or more genes set forth in any one of Tables 5-7 in the plasma sample or the sample including PBMCs that is at or above a reference level of the one or more genes set forth in any one of Tables 5-7 identifies the individual as likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)); and (b) selecting a therapy including a PD-L1 axis binding antagonist for the individual based on the expression level of the one or more genes set forth in any one of Tables 5-7 in the plasma sample or in the sample including PBMCs determined in step (a). In some instances, the sample is a plasma sample. In other instances, the sample includes PBMCs.

In another example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of one or more genes set forth in any one of Tables 5-7 in a sample obtained from the individual at a time point following administration of the anti-cancer therapy, wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy including a PD-L1 axis binding antagonist.

In yet another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 5-7 in a sample obtained from the individual at a time point following administration of the anti-cancer therapy, wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)); and (b) selecting an anti-cancer therapy including a PD-L1 axis binding antagonist for the individual based on the expression level of the one or more genes set forth in any one of Tables 5-7 determined in step (a).

In another example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of one or more genes set forth in any one of Tables 5-7 in a sample obtained from the individual at a time point following administration of the anti-cancer therapy, wherein the sample is a plasma sample, a tumor tissue sample, or a sample including PBMCs, and wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy including a PD-L1 axis binding antagonist.

In yet another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 5-7 in a sample obtained from the individual at a time point following administration of the anti-cancer therapy, wherein the sample is a plasma sample, a tumor tissue sample, or a sample including PBMCs, and wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy including a PD-L1 axis binding antagonist; and (b) selecting an anti-cancer therapy including a PD-L1 axis binding antagonist for the individual based on the expression level of the one or more genes set forth in any one of Tables 5-7 determined in step (a).

Any suitable time point following administration of the anti-cancer therapy may be used. For example, the time point following administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) following administration of the anti-cancer therapy. In some instances, the time point following administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks following administration of the anti-cancer therapy. In some instances, the time point following administration of the anti-cancer therapy is about 4 to about 8 weeks following administration of the anti-cancer therapy. For example, in some aspects, the time point following administration of the anti-cancer therapy is about 6 weeks following administration of the anti-cancer therapy.

In another example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of one or more genes set forth in any one of Tables 5-7 in a sample from the individual that has been obtained at a time point prior to or concurrently with administration of an anti-cancer therapy including a PD-L1 axis binding antagonist, wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy including a PD-L1 axis binding antagonist.

In yet another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 5-7 in a sample from the individual that has been obtained at a time point prior to or concurrently with administration of an anti-cancer therapy including a PD-L1 axis binding antagonist (e.g., an anti-PD-L1 antibody such as atezolizumab) or a PD-1 binding antagonist (e.g., an anti-PD-1 antibody)), wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy including a PD-L1 axis binding antagonist; and (b) selecting an anti-cancer therapy including a PD-L1 axis binding antagonist for the individual based on the expression level of the one or more genes set forth in any one of Tables 5-7 determined in step (a).

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of identifying an individual having a cancer (e.g., bladder cancer (e.g., UC) or kidney cancer (e.g., RCC)) who is likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)), the method including determining the expression level of one or more genes set forth in any one of Tables 5-7 in a sample from the individual that has been obtained at a time point prior to or concurrently with administration of an anti-cancer therapy including a PD-L1 axis binding antagonist, wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy including a PD-L1 axis binding antagonist, wherein the response is indicated by OS or ORR.

In another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 5-7 in a sample from the individual that has been obtained at a time point prior to or concurrently with administration of an anti-cancer therapy including a PD-L1 axis binding antagonist (e.g., an anti-PD-L1 antibody such as atezolizumab) or a PD-1 binding antagonist (e.g., an anti-PD-1 antibody)), wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy including a PD-L1 axis binding antagonist, wherein the response is indicated by OS or ORR; and (b) selecting an anti-cancer therapy including a PD-L1 axis binding antagonist for the individual based on the expression level of the one or more genes set forth in any one of Tables 5-7 determined in step (a).

Any suitable time point prior to administration of the anti-cancer therapy may be used. For example, the time point prior to administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) prior to administration of the anti-cancer therapy. In some instances, the time point prior to administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks prior to administration of the anti-cancer therapy.

In any of the preceding methods, the individual may be likely to have improved response from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). For example, the individual may be likely to have an improved ORR, improved CR rate, improved PR rate, extended PFS, and/or extended OS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In some particular instances, the individual may be likely to have an improved ORR from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In other particular instances, the individual may be likely to have an extended PFS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In yet other particular instances, the individual may be likely to have an extended OS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist).

In other aspects of the present disclosure, decreased or low levels of one or more genes set forth in any one of Tables 5-7 can be used to identify and/or select individuals who are less likely to respond to treatment with an anti-cancer therapy including a PD-L1 binding antagonist monotherapy. For example, decreased or low baseline or on-treatment expression levels of one or more genes set forth in any one of Tables 5-7 (e.g., relative to a reference level of the one or more genes set forth in any one of Tables 5-7) can be used to identify individuals who are less likely to respond to treatment with an anti-cancer therapy including a PD-L1 binding antagonist monotherapy. Such patients may be candidates for treatment with an anti-cancer therapy other than or in addition to a PD-L1 axis binding antagonist (e.g., combination therapy with a PD-L1 axis binding antagonist and one or more additional therapeutic agents (e.g., VEGF antagonists (e.g., anti-VEGF antibodies such as bevacizumab), or an anti-cancer therapy that does not include a PD-L1 axis binding antagonist).

For example, provided herein is a method of identifying an individual having a cancer (e.g., bladder cancer (e.g., UC) or kidney cancer (e.g., RCC)) who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of one or more genes set forth in any one of Tables 5-7 in a sample from the individual, wherein an expression level of the one or more genes set forth in any one of Tables 5-7 in the sample that is below a reference level of IL8 identifies the individual as less likely to respond to treatment with an anti-cancer therapy including a PD-L1 axis binding antagonist monotherapy.

In another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 5-7 in a sample from the individual, wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy; and (b) selecting an anti-cancer therapy other than or in addition to a PD-L1 axis binding antagonist for the individual based on the expression level of the one or more genes set forth in any one of Tables 5-7 determined in step (a).

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of identifying an individual having a cancer (e.g., bladder cancer (e.g., UC) or kidney cancer (e.g., RCC)) who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of one or more genes set forth in any one of Tables 5-7 in a sample from the individual, wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy including a PD-L1 axis binding antagonist monotherapy, and wherein the response is indicated by OS or ORR.

In another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 5-7 in a sample from the individual, wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, wherein the response is indicated by OS or ORR; and (b) selecting an anti-cancer therapy other than or in addition to a PD-L1 axis binding antagonist for the individual based on the expression level of the one or more genes set forth in any one of Tables 5-7 determined in step (a).

For example, provided herein is a method of identifying an individual having a cancer (e.g., bladder cancer (e.g., UC) or kidney cancer (e.g., RCC)) who is less likely to respond to treatment with an anti-cancer therapy including a PD-L1 binding antagonist (e.g., an anti-PD-L1 antibody such as atezolizumab), the method including determining the expression level of one or more genes set forth in any one of Tables 5-7 in a sample from the individual, wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy including a PD-L1 binding antagonist monotherapy, and wherein the response is indicated by OS or ORR.

In another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 5-7 in a sample from the individual, wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy including a PD-L1 binding antagonist (e.g., an anti-PD-L1 antibody such as atezolizumab) monotherapy, wherein the response is indicated by OS or ORR; and (b) selecting an anti-cancer therapy other than or in addition to a PD-L1 binding antagonist for the individual based on the expression level of IL8 determined in step (a).

In another example, provided herein is a method of identifying an individual having a cancer (e.g., bladder cancer (e.g., UC) or kidney cancer (e.g., RCC)) who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of one or more genes set forth in any one of Tables 5-7 in a plasma sample or a sample including PBMCs from the individual, wherein an expression level of the one or more genes set forth in any one of Tables 5-7 in the plasma sample or the sample including PBMCs that is below a reference level of the one or more genes set forth in any one of Tables 5-7 identifies the individual as less likely to respond to treatment with an anti-cancer therapy including a PD-L1 axis binding antagonist monotherapy.

In yet another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 5-7 in a plasma sample or a sample including PBMCs from the individual, wherein an expression level of the one or more genes set forth in any one of Tables 5-7 in the plasma sample or the sample including PBMCs that is below a reference level of the one or more genes set forth in any one of Tables 5-7 identifies the individual as less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy; and (b) selecting a therapy other than or in addition to a PD-L1 axis binding antagonist for the individual based on the expression level of the one or more genes set forth in any one of Tables 5-7 in the plasma sample or in the sample including PBMCs determined in step (a).

In another example, provided herein is a method of identifying an individual having a cancer who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of one or more genes set forth in any one of Tables 5-7 in a sample obtained from the individual at a time point following administration of an anti-cancer therapy including a PD-L1 axis binding antagonist, wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy including a PD-L1 axis binding antagonist monotherapy.

In another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 5-7 in a sample obtained from the individual at a time point following administration of an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy including a PD-L1 axis binding antagonist monotherapy; and (b) selecting an anti-cancer therapy other than or in addition to a PD-L1 axis binding antagonist for the individual based on the expression level of the one or more genes set forth in any one of Tables 5-7 determined in step (a).

In another example, provided herein is a method of identifying an individual having a cancer who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of one or more genes set forth in any one of Tables 5-7 in a sample obtained from the individual at a time point following administration of an anti-cancer therapy including a PD-L1 axis binding antagonist, wherein the sample is a plasma sample, a tumor tissue sample, or a sample including PBMCs, and wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy including a PD-L1 axis binding antagonist monotherapy.

In yet another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 5-7 in a sample obtained from the individual at a time point following administration of an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein the sample is a plasma sample, a tumor tissue sample, or a sample including PBMCs, and wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy including a PD-L1 axis binding antagonist monotherapy; and (b) selecting an anti-cancer therapy other than or in addition to a PD-L1 axis binding antagonist for the individual based on the expression level of the one or more genes set forth in any one of Tables 5-7 determined in step (a).

Any suitable time point following administration of the anti-cancer therapy may be used. For example, the time point following administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) following administration of the anti-cancer therapy. In some instances, the time point following administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks following administration of the anti-cancer therapy. In some instances, the time point following administration of the anti-cancer therapy is about 4 to about 8 weeks following administration of the anti-cancer therapy. For example, in some aspects, the time point following administration of the anti-cancer therapy is about 6 weeks following administration of the anti-cancer therapy.

In another example, provided herein is a method of identifying an individual having a cancer (e.g., bladder cancer (e.g., UC) or kidney cancer (e.g., RCC)) who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of one or more genes set forth in any one of Tables 5-7 in a sample from the individual that has been obtained at a time point prior to or concurrently with administration of an anti-cancer therapy including a PD-L1 axis binding antagonist, wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy including a PD-L1 axis binding antagonist monotherapy.

In yet another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 5-7 in a sample from the individual that has been obtained at a time point prior to or concurrently with administration of an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy including a PD-L1 axis binding antagonist monotherapy; and (b) selecting an anti-cancer therapy other than or in addition to a PD-L1 axis binding antagonist for the individual based on the expression level of IL8 determined in step (a).

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of identifying an individual having a cancer (e.g., bladder cancer (e.g., UC) or kidney cancer (e.g., RCC)) who is less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy, the method including determining the expression level of one or more genes set forth in any one of Tables 5-7 in a sample from the individual that has been obtained at a time point prior to or concurrently with administration of an anti-cancer therapy including a PD-L1 axis binding antagonist, wherein an expression level of IL8 in the sample that is below a reference level of IL8 identifies the individual as less likely to respond to treatment with an anti-cancer therapy including a PD-L1 axis binding antagonist monotherapy, and wherein the response is indicated by OS or ORR.

In another example, provided herein is a method for selecting a therapy for an individual having a cancer (e.g., bladder cancer (e.g., UC) or kidney cancer (e.g., RCC)), the method including: (a) determining the expression level of one or more genes set forth in any one of Tables 5-7 in a sample from the individual that has been obtained at a time point prior to or concurrently with administration of an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy including a PD-L1 axis binding antagonist monotherapy, wherein the response is indicated by OS or ORR; and (b) selecting an anti-cancer therapy other than or in addition to a PD-L1 axis binding antagonist for the individual based on the expression level of the one or more genes set forth in any one of Tables 5-7 determined in step (a).

Any suitable time point prior to administration of the anti-cancer therapy may be used. For example, the time point prior to administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) prior to administration of the anti-cancer therapy. In some instances, the time point prior to administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks prior to administration of the anti-cancer therapy.

In any of the preceding methods, the individual may be likely to have a reduced response from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). For example, the individual may be likely to have a reduced ORR, reduced CR rate, reduced PR rate, shortened PFS, or shortened OS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In some particular instances, the individual may be likely to have a reduced ORR from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In other particular instances, the individual may be likely to have a shortened PFS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In other particular instances, the individual may be likely to have a shortened OS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist).

4. Samples and Reference Levels

Any suitable sample may be used in any of the methods described herein. Exemplary, non-limiting samples include a plasma sample, a tissue sample, a cell sample, a whole blood sample, a serum sample, or a combination thereof. For example, in some instances, the sample is a plasma sample. In another example, in some instances, the tissue sample is a tumor tissue sample. In some instances, the tumor tissue sample includes tumor cells, tumor-infiltrating immune cells, stromal cells, or a combination thereof. In some instances, the tumor-infiltrating immune cells include tumor-infiltrating myeloid cells. In some instances, the tumor tissue sample is a formalin-fixed and paraffin-embedded (FFPE) sample, an archival sample, a fresh sample, or a frozen sample. In some instances, the cell sample includes peripheral blood mononuclear cells (PBMCs).

Any suitable reference level may be used in any of the methods described herein. For example, in some instances, the reference level of a biomarker described herein, e.g., one or more genes set forth in any one of Tables 1-7, e.g., IL8, is determined from a population of individuals having a cancer. For example, in some instances, the reference level of a biomarker described herein, e.g., one or more genes set forth in any one of Tables 1-7, e.g., IL8, is a median expression level, a tertile expression level, or a maximally-selected log-rank reference level determined in a population of patients having a cancer. In some embodiments, the maximally-selected log-rank reference level is 12 pg/mL of IL8. In some instances, the reference level of a biomarker described herein, e.g., one or more genes set forth in any one of Tables 1-7, e.g., IL8, is a median expression level determined in a population of patients having a cancer. In one particular example, the reference level may be the level of a biomarker described herein, e.g., one or more genes set forth in any one of Tables 1-7, e.g., IL8, in a sample obtained from the individual at a time point prior to or concurrently with administration of the anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)). In another particular example, the reference level may be the level of a biomarker described herein, e.g., one or more genes set forth in any one of Tables 1-7, e.g., IL8, in a sample obtained from the individual at a time following administration of the anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)).

In certain instances, the presence and/or expression levels/amount of a biomarker (e.g., a biomarker described herein, e.g., one or more genes set forth in any one of Tables 1-7, e.g., IL8) in a first sample is increased or elevated as compared to presence/absence and/or expression levels/amount in a second sample. In certain instances, the presence/absence and/or expression levels/amount of a biomarker (e.g., a biomarker described herein, e.g., one or more genes set forth in any one of Tables 1-7, e.g., IL8) in a first sample is decreased or reduced as compared to presence and/or expression levels/amount in a second sample. In certain instances, the second sample is a reference sample, reference cell, reference tissue, control sample, control cell, or control tissue. Additional disclosures for determining the presence/absence and/or expression levels/amount of a gene are described herein.

In certain instances, a reference sample, reference cell, reference tissue, control sample, control cell, or control tissue is a single sample or a combination of multiple samples from the same subject or individual that are obtained at one or more different time points than when the test sample is obtained. For example, a reference sample, reference cell, reference tissue, control sample, control cell, or control tissue is obtained at an earlier time point from the same subject or individual than when the test sample is obtained. Such reference sample, reference cell, reference tissue, control sample, control cell, or control tissue may be useful if the reference sample is obtained during initial diagnosis of cancer and the test sample is later obtained when the cancer becomes metastatic.

In certain instances, a reference sample, reference cell, reference tissue, control sample, control cell, or control tissue is a combination of multiple samples from one or more healthy individuals who are not the patient. In certain embodiments, a reference sample, reference cell, reference tissue, control sample, control cell, or control tissue is a combination of multiple samples from one or more individuals with a disease or disorder (e.g., cancer) who are not the subject or individual. In certain embodiments, a reference sample, reference cell, reference tissue, control sample, control cell, or control tissue is pooled RNA samples from normal tissues or pooled plasma or serum samples from one or more individuals who are not the patient. In certain embodiments, a reference sample, reference cell, reference tissue, control sample, control cell, or control tissue is pooled RNA samples from tumor tissues or pooled plasma or serum samples from one or more individuals with a disease or disorder (e.g., cancer) who are not the patient.

In some examples of any of the methods, elevated or increased expression refers to an overall increase of about any of 10%, 20%, 30%, 40%, 50%, 60%, 70%, 80%, 90%, 95%, 96%, 97%, 98%, 99% or greater, in the level of biomarker (e.g., a biomarker described herein, e.g., one or more genes set forth in any one of Tables 1-7, e.g., IL8) (e.g., protein or nucleic acid (e.g., gene or mRNA)), detected by standard art-known methods such as those described herein, as compared to a reference sample, reference cell, reference tissue, control sample, control cell, or control tissue. In certain embodiments, the elevated expression refers to the increase in expression level/amount of a biomarker in the sample wherein the increase is at least about any of 1.5×, 1.75×, 2×, 3×, 4×, 5×, 6×, 7×, 8×, 9×, 10×, 25×, 50×, 75×, or 100× the expression level/amount of the respective biomarker in a reference sample, reference cell, reference tissue, control sample, control cell, or control tissue. In some embodiments, elevated expression refers to an overall increase of greater than about 1.5-fold, about 1.75-fold, about 2-fold, about 2.25-fold, about 2.5-fold, about 2.75-fold, about 3.0-fold, or about 3.25-fold as compared to a reference sample, reference cell, reference tissue, control sample, control cell, control tissue, or internal control (e.g., housekeeping gene).

In some examples of any of the methods, reduced expression refers to an overall reduction of about any of 10%, 20%, 30%, 40%, 50%, 60%, 70%, 80%, 90%, 95%, 96%, 97%, 98%, 99% or greater, in the level of biomarker (e.g., a biomarker described herein, e.g., one or more genes set forth in any one of Tables 1-7, e.g., IL8) (e.g., protein or nucleic acid (e.g., gene or mRNA)), detected by standard art known methods such as those described herein, as compared to a reference sample, reference cell, reference tissue, control sample, control cell, or control tissue. In certain embodiments, reduced expression refers to the decrease in expression level/amount of a biomarker in the sample wherein the decrease is at least about any of 0.9×, 0.8×, 0.7×, 0.6×, 0.5×, 0.4×, 0.3×, 0.2×, 0.1×, 0.05×, or 0.01× the expression level/amount of the respective biomarker in a reference sample, reference cell, reference tissue, control sample, control cell, or control tissue.

5. Determination of the Presence and/or Expression Levels of Biomarkers

The presence and/or expression levels/amount of a biomarker (e.g., a biomarker described herein, e.g., one or more genes set forth in any one of Tables 1-7, e.g., IL8) can be determined qualitatively and/or quantitatively based on any suitable criterion known in the art, including but not limited to DNA, mRNA, cDNA, proteins, protein fragments, and/or gene copy number. The presence and/or expression level/amount of various biomarkers described herein (e.g., a biomarker described herein, e.g., one or more genes set forth in any one of Tables 1-7, e.g., IL8) in a sample can be analyzed by a number of methodologies, many of which are known in the art and understood by the skilled artisan, including, but not limited to, immunohistochemistry (“IHC”), Western blot analysis, immunoprecipitation, molecular binding assays, ELISA, ELIFA, fluorescence activated cell sorting (“FACS”), MassARRAY, proteomics, quantitative blood based assays (e.g., Serum ELISA), biochemical enzymatic activity assays, in situ hybridization, fluorescence in situ hybridization (FISH), Southern analysis, Northern analysis, whole genome sequencing, polymerase chain reaction (PCR) including quantitative real time PCR (qRT-PCR) and other amplification type detection methods, such as, for example, branched DNA, SISBA, TMA and the like, RNA-seq (e.g., scRNA-seq), microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols in Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

In any of methods described herein, the expression level of a biomarker (e.g., a biomarker described herein, e.g., one or more genes set forth in any one of Tables 1-7, e.g., IL8) may be a nucleic acid expression level. In some instances, the nucleic acid expression level is determined using qPCR, rtPCR, RNA-seq (e.g., scRNA-seq), multiplex qPCR or RT-qPCR, microarray analysis, SAGE, MassARRAY technique, or in situ hybridization (e.g., FISH). In some instances, the expression level of a biomarker (e.g., IL8) is determined in tumor cells, tumor infiltrating immune cells, PBMCs, stromal cells, or combinations thereof. In some instances, the expression level of a biomarker (e.g., a biomarker described herein, e.g., one or more genes set forth in any one of Tables 1-7, e.g., IL8) is determined in tumor-infiltrating immune cells. In some instances, the expression level of a biomarker (e.g., a biomarker described herein, e.g., one or more genes set forth in any one of Tables 1-7, e.g., IL8) is determined in tumor cells. In particular instances, the expression level of a biomarker (e.g., a biomarker described herein, e.g., one or more genes set forth in any one of Tables 1-7, e.g., IL8) is determined in PBMCs.

Methods for the evaluation of mRNAs in cells are well known and include, for example, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like). In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined. Optional methods include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of treatment comprising a PD-L1 axis binding antagonist may be arrayed on a solid support. Hybridization of a labeled probe with a particular array member indicates that the sample from which the probe was derived expresses that gene.

In particular instances, the mRNA expression level is determined by RNA sequencing (RNA-seq), real-time-quantitative polymerase chain reaction (RT-qPCR), quantitative PCR (qPCR), multiplex qPCR or RT-qPCR, microarray analysis, serial analysis of gene expression (SAGE), MassARRAY technique, in situ hybridization (ISH), or a combination thereof. In some particular instances, the RNA-seq is single-cell RNA-seq (scRNA-seq).

In any of the methods described herein, the presence and/or expression level/amount of a biomarker (e.g., a biomarker described herein, e.g., one or more genes set forth in any one of Tables 1-7, e.g., IL8) is measured by determining protein expression levels of the biomarker. In certain instances, the method comprises contacting the biological sample with antibodies that specifically bind to a biomarker (e.g., antibodies that specifically bind to any biomarker described herein, e.g., one or more genes set forth in any one of Tables 1-7, e.g., anti-IL8 antibodies) described herein under conditions permissive for binding of the biomarker, and detecting whether a complex is formed between the antibodies and biomarker. Such method may be an in vitro or in vivo method. In some instances, an antibody is used to select subjects eligible for therapy with a PD-L1 axis binding antagonist, e.g., a biomarker for selection of individuals. Any method of measuring protein expression levels known in the art or provided herein may be used. For example, in some instances, a protein expression level of a biomarker (e.g., a biomarker described herein, e.g., one or more genes set forth in any one of Tables 1-7, e.g., IL8) is determined using a method selected from the group consisting of flow cytometry (e.g., fluorescence-activated cell sorting (FACS™)), Western blot, enzyme-linked immunosorbent assay (ELISA), immunoprecipitation, immunohistochemistry (IHC), immunofluorescence, radioimmunoassay, dot blotting, immunodetection methods, HPLC, surface plasmon resonance, optical spectroscopy, mass spectrometry, and HPLC. In some instances, the protein expression level of the biomarker (e.g., a biomarker described herein, e.g., one or more genes set forth in any one of Tables 1-7, e.g., IL8) is determined in tumor-infiltrating immune cells. In some instances, the protein expression level of the biomarker (e.g., a biomarker described herein, e.g., one or more genes set forth in any one of Tables 1-7, e.g., IL8) is determined in tumor cells. In some instances, the protein expression level of the biomarker (e.g., a biomarker described herein, e.g., one or more genes set forth in any one of Tables 1-7, e.g., IL8) is determined in tumor-infiltrating immune cells and/or in tumor cells. In some instances, the protein expression level is determined by immunohistochemistry (IHC), Western blot, enzyme-linked immunosorbent assay (ELISA), immunoprecipitation, immunofluorescence, radioimmunoassay, or mass spectrometry.

In certain instances, the presence and/or expression level/amount of a biomarker protein (e.g., a biomarker described herein, e.g., one or more genes set forth in any one of Tables 1-7, e.g., IL8) in a sample is examined using IHC and staining protocols. IHC staining of tissue sections has been shown to be a reliable method of determining or detecting the presence of proteins in a sample. In some instances of any of the methods, assays and/or kits, the biomarker is IL8. In one instance, expression level of biomarker (e.g., a biomarker described herein, e.g., one or more genes set forth in any one of Tables 1-7, e.g., IL8) is determined using a method comprising: (a) performing IHC analysis of a sample (such as a tumor sample obtained from a patient) with an antibody; and (b) determining expression level of a biomarker in the sample. In some instances, IHC staining intensity is determined relative to a reference. In some instances, the reference is a reference value. In some instances, the reference is a reference sample (e.g., a control cell line staining sample, a tissue sample from non-cancerous patient, or a biomarker-negative (e.g., an IL8-negative) tumor sample).

IHC may be performed in combination with additional techniques such as morphological staining and/or in situ hybridization (e.g., FISH). Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for IHC typically will be labeled with a detectable moiety. Numerous labels are available which can be generally grouped into the following categories: (a) radioisotopes, such as ³⁵S, ¹⁴C, ¹²⁵I, ³H, and ¹³¹I; (b) colloidal gold particles; (c) fluorescent labels including, but are not limited to, rare earth chelates (europium chelates), Texas Red, rhodamine, fluorescein, dansyl, lissamine, umbelliferone, phycocrytherin, phycocyanin, or commercially-available fluorophores such as SPECTRUM ORANGE7 and SPECTRUM GREEN7 and/or derivatives of any one or more of the above; (d) various enzyme-substrate labels are available and U.S. Pat. No. 4,275,149 provides a review of some of these. Examples of enzymatic labels include luciferases (e.g., firefly luciferase and bacterial luciferase; see, e.g., U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Examples of enzyme-substrate combinations include, for example, horseradish peroxidase (HRPO) with hydrogen peroxidase as a substrate; alkaline phosphatase (AP) with para-Nitrophenyl phosphate as chromogenic substrate; and β-D-galactosidase (β-D-Gal) with a chromogenic substrate (e.g., β-nitrophenyl-β-D-galactosidase) or fluorogenic substrate (e.g., 4-methylumbelliferyl-β-D-galactosidase). For a general review of these, see, for example, U.S. Pat. Nos. 4,275,149 and 4,318,980.

Specimens may be prepared, for example, manually, or using an automated staining instrument (e.g., a Ventana BenchMark XT or Benchmark ULTRA instrument). Specimens thus prepared may be mounted and coverslipped. Slide evaluation is then determined, for example, using a microscope, and staining intensity criteria, routinely used in the art, may be employed. In one instance, it is to be understood that when cells and/or tissue from a tumor is examined using IHC, staining is generally determined or assessed in tumor cell(s) and/or tissue (as opposed to stromal or surrounding tissue that may be present in the sample). In some instances, it is understood that when cells and/or tissue from a tumor is examined using IHC, staining includes determining or assessing in tumor-infiltrating immune cells, including intratumoral or peritumoral immune cells. In some instances, the presence of a biomarker (e.g., a biomarker described herein, e.g., one or more genes set forth in any one of Tables 1-7, e.g., IL8) is detected by IHC in >0% of the sample, in at least 1% of the sample, in at least 5% of the sample, in at least 10% of the sample, in at least 15% of the sample, in at least 15% of the sample, in at least 20% of the sample, in at least 25% of the sample, in at least 30% of the sample, in at least 35% of the sample, in at least 40% of the sample, in at least 45% of the sample, in at least 50% of the sample, in at least 55% of the sample, in at least 60% of the sample, in at least 65% of the sample, in at least 70% of the sample, in at least 75% of the sample, in at least 80% of the sample, in at least 85% of the sample, in at least 90% of the sample, in at least 95% of the sample, or more. Samples may be scored using any of the criteria described herein, for example, by a pathologist or automated image analysis.

In some instances of any of the methods described herein, a biomarker (e.g., a biomarker described herein, e.g., one or more genes set forth in any one of Tables 1-7, e.g., IL8) is detected by immunohistochemistry using a diagnostic antibody (i.e., primary antibody). In some instances, the diagnostic antibody specifically binds to human biomarker (e.g., a biomarker described herein, e.g., one or more genes set forth in any one of Tables 1-7, e.g., IL8). In some instances, the diagnostic antibody is a non-human antibody. In some instances, the diagnostic antibody is a rat, mouse, or rabbit antibody. In some instances, the diagnostic antibody is a rabbit antibody. In some instances, the diagnostic antibody is a monoclonal antibody. In some instances, the diagnostic antibody is directly labeled. In other instances, the diagnostic antibody is indirectly labeled.

For example, in some instances of any of the methods described herein, IL8 is detected by immunohistochemistry using an anti-IL8 diagnostic antibody (i.e., primary antibody). In some instances, the IL8 diagnostic antibody specifically binds human IL8. In some instances, the IL8 diagnostic antibody is a non-human antibody. In some instances, the IL8 diagnostic antibody is a rat, mouse, or rabbit antibody. In some instances, the IL8 diagnostic antibody is a rabbit antibody. In some instances, the IL8 diagnostic antibody is a monoclonal antibody. In some instances, the IL8 diagnostic antibody is directly labeled. In other instances, the IL8 diagnostic antibody is indirectly labeled.

In some instances of any of the preceding methods, the expression level of a biomarker (e.g., a biomarker described herein, e.g., one or more genes set forth in any one of Tables 1-7, e.g., IL8) is detected in tumor-infiltrating immune cells, tumor cells, PBMCs, or combinations thereof using IHC. Tumor-infiltrating immune cells include, but are not limited to, intratumoral immune cells, peritumoral immune cells or any combinations thereof, and other tumor stroma cells (e.g., fibroblasts). Such tumor infiltrating immune cells may be T lymphocytes (such as CD8+ T lymphocytes and/or CD4+ T lymphocytes), B lymphocytes, or other bone marrow-lineage cells including granulocytes (neutrophils, eosinophils, basophils), monocytes, macrophages, dendritic cells (e.g., interdigitating dendritic cells), histiocytes, and natural killer cells. In some instances, the staining for a biomarker (e.g., a biomarker described herein, e.g., one or more genes set forth in any one of Tables 1-7, e.g., IL8) is detected by the presence of staining in the sample, for example, membrane staining, cytoplasmic staining, and combinations thereof. In other instances, the absence of a biomarker (e.g., a biomarker described herein, e.g., one or more genes set forth in any one of Tables 1-7, e.g., IL8) is detected as absent or no staining in the sample.

In some examples of any of the methods described herein, the individual may have an expression level of a T effector (T_{eff}) signature in a tumor sample that is at or above a reference level for the T_{eff} signature. In some instances, the T_{eff} signature comprises one or more genes selected from CD8A, GZMA, GZMB, and PRF1. In some instances, the T_{eff} signature comprises two or more genes selected from CD8A, GZMA, GZMB, and PRF1. In some instances, the T_{eff} signature comprises three or more genes selected from CD8A, GZMA, GZMB, and PRF1. In some instances, the T_{eff} signature comprises CD8A, GZMA, GZMB, and PRF1.

In some examples of any of the methods described herein, the individual has not been previously treated for the cancer.

In other examples of any of the methods described herein, the individual has previously been treated for the cancer.

The individual may have any suitable type of cancer. Exemplary, non-limiting cancers include a bladder cancer, a kidney cancer, a breast cancer, a colorectal cancer, a lung cancer, a lymphoma, a prostate cancer, a liver cancer, a head and neck cancer, a melanoma, an ovarian cancer, a mesothelioma, or a myeloma. In some aspects, the bladder cancer is urothelial carcinoma (UC). In some aspects, the UC is locally advanced or metastatic UC. In some aspects, the individual has received a prior platinum-based chemotherapy. In some aspects, the individual has progressed after the prior platinum-based chemotherapy. In some aspects, the individual is previously untreated for the locally advanced or metastatic UC. In some aspects, the individual is ineligible for platinum-based chemotherapy. In some aspects, the individual is cisplatin-ineligible. In some aspects, the kidney cancer is renal cell carcinoma (RCC). In some aspects, the RCC is metastatic RCC (mRCC). In some aspects, the individual is previously untreated for the mRCC. In some aspects, the lung cancer is non-small cell lung cancer or small-cell lung cancer. In some aspects, the breast cancer is triple-negative breast cancer (TNBC) or HER2-positive breast cancer.

5. Patient Selection and Therapy Based on Determination of the Presence and/or Expression Level of Biomarkers

Any of the methods describe herein may further include administering an anti-cancer therapy to the individual. For example, in some instances, the anti-cancer therapy is selected based on the determination of the expression level of a biomarker described herein (e.g., one or more genes set forth in any one of Tables 1-7, e.g., IL8) in a sample from the individual. For example, in some instances, the anti-cancer therapy is selected based on the determination of the expression level of IL8 in a sample from the individual.

For example, in some instances, the method further includes administering an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) to the individual.

In other instances, the method further includes administering an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) to the individual. In some instances, the anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) includes a VEGF antagonist (e.g., an anti-VEGF antibody), an IL8 antagonist (e.g., an anti-IL-8 antibody or a small molecule IL8 inhibitor), an IL1B antagonist (e.g., an anti-IL1B antibody or a small molecule IL1B inhibitor), an IL1R antagonist (e.g., an anti-IL1R antibody or a small molecule IL1R inhibitor), or a combination thereof. Any suitable VEGF antagonist, IL8 antagonist, IL1B antagonist, or IL1R antagonist may be used (see, e.g., Section IV below). In some instances, the anti-cancer therapy includes a VEGF antagonist and a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)). In some instances, the VEGF antagonist is an anti-VEGF antibody or a VEGF receptor (VEGFR) inhibitor. In some aspects, the VEGF antagonist is an anti-VEGF antibody. In some aspects, the anti-VEGF antibody is bevacizumab.

Any suitable PD-L1 axis binding antagonist may be used may be used (see, e.g., Section IV below). For example, in some instances, the PD-L1 axis binding antagonist is selected from the group consisting of a PD-L1 binding antagonist, a PD-1 binding antagonist, and a PD-L2 binding antagonist.

In some examples, the PD-L1 axis binding antagonist is a PD-L1 binding antagonist. Any suitable PD-L1 binding antagonist may be used may be used (see, e.g., Section IV below). In some aspects, the PD-L1 binding antagonist inhibits the binding of PD-L1 to one or more of its ligand binding partners. In some aspects, the PD-L1 binding antagonist inhibits the binding of PD-L1 to PD-1. In some aspects, the PD-L1 binding antagonist inhibits the binding of PD-L1 to B7-1. In some aspects, the PD-L1 binding antagonist inhibits the binding of PD-L1 to both PD-1 and B7-1. In some aspects, the PD-L1 binding antagonist is an anti-PD-L1 antibody. In some aspects, the anti-PD-L1 antibody is selected from the group consisting of: atezolizumab, MDX-1105, durvalumab, and avelumab.

In some aspects of any of the methods described herein, the anti-PD-L1 antibody includes one, two, three, four, five, or all six of following hypervariable regions (HVRs): (a) an HVR-H1 sequence of GFTFSDSWIH (SEQ ID NO: 19); (b) an HVR-H2 sequence of AWISPYGGSTYYADSVKG (SEQ ID NO: 20); (c) an HVR-H3 sequence of RHWPGGFDY (SEQ ID NO: 21); (d) an HVR-L1 sequence of RASQDVSTAVA (SEQ ID NO: 22); (e) an HVR-L2 sequence of SASFLYS (SEQ ID NO: 23); and (f) an HVR-L3 sequence of QQYLYHPAT (SEQ ID NO: 24), or one, two, three, four, five, or six HVRs having at least 90% (e.g., at least 90%, at least 91%, at least 92%, at least 93%, at least 94%, at least 95%, at least 96%, at least 97%, at least 98%, or at least 99%) sequence identity to (a) an HVR-H1 sequence of GFTFSDSWIH (SEQ ID NO: 19); (b) an HVR-H2 sequence of AWISPYGGSTYYADSVKG (SEQ ID NO: 20); (c) an HVR-H3 sequence of RHWPGGFDY (SEQ ID NO: 21); (d) an HVR-L1 sequence of RASQDVSTAVA (SEQ ID NO: 22); (e) an HVR-L2 sequence of SASFLYS (SEQ ID NO: 23); and (f) an HVR-L3 sequence of QQYLYHPAT (SEQ ID NO: 24).

In some particular aspects of any of the methods described herein, the anti-PD-L1 antibody includes the following hypervariable regions (HVRs): (a) an HVR-H1 sequence of GFTFSDSWIH (SEQ ID NO: 19); (b) an HVR-H2 sequence of AWISPYGGSTYYADSVKG (SEQ ID NO: 20); (c) an HVR-H3 sequence of RHWPGGFDY (SEQ ID NO: 21); (d) an HVR-L1 sequence of RASQDVSTAVA (SEQ ID NO: 22); (e) an HVR-L2 sequence of SASFLYS (SEQ ID NO: 23); and (f) an HVR-L3 sequence of QQYLYHPAT (SEQ ID NO: 24).

In some aspects of any of the methods described herein, the anti-PD-L1 antibody includes: (a) a heavy chain variable (VH) domain including an amino acid sequence having at least 90% (e.g., at least 90%, at least 91%, at least 92%, at least 93%, at least 94%, at least 95%, at least 96%, at least 97%, at least 98%, or at least 99%) sequence identity to the amino acid sequence of EVQLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSS (SEQ ID NO: 25); (b) a light chain variable (VL) domain including an amino acid sequence having at least 90% (e.g., at least 90%, at least 91%, at least 92%, at least 93%, at least 94%, at least 95%, at least 96%, at least 97%, at least 98%, or at least 99%) sequence identity to the amino acid sequence of DIQMTQSPSSLSASVGDRVTITCRASQDVSTAVAWYQQKPGKAPKLLIYSASFLYSGVPSRFSGSGSGTD FTLTISSLQPEDFATYYCQQYLYHPATFGQGTKVEIKR (SEQ ID NO: 4); or (c) a VH domain as in (a) and a VL domain as in (b). In some aspects, the anti-PD-L1 antibody includes: (a) a VH domain including an amino acid sequence having at least 95% sequence identity to the amino acid sequence of SEQ ID NO: 25; (b) a VL domain including an amino acid sequence having at least 95% sequence identity to the amino acid sequence of SEQ ID NO: 4; or (c) a VH domain as in (a) and a VL domain as in (b). In some aspects, the anti-PD-L1 antibody includes: (a) a VH domain including an amino acid sequence having at least 96% sequence identity to the amino acid sequence of SEQ ID NO: 25; (b) a VL domain including an amino acid sequence having at least 96% sequence identity to the amino acid sequence of SEQ ID NO: 4; or (c) a VH domain as in (a) and a VL domain as in (b). In some aspects, the anti-PD-L1 antibody includes: (a) a VH domain including an amino acid sequence having at least 97% sequence identity to the amino acid sequence of SEQ ID NO: 25; (b) a VL domain including an amino acid sequence having at least 97% sequence identity to the amino acid sequence of SEQ ID NO: 4; or (c) a VH domain as in (a) and a VL domain as in (b). In some aspects, the anti-PD-L1 antibody includes: (a) a VH domain including an amino acid sequence having at least 98% sequence identity to the amino acid sequence of SEQ ID NO: 25; (b) a VL domain including an amino acid sequence having at least 98% sequence identity to the amino acid sequence of SEQ ID NO: 4; or (c) a VH domain as in (a) and a VL domain as in (b). In some aspects, the anti-PD-L1 antibody includes: (a) a VH domain including an amino acid sequence having at least 99% sequence identity to the amino acid sequence of SEQ ID NO: 25; (b) a VL domain including an amino acid sequence having at least 99% sequence identity to the amino acid sequence of SEQ ID NO: 4; or (c) a VH domain as in (a) and a VL domain as in (b). In some aspects, the anti-PD-L1 antibody includes: (a) a VH domain including the amino acid sequence of SEQ ID NO: 25; (b) a VL domain including the amino acid sequence of SEQ ID NO: 4; or (c) a VH domain as in (a) and a VL domain as in (b). In some aspects, the anti-PD-L1 antibody includes: (a) a VH domain including the amino acid sequence of SEQ ID NO: 25; and (b) a VL domain including the amino acid sequence of SEQ ID NO: 4. In some aspects, the anti-PD-L1 antibody is atezolizumab.

Any of the methods described herein may further include administering an additional therapeutic agent to the individual. In some aspects, the additional therapeutic agent is selected from the group consisting of an immunotherapy agent, a cytotoxic agent, a growth inhibitory agent, a radiation therapy agent, an anti-angiogenic agent, and combinations thereof.

In some aspects of any of the methods described herein, the individual is a human.

B. Therapeutic Methods and Compositions for Use

The present disclosure provides of treating an individual having a cancer (e.g., bladder cancer (e.g., UC) or kidney cancer (e.g., RCC)). In some instances, the methods of the present disclosure include administering to the patient an anti-cancer therapy that includes a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)). Any of the PD-L1 axis binding antagonists described herein (see, for example, Section IV, below) or known in the art may be used in the methods. In some instances, the methods involve determining the presence and/or expression level of a biomarker described herein (e.g., one or more genes set forth in any one of Tables 1-7, e.g., IL8) in a sample obtained from an individual and administering an anti-cancer therapy to the patient based on the presence and/or expression level of the biomarker in the sample, for example, using any of the methods described herein (for example, those described in Section III, Subsection A above or in the Examples below) or known in the art. For example, provided herein are methods of treating an individual having a cancer who has been determined to be likely to respond to treatment with an anti-cancer therapy comprising a PD-L1 axis binding antagonist based on an expression level of IL8 in a sample from the individual that is below a reference level of IL8. Also provided herein are methods of treating an individual having a cancer who has been determined to be less likely to respond to treatment with an anti-cancer therapy comprising a PD-L1 axis binding antagonist monotherapy based on an expression level of IL8 in a sample from the individual that is at or above a reference level of IL8. As is also described herein, the invention also provides genes that are upregulated or downregulated in the context of IL8 high cancer, e.g., in tumor samples and/or in PBMCs. See, e.g., Example 1. The expression level of one or more genes that are upregulated or downregulated in the context of IL8 high cancer, e.g., one or more genes set forth in any one of Tables 2-7, can be used as proxies for IL8 expression, e.g., in methods of treating an individual having a cancer who has been determined to be likely to respond to treatment with an anti-cancer therapy comprising a PD-L1 axis binding antagonist based on an expression level of one or more genes set forth in any one of Tables 2-7 in a sample from the individual that is below a reference level of the one or more genes and in methods of treating an individual having a cancer who has been determined to be less likely to respond to treatment with an anti-cancer therapy comprising a PD-L1 axis binding antagonist monotherapy based on an expression level of one or more genes set forth in any one of Tables 2-7 in a sample from the individual that is at or above a reference level of the one or more genes. Also provided herein are methods of monitoring the response of an individual having a cancer to treatment with an anti-cancer therapy that includes a PD-L1 axis binding antagonist. Also provided are related compositions for use.

1. IL8

In several aspects of the present disclosure, individuals having decreased or low IL8 levels may be treated with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)). For example, individuals having decreased or low baseline or on-treatment IL8 expression levels (e.g., relative to a reference level of IL8) may be treated with an anti-cancer therapy including a PD-L1 axis binding antagonist.

In one aspect, provided herein is a method of treating an individual having a cancer (e.g., bladder cancer (e.g., UC) or kidney cancer (e.g., RCC)), the method comprising: (a) determining the expression level of IL8 in a sample from the individual, wherein an expression level of IL8 in the sample that is below a reference level of IL8 identifies the individual as likely to respond to treatment with an anti-cancer therapy comprising a PD-L1 axis binding antagonist; and (b) administering an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)) to the individual based on the expression level of IL8 determined in step (a).

In another aspect, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 axis binding antagonist based on an expression level of IL8 in a sample from the individual that is below a reference level of IL8, the method comprising administering to the individual an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)).

In another aspect, provided herein is a PD-L1 axis binding antagonist for use in treating an individual having a cancer (e.g., bladder cancer (e.g., UC) or kidney cancer (e.g., RCC)), wherein the individual has been identified as likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)) based on an expression level of IL8 in a sample from the individual that is below a reference level of IL8.

The response may be any suitable response. For example, in some aspects, the response is indicated by ORR, CR rate, PR rate, PFS, OS, and/or DOR. In some instances, the response is indicated by ORR or OS. In some particular instances, the response is indicated by ORR. In other particular instances, the response is indicated by PFS. In yet other particular instances, the response is indicated by OS.

For example, provided herein is a method of treating an individual having a cancer (e.g., bladder cancer (e.g., UC) or kidney cancer (e.g., RCC)), the method comprising: (a) determining the expression level of IL8 in a sample from the individual, wherein an expression level of IL8 in the sample that is below a reference level of IL8 identifies the individual as likely to respond to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)), wherein the response is indicated by overall survival (OS) or overall response rate (ORR); and (b) administering an effective amount of an anti-cancer therapy comprising a PD-L1 binding antagonist to the individual based on the expression level of IL8 determined in step (a).

In another example, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 axis binding antagonist based on an expression level of IL8 in a sample from the individual that is below a reference level of IL8, the method comprising administering to the individual an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)), wherein the response is indicated by OS or ORR.

In another aspect, provided herein is a PD-L1 axis binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the individual has been identified as likely to respond to treatment with an anti-cancer therapy including a PD-L1 binding antagonist based on an expression level of IL8 in a sample from the individual that is below a reference level of IL8, wherein the response is indicated by OS or ORR.

For example, provided herein is a method of treating an individual having a cancer (e.g., bladder cancer (e.g., UC) or kidney cancer (e.g., RCC)), the method comprising: (a) determining the expression level of IL8 in a sample from the individual, wherein an expression level of IL8 in the sample that is below a reference level of IL8 identifies the individual as likely to respond to treatment with an anti-cancer therapy comprising a PD-L1 binding antagonist (e.g., an anti-PD-L1 antibody such as atezolizumab), wherein the response is indicated by overall survival (OS) or overall response rate (ORR); and (b) administering an effective amount of an anti-cancer therapy comprising a PD-L1 binding antagonist to the individual based on the expression level of IL8 determined in step (a).

In another example, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 binding antagonist based on an expression level of IL8 in a sample from the individual that is below a reference level of IL8, the method comprising administering to the individual an effective amount of an anti-cancer therapy comprising a PD-L1 binding antagonist (e.g., an anti-PD-L1 antibody such as atezolizumab), wherein the response is indicated by OS or ORR.

In another aspect, provided herein is a PD-L1 binding antagonist (e.g., an anti-PD-L1 antibody such as atezolizumab) for use in treating an individual having a cancer (e.g., bladder cancer (e.g., UC) or kidney cancer (e.g., RCC)), wherein the individual has been identified as likely to respond to treatment with an anti-cancer therapy including a PD-L1 binding antagonist based on an expression level of IL8 in a sample from the individual that is below a reference level of IL8, wherein the response is indicated by OS or ORR.

In another aspect, provided herein is a method of treating an individual having a cancer (e.g., bladder cancer (e.g., UC) or kidney cancer (e.g., RCC)), the method comprising: (a) determining the expression level of IL8 in a plasma sample or a sample comprising PBMCs from the individual, wherein an expression level of IL8 in the plasma sample or in the sample comprising PBMCs that is below a reference level of IL8 identifies the individual as likely to respond to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)); and (b) administering an effective amount of an anti-cancer therapy comprising a PD-L1 axis binding antagonist to the individual based on the expression level of IL8 determined in the plasma sample or in the sample comprising PBMCs in step (a).

In another aspect, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 axis binding antagonist based on an expression level of IL8 in a plasma sample or a sample comprising PBMCs from the individual that is below a reference level of IL8, the method comprising administering to the individual an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)).

In another aspect, provided herein is a PD-L1 axis binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the individual has been identified as likely to respond to treatment with an anti-cancer therapy including a PD-L1 axis binding antagonist based on an expression level of IL8 in a plasma sample or a sample including PBMCs from the individual that is below a reference level of IL8.

In another aspect, provided herein is a method of treating an individual having a cancer (e.g., bladder cancer (e.g., UC) or kidney cancer (e.g., RCC)), the method comprising: (a) administering an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)) to the individual; (b) determining the expression level of IL8 in a sample obtained from the individual at a time point following administration of the anti-cancer therapy; and (c) continuing to administer the anti-cancer therapy to the individual if the expression level of IL8 in the individual's sample is below the reference level of IL8.

In another aspect, provided herein is a method of monitoring the response of an individual having a cancer (e.g., bladder cancer (e.g., UC) or kidney cancer (e.g., RCC)) to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)), the method comprising: (a) determining the expression level of IL8 in a sample obtained from the individual at a time point following administration of the anti-cancer therapy; (b) comparing the expression level of IL8 in the sample with a reference level of IL8; and (c) continuing to administer the anti-cancer therapy to the individual if the expression level of IL8 in the individual's sample is below the reference level.

In another aspect, provided herein is a PD-L1 axis binding antagonist (e.g., an anti-PD-L1 antibody such as atezolizumab) or a PD-1 binding antagonist (e.g., an anti-PD-1 antibody)) for use in a method of treating an individual having a cancer (e.g., bladder cancer (e.g., UC) or kidney cancer (e.g., RCC)), the method including: (a) administering an effective amount of an anti-cancer therapy including a PD-L1 axis binding antagonist to the individual; (b) determining the expression level of IL8 in a sample obtained from the individual at a time point following administration of the anti-cancer therapy; and (c) continuing to administer the anti-cancer therapy to the individual if the expression level of IL8 in the individual's sample is below the reference level of IL8.

In another aspect, provided herein is a PD-L1 axis binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the expression level of IL-8 in a sample obtained from the individual at a time point following administration of the PD-L1 axis binding antagonist has been determined to be below a reference level of IL8.

In another aspect, provided herein is a method of treating an individual having a cancer (e.g., bladder cancer (e.g., UC) or kidney cancer (e.g., RCC)), the method comprising: (a) administering an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)) to the individual; (b) determining the expression level of IL8 in a sample obtained from the individual at a time point following administration of the anti-cancer therapy, wherein the sample is a plasma sample, a tumor tissue sample, or a sample comprising peripheral blood mononuclear cells (PBMCs); and (c) continuing to administer the anti-cancer therapy to the individual if the expression level of IL8 in the individual's sample is below the reference level of IL8.

In another aspect, provided herein is a method of monitoring the response of an individual having a cancer (e.g., bladder cancer (e.g., UC) or kidney cancer (e.g., RCC)) to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)), the method comprising: (a) determining the expression level of IL8 in a sample obtained from the individual at a time point following administration of the anti-cancer therapy, wherein the sample is a plasma sample, a tumor tissue sample, or a sample comprising PBMCs; (b) comparing the expression level of IL8 in the sample with a reference level of IL8; and (c) continuing to administer the anti-cancer therapy to the individual if the expression level of IL8 in the individual's sample is below the reference level.

In another aspect, provided herein is a PD-L1 axis binding antagonist (e.g., an anti-PD-L1 antibody such as atezolizumab) or a PD-1 binding antagonist (e.g., an anti-PD-1 antibody)) for use in a method of treating an individual having a cancer (e.g., bladder cancer (e.g., UC) or kidney cancer (e.g., RCC)), the method including: (a) administering an effective amount of an anti-cancer therapy including a PD-L1 axis binding antagonist to the individual; (b) determining the expression level of IL8 in a sample obtained from the individual at a time point following administration of the anti-cancer therapy, wherein the sample is a plasma sample, a tumor tissue sample, or a sample including PBMCs; and (b) continuing to administer the anti-cancer therapy to the individual if the expression level of IL8 in the individual's sample is below the reference level of IL8.

In another aspect, provided herein is a PD-L1 axis binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the expression level of IL-8 in a sample obtained from the individual at a time point following administration of the PD-L1 axis binding antagonist has been determined to be below a reference level of IL8, wherein the sample is a plasma sample, a tumor tissue sample, or a sample including PBMCs.

Any suitable time point following administration of the anti-cancer therapy may be used. For example, the time point following administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) following administration of the anti-cancer therapy. In some instances, the time point following administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks following administration of the anti-cancer therapy. In some instances, the time point following administration of the anti-cancer therapy is about 4 to about 8 weeks following administration of the anti-cancer therapy. For example, in some aspects, the time point following administration of the anti-cancer therapy is about 6 weeks following administration of the anti-cancer therapy.

In another aspect, provided herein is a method of treating an individual having a cancer (e.g., bladder cancer (e.g., UC) or kidney cancer (e.g., RCC)), the method comprising: (a) determining the expression level of IL8 in a sample from the individual that has been obtained at a time point prior to or concurrently with administration of an anti-cancer therapy comprising a PD-L1 axis binding antagonist (e.g., an anti-PD-L1 antibody such as atezolizumab) or a PD-1 binding antagonist (e.g., an anti-PD-1 antibody)), wherein an expression level of IL8 in the sample that is below a reference level of IL8 identifies the individual as likely to respond to treatment with an anti-cancer therapy comprising a PD-L1 binding antagonist; and (b) administering an effective amount of an anti-cancer therapy comprising a PD-L1 axis binding antagonist to the individual based on the expression level of IL8 determined in step (a).

In another aspect, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)) based on an expression level of IL8 in a sample from the individual that is below a reference level of IL8, the method comprising administering to the individual an effective amount of an anti-cancer therapy comprising a PD-L1 axis binding antagonist, and wherein the sample has been obtained at a time point prior to or concurrently with administration of the anti-cancer therapy comprising a PD-L1 axis binding antagonist.

In another aspect, provided herein is a PD-L1 axis binding antagonist for use in treating an individual having a cancer (e.g., bladder cancer (e.g., UC) or kidney cancer (e.g., RCC)), wherein the individual has been identified as likely to respond to treatment with an anti-cancer therapy including a PD-L1 binding antagonist (e.g., an anti-PD-L1 antibody such as atezolizumab) or a PD-1 binding antagonist (e.g., an anti-PD-1 antibody)) based on an expression level of IL8 in a sample from the individual that is below a reference level of IL8, wherein the sample is obtained from the individual at a time point prior to or concurrently with administration of the anti-cancer therapy including a PD-L1 axis binding antagonist.

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of treating an individual having a cancer (e.g., bladder cancer (e.g., UC) or kidney cancer (e.g., RCC)), the method comprising: (a) determining the expression level of IL8 in a sample from the individual that has been obtained at a time point prior to or concurrently with administration of an anti-cancer therapy comprising a PD-L1 axis binding antagonist, wherein an expression level of IL8 in the sample that is below a reference level of IL8 identifies the individual as likely to respond to treatment with an anti-cancer therapy comprising a PD-L1 binding antagonist, wherein the response is indicated by OS or ORR; and (b) administering an effective amount of an anti-cancer therapy comprising a PD-L1 axis binding antagonist to the individual based on the expression level of IL8 determined in step (a).

In another example, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 axis binding antagonist based on an expression level of IL8 in a sample from the individual that is below a reference level of IL8, the method comprising administering to the individual an effective amount of an anti-cancer therapy comprising a PD-L1 axis binding antagonist, wherein the response is indicated by OS or ORR, and wherein the sample has been obtained at a time point prior to or concurrently with administration of the anti-cancer therapy comprising a PD-L1 axis binding antagonist.

In another example, provided herein is a PD-L1 axis binding antagonist for use in treating an individual having a cancer (e.g., bladder cancer (e.g., UC) or kidney cancer (e.g., RCC)), wherein the individual has been identified as likely to respond to treatment with an anti-cancer therapy including a PD-L1 axis binding antagonist based on an expression level of IL8 in a sample from the individual that is below a reference level of IL8, wherein the sample is obtained from the individual at a time point prior to or concurrently with administration of the anti-cancer therapy including a PD-L1 axis binding antagonist, and wherein the response is indicated by OS or ORR.

Any suitable time point prior to administration of the anti-cancer therapy may be used. For example, the time point prior to administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) prior to administration of the anti-cancer therapy. In some instances, the time point prior to administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks prior to administration of the anti-cancer therapy.

In any of the preceding methods, the individual may be likely to have improved response from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). For example, the individual may be likely to have an improved ORR, improved CR rate, improved PR rate, extended PFS, and/or extended OS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In some particular instances, the individual may be likely to have an improved ORR from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In other particular instances, the individual may be likely to have an extended PFS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In yet other particular instances, the individual may be likely to have an extended OS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist).

In other aspects of the present disclosure, individuals having elevated or high IL8 levels can be treated with an anti-cancer therapy, e.g., an anti-cancer therapy other than or in addition to a PD-L1 axis binding antagonist. For example, individuals having elevated or high baseline or on-treatment IL8 expression levels (e.g., relative to a reference level of IL8) can be treated with an anti-cancer therapy other than or in addition to a PD-L1 axis binding antagonist (e.g., combination therapy with a PD-L1 axis binding antagonist and one or more additional therapeutic agents (e.g., VEGF antagonists (e.g., anti-VEGF antibodies such as bevacizumab), or an anti-cancer therapy that does not include a PD-L1 axis binding antagonist).

For example, provided herein is a method of treating an individual having a cancer (e.g., bladder cancer (e.g., UC) or kidney cancer (e.g., RCC)), the method comprising: (a) determining the expression level of IL8 in a sample from the individual, wherein an expression level of IL8 in the sample that is at or above a reference level of IL8 identifies the individual as less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy; and (b) administering an effective amount of an anti-cancer therapy other than or in addition to a PD-L1 axis binding antagonist to the individual based on the expression level of IL8 determined in step (a).

In another example, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of IL8 in a sample from the individual that is at or above a reference level of IL8, the method comprising administering to the individual an effective amount of an anti-cancer therapy other than or in addition to a PD-L1 axis binding antagonist.

In yet another example, provided herein is an anti-cancer therapy other than or in addition to a PD-L1 axis binding antagonist for use in treating an individual having a cancer (e.g., bladder cancer (e.g., UC) or kidney cancer (e.g., RCC)), wherein the individual has been identified as less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of IL8 in a sample from the individual that is at or above a reference level of IL8.

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of treating an individual having a cancer (e.g., bladder cancer (e.g., UC) or kidney cancer (e.g., RCC)), the method comprising: (a) determining the expression level of IL8 in a sample from the individual, wherein an expression level of IL8 in the sample that is at or above a reference level of IL8 identifies the individual as less likely to respond to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)) monotherapy, wherein the response is indicated by OS or ORR; and (b) administering an effective amount of an anti-cancer therapy other than or in addition to a PD-L1 axis binding antagonist to the individual based on the expression level of IL8 determined in step (a).

In another example, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)) monotherapy based on an expression level of IL8 in a sample from the individual that is at or above a reference level of IL8, the method comprising administering to the individual an effective amount of an anti-cancer therapy other than or in addition to a PD-L1 axis binding antagonist, wherein the response is indicated by OS or ORR.

In yet another example, provided herein is an anti-cancer therapy other than or in addition to a PD-L1 axis binding antagonist for use in treating an individual having a cancer (e.g., bladder cancer (e.g., UC) or kidney cancer (e.g., RCC)), wherein the individual has been identified as less likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)) monotherapy based on an expression level of IL8 in a sample from the individual that is at or above a reference level of IL8, wherein the response is indicated by OS or ORR.

For example, provided herein is a method of treating an individual having a cancer (e.g., bladder cancer (e.g., UC) or kidney cancer (e.g., RCC)), the method comprising: (a) determining the expression level of IL8 in a sample from the individual, wherein an expression level of IL8 in the sample that is at or above a reference level of IL8 identifies the individual as less likely to respond to treatment with an anti-cancer therapy comprising a PD-L1 binding antagonist (e.g., an anti-PD-L1 antibody such as atezolizumab) monotherapy, wherein the response is indicated by OS or ORR; and (b) administering an effective amount of an anti-cancer therapy other than or in addition to a PD-L1 binding antagonist to the individual based on the expression level of IL8 determined in step (a).

In another example, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 binding antagonist (e.g., an anti-PD-L1 antibody such as atezolizumab) monotherapy based on an expression level of IL8 in a sample from the individual that is at or above a reference level of IL8, the method comprising administering to the individual an effective amount of an anti-cancer therapy other than or in addition to a PD-L1 binding antagonist, wherein the response is indicated by OS or ORR.

In yet another example, provided herein is an anti-cancer therapy other than or in addition to a PD-L1 binding antagonist for use in treating an individual having a cancer (e.g., bladder cancer (e.g., UC) or kidney cancer (e.g., RCC)), wherein the individual has been identified as less likely to respond to treatment with an anti-cancer therapy including a PD-L1 binding antagonist (e.g., an anti-PD-L1 antibody such as atezolizumab) monotherapy based on an expression level of IL8 in a sample from the individual that is at or above a reference level of IL8, wherein the response is indicated by OS or ORR.

In another aspect, provided herein is a method of treating an individual having a cancer (e.g., bladder cancer (e.g., UC) or kidney cancer (e.g., RCC)), the method comprising: (a) determining the expression level of IL8 in a plasma sample or a sample comprising PBMCs from the individual, wherein an expression level of IL8 in the plasma sample or in the sample comprising PBMCs that is at or above a reference level of IL8 identifies the individual as less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy; and (b) administering an effective amount of an anti-cancer therapy other than or in addition to a PD-L1 axis binding antagonist to the individual based on the expression level of IL8 in the plasma sample or in the sample comprising PBMCs determined in step (a).

In another aspect, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of IL8 in a plasma sample or a sample comprising PBMCs from the individual that is at or above a reference level of IL8, the method comprising administering to the individual an effective amount of an anti-cancer therapy other than or in addition to a PD-L1 axis binding antagonist.

In another aspect, provided herein is an anti-cancer therapy other than or in addition to a PD-L1 axis binding antagonist for use in treating an individual having a cancer (e.g., bladder cancer (e.g., UC) or kidney cancer (e.g., RCC)), wherein the individual has been identified as less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of IL8 in a plasma sample or a sample including PBMCs from the individual that is at or above a reference level of IL8.

In another aspect, provided herein is a method of treating an individual having a cancer (e.g., bladder cancer (e.g., UC) or kidney cancer (e.g., RCC)), the method comprising: (a) administering an effective amount of an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) to the individual; (b) determining the expression level of IL8 in a sample obtained from the individual at a time point following administration of the anti-cancer therapy comprising a PD-L1 axis binding antagonist; and (c) administering an anti-cancer therapy other than or in addition to a PD-L1 axis binding antagonist to the individual if the expression level of IL8 in the individual's sample is at or above the reference level of IL8.

In another aspect, provided herein is a method of monitoring the response of an individual having a cancer (e.g., bladder cancer (e.g., UC) or kidney cancer (e.g., RCC)) to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), the method comprising: (a) determining the expression level of IL8 in a sample obtained from the individual at a time point following administration of an anti-cancer therapy comprising a PD-L1 axis binding antagonist; (b) comparing the expression level of IL8 in the sample with a reference level of IL8; and (c) administering an anti-cancer therapy other than or in addition to a PD-L1 axis binding antagonist to the individual if the expression level of IL8 in the individual's sample is at or above the reference level.

In another aspect, provided herein is an anti-cancer therapy other than or in addition to a PD-L1 axis binding antagonist for use in a method of treating an individual having a cancer (e.g., bladder cancer (e.g., UC) or kidney cancer (e.g., RCC)), the method including: (a) administering an effective amount of an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) to the individual; (b) determining the expression level of IL8 in a sample obtained from the individual at a time point following administration of the anti-cancer therapy including a PD-L1 axis binding antagonist; and (c) administering the anti-cancer therapy other than or in addition to a PD-L1 axis binding antagonist to the individual if the expression level of IL8 in the individual's sample is at or above the reference level of IL8.

In another aspect, provided herein is an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) for use in treating an individual having a cancer, wherein the expression level of IL-8 in a sample obtained from the individual at a time point following administration of a PD-L1 axis binding antagonist has been determined to be at or above a reference level of IL8.

In another aspect, provided herein is a method of treating an individual having a cancer (e.g., bladder cancer (e.g., UC) or kidney cancer (e.g., RCC)), the method comprising: (a) administering an effective amount of an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) to the individual; (b) determining the expression level of IL8 in a sample obtained from the individual at a time point following administration of the anti-cancer therapy comprising a PD-L1 binding antagonist, wherein the sample is a plasma sample, a tumor tissue sample, or a sample comprising PBMCs; and (c) administering an anti-cancer therapy other than or in addition to a PD-L1 axis binding antagonist to the individual if the expression level of IL8 in the individual's sample is at or above the reference level of IL8.

In another aspect, provided herein is a method of monitoring the response of an individual having a cancer (e.g., bladder cancer (e.g., UC) or kidney cancer (e.g., RCC)) to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), the method comprising: (a) determining the expression level of IL8 in a sample obtained from the individual at a time point following administration of an anti-cancer therapy comprising a PD-L1 axis binding antagonist, wherein the sample is a plasma sample, a tumor tissue sample, or a sample comprising PBMCs; (b) comparing the expression level of IL8 in the sample with a reference level of IL8; and (c) administering an anti-cancer therapy other than or in addition to a PD-L1 axis binding antagonist to the individual if the expression level of IL8 in the individual's sample is at or above the reference level.

In another aspect, provided herein is an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) for use in a method of treating an individual having a cancer (e.g., bladder cancer (e.g., UC) or kidney cancer (e.g., RCC)), the method including: (a) administering an effective amount of an anti-cancer therapy including a PD-L1 axis binding antagonist to the individual; (b) determining the expression level of IL8 in a sample obtained from the individual at a time point following administration of an anti-cancer therapy including a PD-L1 axis binding antagonist, wherein the sample is a plasma sample, a tumor tissue sample, or a sample including PBMCs; and (c) administering the anti-cancer therapy other than or in addition to a PD-L1 axis binding antagonist to the individual if the expression level of IL8 in the individual's sample is at or above the reference level of IL8.

In another aspect, provided herein is an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the expression level of IL-8 in a sample obtained from the individual at a time point following administration of a PD-L1 axis binding antagonist has been determined to be at or above a reference level of IL8, wherein the sample is a plasma sample, a tumor tissue sample, or a sample including PBMCs.

Any suitable time point following administration of the anti-cancer therapy may be used. For example, the time point following administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) following administration of the anti-cancer therapy. In some instances, the time point following administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks following administration of the anti-cancer therapy. In some instances, the time point following administration of the anti-cancer therapy is about 4 to about 8 weeks following administration of the anti-cancer therapy. For example, in some aspects, the time point following administration of the anti-cancer therapy is about 6 weeks following administration of the anti-cancer therapy.

In another aspect, provided herein is a method of treating an individual having a cancer (e.g., bladder cancer (e.g., UC) or kidney cancer (e.g., RCC)), the method comprising: (a) determining the expression level of IL8 in a sample from the individual that has been obtained at a time point prior to or concurrently with administration of an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein an expression level of IL8 in the sample that is at or above a reference level of IL8 identifies the individual as less likely to respond to treatment with an anti-cancer therapy comprising a PD-L1 axis binding antagonist monotherapy; and (b) administering an effective amount of an anti-cancer therapy other than or in addition to a PD-L1 axis binding antagonist to the individual based on the expression level of IL8 determined in step (a).

In another aspect, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of IL8 in a sample from the individual that is at or above a reference level of IL8, the method comprising administering to the individual an effective amount of an anti-cancer therapy other than or in addition to a PD-L1 axis binding antagonist and wherein the sample has been obtained at a time point prior to or concurrently with administration of an anti-cancer therapy comprising a PD-L1 axis binding antagonist.

In another aspect, provided herein is an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the individual has been identified as less likely to respond to treatment with an anti-cancer therapy including a PD-L1 axis binding antagonist monotherapy based on an expression level of IL8 in a sample from the individual that is at or above a reference level of IL8, wherein the sample is obtained from the individual at a time point prior to or concurrently with administration of the anti-cancer therapy including a PD-L1 axis binding antagonist

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of treating an individual having a cancer (e.g., bladder cancer (e.g., UC) or kidney cancer (e.g., RCC)), the method comprising: (a) determining the expression level of IL8 in a sample from the individual that has been obtained at a time point prior to or concurrently with administration of an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein an expression level of IL8 in the sample that is at or above a reference level of IL8 identifies the individual as less likely to respond to treatment with an anti-cancer therapy comprising a PD-L1 axis binding antagonist monotherapy, wherein the response is indicated by OS or ORR; and (b) administering an effective amount of an anti-cancer therapy other than or in addition to a PD-L1 axis binding antagonist to the individual based on the expression level of IL8 determined in step (a).

In another example, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of IL8 in a sample from the individual that is at or above a reference level of IL8, the method comprising administering to the individual an effective amount of an anti-cancer therapy other than or in addition to a PD-L1 axis binding antagonist, wherein the response is indicated by OS or ORR, and wherein the sample has been obtained at a time point prior to or concurrently with administration of an anti-cancer therapy comprising a PD-L1 axis binding antagonist.

In yet another example, provided herein is an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the individual has been identified as less likely to respond to treatment with an anti-cancer therapy including a PD-L1 axis binding antagonist monotherapy based on an expression level of IL8 in a sample from the individual that is at or above a reference level of IL8, wherein the sample is obtained from the individual at a time point prior to or concurrently with administration of the anti-cancer therapy including a PD-L1 axis binding antagonist, and wherein the response is indicated by OS or ORR.

Any suitable time point prior to administration of the anti-cancer therapy may be used. For example, the time point prior to administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) prior to administration of the anti-cancer therapy. In some instances, the time point prior to administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks prior to administration of the anti-cancer therapy.

In any of the preceding methods, the individual may be likely to have a reduced response from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). For example, the individual may be likely to have a reduced ORR, reduced CR rate, reduced PR rate, shortened PFS, or shortened OS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In some particular instances, the individual may be likely to have a reduced ORR from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In other particular instances, the individual may be likely to have a shortened PFS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In other particular instances, the individual may be likely to have a shortened OS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist).

2. Genes Upregulated in IL8 High Cancer

In several aspects of the present disclosure, individuals having decreased or low levels of one or more genes that are upregulated in IL8 high cancer may be treated with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)). For example, individuals having decreased or low baseline or on-treatment expression levels of one or more genes set forth in any one of Tables 2-4 (e.g., relative to a reference level of one or more genes set forth in any one of Tables 2-4) may be treated with an anti-cancer therapy including a PD-L1 axis binding antagonist.

In one aspect, provided herein is a method of treating an individual having a cancer (e.g., bladder cancer (e.g., UC) or kidney cancer (e.g., RCC)), the method comprising: (a) determining the expression level of one or more genes set forth in any one of Tables 2-4 in a sample from the individual, wherein an expression level of the one or more genes set forth in any one of Tables 2-4 in the sample that is below a reference level of the one or more genes set forth in any one of Tables 2-4 identifies the individual as likely to respond to treatment with an anti-cancer therapy comprising a PD-L1 axis binding antagonist; and (b) administering an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)) to the individual based on the expression level of the one or more genes set forth in any one of Tables 2-4 determined in step (a).

In another aspect, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 axis binding antagonist based on an expression level of one or more genes set forth in any one of Tables 2-4 in a sample from the individual that is below a reference level of the one or more genes set forth in any one of Tables 2-4, the method comprising administering to the individual an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)).

In another aspect, provided herein is a PD-L1 axis binding antagonist for use in treating an individual having a cancer (e.g., bladder cancer (e.g., UC) or kidney cancer (e.g., RCC)), wherein the individual has been identified as likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)) based on an expression level of one or more genes set forth in any one of Tables 2-4 in a sample from the individual that is below a reference level of the one or more genes set forth in any one of Tables 2-4.

The response may be any suitable response. For example, in some aspects, the response is indicated by ORR, CR rate, PR rate, PFS, OS, and/or DOR. In some instances, the response is indicated by ORR or OS. In some particular instances, the response is indicated by ORR. In other particular instances, the response is indicated by PFS. In yet other particular instances, the response is indicated by OS.

For example, provided herein is a method of treating an individual having a cancer (e.g., bladder cancer (e.g., UC) or kidney cancer (e.g., RCC)), the method comprising: (a) determining the expression level of one or more genes set forth in any one of Tables 2-4 in a sample from the individual, wherein an expression level of the one or more genes set forth in any one of Tables 2-4 in the sample that is below a reference level of the one or more genes set forth in any one of Tables 2-4 identifies the individual as likely to respond to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)), wherein the response is indicated by overall survival (OS) or overall response rate (ORR); and (b) administering an effective amount of an anti-cancer therapy comprising a PD-L1 binding antagonist to the individual based on the expression level of the one or more genes set forth in any one of Tables 2-4 determined in step (a).

In another example, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 axis binding antagonist based on an expression level of one or more genes set forth in any one of Tables 2-4 in a sample from the individual that is below a reference level of the one or more genes set forth in any one of Tables 2-4, the method comprising administering to the individual an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)), wherein the response is indicated by OS or ORR.

In another aspect, provided herein is a PD-L1 axis binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the individual has been identified as likely to respond to treatment with an anti-cancer therapy including a PD-L1 binding antagonist based on an expression level of one or more genes set forth in any one of Tables 2-4 in a sample from the individual that is below a reference level of the one or more genes set forth in any one of Tables 2-4, wherein the response is indicated by OS or ORR.

For example, provided herein is a method of treating an individual having a cancer (e.g., bladder cancer (e.g., UC) or kidney cancer (e.g., RCC)), the method comprising: (a) determining the expression level of one or more genes set forth in any one of Tables 2-4 in a sample from the individual, wherein an expression level of the one or more genes set forth in any one of Tables 2-4 in the sample that is below a reference level of the one or more genes set forth in any one of Tables 2-4 identifies the individual as likely to respond to treatment with an anti-cancer therapy comprising a PD-L1 binding antagonist (e.g., an anti-PD-L1 antibody such as atezolizumab), wherein the response is indicated by overall survival (OS) or overall response rate (ORR); and (b) administering an effective amount of an anti-cancer therapy comprising a PD-L1 binding antagonist to the individual based on the expression level of the one or more genes set forth in any one of Tables 2-4 determined in step (a).

In another example, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 binding antagonist based on an expression level of one or more genes set forth in any one of Tables 2-4 in a sample from the individual that is below a reference level of the one or more genes set forth in any one of Tables 2-4, the method comprising administering to the individual an effective amount of an anti-cancer therapy comprising a PD-L1 binding antagonist (e.g., an anti-PD-L1 antibody such as atezolizumab), wherein the response is indicated by OS or ORR.

In another aspect, provided herein is a PD-L1 binding antagonist (e.g., an anti-PD-L1 antibody such as atezolizumab) for use in treating an individual having a cancer (e.g., bladder cancer (e.g., UC) or kidney cancer (e.g., RCC)), wherein the individual has been identified as likely to respond to treatment with an anti-cancer therapy including a PD-L1 binding antagonist based on an expression level of one or more genes set forth in any one of Tables 2-4 in a sample from the individual that is below a reference level of the one or more genes set forth in any one of Tables 2-4, wherein the response is indicated by OS or ORR.

In another aspect, provided herein is a method of treating an individual having a cancer (e.g., bladder cancer (e.g., UC) or kidney cancer (e.g., RCC)), the method comprising: (a) determining the expression level of one or more genes set forth in any one of Tables 2-4 in a plasma sample or a sample comprising PBMCs from the individual, wherein an expression level of the one or more genes set forth in any one of Tables 2-4 in the plasma sample or in the sample comprising PBMCs that is below a reference level of the one or more genes set forth in any one of Tables 2-4 identifies the individual as likely to respond to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)); and (b) administering an effective amount of an anti-cancer therapy comprising a PD-L1 axis binding antagonist to the individual based on the expression level of the one or more genes set forth in any one of Tables 2-4 determined in the plasma sample or in the sample comprising PBMCs in step (a).

In another aspect, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 axis binding antagonist based on an expression level of one or more genes set forth in any one of Tables 2-4 in a plasma sample or a sample comprising PBMCs from the individual that is below a reference level of the one or more genes set forth in any one of Tables 2-4, the method comprising administering to the individual an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)).

In another aspect, provided herein is a PD-L1 axis binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the individual has been identified as likely to respond to treatment with an anti-cancer therapy including a PD-L1 axis binding antagonist based on an expression level of one or more genes set forth in any one of Tables 2-4 in a plasma sample or a sample including PBMCs from the individual that is below a reference level of the one or more genes set forth in any one of Tables 2-4.

In another aspect, provided herein is a method of treating an individual having a cancer (e.g., bladder cancer (e.g., UC) or kidney cancer (e.g., RCC)), the method comprising: (a) administering an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)) to the individual; (b) determining the expression level of one or more genes set forth in any one of Tables 2-4 in a sample obtained from the individual at a time point following administration of the anti-cancer therapy; and (c) continuing to administer the anti-cancer therapy to the individual if the expression level of the one or more genes set forth in any one of Tables 2-4 in the individual's sample is below the reference level of the one or more genes set forth in any one of Tables 2-4.

In another aspect, provided herein is a method of monitoring the response of an individual having a cancer (e.g., bladder cancer (e.g., UC) or kidney cancer (e.g., RCC)) to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)), the method comprising: (a) determining the expression level of one or more genes set forth in any one of Tables 2-4 in a sample obtained from the individual at a time point following administration of the anti-cancer therapy; (b) comparing the expression level of the one or more genes set forth in any one of Tables 2-4 in the sample with a reference level of the one or more genes set forth in any one of Tables 2-4; and (c) continuing to administer the anti-cancer therapy to the individual if the expression level of the one or more genes set forth in any one of Tables 2-4 in the individual's sample is below the reference level.

In another aspect, provided herein is a PD-L1 axis binding antagonist (e.g., an anti-PD-L1 antibody such as atezolizumab) or a PD-1 binding antagonist (e.g., an anti-PD-1 antibody)) for use in a method of treating an individual having a cancer (e.g., bladder cancer (e.g., UC) or kidney cancer (e.g., RCC)), the method including: (a) administering an effective amount of an anti-cancer therapy including a PD-L1 axis binding antagonist to the individual; (b) determining the expression level of one or more genes set forth in any one of Tables 2-4 in a sample obtained from the individual at a time point following administration of the anti-cancer therapy; and (c) continuing to administer the anti-cancer therapy to the individual if the expression level of the one or more genes set forth in any one of Tables 2-4 in the individual's sample is below the reference level of the one or more genes set forth in any one of Tables 2-4.

In another aspect, provided herein is a PD-L1 axis binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the expression level of one or more genes set forth in any one of Tables 2-4 in a sample obtained from the individual at a time point following administration of the PD-L1 axis binding antagonist has been determined to be below a reference level of the one or more genes set forth in any one of Tables 2-4.

In another aspect, provided herein is a method of treating an individual having a cancer (e.g., bladder cancer (e.g., UC) or kidney cancer (e.g., RCC)), the method comprising: (a) administering an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)) to the individual; (b) determining the expression level of one or more genes set forth in any one of Tables 2-4 in a sample obtained from the individual at a time point following administration of the anti-cancer therapy, wherein the sample is a plasma sample, a tumor tissue sample, or a sample comprising peripheral blood mononuclear cells (PBMCs); and (c) continuing to administer the anti-cancer therapy to the individual if the expression level of the one or more genes set forth in any one of Tables 2-4 in the individual's sample is below the reference level of the one or more genes set forth in any one of Tables 2-4.

In another aspect, provided herein is a method of monitoring the response of an individual having a cancer (e.g., bladder cancer (e.g., UC) or kidney cancer (e.g., RCC)) to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)), the method comprising: (a) determining the expression level of one or more genes set forth in any one of Tables 2-4 in a sample obtained from the individual at a time point following administration of the anti-cancer therapy, wherein the sample is a plasma sample, a tumor tissue sample, or a sample comprising PBMCs; (b) comparing the expression level of the one or more genes set forth in any one of Tables 2-4 in the sample with a reference level of the one or more genes set forth in any one of Tables 2-4; and (c) continuing to administer the anti-cancer therapy to the individual if the expression level of the one or more genes set forth in any one of Tables 2-4 in the individual's sample is below the reference level.

In another aspect, provided herein is a PD-L1 axis binding antagonist (e.g., an anti-PD-L1 antibody such as atezolizumab) or a PD-1 binding antagonist (e.g., an anti-PD-1 antibody)) for use in a method of treating an individual having a cancer (e.g., bladder cancer (e.g., UC) or kidney cancer (e.g., RCC)), the method including: (a) administering an effective amount of an anti-cancer therapy including a PD-L1 axis binding antagonist to the individual; (b) determining the expression level of one or more genes set forth in any one of Tables 2-4 in a sample obtained from the individual at a time point following administration of the anti-cancer therapy, wherein the sample is a plasma sample, a tumor tissue sample, or a sample including PBMCs; and (b) continuing to administer the anti-cancer therapy to the individual if the expression level of the one or more genes set forth in any one of Tables 2-4 in the individual's sample is below the reference level of the one or more genes set forth in any one of Tables 2-4.

In another aspect, provided herein is a PD-L1 axis binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the expression level of one or more genes set forth in any one of Tables 2-4 in a sample obtained from the individual at a time point following administration of the PD-L1 axis binding antagonist has been determined to be below a reference level of the one or more genes set forth in any one of Tables 2-4, wherein the sample is a plasma sample, a tumor tissue sample, or a sample including PBMCs.

Any suitable time point following administration of the anti-cancer therapy may be used. For example, the time point following administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) following administration of the anti-cancer therapy. In some instances, the time point following administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks following administration of the anti-cancer therapy. In some instances, the time point following administration of the anti-cancer therapy is about 4 to about 8 weeks following administration of the anti-cancer therapy. For example, in some aspects, the time point following administration of the anti-cancer therapy is about 6 weeks following administration of the anti-cancer therapy.

In another aspect, provided herein is a method of treating an individual having a cancer (e.g., bladder cancer (e.g., UC) or kidney cancer (e.g., RCC)), the method comprising: (a) determining the expression level of one or more genes set forth in any one of Tables 2-4 in a sample from the individual that has been obtained at a time point prior to or concurrently with administration of an anti-cancer therapy comprising a PD-L1 axis binding antagonist (e.g., an anti-PD-L1 antibody such as atezolizumab) or a PD-1 binding antagonist (e.g., an anti-PD-1 antibody)), wherein an expression level of the one or more genes set forth in any one of Tables 2-4 in the sample that is below a reference level of the one or more genes set forth in any one of Tables 2-4 identifies the individual as likely to respond to treatment with an anti-cancer therapy comprising a PD-L1 binding antagonist; and (b) administering an effective amount of an anti-cancer therapy comprising a PD-L1 axis binding antagonist to the individual based on the expression level of the one or more genes set forth in any one of Tables 2-4 determined in step (a).

In another aspect, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)) based on an expression level of one or more genes set forth in any one of Tables 2-4 in a sample from the individual that is below a reference level of the one or more genes set forth in any one of Tables 2-4, the method comprising administering to the individual an effective amount of an anti-cancer therapy comprising a PD-L1 axis binding antagonist, and wherein the sample has been obtained at a time point prior to or concurrently with administration of the anti-cancer therapy comprising a PD-L1 axis binding antagonist.

In another aspect, provided herein is a PD-L1 axis binding antagonist for use in treating an individual having a cancer (e.g., bladder cancer (e.g., UC) or kidney cancer (e.g., RCC)), wherein the individual has been identified as likely to respond to treatment with an anti-cancer therapy including a PD-L1 binding antagonist (e.g., an anti-PD-L1 antibody such as atezolizumab) or a PD-1 binding antagonist (e.g., an anti-PD-1 antibody)) based on an expression level of one or more genes set forth in any one of Tables 2-4 in a sample from the individual that is below a reference level of the one or more genes set forth in any one of Tables 2-4, wherein the sample is obtained from the individual at a time point prior to or concurrently with administration of the anti-cancer therapy including a PD-L1 axis binding antagonist.

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of treating an individual having a cancer (e.g., bladder cancer (e.g., UC) or kidney cancer (e.g., RCC)), the method comprising: (a) determining the expression level of one or more genes set forth in any one of Tables 2-4 in a sample from the individual that has been obtained at a time point prior to or concurrently with administration of an anti-cancer therapy comprising a PD-L1 axis binding antagonist, wherein an expression level of the one or more genes set forth in any one of Tables 2-4 in the sample that is below a reference level of the one or more genes set forth in any one of Tables 2-4 identifies the individual as likely to respond to treatment with an anti-cancer therapy comprising a PD-L1 binding antagonist, wherein the response is indicated by OS or ORR; and (b) administering an effective amount of an anti-cancer therapy comprising a PD-L1 axis binding antagonist to the individual based on the expression level of the one or more genes set forth in any one of Tables 2-4 determined in step (a).

In another example, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 axis binding antagonist based on an expression level of one or more genes set forth in any one of Tables 2-4 in a sample from the individual that is below a reference level of the one or more genes set forth in any one of Tables 2-4, the method comprising administering to the individual an effective amount of an anti-cancer therapy comprising a PD-L1 axis binding antagonist, wherein the response is indicated by OS or ORR, and wherein the sample has been obtained at a time point prior to or concurrently with administration of the anti-cancer therapy comprising a PD-L1 axis binding antagonist.

In another example, provided herein is a PD-L1 axis binding antagonist for use in treating an individual having a cancer (e.g., bladder cancer (e.g., UC) or kidney cancer (e.g., RCC)), wherein the individual has been identified as likely to respond to treatment with an anti-cancer therapy including a PD-L1 axis binding antagonist based on an expression level of one or more genes set forth in any one of Tables 2-4 in a sample from the individual that is below a reference level of the one or more genes set forth in any one of Tables 2-4, wherein the sample is obtained from the individual at a time point prior to or concurrently with administration of the anti-cancer therapy including a PD-L1 axis binding antagonist, and wherein the response is indicated by OS or ORR.

Any suitable time point prior to administration of the anti-cancer therapy may be used. For example, the time point prior to administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) prior to administration of the anti-cancer therapy. In some instances, the time point prior to administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks prior to administration of the anti-cancer therapy.

In any of the preceding methods, the individual may be likely to have improved response from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). For example, the individual may be likely to have an improved ORR, improved CR rate, improved PR rate, extended PFS, and/or extended OS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In some particular instances, the individual may be likely to have an improved ORR from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In other particular instances, the individual may be likely to have an extended PFS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In yet other particular instances, the individual may be likely to have an extended OS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist).

In other aspects of the present disclosure, individuals having elevated or high levels of one or more genes that are upregulated in IL8 high cancer, e.g., one or more genes set forth in any one of Tables 2-4, can be treated with an anti-cancer therapy, e.g., an anti-cancer therapy other than or in addition to a PD-L1 axis binding antagonist. For example, individuals having elevated or high baseline or on-treatment expression levels of one or more genes set forth in any one of Tables 2-4 (e.g., relative to a reference level of the one or more genes set forth in any one of Tables 2-4) can be treated with an anti-cancer therapy other than or in addition to a PD-L1 axis binding antagonist (e.g., combination therapy with a PD-L1 axis binding antagonist and one or more additional therapeutic agents (e.g., VEGF antagonists (e.g., anti-VEGF antibodies such as bevacizumab), or an anti-cancer therapy that does not include a PD-L1 axis binding antagonist).

For example, provided herein is a method of treating an individual having a cancer (e.g., bladder cancer (e.g., UC) or kidney cancer (e.g., RCC)), the method comprising: (a) determining the expression level of one or more genes set forth in any one of Tables 2-4 in a sample from the individual, wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy; and (b) administering an effective amount of an anti-cancer therapy other than or in addition to a PD-L1 axis binding antagonist to the individual based on the expression level of the one or more genes set forth in any one of Tables 2-4 determined in step (a).

In another example, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of one or more genes set forth in any one of Tables 2-4 in a sample from the individual that is at or above a reference level of the one or more genes set forth in any one of Tables 2-4, the method comprising administering to the individual an effective amount of an anti-cancer therapy other than or in addition to a PD-L1 axis binding antagonist.

In yet another example, provided herein is an anti-cancer therapy other than or in addition to a PD-L1 axis binding antagonist for use in treating an individual having a cancer (e.g., bladder cancer (e.g., UC) or kidney cancer (e.g., RCC)), wherein the individual has been identified as less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of one or more genes set forth in any one of Tables 2-4 in a sample from the individual that is at or above a reference level of the one or more genes set forth in any one of Tables 2-4.

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of treating an individual having a cancer (e.g., bladder cancer (e.g., UC) or kidney cancer (e.g., RCC)), the method comprising: (a) determining the expression level of one or more genes set forth in any one of Tables 2-4 in a sample from the individual, wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)) monotherapy, wherein the response is indicated by OS or ORR; and (b) administering an effective amount of an anti-cancer therapy other than or in addition to a PD-L1 axis binding antagonist to the individual based on the expression level of the one or more genes set forth in any one of Tables 2-4 determined in step (a).

In another example, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)) monotherapy based on an expression level of the one or more genes set forth in any one of Tables 2-4 in a sample from the individual that is at or above a reference level of the one or more genes set forth in any one of Tables 2-4, the method comprising administering to the individual an effective amount of an anti-cancer therapy other than or in addition to a PD-L1 axis binding antagonist, wherein the response is indicated by OS or ORR.

In yet another example, provided herein is an anti-cancer therapy other than or in addition to a PD-L1 axis binding antagonist for use in treating an individual having a cancer (e.g., bladder cancer (e.g., UC) or kidney cancer (e.g., RCC)), wherein the individual has been identified as less likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)) monotherapy based on an expression level of one or more genes set forth in any one of Tables 2-4 in a sample from the individual that is at or above a reference level of the one or more genes set forth in any one of Tables 2-4, wherein the response is indicated by OS or ORR.

For example, provided herein is a method of treating an individual having a cancer (e.g., bladder cancer (e.g., UC) or kidney cancer (e.g., RCC)), the method comprising: (a) determining the expression level of one or more genes set forth in any one of Tables 2-4 in a sample from the individual, wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy comprising a PD-L1 binding antagonist (e.g., an anti-PD-L1 antibody such as atezolizumab) monotherapy, wherein the response is indicated by OS or ORR; and (b) administering an effective amount of an anti-cancer therapy other than or in addition to a PD-L1 binding antagonist to the individual based on the expression level of the one or more genes set forth in any one of Tables 2-4 determined in step (a).

In another example, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 binding antagonist (e.g., an anti-PD-L1 antibody such as atezolizumab) monotherapy based on an expression level of one or more genes set forth in any one of Tables 2-4 in a sample from the individual that is at or above a reference level of the one or more genes set forth in any one of Tables 2-4, the method comprising administering to the individual an effective amount of an anti-cancer therapy other than or in addition to a PD-L1 binding antagonist, wherein the response is indicated by OS or ORR.

In yet another example, provided herein is an anti-cancer therapy other than or in addition to a PD-L1 binding antagonist for use in treating an individual having a cancer (e.g., bladder cancer (e.g., UC) or kidney cancer (e.g., RCC)), wherein the individual has been identified as less likely to respond to treatment with an anti-cancer therapy including a PD-L1 binding antagonist (e.g., an anti-PD-L1 antibody such as atezolizumab) monotherapy based on an expression level of one or more genes set forth in any one of Tables 2-4 in a sample from the individual that is at or above a reference level of the one or more genes set forth in any one of Tables 2-4, wherein the response is indicated by OS or ORR.

In another aspect, provided herein is a method of treating an individual having a cancer (e.g., bladder cancer (e.g., UC) or kidney cancer (e.g., RCC)), the method comprising: (a) determining the expression level of one or more genes set forth in any one of Tables 2-4 in a plasma sample or a sample comprising PBMCs from the individual, wherein an expression level of the one or more genes set forth in any one of Tables 2-4 in the plasma sample or in the sample comprising PBMCs that is at or above a reference level of the one or more genes set forth in any one of Tables 2-4 identifies the individual as less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy; and (b) administering an effective amount of an anti-cancer therapy other than or in addition to a PD-L1 axis binding antagonist to the individual based on the expression level of the one or more genes set forth in any one of Tables 2-4 in the plasma sample or in the sample comprising PBMCs determined in step (a).

In another aspect, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of one or more genes set forth in any one of Tables 2-4 in a plasma sample or a sample comprising PBMCs from the individual that is at or above a reference level of the one or more genes set forth in any one of Tables 2-4, the method comprising administering to the individual an effective amount of an anti-cancer therapy other than or in addition to a PD-L1 axis binding antagonist.

In another aspect, provided herein is an anti-cancer therapy other than or in addition to a PD-L1 axis binding antagonist for use in treating an individual having a cancer (e.g., bladder cancer (e.g., UC) or kidney cancer (e.g., RCC)), wherein the individual has been identified as less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of one or more genes set forth in any one of Tables 2-4 in a plasma sample or a sample including PBMCs from the individual that is at or above a reference level of the one or more genes set forth in any one of Tables 2-4.

In another aspect, provided herein is a method of treating an individual having a cancer (e.g., bladder cancer (e.g., UC) or kidney cancer (e.g., RCC)), the method comprising: (a) administering an effective amount of an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) to the individual; (b) determining the expression level of one or more genes set forth in any one of Tables 2-4 in a sample obtained from the individual at a time point following administration of the anti-cancer therapy comprising a PD-L1 axis binding antagonist; and (c) administering an anti-cancer therapy other than or in addition to a PD-L1 axis binding antagonist to the individual if the expression level of the one or more genes set forth in any one of Tables 2-4 in the individual's sample is at or above the reference level of the one or more genes set forth in any one of Tables 2-4.

In another aspect, provided herein is a method of monitoring the response of an individual having a cancer (e.g., bladder cancer (e.g., UC) or kidney cancer (e.g., RCC)) to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), the method comprising: (a) determining the expression level of one or more genes set forth in any one of Tables 2-4 in a sample obtained from the individual at a time point following administration of an anti-cancer therapy comprising a PD-L1 axis binding antagonist; (b) comparing the expression level of the one or more genes set forth in any one of Tables 2-4 in the sample with a reference level of the one or more genes set forth in any one of Tables 2-4; and (c) administering an anti-cancer therapy other than or in addition to a PD-L1 axis binding antagonist to the individual if the expression level of the one or more genes set forth in any one of Tables 2-4 in the individual's sample is at or above the reference level.

In another aspect, provided herein is an anti-cancer therapy other than or in addition to a PD-L1 axis binding antagonist for use in a method of treating an individual having a cancer (e.g., bladder cancer (e.g., UC) or kidney cancer (e.g., RCC)), the method including: (a) administering an effective amount of an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) to the individual; (b) determining the expression level of one or more genes set forth in any one of Tables 2-4 in a sample obtained from the individual at a time point following administration of the anti-cancer therapy including a PD-L1 axis binding antagonist; and (c) administering the anti-cancer therapy other than or in addition to a PD-L1 axis binding antagonist to the individual if the expression level of the one or more genes set forth in any one of Tables 2-4 in the individual's sample is at or above the reference level of the one or more genes set forth in any one of Tables 2-4.

In another aspect, provided herein is an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) for use in treating an individual having a cancer, wherein the expression level of one or more genes set forth in any one of Tables 2-4in a sample obtained from the individual at a time point following administration of a PD-L1 axis binding antagonist has been determined to be at or above a reference level of the one or more genes set forth in any one of Tables 2-4.

In another aspect, provided herein is a method of treating an individual having a cancer (e.g., bladder cancer (e.g., UC) or kidney cancer (e.g., RCC)), the method comprising: (a) administering an effective amount of an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) to the individual; (b) determining the expression level of one or more genes set forth in any one of Tables 2-4 in a sample obtained from the individual at a time point following administration of the anti-cancer therapy comprising a PD-L1 binding antagonist, wherein the sample is a plasma sample, a tumor tissue sample, or a sample comprising PBMCs; and (c) administering an anti-cancer therapy other than or in addition to a PD-L1 axis binding antagonist to the individual if the expression level of the one or more genes set forth in any one of Tables 2-4 in the individual's sample is at or above the reference level of the one or more genes set forth in any one of Tables 2-4.

In another aspect, provided herein is a method of monitoring the response of an individual having a cancer (e.g., bladder cancer (e.g., UC) or kidney cancer (e.g., RCC)) to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), the method comprising: (a) determining the expression level of one or more genes set forth in any one of Tables 2-4 in a sample obtained from the individual at a time point following administration of an anti-cancer therapy comprising a PD-L1 axis binding antagonist, wherein the sample is a plasma sample, a tumor tissue sample, or a sample comprising PBMCs; (b) comparing the expression level of the one or more genes set forth in any one of Tables 2-4 in the sample with a reference level of the one or more genes set forth in any one of Tables 2-4; and (c) administering an anti-cancer therapy other than or in addition to a PD-L1 axis binding antagonist to the individual if the expression level of the one or more genes set forth in any one of Tables 2-4 in the individual's sample is at or above the reference level.

In another aspect, provided herein is an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) for use in a method of treating an individual having a cancer (e.g., bladder cancer (e.g., UC) or kidney cancer (e.g., RCC)), the method including: (a) administering an effective amount of an anti-cancer therapy including a PD-L1 axis binding antagonist to the individual; (b) determining the expression level of one or more genes set forth in any one of Tables 2-4 in a sample obtained from the individual at a time point following administration of an anti-cancer therapy including a PD-L1 axis binding antagonist, wherein the sample is a plasma sample, a tumor tissue sample, or a sample including PBMCs; and (c) administering the anti-cancer therapy other than or in addition to a PD-L1 axis binding antagonist to the individual if the expression level of the one or more genes set forth in any one of Tables 2-4 in the individual's sample is at or above the reference level of the one or more genes set forth in any one of Tables 2-4.

In another aspect, provided herein is an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the expression level of one or more genes set forth in any one of Tables 2-4 in a sample obtained from the individual at a time point following administration of a PD-L1 axis binding antagonist has been determined to be at or above a reference level of the one or more genes set forth in any one of Tables 2-4, wherein the sample is a plasma sample, a tumor tissue sample, or a sample including PBMCs.

Any suitable time point following administration of the anti-cancer therapy may be used. For example, the time point following administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) following administration of the anti-cancer therapy. In some instances, the time point following administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks following administration of the anti-cancer therapy. In some instances, the time point following administration of the anti-cancer therapy is about 4 to about 8 weeks following administration of the anti-cancer therapy. For example, in some aspects, the time point following administration of the anti-cancer therapy is about 6 weeks following administration of the anti-cancer therapy.

In another aspect, provided herein is a method of treating an individual having a cancer (e.g., bladder cancer (e.g., UC) or kidney cancer (e.g., RCC)), the method comprising: (a) determining the expression level of one or more genes set forth in any one of Tables 2-4 in a sample from the individual that has been obtained at a time point prior to or concurrently with administration of an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy comprising a PD-L1 axis binding antagonist monotherapy; and (b) administering an effective amount of an anti-cancer therapy other than or in addition to a PD-L1 axis binding antagonist to the individual based on the expression level of the one or more genes set forth in any one of Tables 2-4 determined in step (a).

In another aspect, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of one or more genes set forth in any one of Tables 2-4 in a sample from the individual that is at or above a reference level of the one or more genes set forth in any one of Tables 2-4, the method comprising administering to the individual an effective amount of an anti-cancer therapy other than or in addition to a PD-L1 axis binding antagonist and wherein the sample has been obtained at a time point prior to or concurrently with administration of an anti-cancer therapy comprising a PD-L1 axis binding antagonist.

In another aspect, provided herein is an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the individual has been identified as less likely to respond to treatment with an anti-cancer therapy including a PD-L1 axis binding antagonist monotherapy based on an expression level of one or more genes set forth in any one of Tables 2-4 in a sample from the individual that is at or above a reference level of the one or more genes set forth in any one of Tables 2-4, wherein the sample is obtained from the individual at a time point prior to or concurrently with administration of the anti-cancer therapy including a PD-L1 axis binding antagonist

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of treating an individual having a cancer (e.g., bladder cancer (e.g., UC) or kidney cancer (e.g., RCC)), the method comprising: (a) determining the expression level of one or more genes set forth in any one of Tables 2-4 in a sample from the individual that has been obtained at a time point prior to or concurrently with administration of an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein an expression level of the one or more genes set forth in any one of Tables 2-4 in the sample that is at or above a reference level of the one or more genes set forth in any one of Tables 2-4 identifies the individual as less likely to respond to treatment with an anti-cancer therapy comprising a PD-L1 axis binding antagonist monotherapy, wherein the response is indicated by OS or ORR; and (b) administering an effective amount of an anti-cancer therapy other than or in addition to a PD-L1 axis binding antagonist to the individual based on the expression level of the one or more genes set forth in any one of Tables 2-4 determined in step (a).

In another example, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of one or more genes set forth in any one of Tables 2-4 in a sample from the individual that is at or above a reference level of the one or more genes set forth in any one of Tables 2-4, the method comprising administering to the individual an effective amount of an anti-cancer therapy other than or in addition to a PD-L1 axis binding antagonist, wherein the response is indicated by OS or ORR, and wherein the sample has been obtained at a time point prior to or concurrently with administration of an anti-cancer therapy comprising a PD-L1 axis binding antagonist.

In yet another example, provided herein is an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the individual has been identified as less likely to respond to treatment with an anti-cancer therapy including a PD-L1 axis binding antagonist monotherapy based on an expression level of one or more genes set forth in any one of Tables 2-4 in a sample from the individual that is at or above a reference level of the one or more genes set forth in any one of Tables 2-4, wherein the sample is obtained from the individual at a time point prior to or concurrently with administration of the anti-cancer therapy including a PD-L1 axis binding antagonist, and wherein the response is indicated by OS or ORR.

Any suitable time point prior to administration of the anti-cancer therapy may be used. For example, the time point prior to administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) prior to administration of the anti-cancer therapy. In some instances, the time point prior to administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks prior to administration of the anti-cancer therapy.

In any of the preceding methods, the individual may be likely to have a reduced response from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). For example, the individual may be likely to have a reduced ORR, reduced CR rate, reduced PR rate, shortened PFS, or shortened OS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In some particular instances, the individual may be likely to have a reduced ORR from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In other particular instances, the individual may be likely to have a shortened PFS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In other particular instances, the individual may be likely to have a shortened OS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist).

3. Genes Downregulated in IL8 High Cancer

In several aspects of the present disclosure, individuals having increased or high levels of one or more genes that are downregulated in IL8 high cancer may be treated with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)). For example, individuals having increased or high baseline or on-treatment expression levels of one or more genes set forth in any one of Tables 5-7 (e.g., relative to a reference level of one or more genes set forth in any one of Tables 5-7) may be treated with an anti-cancer therapy including a PD-L1 axis binding antagonist.

In one aspect, provided herein is a method of treating an individual having a cancer (e.g., bladder cancer (e.g., UC) or kidney cancer (e.g., RCC)), the method comprising: (a) determining the expression level of one or more genes set forth in any one of Tables 5-7 in a sample from the individual, wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy comprising a PD-L1 axis binding antagonist; and (b) administering an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)) to the individual based on the expression level of the one or more genes set forth in any one of Tables 5-7 determined in step (a).

In another aspect, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 axis binding antagonist based on an expression level of one or more genes set forth in any one of Tables 5-7 in a sample from the individual that is at or above a reference level of the one or more genes set forth in any one of Tables 5-7, the method comprising administering to the individual an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)).

In another aspect, provided herein is a PD-L1 axis binding antagonist for use in treating an individual having a cancer (e.g., bladder cancer (e.g., UC) or kidney cancer (e.g., RCC)), wherein the individual has been identified as likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)) based on an expression level of one or more genes set forth in any one of Tables 5-7 in a sample from the individual that is at or above a reference level of the one or more genes set forth in any one of Tables 5-7.

The response may be any suitable response. For example, in some aspects, the response is indicated by ORR, CR rate, PR rate, PFS, OS, and/or DOR. In some instances, the response is indicated by ORR or OS. In some particular instances, the response is indicated by ORR. In other particular instances, the response is indicated by PFS. In yet other particular instances, the response is indicated by OS.

For example, provided herein is a method of treating an individual having a cancer (e.g., bladder cancer (e.g., UC) or kidney cancer (e.g., RCC)), the method comprising: (a) determining the expression level of one or more genes set forth in any one of Tables 5-7 in a sample from the individual, wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)), wherein the response is indicated by overall survival (OS) or overall response rate (ORR); and (b) administering an effective amount of an anti-cancer therapy comprising a PD-L1 binding antagonist to the individual based on the expression level of the one or more genes set forth in any one of Tables 5-7 determined in step (a).

In another example, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 axis binding antagonist based on an expression level of one or more genes set forth in any one of Tables 5-7 in a sample from the individual that is at or above a reference level of the one or more genes set forth in any one of Tables 5-7, the method comprising administering to the individual an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)), wherein the response is indicated by OS or ORR.

In another aspect, provided herein is a PD-L1 axis binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the individual has been identified as likely to respond to treatment with an anti-cancer therapy including a PD-L1 binding antagonist based on an expression level of one or more genes set forth in any one of Tables 5-7 in a sample from the individual that is at or above a reference level of the one or more genes set forth in any one of Tables 5-7, wherein the response is indicated by OS or ORR.

For example, provided herein is a method of treating an individual having a cancer (e.g., bladder cancer (e.g., UC) or kidney cancer (e.g., RCC)), the method comprising: (a) determining the expression level of one or more genes set forth in any one of Tables 5-7 in a sample from the individual, wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy comprising a PD-L1 binding antagonist (e.g., an anti-PD-L1 antibody such as atezolizumab), wherein the response is indicated by overall survival (OS) or overall response rate (ORR); and (b) administering an effective amount of an anti-cancer therapy comprising a PD-L1 binding antagonist to the individual based on the expression level of the one or more genes set forth in any one of Tables 5-7 determined in step (a).

In another example, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 binding antagonist based on an expression level of one or more genes set forth in any one of Tables 5-7 in a sample from the individual that is at or above a reference level of the one or more genes set forth in any one of Tables 5-7, the method comprising administering to the individual an effective amount of an anti-cancer therapy comprising a PD-L1 binding antagonist (e.g., an anti-PD-L1 antibody such as atezolizumab), wherein the response is indicated by OS or ORR.

In another aspect, provided herein is a PD-L1 binding antagonist (e.g., an anti-PD-L1 antibody such as atezolizumab) for use in treating an individual having a cancer (e.g., bladder cancer (e.g., UC) or kidney cancer (e.g., RCC)), wherein the individual has been identified as likely to respond to treatment with an anti-cancer therapy including a PD-L1 binding antagonist based on an expression level of one or more genes set forth in any one of Tables 5-7 in a sample from the individual that is at or above a reference level of the one or more genes set forth in any one of Tables 5-7, wherein the response is indicated by OS or ORR.

In another aspect, provided herein is a method of treating an individual having a cancer (e.g., bladder cancer (e.g., UC) or kidney cancer (e.g., RCC)), the method comprising: (a) determining the expression level of one or more genes set forth in any one of Tables 5-7 in a plasma sample or a sample comprising PBMCs from the individual, wherein an expression level of the one or more genes set forth in any one of Tables 5-7 in the plasma sample or in the sample comprising PBMCs that is at or above a reference level of the one or more genes set forth in any one of Tables 5-7 identifies the individual as likely to respond to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)); and (b) administering an effective amount of an anti-cancer therapy comprising a PD-L1 axis binding antagonist to the individual based on the expression level of the one or more genes set forth in any one of Tables 5-7 determined in the plasma sample or in the sample comprising PBMCs in step (a).

In another aspect, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 axis binding antagonist based on an expression level of one or more genes set forth in any one of Tables 5-7 in a plasma sample or a sample comprising PBMCs from the individual that is at or above a reference level of the one or more genes set forth in any one of Tables 5-7, the method comprising administering to the individual an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)).

In another aspect, provided herein is a PD-L1 axis binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the individual has been identified as likely to respond to treatment with an anti-cancer therapy including a PD-L1 axis binding antagonist based on an expression level of one or more genes set forth in any one of Tables 5-7 in a plasma sample or a sample including PBMCs from the individual that is at or above a reference level of the one or more genes set forth in any one of Tables 5-7.

In another aspect, provided herein is a method of treating an individual having a cancer (e.g., bladder cancer (e.g., UC) or kidney cancer (e.g., RCC)), the method comprising: (a) administering an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)) to the individual; (b) determining the expression level of one or more genes set forth in any one of Tables 5-7 in a sample obtained from the individual at a time point following administration of the anti-cancer therapy; and (c) continuing to administer the anti-cancer therapy to the individual if the expression level of the one or more genes set forth in any one of Tables 5-7 in the individual's sample is at or above the reference level of the one or more genes set forth in any one of Tables 5-7.

In another aspect, provided herein is a method of monitoring the response of an individual having a cancer (e.g., bladder cancer (e.g., UC) or kidney cancer (e.g., RCC)) to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)), the method comprising: (a) determining the expression level of one or more genes set forth in any one of Tables 5-7 in a sample obtained from the individual at a time point following administration of the anti-cancer therapy; (b) comparing the expression level of the one or more genes set forth in any one of Tables 5-7 in the sample with a reference level of the one or more genes set forth in any one of Tables 5-7; and (c) continuing to administer the anti-cancer therapy to the individual if the expression level of the one or more genes set forth in any one of Tables 5-7 in the individual's sample is at or above the reference level.

In another aspect, provided herein is a PD-L1 axis binding antagonist (e.g., an anti-PD-L1 antibody such as atezolizumab) or a PD-1 binding antagonist (e.g., an anti-PD-1 antibody)) for use in a method of treating an individual having a cancer (e.g., bladder cancer (e.g., UC) or kidney cancer (e.g., RCC)), the method including: (a) administering an effective amount of an anti-cancer therapy including a PD-L1 axis binding antagonist to the individual; (b) determining the expression level of one or more genes set forth in any one of Tables 5-7 in a sample obtained from the individual at a time point following administration of the anti-cancer therapy; and (c) continuing to administer the anti-cancer therapy to the individual if the expression level of the one or more genes set forth in any one of Tables 5-7 in the individual's sample is at or above the reference level of the one or more genes set forth in any one of Tables 5-7.

In another aspect, provided herein is a PD-L1 axis binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the expression level of one or more genes set forth in any one of Tables 5-7 in a sample obtained from the individual at a time point following administration of the PD-L1 axis binding antagonist has been determined to be at or above a reference level of the one or more genes set forth in any one of Tables 5-7.

In another aspect, provided herein is a method of treating an individual having a cancer (e.g., bladder cancer (e.g., UC) or kidney cancer (e.g., RCC)), the method comprising: (a) administering an effective amount of an anti-cancer therapy comprising a PD-L1 axis binding antagonist (e.g., an anti-PD-L1 antibody such as atezolizumab) or a PD-1 binding antagonist (e.g., an anti-PD-1 antibody)) to the individual; (b) determining the expression level of one or more genes set forth in any one of Tables 5-7 in a sample obtained from the individual at a time point following administration of the anti-cancer therapy, wherein the sample is a plasma sample, a tumor tissue sample, or a sample comprising peripheral blood mononuclear cells (PBMCs); and (c) continuing to administer the anti-cancer therapy to the individual if the expression level of the one or more genes set forth in any one of Tables 5-7 in the individual's sample is at or above the reference level of the one or more genes set forth in any one of Tables 5-7.

In another aspect, provided herein is a method of monitoring the response of an individual having a cancer (e.g., bladder cancer (e.g., UC) or kidney cancer (e.g., RCC)) to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)), the method comprising: (a) determining the expression level of one or more genes set forth in any one of Tables 5-7 in a sample obtained from the individual at a time point following administration of the anti-cancer therapy, wherein the sample is a plasma sample, a tumor tissue sample, or a sample comprising PBMCs; (b) comparing the expression level of the one or more genes set forth in any one of Tables 5-7 in the sample with a reference level of the one or more genes set forth in any one of Tables 5-7; and (c) continuing to administer the anti-cancer therapy to the individual if the expression level of the one or more genes set forth in any one of Tables 5-7 in the individual's sample is at or above the reference level.

In another aspect, provided herein is a PD-L1 axis binding antagonist (e.g., an anti-PD-L1 antibody such as atezolizumab) or a PD-1 binding antagonist (e.g., an anti-PD-1 antibody)) for use in a method of treating an individual having a cancer (e.g., bladder cancer (e.g., UC) or kidney cancer (e.g., RCC)), the method including: (a) administering an effective amount of an anti-cancer therapy including a PD-L1 axis binding antagonist to the individual; (b) determining the expression level of one or more genes set forth in any one of Tables 5-7 in a sample obtained from the individual at a time point following administration of the anti-cancer therapy, wherein the sample is a plasma sample, a tumor tissue sample, or a sample including PBMCs; and (b) continuing to administer the anti-cancer therapy to the individual if the expression level of the one or more genes set forth in any one of Tables 5-7 in the individual's sample is at or above the reference level of the one or more genes set forth in any one of Tables 5-7.

In another aspect, provided herein is a PD-L1 axis binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the expression level of one or more genes set forth in any one of Tables 5-7 in a sample obtained from the individual at a time point following administration of the PD-L1 axis binding antagonist has been determined to be at or above a reference level of the one or more genes set forth in any one of Tables 5-7, wherein the sample is a plasma sample, a tumor tissue sample, or a sample including PBMCs.

Any suitable time point following administration of the anti-cancer therapy may be used. For example, the time point following administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) following administration of the anti-cancer therapy. In some instances, the time point following administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks following administration of the anti-cancer therapy. In some instances, the time point following administration of the anti-cancer therapy is about 4 to about 8 weeks following administration of the anti-cancer therapy. For example, in some aspects, the time point following administration of the anti-cancer therapy is about 6 weeks following administration of the anti-cancer therapy.

In another aspect, provided herein is a method of treating an individual having a cancer (e.g., bladder cancer (e.g., UC) or kidney cancer (e.g., RCC)), the method comprising: (a) determining the expression level of one or more genes set forth in any one of Tables 5-7 in a sample from the individual that has been obtained at a time point prior to or concurrently with administration of an anti-cancer therapy comprising a PD-L1 axis binding antagonist (e.g., an anti-PD-L1 antibody such as atezolizumab) or a PD-1 binding antagonist (e.g., an anti-PD-1 antibody)), wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy comprising a PD-L1 binding antagonist; and (b) administering an effective amount of an anti-cancer therapy comprising a PD-L1 axis binding antagonist to the individual based on the expression level of the one or more genes set forth in any one of Tables 5-7 determined in step (a).

In another aspect, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)) based on an expression level of one or more genes set forth in any one of Tables 5-7 in a sample from the individual that is at or above a reference level of the one or more genes set forth in any one of Tables 5-7, the method comprising administering to the individual an effective amount of an anti-cancer therapy comprising a PD-L1 axis binding antagonist, and wherein the sample has been obtained at a time point prior to or concurrently with administration of the anti-cancer therapy comprising a PD-L1 axis binding antagonist.

In another aspect, provided herein is a PD-L1 axis binding antagonist for use in treating an individual having a cancer (e.g., bladder cancer (e.g., UC) or kidney cancer (e.g., RCC)), wherein the individual has been identified as likely to respond to treatment with an anti-cancer therapy including a PD-L1 binding antagonist (e.g., an anti-PD-L1 antibody such as atezolizumab) or a PD-1 binding antagonist (e.g., an anti-PD-1 antibody)) based on an expression level of one or more genes set forth in any one of Tables 5-7 in a sample from the individual that is at or above a reference level of the one or more genes set forth in any one of Tables 5-7, wherein the sample is obtained from the individual at a time point prior to or concurrently with administration of the anti-cancer therapy including a PD-L1 axis binding antagonist.

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of treating an individual having a cancer (e.g., bladder cancer (e.g., UC) or kidney cancer (e.g., RCC)), the method comprising: (a) determining the expression level of one or more genes set forth in any one of Tables 5-7 in a sample from the individual that has been obtained at a time point prior to or concurrently with administration of an anti-cancer therapy comprising a PD-L1 axis binding antagonist, wherein an expression level of the one or more genes set forth in any one of Tables 5-7 in the sample that is at or above a reference level of the one or more genes set forth in any one of Tables 5-7 identifies the individual as likely to respond to treatment with an anti-cancer therapy comprising a PD-L1 binding antagonist, wherein the response is indicated by OS or ORR; and (b) administering an effective amount of an anti-cancer therapy comprising a PD-L1 axis binding antagonist to the individual based on the expression level of the one or more genes set forth in any one of Tables 5-7 determined in step (a).

In another example, provided herein is a method of treating an individual having a cancer (e.g., bladder cancer (e.g., UC) or kidney cancer (e.g., RCC)) who has been determined to be likely to respond to treatment with an anti-cancer therapy comprising a PD-L1 axis binding antagonist based on an expression level of one or more genes set forth in any one of Tables 5-7 in a sample from the individual that is at or above a reference level of the one or more genes set forth in any one of Tables 5-7, the method comprising administering to the individual an effective amount of an anti-cancer therapy comprising a PD-L1 axis binding antagonist, wherein the response is indicated by OS or ORR, and wherein the sample has been obtained at a time point prior to or concurrently with administration of the anti-cancer therapy comprising a PD-L1 axis binding antagonist.

In another example, provided herein is a PD-L1 axis binding antagonist for use in treating an individual having a cancer (e.g., bladder cancer (e.g., UC) or kidney cancer (e.g., RCC)), wherein the individual has been identified as likely to respond to treatment with an anti-cancer therapy including a PD-L1 axis binding antagonist based on an expression level of one or more genes set forth in any one of Tables 5-7 in a sample from the individual that is at or above a reference level of the one or more genes set forth in any one of Tables 5-7, wherein the sample is obtained from the individual at a time point prior to or concurrently with administration of the anti-cancer therapy including a PD-L1 axis binding antagonist, and wherein the response is indicated by OS or ORR.

Any suitable time point prior to administration of the anti-cancer therapy may be used. For example, the time point prior to administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) prior to administration of the anti-cancer therapy. In some instances, the time point prior to administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks prior to administration of the anti-cancer therapy.

In any of the preceding methods, the individual may be likely to have improved response from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). For example, the individual may be likely to have an improved ORR, improved CR rate, improved PR rate, extended PFS, and/or extended OS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In some particular instances, the individual may be likely to have an improved ORR from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In other particular instances, the individual may be likely to have an extended PFS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist). In yet other particular instances, the individual may be likely to have an extended OS from treatment with the anti-cancer therapy including the PD-L1 axis binding antagonist (e.g., the PD-L1 binding antagonist) compared to treatment with an anti-cancer therapy that does not include the PD-L1 axis binding antagonist (e.g., the PD-L1 binding antagonist).

In other aspects of the present disclosure, individuals having decreased or low levels of one or more genes that are downregulated in IL8 high cancer, e.g., one or more genes set forth in any one of Tables 5-7 can be treated with an anti-cancer therapy, e.g., an anti-cancer therapy other than or in addition to a PD-L1 axis binding antagonist. For example, individuals having decreased or low baseline or on-treatment expression levels of one or more genes set forth in any one of Tables 5-7 (e.g., relative to a reference level of the one or more genes set forth in any one of Tables 5-7) can be treated with an anti-cancer therapy other than or in addition to a PD-L1 axis binding antagonist (e.g., combination therapy with a PD-L1 axis binding antagonist and one or more additional therapeutic agents (e.g., VEGF antagonists (e.g., anti-VEGF antibodies such as bevacizumab), or an anti-cancer therapy that does not include a PD-L1 axis binding antagonist).

For example, provided herein is a method of treating an individual having a cancer (e.g., bladder cancer (e.g., UC) or kidney cancer (e.g., RCC)), the method comprising: (a) determining the expression level of one or more genes set forth in any one of Tables 5-7 in a sample from the individual, wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy; and (b) administering an effective amount of an anti-cancer therapy other than or in addition to a PD-L1 axis binding antagonist to the individual based on the expression level of the one or more genes set forth in any one of Tables 5-7 determined in step (a).

In another example, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of one or more genes set forth in any one of Tables 5-7 in a sample from the individual that is below a reference level of the one or more genes set forth in any one of Tables 5-7, the method comprising administering to the individual an effective amount of an anti-cancer therapy other than or in addition to a PD-L1 axis binding antagonist.

In yet another example, provided herein is an anti-cancer therapy other than or in addition to a PD-L1 axis binding antagonist for use in treating an individual having a cancer (e.g., bladder cancer (e.g., UC) or kidney cancer (e.g., RCC)), wherein the individual has been identified as less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of one or more genes set forth in any one of Tables 5-7 in a sample from the individual that is below a reference level of the one or more genes set forth in any one of Tables 5-7.

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of treating an individual having a cancer (e.g., bladder cancer (e.g., UC) or kidney cancer (e.g., RCC)), the method comprising: (a) determining the expression level of one or more genes set forth in any one of Tables 5-7 in a sample from the individual, wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)) monotherapy, wherein the response is indicated by OS or ORR; and (b) administering an effective amount of an anti-cancer therapy other than or in addition to a PD-L1 axis binding antagonist to the individual based on the expression level of the one or more genes set forth in any one of Tables 5-7 determined in step (a).

In another example, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 axis binding antagonist (e.g., an anti-PD-L1 antibody such as atezolizumab) or a PD-1 binding antagonist (e.g., an anti-PD-1 antibody)) monotherapy based on an expression level of the one or more genes set forth in any one of Tables 5-7 in a sample from the individual that is below a reference level of the one or more genes set forth in any one of Tables 5-7, the method comprising administering to the individual an effective amount of an anti-cancer therapy other than or in addition to a PD-L1 axis binding antagonist, wherein the response is indicated by OS or ORR.

In yet another example, provided herein is an anti-cancer therapy other than or in addition to a PD-L1 axis binding antagonist for use in treating an individual having a cancer (e.g., bladder cancer (e.g., UC) or kidney cancer (e.g., RCC)), wherein the individual has been identified as less likely to respond to treatment with an anti-cancer therapy including a PD-L1 axis binding antagonist (e.g., an anti-PD-L1 antibody such as atezolizumab) or a PD-1 binding antagonist (e.g., an anti-PD-1 antibody)) monotherapy based on an expression level of one or more genes set forth in any one of Tables 5-7 in a sample from the individual that is below a reference level of the one or more genes set forth in any one of Tables 5-7, wherein the response is indicated by OS or ORR.

For example, provided herein is a method of treating an individual having a cancer (e.g., bladder cancer (e.g., UC) or kidney cancer (e.g., RCC)), the method comprising: (a) determining the expression level of one or more genes set forth in any one of Tables 5-7 in a sample from the individual, wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy comprising a PD-L1 binding antagonist (e.g., an anti-PD-L1 antibody such as atezolizumab) monotherapy, wherein the response is indicated by OS or ORR; and (b) administering an effective amount of an anti-cancer therapy other than or in addition to a PD-L1 binding antagonist to the individual based on the expression level of the one or more genes set forth in any one of Tables 5-7 determined in step (a).

In another example, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 binding antagonist (e.g., an anti-PD-L1 antibody such as atezolizumab) monotherapy based on an expression level of one or more genes set forth in any one of Tables 5-7 in a sample from the individual that is below a reference level of the one or more genes set forth in any one of Tables 5-7, the method comprising administering to the individual an effective amount of an anti-cancer therapy other than or in addition to a PD-L1 binding antagonist, wherein the response is indicated by OS or ORR.

In yet another example, provided herein is an anti-cancer therapy other than or in addition to a PD-L1 binding antagonist for use in treating an individual having a cancer (e.g., bladder cancer (e.g., UC) or kidney cancer (e.g., RCC)), wherein the individual has been identified as less likely to respond to treatment with an anti-cancer therapy including a PD-L1 binding antagonist (e.g., an anti-PD-L1 antibody such as atezolizumab) monotherapy based on an expression level of one or more genes set forth in any one of Tables 5-7 in a sample from the individual that is below a reference level of the one or more genes set forth in any one of Tables 5-7, wherein the response is indicated by OS or ORR.

In another aspect, provided herein is a method of treating an individual having a cancer (e.g., bladder cancer (e.g., UC) or kidney cancer (e.g., RCC)), the method comprising: (a) determining the expression level of one or more genes set forth in any one of Tables 5-7 in a plasma sample or a sample comprising PBMCs from the individual, wherein an expression level of the one or more genes set forth in any one of Tables 5-7 in the plasma sample or in the sample comprising PBMCs that is below a reference level of the one or more genes set forth in any one of Tables 5-7 identifies the individual as less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy; and (b) administering an effective amount of an anti-cancer therapy other than or in addition to a PD-L1 axis binding antagonist to the individual based on the expression level of the one or more genes set forth in any one of Tables 5-7 in the plasma sample or in the sample comprising PBMCs determined in step (a).

In another aspect, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of one or more genes set forth in any one of Tables 5-7 in a plasma sample or a sample comprising PBMCs from the individual that is below a reference level of the one or more genes set forth in any one of Tables 5-7, the method comprising administering to the individual an effective amount of an anti-cancer therapy other than or in addition to a PD-L1 axis binding antagonist.

In another aspect, provided herein is an anti-cancer therapy other than or in addition to a PD-L1 axis binding antagonist for use in treating an individual having a cancer (e.g., bladder cancer (e.g., UC) or kidney cancer (e.g., RCC)), wherein the individual has been identified as less likely to respond to treatment with an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of one or more genes set forth in any one of Tables 5-7 in a plasma sample or a sample including PBMCs from the individual that is below a reference level of the one or more genes set forth in any one of Tables 5-7.

In another aspect, provided herein is a method of treating an individual having a cancer (e.g., bladder cancer (e.g., UC) or kidney cancer (e.g., RCC)), the method comprising: (a) administering an effective amount of an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) to the individual; (b) determining the expression level of one or more genes set forth in any one of Tables 5-7 in a sample obtained from the individual at a time point following administration of the anti-cancer therapy comprising a PD-L1 axis binding antagonist; and (c) administering an anti-cancer therapy other than or in addition to a PD-L1 axis binding antagonist to the individual if the expression level of the one or more genes set forth in any one of Tables 5-7 in the individual's sample is below the reference level of the one or more genes set forth in any one of Tables 5-7.

In another aspect, provided herein is a method of monitoring the response of an individual having a cancer (e.g., bladder cancer (e.g., UC) or kidney cancer (e.g., RCC)) to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), the method comprising: (a) determining the expression level of one or more genes set forth in any one of Tables 5-7 in a sample obtained from the individual at a time point following administration of an anti-cancer therapy comprising a PD-L1 axis binding antagonist; (b) comparing the expression level of the one or more genes set forth in any one of Tables 5-7 in the sample with a reference level of the one or more genes set forth in any one of Tables 5-7; and (c) administering an anti-cancer therapy other than or in addition to a PD-L1 axis binding antagonist to the individual if the expression level of the one or more genes set forth in any one of Tables 5-7 in the individual's sample is below the reference level.

In another aspect, provided herein is an anti-cancer therapy other than or in addition to a PD-L1 axis binding antagonist for use in a method of treating an individual having a cancer (e.g., bladder cancer (e.g., UC) or kidney cancer (e.g., RCC)), the method including: (a) administering an effective amount of an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) to the individual; (b) determining the expression level of one or more genes set forth in any one of Tables 5-7 in a sample obtained from the individual at a time point following administration of the anti-cancer therapy including a PD-L1 axis binding antagonist; and (c) administering the anti-cancer therapy other than or in addition to a PD-L1 axis binding antagonist to the individual if the expression level of the one or more genes set forth in any one of Tables 5-7 in the individual's sample is below the reference level of the one or more genes set forth in any one of Tables 5-7.

In another aspect, provided herein is an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) for use in treating an individual having a cancer, wherein the expression level of one or more genes set forth in any one of Tables 5-7 in a sample obtained from the individual at a time point following administration of a PD-L1 axis binding antagonist has been determined to be below a reference level of the one or more genes set forth in any one of Tables 5-7.

In another aspect, provided herein is a method of treating an individual having a cancer (e.g., bladder cancer (e.g., UC) or kidney cancer (e.g., RCC)), the method comprising: (a) administering an effective amount of an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) to the individual; (b) determining the expression level of one or more genes set forth in any one of Tables 5-7 in a sample obtained from the individual at a time point following administration of the anti-cancer therapy comprising a PD-L1 binding antagonist, wherein the sample is a plasma sample, a tumor tissue sample, or a sample comprising PBMCs; and (c) administering an anti-cancer therapy other than or in addition to a PD-L1 axis binding antagonist to the individual if the expression level of the one or more genes set forth in any one of Tables 5-7 in the individual's sample is below the reference level of the one or more genes set forth in any one of Tables 5-7.

In another aspect, provided herein is a method of monitoring the response of an individual having a cancer (e.g., bladder cancer (e.g., UC) or kidney cancer (e.g., RCC)) to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), the method comprising: (a) determining the expression level of one or more genes set forth in any one of Tables 5-7 in a sample obtained from the individual at a time point following administration of an anti-cancer therapy comprising a PD-L1 axis binding antagonist, wherein the sample is a plasma sample, a tumor tissue sample, or a sample comprising PBMCs; (b) comparing the expression level of the one or more genes set forth in any one of Tables 5-7 in the sample with a reference level of the one or more genes set forth in any one of Tables 5-7; and (c) administering an anti-cancer therapy other than or in addition to a PD-L1 axis binding antagonist to the individual if the expression level of the one or more genes set forth in any one of Tables 5-7 in the individual's sample is below the reference level.

In another aspect, provided herein is an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) for use in a method of treating an individual having a cancer (e.g., bladder cancer (e.g., UC) or kidney cancer (e.g., RCC)), the method including: (a) administering an effective amount of an anti-cancer therapy including a PD-L1 axis binding antagonist to the individual; (b) determining the expression level of one or more genes set forth in any one of Tables 5-7 in a sample obtained from the individual at a time point following administration of an anti-cancer therapy including a PD-L1 axis binding antagonist, wherein the sample is a plasma sample, a tumor tissue sample, or a sample including PBMCs; and (c) administering the anti-cancer therapy other than or in addition to a PD-L1 axis binding antagonist to the individual if the expression level of the one or more genes set forth in any one of Tables 5-7 in the individual's sample is below the reference level of the one or more genes set forth in any one of Tables 5-7.

In another aspect, provided herein is an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the expression level of one or more genes set forth in any one of Tables 5-7 in a sample obtained from the individual at a time point following administration of a PD-L1 axis binding antagonist has been determined to be below a reference level of the one or more genes set forth in any one of Tables 5-7, wherein the sample is a plasma sample, a tumor tissue sample, or a sample including PBMCs.

Any suitable time point following administration of the anti-cancer therapy may be used. For example, the time point following administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) following administration of the anti-cancer therapy. In some instances, the time point following administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks following administration of the anti-cancer therapy. In some instances, the time point following administration of the anti-cancer therapy is about 4 to about 8 weeks following administration of the anti-cancer therapy. For example, in some aspects, the time point following administration of the anti-cancer therapy is about 6 weeks following administration of the anti-cancer therapy.

In another aspect, provided herein is a method of treating an individual having a cancer (e.g., bladder cancer (e.g., UC) or kidney cancer (e.g., RCC)), the method comprising: (a) determining the expression level of one or more genes set forth in any one of Tables 5-7 in a sample from the individual that has been obtained at a time point prior to or concurrently with administration of an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy comprising a PD-L1 axis binding antagonist monotherapy; and (b) administering an effective amount of an anti-cancer therapy other than or in addition to a PD-L1 axis binding antagonist to the individual based on the expression level of the one or more genes set forth in any one of Tables 5-7 determined in step (a).

In another aspect, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of one or more genes set forth in any one of Tables 5-7 in a sample from the individual that is below a reference level of the one or more genes set forth in any one of Tables 5-7, the method comprising administering to the individual an effective amount of an anti-cancer therapy other than or in addition to a PD-L1 axis binding antagonist and wherein the sample has been obtained at a time point prior to or concurrently with administration of an anti-cancer therapy comprising a PD-L1 axis binding antagonist.

In another aspect, provided herein is an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the individual has been identified as less likely to respond to treatment with an anti-cancer therapy including a PD-L1 axis binding antagonist monotherapy based on an expression level of one or more genes set forth in any one of Tables 5-7 in a sample from the individual that is below a reference level of the one or more genes set forth in any one of Tables 5-7, wherein the sample is obtained from the individual at a time point prior to or concurrently with administration of the anti-cancer therapy including a PD-L1 axis binding antagonist

The response may be any suitable response. For example, in some aspects, the response is indicated by ORR, complete response (CR) rate, partial response (PR) rate, PFS, OS, and/or duration of response (DOR). In some instances, the response is indicated by ORR or OS. In some particular instances, the response is indicated by ORR. In other particular instances, the response is indicated by PFS. In yet other particular instances, the response is indicated by OS.

For example, provided herein is a method of treating an individual having a cancer (e.g., bladder cancer (e.g., UC) or kidney cancer (e.g., RCC)), the method comprising: (a) determining the expression level of one or more genes set forth in any one of Tables 5-7 in a sample from the individual that has been obtained at a time point prior to or concurrently with administration of an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), wherein an expression level of the one or more genes set forth in any one of Tables 5-7 in the sample that is below a reference level of the one or more genes set forth in any one of Tables 5-7 identifies the individual as less likely to respond to treatment with an anti-cancer therapy comprising a PD-L1 axis binding antagonist monotherapy, wherein the response is indicated by OS or ORR; and (b) administering an effective amount of an anti-cancer therapy other than or in addition to a PD-L1 axis binding antagonist to the individual based on the expression level of the one or more genes set forth in any one of Tables 5-7 determined in step (a).

In another example, provided herein is a method of treating an individual having a cancer (e.g., bladder cancer (e.g., UC) or kidney cancer (e.g., RCC)) who has been determined to be less likely to respond to treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) monotherapy based on an expression level of one or more genes set forth in any one of Tables 5-7 in a sample from the individual that is below a reference level of the one or more genes set forth in any one of Tables 5-7, the method comprising administering to the individual an effective amount of an anti-cancer therapy other than or in addition to a PD-L1 axis binding antagonist, wherein the response is indicated by OS or ORR, and wherein the sample has been obtained at a time point prior to or concurrently with administration of an anti-cancer therapy comprising a PD-L1 axis binding antagonist.

In yet another example, provided herein is an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) for use in treating an individual having a cancer (e.g., bladder cancer (e.g., UC) or kidney cancer (e.g., RCC)), wherein the individual has been identified as less likely to respond to treatment with an anti-cancer therapy including a PD-L1 axis binding antagonist monotherapy based on an expression level of one or more genes set forth in any one of Tables 5-7 in a sample from the individual that is below a reference level of the one or more genes set forth in any one of Tables 5-7, wherein the sample is obtained from the individual at a time point prior to or concurrently with administration of the anti-cancer therapy including a PD-L1 axis binding antagonist, and wherein the response is indicated by OS or ORR.

Any suitable time point prior to administration of the anti-cancer therapy may be used. For example, the time point prior to administration of the anti-cancer therapy may be hours (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours), days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days), weeks (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, or about 52 weeks), months (e.g., about 1, about 2, about 3, about 4, about 5, about 6, about 7, about 8, about 9, about 10, about 11, or about 12 months), or years (e.g., about 1, about 2, about 3, about 4, about 5, about 6, about 7, about 8, about 9, about 10 years) prior to administration of the anti-cancer therapy. In some instances, the time point prior to administration of the anti-cancer therapy is about 1 to about 16 weeks, about 2 to about 16 weeks, about 3 to about 16 weeks, about 4 to about 16 weeks, about 5 to about 16 weeks, about 6 to about 16 weeks, about 7 to about 16 weeks, about 8 to about 16 weeks, about 9 to about 16 weeks, about 10 to about 16 weeks, about 11 to about 16 weeks, about 12 to about 16 weeks, about 13 to about 16 weeks, about 14 to about 16 weeks, about 15 to about 16 weeks, about 1 to about 15 weeks, about 2 to about 15 weeks, about 3 to about 15 weeks, about 4 to about 15 weeks, about 5 to about 15 weeks, about 6 to about 15 weeks, about 7 to about 15 weeks, about 8 to about 15 weeks, about 9 to about 15 weeks, about 10 to about 15 weeks, about 11 to about 15 weeks, about 12 to about 15 weeks, about 13 to about 15 weeks, about 14 to about 15 weeks, about 1 to about 14 weeks, about 2 to about 14 weeks, about 3 to about 14 weeks, about 4 to about 14 weeks, about 5 to about 14 weeks, about 6 to about 14 weeks, about 7 to about 14 weeks, about 8 to about 14 weeks, about 9 to about 14 weeks, about 10 to about 14 weeks, about 11 to about 14 weeks, about 12 to about 14 weeks, about 13 to about 14 weeks, about 1 to about 13 weeks, about 2 to about 13 weeks, about 3 to about 13 weeks, about 4 to about 13 weeks, about 5 to about 13 weeks, about 6 to about 13 weeks, about 7 to about 13 weeks, about 8 to about 13 weeks, about 9 to about 13 weeks, about 10 to about 13 weeks, about 11 to about 13 weeks, about 12 to about 13 weeks, about 1 to about 12 weeks, about 2 to about 12 weeks, about 3 to about 12 weeks, about 4 to about 12 weeks, about 5 to about 12 weeks, about 6 to about 12 weeks, about 7 to about 12 weeks, about 8 to about 12 weeks, about 9 to about 12 weeks, about 10 to about 12 weeks, about 11 to about 12 weeks, about 1 to about 11 weeks, about 2 to about 11 weeks, about 3 to about 11 weeks, about 4 to about 11 weeks, about 5 to about 11 weeks, about 6 to about 11 weeks, about 7 to about 11 weeks, about 8 to about 11 weeks, about 9 to about 11 weeks, about 10 to about 11 weeks, about 1 to about 10 weeks, about 2 to about 10 weeks, about 3 to about 10 weeks, about 4 to about 10 weeks, about 5 to about 10 weeks, about 6 to about 10 weeks, about 7 to about 10 weeks, about 8 to about 10 weeks, about 9 to about 10 weeks, about 1 to about 9 weeks, about 2 to about 9 weeks, about 3 to about 9 weeks, about 4 to about 9 weeks, about 5 to about 9 weeks, about 6 to about 9 weeks, about 7 to about 9 weeks, about 8 to about 9 weeks, about 1 to about 8 weeks, about 2 to about 8 weeks, about 3 to about 8 weeks, about 4 to about 8 weeks, about 5 to about 8 weeks, about 6 to about 8 weeks, about 7 to about 8 weeks, about 1 to about 7 weeks, about 2 to about 7 weeks, about 3 to about 7 weeks, about 4 to about 7 weeks, about 5 to about 7 weeks, about 6 to about 7 weeks, about 1 to about 6 weeks, about 2 to about 6 weeks, about 3 to about 6 weeks, about 4 to about 6 weeks, about 5 to about 6 weeks, about 1 to about 5 weeks, about 2 to about 5 weeks, about 3 to about 5 weeks, about 4 to about 5 weeks, about 1 to about 4 weeks, about 2 to about 4 weeks, about 3 to about 4 weeks, about 1 to about 3 weeks, about 2 to about 3 weeks, or about 1 to about 2 weeks prior to administration of the anti-cancer therapy.

In any of the preceding methods, the individual may be likely to have a reduced response from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). For example, the individual may be likely to have a reduced ORR, reduced CR rate, reduced PR rate, shortened PFS, or shortened OS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In some particular instances, the individual may be likely to have a reduced ORR from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In other particular instances, the individual may be likely to have a shortened PFS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist). In other particular instances, the individual may be likely to have a shortened OS from treatment with the anti-cancer therapy including the PD-L1 axis binding antagonist (e.g., the PD-L1 binding antagonist) monotherapy compared to treatment with an anti-cancer therapy other than or in addition to a PD-L1 axis binding antagonist (e.g., a PD-L1 binding antagonist).

4. Samples and Reference Levels

Any suitable sample may be used in any of the methods described herein. Exemplary, non-limiting samples include a plasma sample, a tissue sample, a cell sample, a whole blood sample, a serum sample, or a combination thereof. For example, in some instances, the sample is a plasma sample. In another example, in some instances, the tissue sample is a tumor tissue sample. In some instances, the tumor tissue sample includes tumor cells, tumor-infiltrating immune cells, stromal cells, or a combination thereof. In some instances, the tumor-infiltrating immune cells include tumor-infiltrating myeloid cells. In some instances, the tumor tissue sample is a formalin-fixed and paraffin-embedded (FFPE) sample, an archival sample, a fresh sample, or a frozen sample. In some instances, the cell sample includes peripheral blood mononuclear cells (PBMCs).

Any suitable reference level may be used in any of the methods described herein. For example, in some instances, the reference level of a biomarker described herein, e.g., one or more genes set forth in any one of Tables 1-7, e.g., IL8, is determined from a population of individuals having a cancer. For example, in some instances, the reference level of a biomarker described herein, e.g., one or more genes set forth in any one of Tables 1-7, e.g., IL8, is a median expression level, a tertile expression level, or a maximally-selected log-rank reference level determined in a population of patients having a cancer. In some embodiments, the maximally-selected log-rank reference level is 12 pg/mL of IL8. In some instances, the reference level of a biomarker described herein, e.g., one or more genes set forth in any one of Tables 1-7, e.g., IL8, is a median expression level determined in a population of patients having a cancer. In one particular example, the reference level may be the level of a biomarker described herein, e.g., one or more genes set forth in any one of Tables 1-7, e.g., IL8, in a sample obtained from the individual at a time point prior to or concurrently with administration of the anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)). In another particular example, the reference level may be the level of a biomarker described herein, e.g., one or more genes set forth in any one of Tables 1-7, e.g., IL8, in a sample obtained from the individual at a time following administration of the anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)).

In certain instances, the presence and/or expression levels/amount of a biomarker (e.g., a biomarker described herein, e.g., one or more genes set forth in any one of Tables 1-7, e.g., IL8) in a first sample is increased or elevated as compared to presence/absence and/or expression levels/amount in a second sample. In certain instances, the presence/absence and/or expression levels/amount of a biomarker (e.g., a biomarker described herein, e.g., one or more genes set forth in any one of Tables 1-7, e.g., IL8) in a first sample is decreased or reduced as compared to presence and/or expression levels/amount in a second sample. In certain instances, the second sample is a reference sample, reference cell, reference tissue, control sample, control cell, or control tissue. Additional disclosures for determining the presence/absence and/or expression levels/amount of a gene are described herein.

In certain instances, a reference sample, reference cell, reference tissue, control sample, control cell, or control tissue is a single sample or a combination of multiple samples from the same subject or individual that are obtained at one or more different time points than when the test sample is obtained. For example, a reference sample, reference cell, reference tissue, control sample, control cell, or control tissue is obtained at an earlier time point from the same subject or individual than when the test sample is obtained. Such reference sample, reference cell, reference tissue, control sample, control cell, or control tissue may be useful if the reference sample is obtained during initial diagnosis of cancer and the test sample is later obtained when the cancer becomes metastatic.

In certain instances, a reference sample, reference cell, reference tissue, control sample, control cell, or control tissue is a combination of multiple samples from one or more healthy individuals who are not the patient. In certain embodiments, a reference sample, reference cell, reference tissue, control sample, control cell, or control tissue is a combination of multiple samples from one or more individuals with a disease or disorder (e.g., cancer) who are not the subject or individual. In certain embodiments, a reference sample, reference cell, reference tissue, control sample, control cell, or control tissue is pooled RNA samples from normal tissues or pooled plasma or serum samples from one or more individuals who are not the patient. In certain embodiments, a reference sample, reference cell, reference tissue, control sample, control cell, or control tissue is pooled RNA samples from tumor tissues or pooled plasma or serum samples from one or more individuals with a disease or disorder (e.g., cancer) who are not the patient.

In some examples of any of the methods, elevated or increased expression refers to an overall increase of about any of 10%, 20%, 30%, 40%, 50%, 60%, 70%, 80%, 90%, 95%, 96%, 97%, 98%, 99% or greater, in the level of biomarker (e.g., a biomarker described herein, e.g., one or more genes set forth in any one of Tables 1-7, e.g., IL8) (e.g., protein or nucleic acid (e.g., gene or mRNA)), detected by standard art-known methods such as those described herein, as compared to a reference sample, reference cell, reference tissue, control sample, control cell, or control tissue. In certain embodiments, the elevated expression refers to the increase in expression level/amount of a biomarker in the sample wherein the increase is at least about any of 1.5×, 1.75×, 2×, 3×, 4×, 5×, 6×, 7×, 8×, 9×, 10×, 25×, 50×, 75×, or 100× the expression level/amount of the respective biomarker in a reference sample, reference cell, reference tissue, control sample, control cell, or control tissue. In some embodiments, elevated expression refers to an overall increase of greater than about 1.5-fold, about 1.75-fold, about 2-fold, about 2.25-fold, about 2.5-fold, about 2.75-fold, about 3.0-fold, or about 3.25-fold as compared to a reference sample, reference cell, reference tissue, control sample, control cell, control tissue, or internal control (e.g., housekeeping gene).

In some examples of any of the methods, reduced expression refers to an overall reduction of about any of 10%, 20%, 30%, 40%, 50%, 60%, 70%, 80%, 90%, 95%, 96%, 97%, 98%, 99% or greater, in the level of biomarker (e.g., a biomarker described herein, e.g., one or more genes set forth in any one of Tables 1-7, e.g., IL8) (e.g., protein or nucleic acid (e.g., gene or mRNA)), detected by standard art known methods such as those described herein, as compared to a reference sample, reference cell, reference tissue, control sample, control cell, or control tissue. In certain embodiments, reduced expression refers to the decrease in expression level/amount of a biomarker in the sample wherein the decrease is at least about any of 0.9×, 0.8×, 0.7×, 0.6×, 0.5×, 0.4×, 0.3×, 0.2×, 0.1×, 0.05×, or 0.01× the expression level/amount of the respective biomarker in a reference sample, reference cell, reference tissue, control sample, control cell, or control tissue.

In some examples of any of the methods described herein, the individual may have an expression level of a T effector (T_{eff}) signature in a tumor sample that is at or above a reference level for the T_{eff} signature. In some instances, the T_{eff} signature comprises one or more genes selected from CD8A, GZMA, GZMB, and PRF1. In some instances, the T_{eff} signature comprises two or more genes selected from CD8A, GZMA, GZMB, and PRF1. In some instances, the T_{eff} signature comprises three or more genes selected from CD8A, GZMA, GZMB, and PRF1. In some instances, the T_{eff} signature comprises CD8A, GZMA, GZMB, and PRF1.

In some examples of any of the methods described herein, the individual has not been previously treated for the cancer.

In other examples of any of the methods described herein, the individual has previously been treated for the cancer.

The individual may have any suitable type of cancer. Exemplary, non-limiting cancers include a bladder cancer, a kidney cancer, a breast cancer, a colorectal cancer, a lung cancer, a lymphoma, a prostate cancer, a liver cancer, a head and neck cancer, a melanoma, an ovarian cancer, a mesothelioma, or a myeloma. In some aspects, the bladder cancer is urothelial carcinoma (UC). In some aspects, the UC is locally advanced or metastatic UC. In some aspects, the individual has received a prior platinum-based chemotherapy. In some aspects, the individual has progressed after the prior platinum-based chemotherapy. In some aspects, the individual is previously untreated for the locally advanced or metastatic UC. In some aspects, the individual is ineligible for platinum-based chemotherapy. In some aspects, the individual is cisplatin-ineligible. In some aspects, the kidney cancer is renal cell carcinoma (RCC). In some aspects, the RCC is metastatic RCC (mRCC). In some aspects, the individual is previously untreated for the mRCC. In some aspects, the lung cancer is non-small cell lung cancer or small-cell lung cancer. In some aspects, the breast cancer is triple-negative breast cancer TNBC or HER2-positive breast cancer.

5. Exemplary Therapeutic Agents, Administration Routes, Combination Therapies, and Dosing Regimens

Any of the methods describe herein may further include administering an anti-cancer therapy to the individual. For example, in some instances, the anti-cancer therapy is selected based on the determination of the expression level of a biomarker described herein, e.g., one or more genes set forth in any one of Tables 1-7, e.g., IL8, in a sample from the individual.

For example, in some instances, the method further includes administering an anti-cancer therapy including a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) to the individual.

In other instances, the method further includes administering an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) to the individual. In some instances, the anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)) includes a VEGF antagonist (e.g., an anti-VEGF antibody), an IL8 antagonist (e.g., an anti-IL-8 antibody or a small molecule IL8 inhibitor), an IL1B antagonist (e.g., an anti-IL1B antibody or a small molecule IL1B inhibitor), an IL1R antagonist (e.g., an anti-IL1R antibody or a small molecule IL1R inhibitor), or a combination thereof. Any suitable VEGF antagonist, IL8 antagonist, IL1B antagonist, or IL1R antagonist may be used (see, e.g., Section IV below). In some instances, the anti-cancer therapy includes a VEGF antagonist and a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)). In some instances, the VEGF antagonist is an anti-VEGF antibody or a VEGF receptor (VEGFR) inhibitor. In some aspects, the VEGF antagonist is an anti-VEGF antibody. In some aspects, the anti-VEGF antibody is bevacizumab.

Any suitable PD-L1 axis binding antagonist may be used may be used (see, e.g., Section IV below). For example, in some instances, the PD-L1 axis binding antagonist is selected from the group consisting of a PD-L1 binding antagonist, a PD-1 binding antagonist, and a PD-L2 binding antagonist.

In some examples, the PD-L1 axis binding antagonist is a PD-L1 binding antagonist. Any suitable PD-L1 binding antagonist may be used may be used (see, e.g., Section IV below). In some aspects, the PD-L1 binding antagonist inhibits the binding of PD-L1 to one or more of its ligand binding partners. In some aspects, the PD-L1 binding antagonist inhibits the binding of PD-L1 to PD-1. In some aspects, the PD-L1 binding antagonist inhibits the binding of PD-L1 to B7-1. In some aspects, the PD-L1 binding antagonist inhibits the binding of PD-L1 to both PD-1 and B7-1. In some aspects, the PD-L1 binding antagonist is an anti-PD-L1 antibody. In some aspects, the anti-PD-L1 antibody is selected from the group consisting of: atezolizumab, MDX-1105, durvalumab, and avelumab.

In some aspects of any of the methods described herein, the anti-PD-L1 antibody includes one, two, three, four, five, or all six of following hypervariable regions (HVRs): (a) an HVR-H1 sequence of GFTFSDSWIH (SEQ ID NO: 19); (b) an HVR-H2 sequence of AWISPYGGSTYYADSVKG (SEQ ID NO: 20); (c) an HVR-H3 sequence of RHWPGGFDY (SEQ ID NO: 21); (d) an HVR-L1 sequence of RASQDVSTAVA (SEQ ID NO: 22); (e) an HVR-L2 sequence of SASFLYS (SEQ ID NO: 23); and (f) an HVR-L3 sequence of QQYLYHPAT (SEQ ID NO: 24), or one, two, three, four, five, or six HVRs having at least 90% (e.g., at least 90%, at least 91%, at least 92%, at least 93%, at least 94%, at least 95%, at least 96%, at least 97%, at least 98%, or at least 99%) sequence identity to (a) an HVR-H1 sequence of GFTFSDSWIH (SEQ ID NO: 19); (b) an HVR-H2 sequence of AWISPYGGSTYYADSVKG (SEQ ID NO: 20); (c) an HVR-H3 sequence of RHWPGGFDY (SEQ ID NO: 21); (d) an HVR-L1 sequence of RASQDVSTAVA (SEQ ID NO: 22); (e) an HVR-L2 sequence of SASFLYS (SEQ ID NO: 23); and/or (f) an HVR-L3 sequence of QQYLYHPAT (SEQ ID NO: 24).

In some particular aspects of any of the methods described herein, the anti-PD-L1 antibody includes the following hypervariable regions (HVRs): (a) an HVR-H1 sequence of GFTFSDSWIH (SEQ ID NO: 19); (b) an HVR-H2 sequence of AWISPYGGSTYYADSVKG (SEQ ID NO: 20); (c) an HVR-H3 sequence of RHWPGGFDY (SEQ ID NO: 21); (d) an HVR-L1 sequence of RASQDVSTAVA (SEQ ID NO: 22); (e) an HVR-L2 sequence of SASFLYS (SEQ ID NO: 23); and (f) an HVR-L3 sequence of QQYLYHPAT (SEQ ID NO: 24).

In some aspects of any of the methods described herein, the anti-PD-L1 antibody includes: (a) a heavy chain variable (VH) domain including an amino acid sequence having at least 90% (e.g., at least 90%, at least 91%, at least 92%, at least 93%, at least 94%, at least 95%, at least 96%, at least 97%, at least 98%, or at least 99%) sequence identity to the amino acid sequence of EVQLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSS (SEQ ID NO: 25); (b) a light chain variable (VL) domain including an amino acid sequence having at least 90% (e.g., at least 90%, at least 91%, at least 92%, at least 93%, at least 94%, at least 95%, at least 96%, at least 97%, at least 98%, or at least 99%) sequence identity to the amino acid sequence of DIQMTQSPSSLSASVGDRVTITCRASQDVSTAVAWYQQKPGKAPKLLIYSASFLYSGVPSRFSGSGSGTD FTLTISSLQPEDFATYYCQQYLYHPATFGQGTKVEIKR (SEQ ID NO: 4); or (c) a VH domain as in (a) and a VL domain as in (b). In some aspects, the anti-PD-L1 antibody includes: (a) a VH domain including an amino acid sequence having at least 95% sequence identity to the amino acid sequence of SEQ ID NO: 25; (b) a VL domain including an amino acid sequence having at least 95% sequence identity to the amino acid sequence of SEQ ID NO: 4; or (c) a VH domain as in (a) and a VL domain as in (b). In some aspects, the anti-PD-L1 antibody includes: (a) a VH domain including an amino acid sequence having at least 96% sequence identity to the amino acid sequence of SEQ ID NO: 25; (b) a VL domain including an amino acid sequence having at least 96% sequence identity to the amino acid sequence of SEQ ID NO: 4; or (c) a VH domain as in (a) and a VL domain as in (b). In some aspects, the anti-PD-L1 antibody includes: (a) a VH domain including an amino acid sequence having at least 97% sequence identity to the amino acid sequence of SEQ ID NO: 25; (b) a VL domain including an amino acid sequence having at least 97% sequence identity to the amino acid sequence of SEQ ID NO: 4; or (c) a VH domain as in (a) and a VL domain as in (b). In some aspects, the anti-PD-L1 antibody includes: (a) a VH domain including an amino acid sequence having at least 98% sequence identity to the amino acid sequence of SEQ ID NO: 25; (b) a VL domain including an amino acid sequence having at least 98% sequence identity to the amino acid sequence of SEQ ID NO: 4; or (c) a VH domain as in (a) and a VL domain as in (b). In some aspects, the anti-PD-L1 antibody includes: (a) a VH domain including an amino acid sequence having at least 99% sequence identity to the amino acid sequence of SEQ ID NO: 25; (b) a VL domain including an amino acid sequence having at least 99% sequence identity to the amino acid sequence of SEQ ID NO: 4; or (c) a VH domain as in (a) and a VL domain as in (b). In some aspects, the anti-PD-L1 antibody includes: (a) a VH domain including the amino acid sequence of SEQ ID NO: 25; (b) a VL domain including the amino acid sequence of SEQ ID NO: 4; or (c) a VH domain as in (a) and a VL domain as in (b). In some aspects, the anti-PD-L1 antibody includes: (a) a VH domain including the amino acid sequence of SEQ ID NO: 25; and (b) a VL domain including the amino acid sequence of SEQ ID NO: 4. In some aspects, the anti-PD-L1 antibody is atezolizumab.

Any of the methods described herein may further include administering an additional therapeutic agent to the individual. In some aspects, the additional therapeutic agent is selected from the group consisting of an immunotherapy agent, a cytotoxic agent, a growth inhibitory agent, a radiation therapy agent, an anti-angiogenic agent, and combinations thereof. In some instances, the second therapeutic agent is an agonist directed against an activating co-stimulatory molecule. In some instances, the second therapeutic agent is an antagonist directed against an inhibitory co-stimulatory molecule.

In some aspects of any of the methods described herein, the individual is a human.

The compositions utilized in the methods described herein (e.g., PD-L1 axis binding antagonists, VEGF antagonists, and other anti-cancer therapeutic agents) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions described herein can also be administered systemically or locally. The method of administration can vary depending on various factors (e.g., the compound or composition being administered and the severity of the condition, disease, or disorder being treated). In some instances, the PD-L1 axis binding antagonist is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Therapeutic agents, including, e.g., PD-L1 axis binding antagonists, VEGF antagonists, and other anti-cancer therapeutic agents described herein (or any additional therapeutic agent) (e.g., an antibody, binding polypeptide, and/or small molecule) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The therapeutic agent need not be, but is optionally formulated with and/or administered concurrently with one or more agents currently used to prevent or treat the disorder in question. The effective amount of such other agents depends on the amount of the therapeutic agent present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of a cancer (e.g., a bladder cancer (e.g., an urothelial bladder cancer)), the appropriate dosage of a therapeutic agent (e.g., a PD-L1 axis binding antagonist, a VEGF antagonist, or any other anti-cancer therapeutic agent) described herein (when used alone or in combination with one or more other additional therapeutic agents) will depend on the type of disease to be treated, the severity and course of the disease, whether the therapeutic agent is administered for preventive or therapeutic purposes, previous therapy, the patient's clinical history and response to the PD therapeutic agent, and the discretion of the attending physician. The therapeutic agent is suitably administered to the patient at one time or over a series of treatments. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g., every week or every three weeks (e.g., such that the patient receives, for example, from about two to about twenty, or e.g., about six doses of the therapeutic agent). An initial higher loading dose followed by one or more lower doses may be administered. However, other dosage regimens may be useful. The progress of this therapy is easily monitored by conventional techniques and assays.

For example, as a general proposition, the therapeutically effective amount of an antibody (e.g., a PD-L1 axis binding antagonist antibody or a VEGF antagonist antibody) administered to human will be in the range of about 0.01 to about 50 mg/kg of patient body weight, whether by one or more administrations. In some instances, the antibody used is about 0.01 mg/kg to about 45 mg/kg, about 0.01 mg/kg to about 40 mg/kg, about 0.01 mg/kg to about 35 mg/kg, about 0.01 mg/kg to about 30 mg/kg, about 0.01 mg/kg to about 25 mg/kg, about 0.01 mg/kg to about 20 mg/kg, about 0.01 mg/kg to about 15 mg/kg, about 0.01 mg/kg to about 10 mg/kg, about 0.01 mg/kg to about 5 mg/kg, or about 0.01 mg/kg to about 1 mg/kg administered daily, weekly, every two weeks, every three weeks, or monthly, for example. In some instances, the antibody is administered at 15 mg/kg. However, other dosage regimens may be useful. In one instance, an anti-PD-L1 antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, or about 1800 mg on day 1 of 21-day cycles (every three weeks, q3w). In some instances, anti-PD-L1 antibody atezolizumab is administered at 1200 mg intravenously every three weeks (q3w). In some instances, anti-PD-L1 antibody atezolizumab is administered at 840 mg intravenously every two weeks (q2w). In some instances, anti-PD-L1 antibody atezolizumab is administered at 1680 mg intravenously every four weeks (q4w). The dose may be administered as a single dose or as multiple doses (e.g., 2 or 3 doses), such as infusions. The dose of the antibody administered in a combination treatment may be reduced as compared to a single treatment. The progress of this therapy is easily monitored by conventional techniques.

In some instances, the methods further involve administering to the patient an effective amount of an additional therapeutic agent. In some instances, the second therapeutic agent is selected from the group consisting of a cytotoxic agent, a chemotherapeutic agent, a growth-inhibitory agent, a radiation therapy agent, an anti-angiogenic agent, and combinations thereof. In some instances, a PD-L1 axis binding antagonist may be administered in conjunction with a chemotherapy or chemotherapeutic agent. In some instances, a PD-L1 axis binding antagonist may be administered in conjunction with a radiation therapy agent. In some instances, a PD-L1 axis binding antagonist may be administered in conjunction with a targeted therapy or targeted therapeutic agent. In some instances, a PD-L1 axis binding antagonist may be administered in conjunction with an immunotherapy or immunotherapeutic agent, for example a monoclonal antibody. In some instances, the second therapeutic agent is an agonist directed against an activating co-stimulatory molecule. In some instances, the second therapeutic agent is an antagonist directed against an inhibitory co-stimulatory molecule.

Such combination therapies noted above encompass combined administration (where two or more therapeutic agents are included in the same or separate formulations), and separate administration, in which case, administration of a PD-L1 axis binding antagonist can occur prior to, simultaneously, and/or following, administration of the additional therapeutic agent or agents. In one instance, administration of PD-L1 axis binding antagonist and administration of an additional therapeutic agent occur within about one month, or within about one, two or three weeks, or within about one, two, three, four, five, or six days, of each other.

Without wishing to be bound to theory, it is thought that enhancing T-cell stimulation, by promoting an activating co-stimulatory molecule or by inhibiting a negative co-stimulatory molecule, may promote tumor cell death thereby treating or delaying progression of cancer. In some instances, a PD-L1 axis binding antagonist may be administered in conjunction with an agonist directed against an activating co-stimulatory molecule. In some instances, an activating co-stimulatory molecule may include CD40, CD226, CD28, OX40, GITR, CD137, CD27, HVEM, or CD127. In some instances, the agonist directed against an activating co-stimulatory molecule is an agonist antibody that binds to CD40, CD226, CD28, OX40, GITR, CD137, CD27, HVEM, or CD127. In some instances, a PD-L1 axis binding antagonist may be administered in conjunction with an antagonist directed against an inhibitory co-stimulatory molecule. In some instances, an inhibitory co-stimulatory molecule may include CTLA-4 (also known as CD152), TIM-3, BTLA, VISTA, LAG-3, B7-H3, B7-H4, IDO, TIGIT, MICA/B, or arginase. In some instances, the antagonist directed against an inhibitory co-stimulatory molecule is an antagonist antibody that binds to CTLA-4, TIM-3, BTLA, VISTA, LAG-3, B7-H3, B7-H4, IDO, TIGIT, MICA/B, or arginase.

In some instances, a PD-L1 axis binding antagonist may be administered in conjunction with an antagonist directed against CTLA-4 (also known as CD152), e.g., a blocking antibody. In some instances, a PD-L1 axis binding antagonist may be administered in conjunction with ipilimumab (also known as MDX-010, MDX-101, or YERVOY®). In some instances, a PD-L1 axis binding antagonist may be administered in conjunction with tremelimumab (also known as ticilimumab or CP-675,206). In some instances, a PD-L1 axis binding antagonist may be administered in conjunction with an antagonist directed against B7-H3 (also known as CD276), e.g., a blocking antibody. In some instances, a PD-L1 axis binding antagonist may be administered in conjunction with MGA271. In some instances, a PD-L1 axis binding antagonist may be administered in conjunction with an antagonist directed against a TGF-beta, e.g., metelimumab (also known as CAT-192), fresolimumab (also known as GC1008), or LY2157299.

In some instances, a PD-L1 axis binding antagonist may be administered in conjunction with a treatment comprising adoptive transfer of a T-cell (e.g., a cytotoxic T-cell or CTL) expressing a chimeric antigen receptor (CAR). In some instances, a PD-L1 axis binding antagonist may be administered in conjunction with a treatment comprising adoptive transfer of a T-cell comprising a dominant-negative TGF beta receptor, e.g., a dominant-negative TGF beta type II receptor. In some instances, a PD-L1 axis binding antagonist may be administered in conjunction with a treatment comprising a HERCREEM protocol (see, e.g., ClinicalTrials.gov Identifier NCT00889954).

In some instances, a PD-L1 axis binding antagonist may be administered in conjunction with an agonist directed against CD137 (also known as TNFRSF9, 4-1BB, or ILA), e.g., an activating antibody. In some instances, a PD-L1 axis binding antagonist may be administered in conjunction with urelumab (also known as BMS-663513). In some instances, a PD-L1 axis binding antagonist may be administered in conjunction with an agonist directed against CD40, e.g., an activating antibody. In some instances, a PD-L1 axis binding antagonist may be administered in conjunction with CP-870893. In some instances, a PD-L1 axis binding antagonist may be administered in conjunction with an agonist directed against OX40 (also known as CD134), e.g., an activating antibody. In some instances, a PD-L1 axis binding antagonist may be administered in conjunction with an anti-OX40 antibody (e.g., AgonOX). In some instances, a PD-L1 axis binding antagonist may be administered in conjunction with an agonist directed against CD27, e.g., an activating antibody. In some instances, a PD-L1 axis binding antagonist may be administered in conjunction with CDX-1127. In some instances, a PD-L1 axis binding antagonist may be administered in conjunction with an antagonist directed against indoleamine-2,3-dioxygenase (IDO). In some instances, with the IDO antagonist is 1-methyl-D-tryptophan (also known as 1-D-MT).

In some instances, a PD-L1 axis binding antagonist may be administered in conjunction with an antibody-drug conjugate. In some instances, the antibody-drug conjugate comprises mertansine or monomethyl auristatin E (MMAE). In some instances, a PD-L1 axis binding antagonist may be administered in conjunction with an anti-NaPi2b antibody-MMAE conjugate (also known as DNIB0600A or RG7599). In some instances, a PD-L1 axis binding antagonist may be administered in conjunction with trastuzumab emtansine (also known as T-DM1, ado-trastuzumab emtansine, or KADCYLA®, Genentech). In some instances, a PD-L1 axis binding antagonist may be administered in conjunction with DMUC5754A. In some instances, a PD-L1 axis binding antagonist may be administered in conjunction with an antibody-drug conjugate targeting the endothelin B receptor (EDNBR), e.g., an antibody directed against EDNBR conjugated with MMAE.

In some instances, a PD-L1 axis binding antagonist may be administered in conjunction with an anti-angiogenesis agent. In some instances, a PD-L1 axis binding antagonist may be administered in conjunction with an antibody directed against a VEGF, e.g., VEGF-A. In some instances, a PD-L1 axis binding antagonist may be administered in conjunction with bevacizumab (also known as AVASTIN®, Genentech). In some instances, a PD-L1 axis binding antagonist may be administered in conjunction with an antibody directed against angiopoietin 2 (also known as Ang2). In some instances, a PD-L1 axis binding antagonist may be administered in conjunction with MEDI3617.

In some instances, a PD-L1 axis binding antagonist may be administered in conjunction with an antineoplastic agent. In some instances, a PD-L1 axis binding antagonist may be administered in conjunction with an agent targeting CSF-1R (also known as M-CSFR or CD115). In some instances, a PD-L1 axis binding antagonist may be administered in conjunction with anti-CSF-1R (also known as IMC-CS4). In some instances, a PD-L1 axis binding antagonist may be administered in conjunction with an interferon, for example interferon alpha or interferon gamma. In some instances, a PD-L1 axis binding antagonist may be administered in conjunction with Roferon-A (also known as recombinant Interferon alpha-2a). In some instances, a PD-L1 axis binding antagonist may be administered in conjunction with GM-CSF (also known as recombinant human granulocyte macrophage colony stimulating factor, rhu GM-CSF, sargramostim, or LEUKINE®). In some instances, a PD-L1 axis binding antagonist may be administered in conjunction with IL-2 (also known as aldesleukin or PROLEUKIN®). In some instances, a PD-L1 axis binding antagonist may be administered in conjunction with IL-12. In some instances, a PD-L1 axis binding antagonist may be administered in conjunction with an antibody targeting CD20. In some instances, the antibody targeting CD20 is obinutuzumab (also known as GA101 or GAZYVA®) or rituximab. In some instances, a PD-L1 axis binding antagonist may be administered in conjunction with an antibody targeting GITR. In some instances, the antibody targeting GITR is TRX518.

In some instances, a PD-L1 axis binding antagonist may be administered in conjunction with a cancer vaccine. In some instances, the cancer vaccine is a peptide cancer vaccine, which in some instances is a personalized peptide vaccine. In some instances, the peptide cancer vaccine is a multivalent long peptide, a multi-peptide, a peptide cocktail, a hybrid peptide, or a peptide-pulsed dendritic cell vaccine (see, e.g., Yamada et al., Cancer Sci. 104:14-21, 2013). In some instances, a PD-L1 axis binding antagonist may be administered in conjunction with an adjuvant. In some instances, a PD-L1 axis binding antagonist may be administered in conjunction with a treatment comprising a TLR agonist, e.g., Poly-ICLC (also known as HILTONOL®), LPS, MPL, or CpG ODN. In some instances, a PD-L1 axis binding antagonist may be administered in conjunction with tumor necrosis factor (TNF) alpha. In some instances, a PD-L1 axis binding antagonist may be administered in conjunction with IL-1. In some instances, a PD-L1 axis binding antagonist may be administered in conjunction with HMGB1. In some instances, a PD-L1 axis binding antagonist may be administered in conjunction with an IL-10 antagonist. In some instances, a PD-L1 axis binding antagonist may be administered in conjunction with an IL-4 antagonist. In some instances, a PD-L1 axis binding antagonist may be administered in conjunction with an IL-13 antagonist. In some instances, a PD-L1 axis binding antagonist may be administered in conjunction with an HVEM antagonist. In some instances, a PD-L1 axis binding antagonist may be administered in conjunction with an ICOS agonist, e.g., by administration of ICOS-L, or an agonistic antibody directed against ICOS. In some instances, a PD-L1 axis binding antagonist may be administered in conjunction with a treatment targeting CX3CL1. In some instances, a PD-L1 axis binding antagonist may be administered in conjunction with a treatment targeting CXCL9. In some instances, a PD-L1 axis binding antagonist may be administered in conjunction with a treatment targeting CXCL10. In some instances, a PD-L1 axis binding antagonist may be administered in conjunction with a treatment targeting CCL5. In some instances, a PD-L1 axis binding antagonist may be administered in conjunction with an LFA-1 or ICAM1 agonist. In some instances, a PD-L1 axis binding antagonist may be administered in conjunction with a Selectin agonist.

In some instances, a PD-L1 axis binding antagonist may be administered in conjunction with a targeted therapy. In some instances, a PD-L1 axis binding antagonist may be administered in conjunction with an inhibitor of B-Raf. In some instances, a PD-L1 axis binding antagonist may be administered in conjunction with vemurafenib (also known as ZELBORAF®). In some instances, a PD-L1 axis binding antagonist may be administered in conjunction with dabrafenib (also known as TAFINLAR®). In some instances, a PD-L1 axis binding antagonist may be administered in conjunction with erlotinib (also known as TARCEVA®). In some instances, a PD-L1 axis binding antagonist may be administered in conjunction with an inhibitor of a MEK, such as MEK1 (also known as MAP2K1) or MEK2 (also known as MAP2K2). In some instances, a PD-L1 axis binding antagonist may be administered in conjunction with cobimetinib (also known as GDC-0973 or XL-518). In some instances, a PD-L1 axis binding antagonist may be administered in conjunction with trametinib (also known as MEKINIST®). In some instances, a PD-L1 axis binding antagonist may be administered in conjunction with an inhibitor of K-Ras. In some instances, a PD-L1 axis binding antagonist may be administered in conjunction with an inhibitor of c-Met. In some instances, a PD-L1 axis binding antagonist may be administered in conjunction with onartuzumab (also known as MetMAb). In some instances, a PD-L1 axis binding antagonist may be administered in conjunction with an inhibitor of Alk. In some instances, a PD-L1 axis binding antagonist may be administered in conjunction with AF802 (also known as CH5424802 or alectinib). In some instances, a PD-L1 axis binding antagonist may be administered in conjunction with an inhibitor of a phosphatidylinositol 3-kinase (PI3K). In some instances, a PD-L1 axis binding antagonist may be administered in conjunction with BKM120. In some instances, a PD-L1 axis binding antagonist may be administered in conjunction with idelalisib (also known as GS-1101 or CAL-101). In some embodiments, a PD-L1 axis binding antagonist may be administered in conjunction with perifosine (also known as KRX-0401). In some embodiments, a PD-L1 axis binding antagonist may be administered in conjunction with an inhibitor of an Akt. In some embodiments, a PD-L1 axis binding antagonist may be administered in conjunction with MK2206. In some instances, a PD-L1 axis binding antagonist may be administered in conjunction with GSK690693. In some instances, a PD-L1 axis binding antagonist may be administered in conjunction with GDC-0941. In some instances, a PD-L1 axis binding antagonist may be administered in conjunction with an inhibitor of mTOR. In some instances, a PD-L1 axis binding antagonist may be administered in conjunction with sirolimus (also known as rapamycin). In some instances, a PD-L1 axis binding antagonist may be administered in conjunction with temsirolimus (also known as CCI-779 or TORISEL®). In some instances, a PD-L1 axis binding antagonist may be administered in conjunction with everolimus (also known as RAD001). In some instances, a PD-L1 axis binding antagonist may be administered in conjunction with ridaforolimus (also known as AP-23573, MK-8669, or deforolimus). In some instances, a PD-L1 axis binding antagonist may be administered in conjunction with OSI-027. In some instances, a PD-L1 axis binding antagonist may be administered in conjunction with AZD8055. In some instances, a PD-L1 axis binding antagonist may be administered in conjunction with INK128. In some instances, a PD-L1 axis binding antagonist may be administered in conjunction with a dual PI3K/mTOR inhibitor. In some instances, a PD-L1 axis binding antagonist may be administered in conjunction with XL765. In some instances, a PD-L1 axis binding antagonist may be administered in conjunction with GDC-0980. In some instances, a PD-L1 axis binding antagonist may be administered in conjunction with BEZ235 (also known as NVP-BEZ235). In some instances, a PD-L1 axis binding antagonist may be administered in conjunction with BGT226. In some instances, a PD-L1 axis binding antagonist may be administered in conjunction with GSK2126458. In some instances, a PD-L1 axis binding antagonist may be administered in conjunction with PF-04691502. In some instances, a PD-L1 axis binding antagonist may be administered in conjunction with PF-05212384 (also known as PKI-587).

### IV. Compositions and Pharmaceutical Formulations

Provided herein are methods for treating or delaying progression of a cancer (e.g., bladder cancer (e.g., UC) and kidney cancer (e.g., RCC)) in an individual that include administering to the individual an anti-cancer therapy comprising a PD-L1 axis binding antagonist. Also provided herein are methods for identifying an individual having a cancer (e.g., bladder cancer (e.g., UC) and kidney cancer (e.g., RCC)) who is likely to respond to treatment with an anti-cancer therapy comprising a PD-L1 axis binding antagonist. Further provided herein are methods for determining whether a patient suffering from a cancer (e.g., bladder cancer (e.g., UC) and kidney cancer (e.g., RCC)) is likely to respond to treatment co treatment with an anti-cancer therapy comprising a PD-L1 axis binding antagonist. Provided herein are methods for predicting responsiveness of a patient suffering from a cancer (e.g., bladder cancer (e.g., UC) and kidney cancer (e.g., RCC)) to treatment with an anti-cancer therapy comprising a PD-L1 axis binding antagonist. Provided herein are methods for selecting a therapy for a patient suffering from a cancer (e.g., bladder cancer (e.g., UC) and kidney cancer (e.g., RCC)). Any of the methods may be based on the expression level of a biomarker provided herein, for example, the expression level of one or more genes set forth in any one of Tables 1-7, e.g., IL8 expression in a sample from an individual having or suspected of having a cancer. For example, individuals with decreased IL8 expression relative to a reference level of IL8, or an on-treatment decrease in IL8 expression relative to a reference level of IL8, may be candidates for treatment with an anti-cancer therapy comprising a PD-L1 axis binding antagonist. In another example, individuals with decreased expression of one or more genes set forth in any one of Tables 2-4 relative to a reference level of the one or more genes, or an on-treatment decrease in expression of one or more genes set forth in any one of Tables 2-4 relative to a reference level of the one or more genes, may be candidates for treatment with an anti-cancer therapy comprising a PD-L1 axis binding antagonist. In yet another example, individuals with increased expression of one or more genes set forth in any one of Tables 5-7 relative to a reference level of the one or more genes, or an on-treatment increase in expression of one or more genes set forth in any one of Tables 5-7 relative to a reference level of the one or more genes, may be candidates for treatment with an anti-cancer therapy comprising a PD-L1 axis binding antagonist.

In other aspects, provided herein are methods for treating or delaying progression of a cancer (e.g., bladder cancer (e.g., UC) and kidney cancer (e.g., RCC)) in an individual that include administering to the individual a therapeutically effective amount of an anti-cancer therapy other than or in addition to a PD-L1 axis binding antagonist. Also provided herein are methods of identifying an individual having a cancer (e.g., bladder cancer (e.g., UC) and kidney cancer (e.g., RCC)) who is less likely to respond to treatment with an anti-cancer therapy comprising a PD-L1 binding antagonist monotherapy. Provided herein are methods for determining whether a patient suffering from a cancer (e.g., bladder cancer (e.g., UC) and kidney cancer (e.g., RCC)) is likely to respond to treatment with an anti-cancer therapy other than or in addition to a PD-L1 axis binding antagonist. Provided herein are methods for predicting responsiveness of a patient suffering from a cancer (e.g., bladder cancer (e.g., UC) and kidney cancer (e.g., RCC)) to treatment with an anti-cancer therapy comprising a PD-L1 binding antagonist monotherapy. Provided herein are methods for selecting a therapy for a patient suffering from a cancer (e.g., bladder cancer (e.g., UC) and kidney cancer (e.g., RCC)), e.g., for treatment with an anti-cancer therapy other than or in addition to a PD-L1 axis binding antagonist. Any of the preceding methods may be based on the expression level of a biomarker provided herein, for example, the expression level of one or more genes set forth in any one of Tables 1-7, e.g., IL8 expression in a sample from an individual having or suspected of having a cancer. For example, individuals with elevated IL8 expression relative to a reference level of IL8, or an on-treatment increase in IL8 expression relative to a reference level of IL8, may be candidates for treatment with an anti-cancer therapy other than or in addition to a PD-L1 axis binding antagonist. In another example, individuals with elevated expression of one or more genes set forth in any one of Tables 2-4 relative to a reference level of the one or more genes, or an on-treatment increase in expression of one or more genes set forth in any one of Tables 2-4 relative to a reference level of the one or more genes, may be candidates for treatment with an anti-cancer therapy other than or in addition to a PD-L1 axis binding antagonist. In yet another example, individuals with decreased expression of one or more genes set forth in any one of Tables 5-7 relative to a reference level of the one or more genes, or an on-treatment decrease in expression of one or more genes set forth in any one of Tables 5-7 relative to a reference level of the one or more genes, may be candidates for treatment with an anti-cancer therapy other than or in addition to a PD-L1 axis binding antagonist.

These agents, and combinations thereof, are useful for the treatment of cancer, e.g., as part of any of the methods, compositions for use, or uses of the present disclosure. Any suitable PD-L1 axis binding antagonist, VEGF antagonist, and/or additional anti-cancer agent can be used in the methods and assays described herein. Non-limiting examples suitable for use in the methods, compositions for use, or uses of the present disclosure are described further below. Any of these examples may be used, e.g., in the preparation of a medicament, e.g., a medicament for treating cancer in an individual.

A. Exemplary PD-L1 Axis Binding Antagonists

Any suitable PD-L1 axis binding antagonist may be used. For example, a PD-L1 axis binding antagonist includes a PD-1 binding antagonist, a PD-L1 binding antagonist, and a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1LG1,” “CD274,” “B7-H,” and “PDL1.” An exemplary human PD-L1 is shown in UniProtKB/Swiss-Prot Accession No.Q9NZQ7.1. PD-L2 (programmed death ligand 2) is also referred to in the art as “programmed cell death 1 ligand 2,” “PDCD1LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

1. Exemplary PD-L1 Binding Antagonists

Any suitable PD-L1 binding antagonist described herein or known in the art can be used. In some instances, the PD-L1 binding antagonist is an anti-PD-L1 antibody, for example, as described below. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)₂ fragments. In some instances, the anti-PD-L1 antibody is a humanized antibody. In some instances, the anti-PD-L1 antibody is a human antibody. In some instances, the anti-PD-L1 antibody is selected from the group consisting of atezolizumab, YW243.55.S70, atezolizumab, MDX-1105, and MEDI4736 (durvalumab), and MSB0010718C (avelumab). Antibody YW243.55.S70 is an anti-PD-L1 described in WO 2010/077634. MDX-1105, also known as BMS-936559, is an anti-PD-L1 antibody described in WO2007/005874. MEDI4736 (durvalumab) is an anti-PD-L1 monoclonal antibody described in WO2011/066389 and US2013/034559. Examples of anti-PD-L1 antibodies useful for the methods of this invention, and methods for making thereof, are described in WO 2010/077634, WO 2007/005874, WO 2011/066389, U.S. Pat. No. 8,217,149, and US 2013/034559, which are incorporated herein by reference.

Anti-PD-L1 antibodies described in WO 2010/077634 A1 and U.S. Pat. No. 8,217,149 may be used in the methods described herein. In some instances, the anti-PD-L1 antibody comprises a heavy chain variable region sequence of SEQ ID NO:3 and/or a light chain variable region sequence of SEQ ID NO:4. In a still further instance, provided is an isolated anti-PD-L1 antibody comprising a heavy chain variable region and/or a light chain variable region sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence: EVQLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSA (SEQ ID NO:3), and
(b) the light chain sequence has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 4)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR.

In one instance, the anti-PD-L1 antibody comprises a heavy chain variable region comprising an HVR-H1, HVR-H2 and HVR-H3 sequence, wherein:
(a) the HVR-H1 sequence is GFTFSX₁SWIH (SEQ ID NO:5);
(b) the HVR-H2 sequence is AWIX₂PYGGSX₃YYADSVKG (SEQ ID NO:6);
(c) the HVR-H3 sequence is RHWPGGFDY (SEQ ID NO:7);

further wherein: X₁ is D or G; X₂ is S or L; X₃ is T or S. In one specific aspect, X₁ is D; X₂ is S and X₃ is T. In another aspect, the polypeptide further comprises variable region heavy chain framework sequences juxtaposed between the HVRs according to the formula: (FR-H1)-(HVR-H1)-(FR-H2)-(HVR-H2)-(FR-H3)-(HVR-H3)-(FR-H4). In yet another aspect, the framework sequences are derived from human consensus framework sequences. In a further aspect, the framework sequences are VH subgroup III consensus framework. In a still further aspect, at least one of the framework sequences is the following:

FR-H1 is
(SEQ ID NO: 8)
EVQLVESGGGLVQPGGSLRLSCAAS
FR-H2 is
(SEQ ID NO: 9)
WVRQAPGKGLEWV
FR-H3 is
(SEQ ID NO: 10)
RFTISADTSKNTAYLQMNSLRAEDTAVYYCAR
FR-H4 is
(SEQ ID NO: 11)
WGQGTLVTVSA.

In a still further aspect, the heavy chain polypeptide is further combined with a variable region light chain comprising an HVR-L1, HVR-L2 and HVR-L3, wherein:
(a) the HVR-L1 sequence is RASQX₄X₅X₆TX₇X₈A (SEQ ID NO:12);
(b) the HVR-L2 sequence is SASX₉LX₁₀S, (SEQ ID NO:13);
(c) the HVR-L3 sequence is QQX₁₁X₁₂X₁₃X₁₄PX₁₅T (SEQ ID NO:14);
   wherein: X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, G, For I; X₁₄ is H, V, P, T or I; X₁₅ is A, W, R, P or T. In a still further aspect, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H; X₁₅ is A.

In a still further aspect, the light chain further comprises variable region light chain framework sequences juxtaposed between the HVRs according to the formula: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the framework sequences are VL kappa I consensus framework. In a still further aspect, at least one of the framework sequence is the following:

FR-L1 is
(SEQ ID NO: 15)
DIQMTQSPSSLSASVGDRVTITC
FR-L2 is
(SEQ ID NO: 16)
WYQQKPGKAPKLLIY
FR-L3 is
(SEQ ID NO: 17)
GVPSRFSGSGSGTDFTLTISSLQPEDFATYYC
FR-L4 is
(SEQ ID NO: 18)
FGQGTKVEIKR.

In another instance, provided is an isolated anti-PD-L1 antibody or antigen binding fragment comprising a heavy chain and a light chain variable region sequence, wherein:
(a) the heavy chain comprises an HVR-H1, HVR-H2 and HVR-H3, wherein further:
   (i) the HVR-H1 sequence is GFTFSX₁SWIH; (SEQ ID NO:5)
   (ii) the HVR-H2 sequence is AWIX₂PYGGSX₃YYADSVKG (SEQ ID NO:6)
   (iii) the HVR-H3 sequence is RHWPGGFDY, and (SEQ ID NO:7)
(b) the light chain comprises an HVR-L1, HVR-L2 and HVR-L3, wherein further:
   (i) the HVR-L1 sequence is RASQX₄X₅X₆TX₇X₈A (SEQ ID NO:12)
   (ii) the HVR-L2 sequence is SASX₉LX₁₀S; and (SEQ ID NO:13)
   (iii) the HVR-L3 sequence is QQX₁₁X₁₂X₁₃X₁₄PX₁₅T; (SEQ ID NO:14)
      wherein: X₁ is D or G; X₂ is S or L; X₃ is T or S; X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, G, F or I; X₁₄ is H, V, P, T or I; X₁₅ is A, W, R, P or T. In a specific aspect, X₁ is D; X₂ is S and X₃ is T. In another aspect, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H; X₁₅ is A. In yet another aspect, X₁ is D; X₂ is S and X₃ is T, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H and X₁₅ is A.

In a further aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the heavy chain framework sequences are derived from a Kabat subgroup I, 11, or III sequence. In a still further aspect, the heavy chain framework sequence is a VH subgroup III consensus framework. In a still further aspect, one or more of the heavy chain framework sequences are set forth as SEQ ID NOs:8, 9, 10 and 11. In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences are set forth as SEQ ID NOs:15, 16, 17 and 18.

In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect, the minimal effector function results from an “effector-less Fc mutation” or aglycosylation. In still a further instance, the effector-less Fc mutation is an N297A or D265A/N297A substitution in the constant region.

In yet another instance, provided is an anti-PD-L1 antibody comprising a heavy chain and a light chain variable region sequence, wherein:
(a) the heavy chain further comprises an HVR-H1, HVR-H2 and an HVR-H3 sequence having at least 85% sequence identity to GFTFSDSWIH (SEQ ID NO:19), AWISPYGGSTYYADSVKG (SEQ ID NO:20) and RHWPGGFDY (SEQ ID NO:21), respectively, or
(b) the light chain further comprises an HVR-L1, HVR-L2 and an HVR-L3 sequence having at least 85% sequence identity to RASQDVSTAVA (SEQ ID NO:22), SASFLYS (SEQ ID NO:23) and QQYLYHPAT (SEQ ID NO:24), respectively.
   In a specific aspect, the sequence identity is 86%, 87%, 88%, 89%, 90%, 91%, 92%, 93%, 94%, 95%, 96%, 97%, 98%, 99% or 100%.

In another aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In yet another aspect, the framework sequences are derived from human consensus framework sequences. In a still further aspect, the heavy chain framework sequences are derived from a Kabat subgroup I, 11, or III sequence. In a still further aspect, the heavy chain framework sequence is a VH subgroup III consensus framework. In a still further aspect, one or more of the heavy chain framework sequences are set forth as SEQ ID NOs:8, 9, 10 and 11. In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences are set forth as SEQ ID NOs:15, 16, 17 and 18.

In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect, the minimal effector function results from an “effector-less Fc mutation” or aglycosylation. In still a further instance, the effector-less Fc mutation is an N297A or D265A/N297A substitution in the constant region.

In another further instance, provided is an isolated anti-PD-L1 antibody comprising a heavy chain and a light chain variable region sequence, wherein:
(a) the heavy chain sequence has at least 85% sequence identity to the heavy chain sequence: EVQLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSS (SEQ ID NO:25), and/or
(b) the light chain sequences has at least 85% sequence identity to the light chain sequence:

(SEQ ID NO: 4)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR.

In a specific aspect, the sequence identity is 86%, 87%, 88%, 89%, 90%, 91%, 92%, 93%, 94%, 95%, 96%, 97%, 98%, 99% or 100%. In another aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In yet another aspect, the framework sequences are derived from human consensus framework sequences. In a further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences are set forth as SEQ ID NOs:8, 9, 10 and WGQGTLVTVSS (SEQ ID NO:27).

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences are set forth as SEQ ID NOs:15, 16, 17 and 18.

In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect, the minimal effector function results from production in prokaryotic cells. In a still further specific aspect, the minimal effector function results from an “effector-less Fc mutation” or aglycosylation. In still a further instance, the effector-less Fc mutation is an N297A or D265A/N297A substitution in the constant region.

In a further aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the heavy chain framework sequences are derived from a Kabat subgroup I, 11, or III sequence. In a still further aspect, the heavy chain framework sequence is a VH subgroup III consensus framework. In a still further aspect, one or more of the heavy chain framework sequences is the following:

FR-H1
(SEQ ID NO: 29)
EVQLVESGGGLVQPGGSLRLSCAASGFTFS
FR-H2
(SEQ ID NO: 30)
WVRQAPGKGLEWVA
FR-H3
(SEQ ID NO: 10)
RFTISADTSKNTAYLQMNSLRAEDTAVYYCAR
FR-H4
(SEQ ID NO: 27)
WGQGTLVTVSS.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

FR-L1
(SEQ ID NO: 15)
DIQMTQSPSSLSASVGDRVTITC
FR-L2
(SEQ ID NO: 16)
WYQQKPGKAPKLLIY
FR-L3
(SEQ ID NO: 17)
GVPSRFSGSGSGTDFTLTISSLQPEDFATYYC
FR-L4
(SEQ ID NO: 28)
FGQGTKVEIK.

In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific, aspect the minimal effector function results from an “effector-less Fc mutation” or aglycosylation. In still a further instance, the effector-less Fc mutation is an N297A or D265A/N297A substitution in the constant region.

In yet another instance, provided is an anti-PD-L1 antibody comprising a heavy chain and a light chain variable region sequence, wherein:
(c) the heavy chain further comprises an HVR-H1, HVR-H2 and an HVR-H3 sequence having at least 85% sequence identity to GFTFSDSWIH (SEQ ID NO:19), AWISPYGGSTYYADSVKG (SEQ ID NO:20) and RHWPGGFDY (SEQ ID NO:21), respectively, and/or
(d) the light chain further comprises an HVR-L1, HVR-L2 and an HVR-L3 sequence having at least 85% sequence identity to RASQDVSTAVA (SEQ ID NO:22), SASFLYS (SEQ ID NO:23) and QQYLYHPAT (SEQ ID NO:24), respectively.

In a specific aspect, the sequence identity is 86%, 87%, 88%, 89%, 90%, 91%, 92%, 93%, 94%, 95%, 96%, 97%, 98%, 99% or 100%.

In another aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In yet another aspect, the framework sequences are derived from human consensus framework sequences. In a still further aspect, the heavy chain framework sequences are derived from a Kabat subgroup I, 11, or III sequence. In a still further aspect, the heavy chain framework sequence is a VH subgroup III consensus framework. In a still further aspect, one or more of the heavy chain framework sequences are set forth as SEQ ID NOs:8, 9, 10 and WGQGTLVTVSSASTK (SEQ ID NO:31).

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences are set forth as SEQ ID NOs:15, 16, 17 and 18. In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect, the minimal effector function results from an “effector-less Fc mutation” or aglycosylation. In still a further instance, the effector-less Fc mutation is an N297A or D265A/N297A substitution in the constant region.

In a still further instance, provided is an isolated anti-PD-L1 antibody comprising a heavy chain and a light chain variable region sequence, wherein:
(a) the heavy chain sequence has at least 85% sequence identity to the heavy chain sequence: EVQLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSSASTK (SEQ ID NO:26), or
(b) the light chain sequences has at least 85% sequence identity to the light chain sequence:

(SEQ ID NO: 4)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR.

In some instances, provided is an isolated anti-PD-L1 antibody comprising a heavy chain and a light chain variable region sequence, wherein the light chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence of SEQ ID NO:4. In some instances, provided is an isolated anti-PD-L1 antibody comprising a heavy chain and a light chain variable region sequence, wherein the heavy chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence of SEQ ID NO:26. In some instances, provided is an isolated anti-PD-L1 antibody comprising a heavy chain and a light chain variable region sequence, wherein the light chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:4 and the heavy chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:26. In some instances, one, two, three, four or five amino acid residues at the N-terminal of the heavy and/or light chain may be deleted, substituted or modified.

In a still further instance, provided is an isolated anti-PD-L1 antibody comprising a heavy chain and a light chain sequence, wherein:

(a)
the heavy chain sequence has at least 85%
sequence identity to the heavy chain sequence:
(SEQ ID NO: 32)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYASTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPG,
and/or
(b)
the light chain sequences has at least 85%
sequence identity to the light chain sequence:
(SEQ ID NO: 33)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In some instances, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO:33. In some instances, provided is an isolated anti-PD-L1 antibody comprising a heavy chain and a light chain sequence, wherein the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO:32. In some instances, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO:33 and the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO:32.

In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

In any of the instances herein, the isolated anti-PD-L1 antibody can bind to a human PD-L1, for example a human PD-L1 as shown in UniProtKB/Swiss-Prot Accession No.Q9NZQ7.1, or a variant thereof.

It is expressly contemplated that such PD-L1 antagonist antibodies or other antibodies described herein (e.g., anti-PD-L1 antibodies for detection of PD-L1 expression levels) for use in any of the embodiments enumerated above may have any of the features, singly or in combination, described in Sections 1-7 of Subsection F below.

2. Exemplary PD-1 Binding Antagonists

Any suitable PD-1 binding antagonist described herein or known in the art can be used. In some instances, the PD-1 binding antagonist is a molecule that inhibits the binding of PD-1 to its ligand binding partners. In a specific aspect the PD-1 ligand binding partners are PD-L1 and/or PD-L2. In another instance, a PD-L1 binding antagonist is a molecule that inhibits the binding of PD-L1 to its binding ligands. In a specific aspect, PD-L1 binding partners are PD-1 and/or B7-1. In another instance, the PD-L2 binding antagonist is a molecule that inhibits the binding of PD-L2 to its ligand binding partners. In a specific aspect, the PD-L2 binding ligand partner is PD-1. The antagonist may be an antibody, an antigen binding fragment thereof, an immunoadhesin, a fusion protein, or oligopeptide.

In some instances, the PD-1 binding antagonist is an anti-PD-1 antibody (e.g., a human antibody, a humanized antibody, or a chimeric antibody), for example, as described below. In some instances, the anti-PD-1 antibody is selected from the group consisting of MDX-1106 (nivolumab), MK-3475 (pembrolizumab), MEDI-0680 (AMP-514), PDR001, REGN2810, and BGB-108. MDX-1106, also known as MDX-1106-04, ONO-4538, BMS-936558, or nivolumab, is an anti-PD-1 antibody described in WO2006/121168. MK-3475, also known as pembrolizumab or lambrolizumab, is an anti-PD-1 antibody described in WO 2009/114335. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In some instances, the PD-1 binding antagonist is AMP-224. AMP-224, also known as B7-DCIg, is a PD-L2-Fc fusion soluble receptor described in WO 2010/027827 and WO 2011/066342.

In some instances, the anti-PD-1 antibody is MDX-1106. Alternative names for “MDX-1106” include MDX-1106-04, ONO-4538, BMS-936558, and nivolumab. In some instances, the anti-PD-1 antibody is nivolumab (CAS Registry Number: 946414-94-4). In a still further instance, provided is an isolated anti-PD-1 antibody comprising a heavy chain variable region comprising the heavy chain variable region amino acid sequence from SEQ ID NO:1 and/or a light chain variable region comprising the light chain variable region amino acid sequence from SEQ ID NO:2. In a still further instance, provided is an isolated anti-PD-1 antibody comprising a heavy chain and/or a light chain sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence: QVQLVESGGGVVQPGRSLRLDCKASGITFSNSGMHWVRQAPGKGLEWVAVIWYDGSKRYYADSVKGR FTISRDNSKNTLFLQMNSLRAEDTAVYYCATNDDYWGQGTLVTVSSASTKGPSVFPLAPCSRSTSESTAA LGCLVKDYFPEPVTVSWNSGALTSGVHTFPAVLQSSGLYSLSSVVTVPSSSLGTKTYTCNVDHKPSNTKV DKRVESKYGPPCPPCPAPEFLGGPSVFLFPPKPKDTLMISRTPEVTCVVVDVSQEDPEVQFNWYVDGVE VHNAKTKPREEQFNSTYRVVSVLTVLHQDWLNGKEYKCKVSNKGLPSSIEKTISKAKGQPREPQVYTLPP SQEEMTKNQVSLTCLVKGFYPSDIAVEWESNGQPENNYKTTPPVLDSDGSFFLYSRLTVDKSRWQEGN VFSCSVMHEALHNHYTQKSLSLSLGK (SEQ ID NO:1), and
(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 2)
EIVLTQSPATLSLSPGERATLSCRASQSVSSYLAWYQQKPGQAPRLLIY
DASNRATGIPARFSGSGSGTDFTLTISSLEPEDFAVYYCQQSSNWPRTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

It is expressly contemplated that such PD-1 antagonist antibodies or other antibodies described herein (e.g., anti-PD-1 antibodies for detection of PD-1 expression levels) for use in any of the embodiments enumerated above may have any of the features, singly or in combination, described in Sections 1-7 of Subsection F below.

3. Exemplary PD-L2 Binding Antagonists

In some instances, the PD-L1 axis binding antagonist is a PD-L2 binding antagonist. Any suitable PD-L2 binding antagonist described herein or known in the art can be used. In some instances, the PD-L2 binding antagonist is an anti-PD-L2 antibody (e.g., a human antibody, a humanized antibody, or a chimeric antibody). In some instances, the PD-L2 binding antagonist is an immunoadhesin.

It is expressly contemplated that such PD-L2 antagonist antibodies or other antibodies described herein (e.g., anti-PD-L2 antibodies for detection of PD-L2 expression levels) for use in any of the embodiments enumerated above may have any of the features, singly or in combination, described in Sections 1-7 of Subsection F below.

B. Exemplary VEGF Antagonists

VEGF antagonists include any molecule capable of binding VEGF, reducing VEGF expression levels, or neutralizing, blocking, inhibiting, abrogating, reducing, or interfering with VEGF biological activities. An exemplary human VEGF is shown under UniProtKB/Swiss-Prot Accession No. P15692, Gene ID (NCBI): 7422.

Any suitable VEGF antagonist may be used. In some instances, the VEGF antagonist is an anti-VEGF antibody. In some embodiments, the anti-VEGF antibody is bevacizumab, also known as “rhuMab VEGF” or “AVASTIN®.” Bevacizumab is a recombinant humanized anti-VEGF monoclonal antibody generated according to Presta et al. (Cancer Res. 57:4593-4599, 1997). Bevacizumab comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879, the entire disclosure of which is expressly incorporated herein by reference. Additional preferred antibodies include the G6 or B20 series antibodies (e.g., G6-31, B20-4.1), as described in PCT Application Publication No. WO 2005/012359. For additional preferred antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al. (Journal of Immunological Methods 288:149-164, 2004). Other preferred antibodies include those that bind to a functional epitope on human VEGF comprising of residues F17, M18, D19, Y21, Y25, Q89, 191, K101, E103, and C104 or, alternatively, comprising residues F17, Y21, Q22, Y25, D63, 183, and Q89.

In other instances, the VEGF antagonist is an anti-VEGFR2 antibody or related molecule (e.g., ramucirumab, tanibirumab, aflibercept); an anti-VEGFR1 antibody or related molecules (e.g., icrucumab, aflibercept (VEGF Trap-Eye; EYLEA®), or ziv-aflibercept (VEGF Trap; ZALTRAP®)); a bispecific VEGF antibody (e.g., MP-0250, vanucizumab (VEGF-ANG2), or bispecific antibodies disclosed in US 2001/0236388); a bispecific antibody including a combination of two of anti-VEGF, anti-VEGFR1, and anti-VEGFR2 arms; an anti-VEGFA antibody (e.g., bevacizumab, sevacizumab); an anti-VEGFB antibody; an anti-VEGFC antibody (e.g., VGX-100), an anti-VEGFD antibody; or a nonpeptide small molecule VEGF antagonist (e.g., pazopanib, axitinib, vandetanib, stivarga, cabozantinib, lenvatinib, nintedanib, orantinib, telatinib, dovitinib, cediranib, motesanib, sulfatinib, apatinib, foretinib, famitinib, or tivozanib).

It is expressly contemplated that such VEGF antagonist antibodies or other antibodies described herein (e.g., anti-VEGF antibodies for detection of VEGF expression levels) for use in any of the embodiments enumerated above may have any of the features, singly or in combination, described in Sections 1-7 of Subsection F below.

C. Exemplary IL8 Antagonists

IL8 antagonists include any molecule capable of binding IL8, reducing IL8 expression levels, or neutralizing, blocking, inhibiting, abrogating, reducing, or interfering with IL8 biological activities. An exemplary human IL8 is shown under UniProtKB/Swiss-Prot Accession No. P10145.

Any suitable IL8 antagonist may be used. In some instances, the IL8 antagonist is an anti-IL8 antibody. Exemplary, non-limiting anti-IL8 antibodies include HuMax-IL8 (also known as BMS-986253). In other instances, the IL8 antagonist is a small molecule IL8 inhibitor. Exemplary, non-limiting small molecule IL8 inhibitors include reparixin (see, e.g., Leitner et al. Int. J. Immunopathol. Pharmacol. 20(1):25-36, 2007).

D. Exemplary IL1B Antagonists

IL1B antagonists include any molecule capable of binding IL1B, reducing IL1B expression levels, or neutralizing, blocking, inhibiting, abrogating, reducing, or interfering with IL1B biological activities. An exemplary human IL1B is shown under UniProtKB/Swiss-Prot Accession No. P01584.

Any suitable IL1B antagonist may be used. In some instances, the IL1B antagonist is an anti-IL1B antibody. Exemplary, non-limiting anti-IL1B antibodies include canakinumab, gevokizumab, LY2189102, XOMA 052, and 2H (see, e.g., Goh et al. MAbs 6(3):764-772, 2014). In other instances, the IL1B antagonist is a small molecule IL1B inhibitor (e.g., a small molecule that inhibits IL1B release, e.g., a caspase 1 inhibitor (e.g., pralnacasan (VX-740) and VX-765)). Exemplary IL1B antagonists are described, e.g., in Dinarello et al. Nat. Rev. Drug Discov. 11(8):633-652, 2012.

E. Exemplary IL1R Antagonists

IL1R antagonists include any molecule capable of binding IL1R, reducing IL1R expression levels, or neutralizing, blocking, inhibiting, abrogating, reducing, or interfering with IL1R biological activities. An exemplary human IL1R, type 1 is shown under UniProtKB/Swiss-Prot Accession No. P14778. An exemplary human IL1R, type 2 is shown under UniProtKB/Swiss-Prot Accession No. P27930.

Any suitable IL1R antagonist may be used. In some instances, the IL1R antagonist is an anti-IL1R antibody. Exemplary, non-limiting anti-IL1R antibodies include AMG108. In other instances, the IL1R antagonist is a recombinant IL-1 RA protein or an engineered version thereof, e.g., anakinra (KINERET®). In yet other instances, the IL1R antagonist is a soluble decoy receptor, e.g., rilonacept (ARCALYST®), which is a dimeric fusion protein that includes the ligand-binding domains of the extracellular portions of the human IL1R (IL1R1) and an IL1 receptor accessory protein (IL-1 RAcP) linked in-line to the Fc region of human IgG1. In other instances, the IL1B antagonist is a small molecule IL1B inhibitor. Exemplary IL1R antagonists are described, e.g., in Dinarello et al. Nat. Rev. Drug Discov. 11(8):633-652, 2012.

F. Antibodies

Provided herein are antibodies (e.g., anti-PD-L1 antibodies, anti-PD-1 antibodies, anti-VEGF antibodies, anti-IL8 antibodies, anti-IL1B antibodies, and anti-IL1R antibodies). In a still further instance, provided is an isolated nucleic acid encoding any of the antibodies described herein. In some instances, the nucleic acid further comprises a vector suitable for expression of the nucleic acid encoding any of the antibodies described herein. In a still further specific aspect, the vector is in a host cell suitable for expression of the nucleic acid. In a still further specific aspect, the host cell is a eukaryotic cell or a prokaryotic cell. In a still further specific aspect, the eukaryotic cell is a mammalian cell, such as Chinese hamster ovary (CHO) cell.

The antibody or antigen binding fragment thereof, may be made using methods known in the art, for example, by a process comprising culturing a host cell containing nucleic acid encoding any of the antibodies or antigen-binding fragments described herein or known in the art in a form suitable for expression, under conditions suitable to produce such antibody or fragment, and recovering the antibody or fragment.

It is expressly contemplated that such antibodies (e.g., PD-L1 axis binding antagonist antibodies (e.g., anti-PD-L1 antibodies, anti-PD-1 antibodies, and anti-PD-L2 antibodies), anti-VEGF antibodies, anti-IL8 antibodies, anti-IL1B antibodies, anti-IL1R antibodies, or other antibodies described herein for use in any of the instances enumerated above may have any of the features, singly or in combination, described in Sections 1-7 below.

1. Antibody Affinity

In certain instances, an antibody provided herein (e.g., an anti-PD-L1 antibody, an anti-PD-1 antibody, an anti-VEGF antibody, an anti-IL8 antibody, an anti-IL1B antibody, or an anti-IL1R antibody) has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

In one instance, Kd is measured by a radiolabeled antigen binding assay (RIA). In one instance, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in phosphate-buffered saline (PBS) for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another instance, Kd is measured using a BIACORE® surface plasmon resonance assay. For example, an assay using a BIACORE®-2000 or a BIACORE®-3000 (BIAcore, Inc.) is performed at 25° C. with immobilized antigen CM5 chips at ˜10 response units (RU). In one instance, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (kₒₙ) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio kₒₙ/kₒₙ. See, for example, Chen et al., J. Mol. Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain instances, an antibody (e.g., an anti-PD-L1 antibody, an anti-PD-1 antibody, an anti-VEGF antibody, an anti-IL8 antibody, an anti-IL1B antibody, or an anti-IL1R antibody) provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain instances, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

3. Chimeric and Humanized Antibodies

In certain instances, an antibody (e.g., an anti-PD-L1 antibody, an anti-PD-1 antibody, an anti-VEGF antibody, an anti-IL8 antibody, an anti-IL1B antibody, or an anti-IL1R antibody) provided herein is a chimeric antibody. Certain chimeric antibodies are described, e.g., in U.S. Pat. No. 4,816,567; and Morrison et al. Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain instances, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some instances, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Natl. Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Nat. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain instances, an antibody (e.g., an anti-PD-L1 antibody, an anti-PD-1 antibody, an anti-VEGF antibody, an anti-IL8 antibody, an anti-IL1B antibody, or an anti-IL1R antibody) provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies of the invention (e.g., an anti-PD-L1 antibody, an anti-PD-1 antibody, an anti-VEGF antibody, an anti-IL8 antibody, an anti-IL1B antibody, or an anti-IL1R antibody) may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N J, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In any one of the above aspects, an antibody (e.g., an anti-PD-L1 antibody, an anti-PD-1 antibody, an anti-VEGF antibody, an anti-IL8 antibody, an anti-IL1B antibody, or an anti-IL1R antibody) provided herein may be a multispecific antibody, for example, a bispecific antibody. Multispecific antibodies are monoclonal antibodies that have binding specificities for at least two different sites. In certain instances, an antibody provided herein is a multispecific antibody, e.g., a bispecific antibody. Bispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (see, e.g., WO 2009/089004A1); cross-linking two or more antibodies or fragments (see, e.g., U.S. Pat. No. 4,676,980, and Brennan et al., Science 229: 81 (1985)); using leucine zippers to produce bi-specific antibodies (see, e.g., Kostelny et al., J. Immunol. 148(5): 1547-1553 (1992)); using “diabody” technology for making bispecific antibody fragments (see, e.g., Hollinger et al., Proc. Nat. Acad. Sci. USA 90:6444-6448 (1993)); using single-chain Fv (sFv) dimers (see, e.g., Gruber et al., J. Immunol. 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The antibody or fragment herein also includes a “Dual Acting FAb” or “DAF” comprising an antigen binding site that binds to PD-L1 as well as another, different antigen.

7. Antibody Variants

In certain instances, amino acid sequence variants of the antibodies provided herein (e.g., anti-PD-L1 antibodies, anti-PD-1 antibodies, anti-VEGF antibodies, anti-IL8 antibodies, anti-IL1B antibodies, or anti-IL1R antibodies) are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

I. Substitution, Insertion, and Deletion Variants

In certain instances, antibody variants having one or more amino acid substitutions are provided. Sites of interest for substitutional mutagenesis include the HVRs and FRs. Conservative substitutions are shown in Table 8 under the heading of “preferred substitutions.” More substantial changes are provided in Table 8 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved Antibody-Dependent Cell-Mediated Cytotoxicity (ADCC) or Complement Dependent Cytotoxicity (CDC).

TABLE 8
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, lie;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity and/or reduced immunogenicity) relative to the parent antibody and/or will have substantially retained certain biological properties of the parent antibody. An exemplary substitutional variant is an affinity matured antibody, which may be conveniently generated, for example, using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some instances of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain instances, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen-contacting residues in the HVRs. In certain instances of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

II. Glycosylation Variants

In certain instances, an antibody provided herein (e.g., an anti-PD-L1 antibody, an anti-PD-1 antibody, an anti-VEGF antibody, an anti-IL8 antibody, an anti-IL1B antibody, or an anti-IL1R antibody) can be altered to increase or decrease the extent to which the antibody is glycosylated. Addition or deletion of glycosylation sites to an antibody provided herein may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some instances, modifications of the oligosaccharide in an antibody of the invention may be made in order to create antibody variants with certain improved properties.

In one instance, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, for example, U.S. Patent Publication Nos. US 2003/0157108 and US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); U.S. Pat. Appl. No. US 2003/0157108 A1; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

III. Fc Region Variants

In certain instances, one or more amino acid modifications may be introduced into the Fc region of an antibody provided herein (e.g., an anti-PD-L1 antibody, an anti-PD-1 antibody, an anti-VEGF antibody, an anti-IL8 antibody, an anti-IL1B antibody, or an anti-IL1R antibody), thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain instances, the present disclosure contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g., Hellstrom, I. et al. Proc. Natl. Acad. Sci. USA 83:7059-7063 (1986)) and Hellstrom, I et al., Proc. Natl. Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CYTOTOX 96® non-radioactive cytotoxicity assay (Promega, Madison, WI))). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Natl. Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, e.g., Gazzano-Santoro et al., J. Immunol. Methods 202:163 (1996); Cragg et al., Blood. 101:1045-1052 (2003); and Cragg et al., Blood. 103:2738-2743 (2004)). FcRn binding and in vivo clearance/half-life determinations can also be performed using methods known in the art (see, e.g., Petkova et al. Int'l. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain instances, an antibody variant comprises an Fc region with one or more amino acid substitutions which improve ADCC, e.g., substitutions at positions 298, 333, and/or 334 of the Fc region (EU numbering of residues).

In some instances, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

IV. Cysteine Engineered Antibody Variants

In certain instances, it may be desirable to create cysteine engineered antibodies, e.g., “thioMAbs,” in which one or more residues of an antibody are substituted with cysteine residues. In particular instances, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain instances, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

V. Antibody Derivatives

In certain instances, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another instance, conjugates of an antibody and nonproteinaceous moiety that may be selectively heated by exposure to radiation are provided. In one instance, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

VI. Immunoconjugates

The invention also provides immunoconjugates comprising an antibody provided herein (e.g., an anti-PD-L1 antibody, an anti-PD-1 antibody, an anti-VEGF antibody, an anti-IL8 antibody, an anti-IL1B antibody, or an anti-IL1R antibody) conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one instance, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another instance, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Saponaria officinalis inhibitor, gelonin, mitogellin, restrictocin, phenomycin, enomycin, and the tricothecenes.

In another instance, an immunoconjugate comprises an antibody as described herein conjugated to a radioactive atom to form a radioconjugate. A variety of radioactive isotopes are available for the production of radioconjugates. Examples include At²¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron. Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate) which are commercially available (e.g., from Pierce Biotechnology, Inc., Rockford, IL., U.S.A).

D. Pharmaceutical Formulations

Therapeutic formulations of the therapeutic agent(s) provided herein (e.g., PD-L1 axis binding antagonists (e.g., an anti-PD-L1 antibody (e.g., atezolizumab) or an anti-PD-1 antibody), a VEGF antagonist, an IL8 antagonist, an IL1B antagonist, an IL1R antagonist, or any other anti-cancer therapeutic agent) are prepared for storage by mixing the therapeutic agent(s) having the desired degree of purity with optional pharmaceutically acceptable carriers, excipients, or stabilizers in the form of lyophilized formulations or aqueous solutions. For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 119, Marcel Dekker, 2002.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

The formulation herein may also contain more than one active compound, preferably those with complementary activities that do not adversely affect each other. The type and effective amounts of such medicaments depend, for example, on the amount and type of antagonist present in the formulation, and clinical parameters of the subjects.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antagonist, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

It is to be understood that any of the above articles of manufacture may include an immunoconjugate described herein in place of or in addition to a PD-L1 axis binding antagonist.

### V. Kits and Articles of Manufacture

Provided herein are kits comprising one or more reagents for determining the presence of a biomarker (e.g., a biomarker described herein, e.g., one or more genes set forth in any one of Tables 1-7, e.g., IL8) in a sample from an individual with a disease or disorder (e.g., cancer, including UC and RCC). In some instances, the absence of the biomarker in the sample, or a decreased expression level of the biomarker relative to a reference level, indicates a higher likelihood of efficacy when the individual with the disease is treated with the PD-L1 axis binding antagonist. In some instances, the presence of the biomarker in the sample, or an elevated expression level of the biomarker relative to a reference level, indicates a lower likelihood of efficacy when the individual is treated with a PD-L1 axis binding antagonist monotherapy. Optionally, the kit may further include instructions to use the kit to select a medicament (e.g., a PD-L1 axis binding antagonist (e.g., an anti-PD-L1 antibody such as atezolizumab) or a PD-1 binding antagonist (e.g., an anti-PD-1 antibody) or an anti-cancer therapy other than or in addition to a PD-L1 axis binding antagonist) for treating the disease or disorder (e.g., cancer (e.g., bladder cancer (e.g., UC) or kidney cancer (e.g., RCC))).

Provided herein are also articles of manufacture including, packaged together, a PD-L1 axis binding antagonist in a pharmaceutically acceptable carrier and a package insert indicating that the PD-L1 axis binding antagonist is for treating a patient with a disease or disorder (e.g., cancer) based on the expression of a biomarker (e.g., a biomarker described herein, e.g., one or more genes set forth in any one of Tables 1-7, e.g., IL8). Also provided herein are also articles of manufacture including, packaged together, an anti-cancer therapy other than or in addition to a PD-L1 axis binding antagonist in a pharmaceutically acceptable carrier and a package insert indicating that the anti-cancer therapy other than or in addition to a PD-L1 axis binding antagonist is for treating a patient with a disease or disorder (e.g., cancer) based on the expression of a biomarker (e.g., a biomarker described herein, e.g., one or more genes set forth in any one of Tables 1-7, e.g., IL8). Treatment methods include any of the treatment methods disclosed herein. The present disclosure also concerns a method for manufacturing an article of manufacture comprising combining in a package a pharmaceutical composition comprising a PD-L1 axis binding antagonist or an anti-cancer therapy other than or in addition to a PD-L1 axis binding antagonist and a package insert indicating that the pharmaceutical composition is for treating a patient with a disease or disorder based on expression of a biomarker (e.g., a biomarker described herein, e.g., one or more genes set forth in any one of Tables 1-7, e.g., IL8).

The kit or article of manufacture may include, for example, a container and a label or package insert on or associated with the container. Suitable containers include, for example, bottles, vials, syringes, and the like. The container may be formed from a variety of materials such as glass or plastic. The container holds or contains a composition comprising the cancer medicament as the active agent and may have a sterile access port (e.g., the container may be an intravenous solution bag or a vial having a stopper pierceable by a hypodermic injection needle).

The kit or article of manufacture may further include a second container comprising a pharmaceutically-acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

The kit or article of manufacture of the present invention also includes information, for example in the form of a package insert, indicating that the composition is used for treating cancer based on the expression level of the biomarker(s) herein (e.g., a biomarker described herein, e.g., one or more genes set forth in any one of Tables 1-7, e.g., IL8). The insert or label may take any form, such as paper or on electronic media such as a magnetically recorded medium (e.g., floppy disk), a CD-ROM, a Universal Serial Bus (USB) flash drive, and the like. The label or insert may also include other information concerning the pharmaceutical compositions and dosage forms in the kit or article of manufacture.

In one example, provided herein is a kit for identifying an individual having a cancer (e.g., bladder cancer (e.g., UC) or kidney cancer (e.g., RCC)) who may benefit from treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), the kit comprising: (a) reagents for determining the expression level of IL8; and, optionally, (b) instructions for using the reagents to identify an individual having a cancer who is likely to respond to treatment with an anti-cancer therapy comprising a PD-L1 axis binding antagonist in accordance with any one of the methods provided herein.

In another aspect, provided herein is a kit for identifying an individual having a cancer (e.g., bladder cancer (e.g., UC) or kidney cancer (e.g., RCC)) who may benefit from treatment with an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), the kit comprising: (a) reagents for determining the expression level of IL8; and, optionally, (b) instructions for using the reagents to identify an individual having a cancer who is less likely to respond to treatment with an anti-cancer therapy comprising a PD-L1 binding antagonist monotherapy in accordance with any one of the methods provided herein, thereby identifying the individual as one who may benefit from treatment with an anti-cancer therapy other than or in addition to a PD-L1 binding antagonist.

In another example, provided herein is a kit for identifying an individual having a cancer (e.g., bladder cancer (e.g., UC) or kidney cancer (e.g., RCC)) who may benefit from treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), the kit comprising: (a) reagents for determining the expression level of one or more genes set forth in any one of Tables 2-4; and, optionally, (b) instructions for using the reagents to identify an individual having a cancer who is likely to respond to treatment with an anti-cancer therapy comprising a PD-L1 axis binding antagonist in accordance with any one of the methods provided herein.

In yet another example, provided herein is a kit for identifying an individual having a cancer (e.g., bladder cancer (e.g., UC) or kidney cancer (e.g., RCC)) who may benefit from treatment with an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), the kit comprising: (a) reagents for determining the expression level of one or more genes set forth in any one of Tables 2-4; and, optionally, (b) instructions for using the reagents to identify an individual having a cancer who is less likely to respond to treatment with an anti-cancer therapy comprising a PD-L1 binding antagonist monotherapy in accordance with any one of the methods provided herein, thereby identifying the individual as one who may benefit from treatment with an anti-cancer therapy other than or in addition to a PD-L1 binding antagonist.

In a further example, provided herein is a kit for identifying an individual having a cancer (e.g., bladder cancer (e.g., UC) or kidney cancer (e.g., RCC)) who may benefit from treatment with an anti-cancer therapy comprising a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), the kit comprising: (a) reagents for determining the expression level of one or more genes set forth in any one of Tables 5-7; and, optionally, (b) instructions for using the reagents to identify an individual having a cancer who is likely to respond to treatment with an anti-cancer therapy comprising a PD-L1 axis binding antagonist in accordance with any one of the methods provided herein.

In another example, provided herein is a kit for identifying an individual having a cancer (e.g., bladder cancer (e.g., UC) or kidney cancer (e.g., RCC)) who may benefit from treatment with an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)), the kit comprising: (a) reagents for determining the expression level of one or more genes set forth in any one of Tables 5-7; and, optionally, (b) instructions for using the reagents to identify an individual having a cancer who is less likely to respond to treatment with an anti-cancer therapy comprising a PD-L1 binding antagonist monotherapy in accordance with any one of the methods provided herein, thereby identifying the individual as one who may benefit from treatment with an anti-cancer therapy other than or in addition to a PD-L1 binding antagonist.

In another aspect, provided herein is a kit for treating an individual having a cancer (e.g., bladder cancer (e.g., UC) or kidney cancer (e.g., RCC)), the kit comprising: (a) a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)); and, optionally, (b) instructions to administer the PD-L1 axis binding antagonist to an individual having a cancer who has been identified as likely to respond to an anti-cancer therapy comprising a PD-L1 axis binding antagonist in accordance with any one of the methods provided herein.

In another aspect, provided herein is a kit for treating an individual having a cancer (e.g., bladder cancer (e.g., UC) or kidney cancer (e.g., RCC)), the kit comprising: (a) an anti-cancer therapy other than or in addition to a PD-L1 axis binding antagonist (e.g., a PD-L1 binding antagonist (e.g., an anti-PD-L1 antibody such as atezolizumab) or a PD-1 binding antagonist (e.g., an anti-PD-1 antibody)); and, optionally, (b) instructions to administer the anti-cancer therapy other than or in addition to a PD-L1 axis binding antagonist to an individual having a cancer who has been identified as less likely to respond to an anti-cancer therapy comprising a PD-L1 axis binding antagonist monotherapy in accordance with any one of the methods provided herein.

### EXAMPLES

The following examples are provided to illustrate, but not to limit the presently claimed invention.

### Example 1: Systemic and Tumor-Associated IL8 Correlates with Lack of Clinical Benefit to PD-L1 Checkpoint Inhibition

Interleukin 8 (IL8), also known as CXCL8, is a pro-inflammatory multifunctional cysteine-X-cysteine (CXC) chemokine that acts as a chemoattractant factor for neutrophils and myeloid leukocytes, and induces neutrophil degranulation. The association of IL8 with clinical outcomes to checkpoint inhibition (e.g., treatment with an anti-cancer therapy including a PD-L1 axis binding antagonist) has not been comprehensively evaluated in randomized studies. Moreover, the source of IL8 and the underlying biology that influences resistance to immune checkpoint inhibitors remain unknown. In this study, we analyzed IL8 expression, including circulating IL8 protein in plasma and IL8 gene expression in peripheral blood mononuclear cells (PBMC) and tumors, of patients treated with atezolizumab (anti-PD-L1 monoclonal antibody (mAb)), from multiple randomized trials representing 1581 patients with metastatic urothelial carcinoma (mUC) and metastatic renal cell carcinoma (mRCC).

A. Results

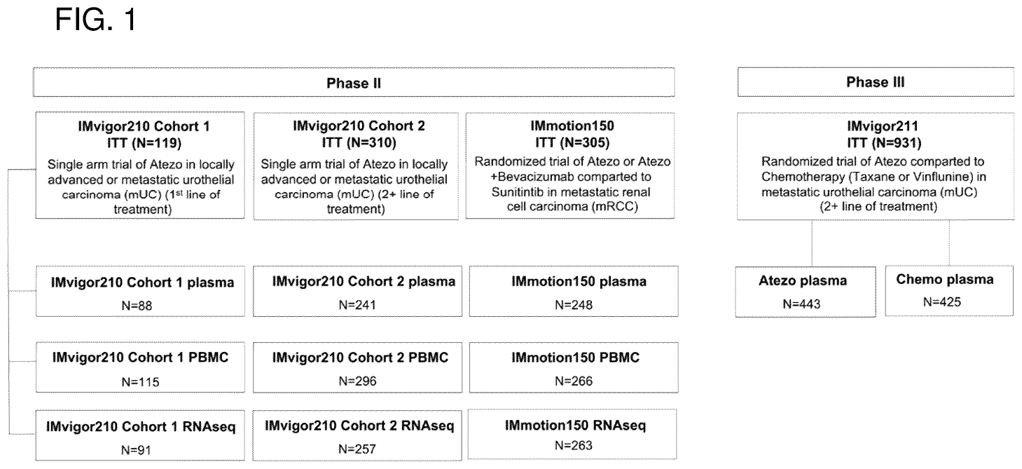

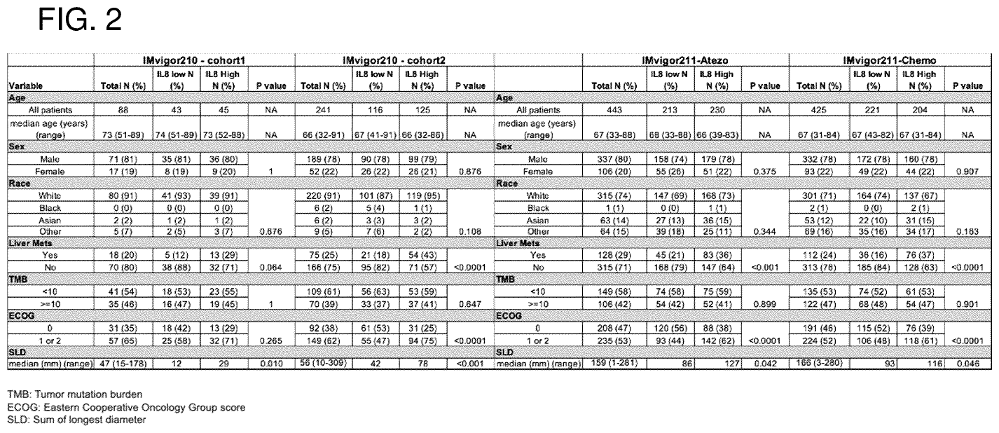

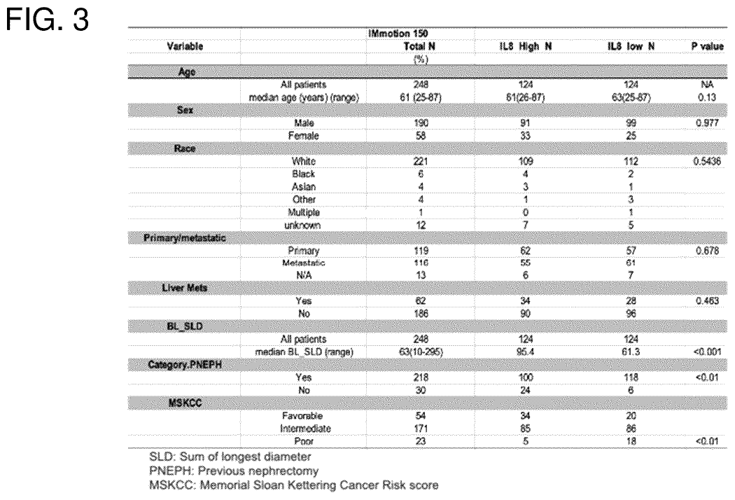

We interrogated the association of plasma, PBMC, and intra-tumoral IL8 expression with clinical outcome in large clinical studies that evaluated clinical activity of the PD-L1 blocking monoclonal antibody atezolizumab in mUC and mRCC (FIG. 1). IMvigor210 (NCT02108652) was a single arm Phase II study in mUC that included two cohorts: cohort 2 enrolled patients (n=254) with locally advanced or metastatic urothelial carcinoma whose disease had progressed after prior platinum-based chemotherapy, whereas Cohort 1 (n=91) enrolled patients who were treatment-naive in the metastatic setting and considered to be cisplatin-ineligible. IMvigor211 was a randomized Phase 3 mUC trial (NCT02302807) (n=1068) in which prior platinum-treated patients were treated with either chemotherapy (taxane or vinflunine) or atezolizumab. IMmotion150 (NCT01984242) was a randomized Phase II trial which investigated the clinical activity of atezolizumab with or without bevacizumab (anti-VEGF mAb) against an anti-angiogenic standard of care tyrosine kinase inhibitor (TKI), sunitinib, in patients with untreated mRCC (n=248). The demographics and baseline characteristics of biomarker evaluable patients, between the groups of high and low levels of plasma IL8 are shown in FIGS. 2 and 3. We conducted multi-variate analyses for associations with clinical outcome and adjusted for Eastern Cooperative Oncology Group (ECOG) performance status, presence of liver metastasis, and tumor burden (sum of longest diameter, SLD) in mUC; and Memorial Sloan Kettering Cancer Risk (MSKCC) prognostic risk score, previous nephrectomy, and SLD in mRCC data sets.

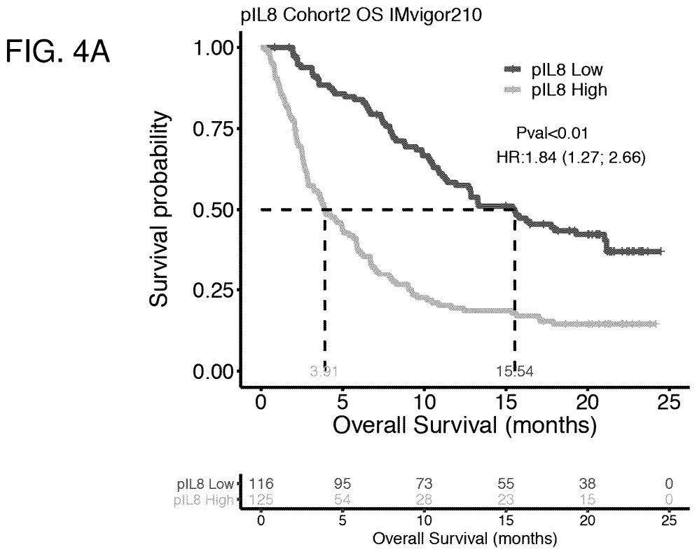

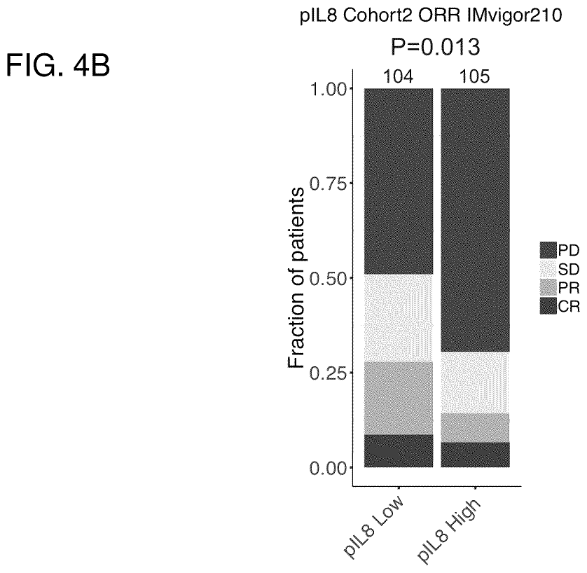

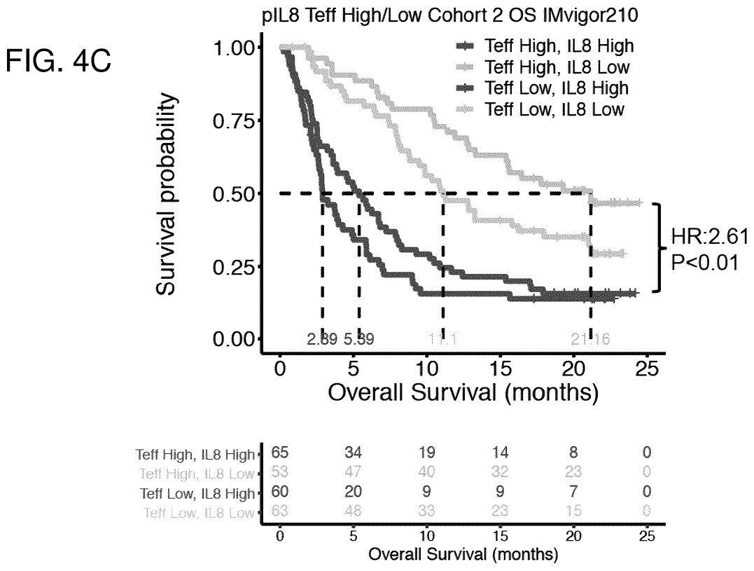

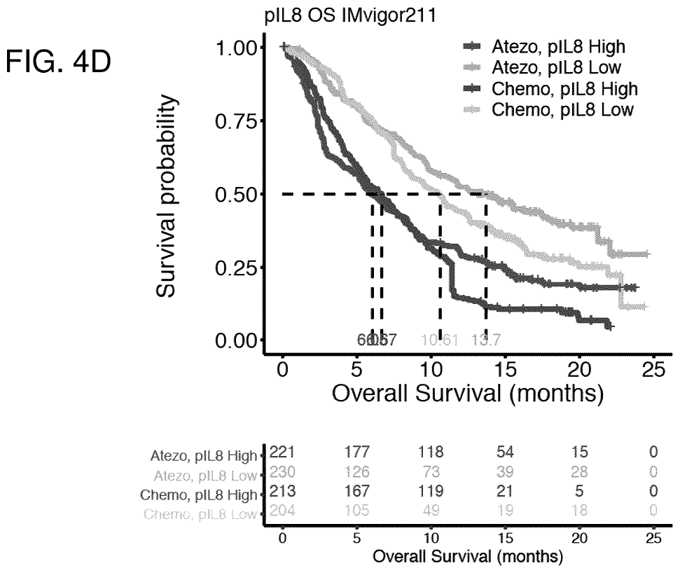

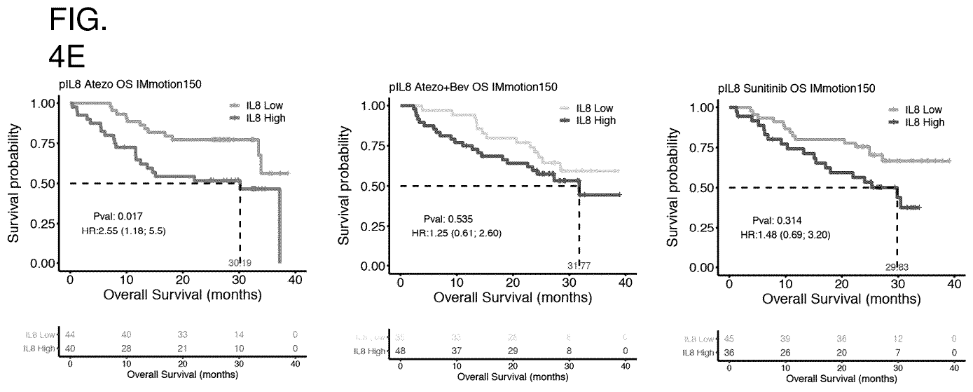

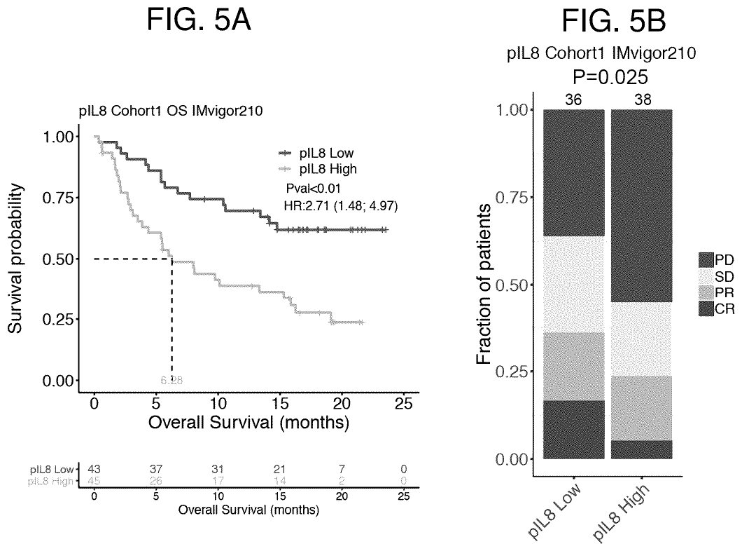

High baseline plasma IL8 (median cutoff) was significantly associated with worse overall survival (OS) (Hazard Ratio (HR)=1.90, 95% CI: 1.31, 2.74, P<0.0001) and objective response rate (ORR) (FIGS. 4A and 4B) in the IMvigor 210 Cohort 2. High plasma IL8 remained significantly associated with worse OS upon multivariate analysis (MVA), suggesting that the association of plasma IL8 with poor OS is independent of clinical prognostic factors. Similar to Cohort 2, an association of high plasma IL8 with worse OS and ORR was also observed in mUC patients in IMvigor 210 cohort 1 (FIGS. 5A and 5B).

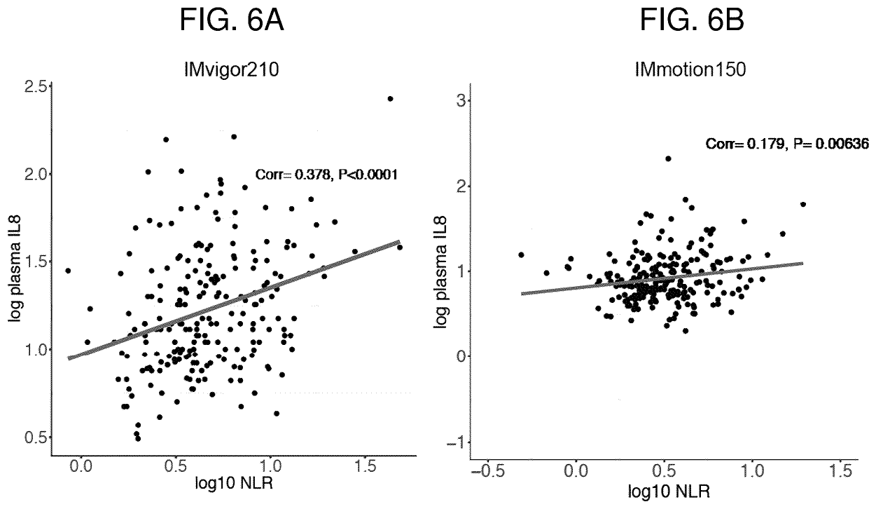

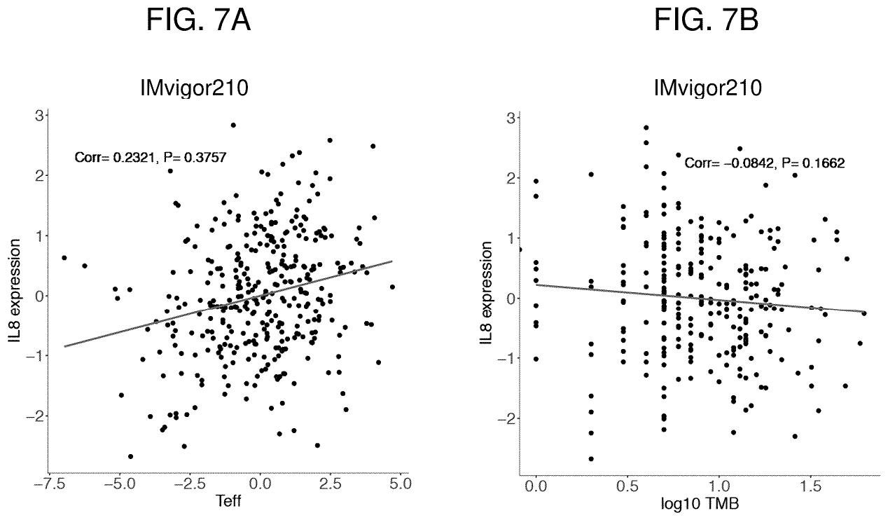

Plasma IL8 had a moderate correlation with neutrophil to lymphoid ratio (NLR) (FIGS. 6A and 6B). Plasma IL8 did not correlate with markers of high tumoral immune presence such as tumor T-effector (T_{eff}) gene expression (the T_{eff} signature included CD8A, GZMA, GZMB, and PRF1) or tumor mutation burden (FIGS. 7A and 7B). Unexpectedly though, high plasma IL8 had a negative impact on the therapeutic outcome to checkpoint inhibitors (CPI). Patients who had elevated plasma IL8 had worse OS compared to those with low plasma IL8, even if their tumors had a high T_{eff} signature that is broadly associated with response to CPI (FIG. 4C) (HR: 1.71; 95% CI: 1.12, 326, P=0.017).

To confirm the robustness of the above finding, we subsequently evaluated plasma IL8 in IMvigor211. MVA showed that patients with high plasma IL8 had significantly worse OS in both atezolizumab and chemotherapy arms (FIG. 4D), indicating that high plasma IL8 is prognostic in mUC. However, in patients with low plasma IL8, atezolizumab improved OS compared to chemotherapy (HR: 0.76. 95% CI: 0.61. 0.84, P=0.01) (FIG. 4D). In the IMmotion150 study, high plasma IL8 level was associated with reduced OS in patients treated with atezolizumab (HR: 2.55, 95% CI: 1.18, 5.5, P=0.017) and showed a trend toward worse OS in atezolizumab+bevacizumab (HR: 1.25, 95% CI: 0.61, 2.60, P=0.535) and sunitinib-treated (HR: 1.48, 95% CI: 0.69, 3.20, P=0.314) patients (FIG. 4E).

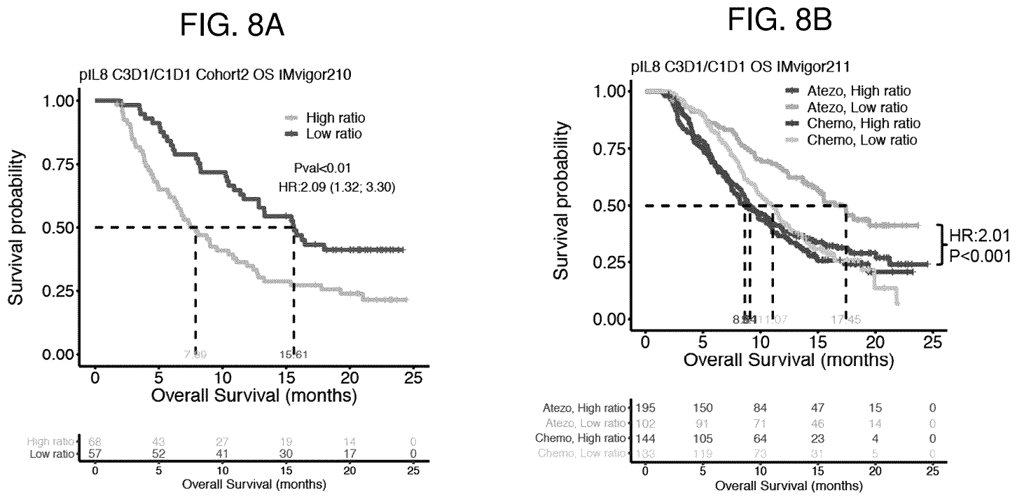

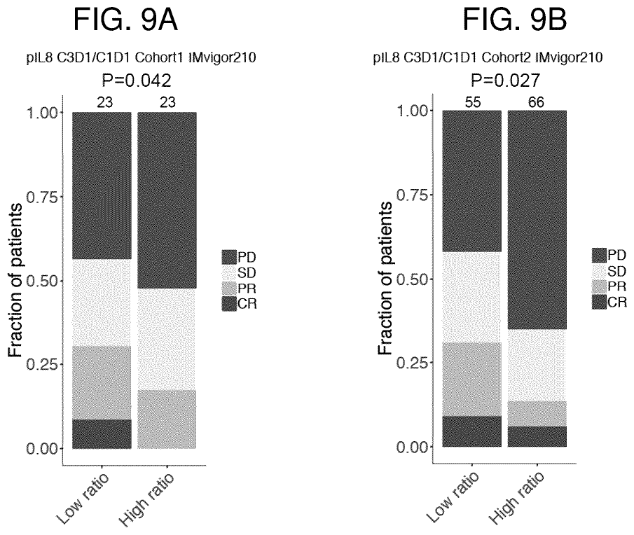

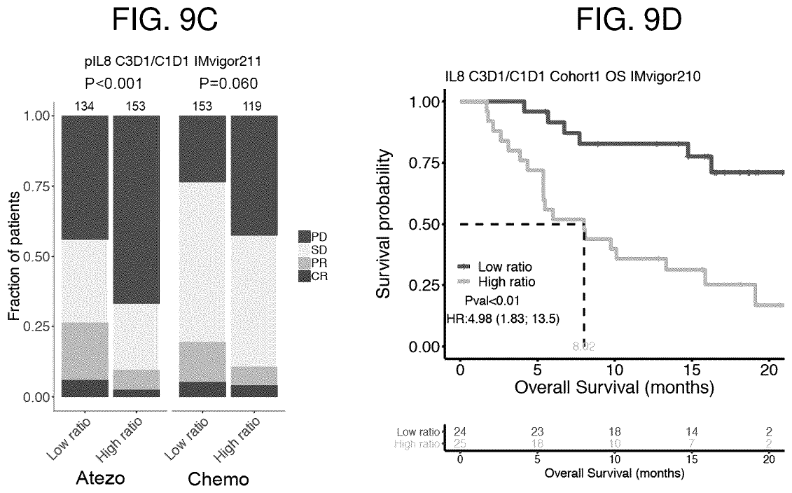

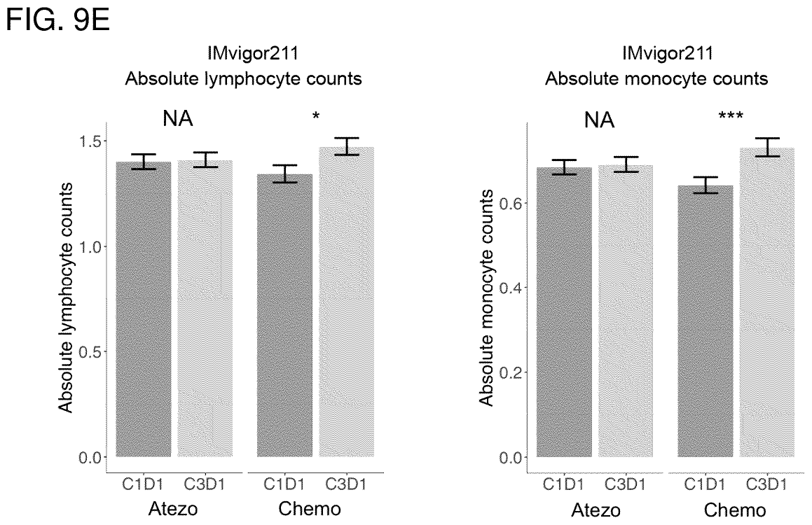

Next, we evaluated on-treatment (6 weeks after atezolizumab or chemotherapy treatment) plasma IL8 levels compared to baseline in IMvigor210 and IMvigor211 trials. On-treatment decrease in plasma IL8 was associated with significantly better OS (HR: 0.48; 95% CI: 0.31, 0.76, P<0.01) and ORR in in both cohort 2 (FIGS. 8A and 9A-9E) and cohort 1 (FIGS. 9A-9E) of IMvigor210. A similar trend was observed in the IMvigor211 trial in patients treated with atezolizumab (HR: 0.49, 95% CI: 0.36; 0.66, P<0.001) but not with chemotherapy, suggesting that on-treatment decreases of plasma IL8 predicted better clinical outcome to atezolizumab (FIG. 8B).

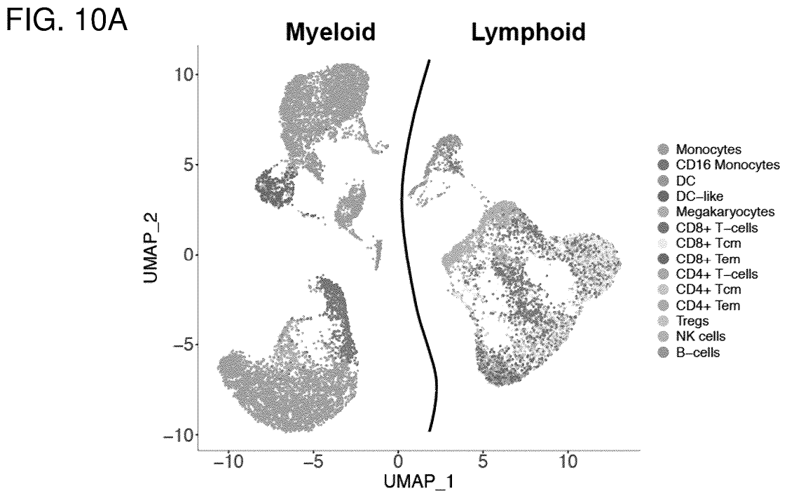

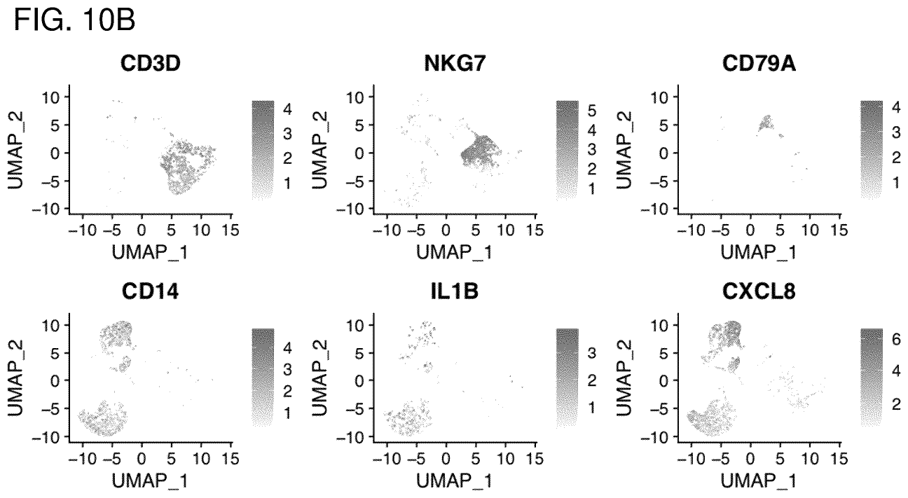

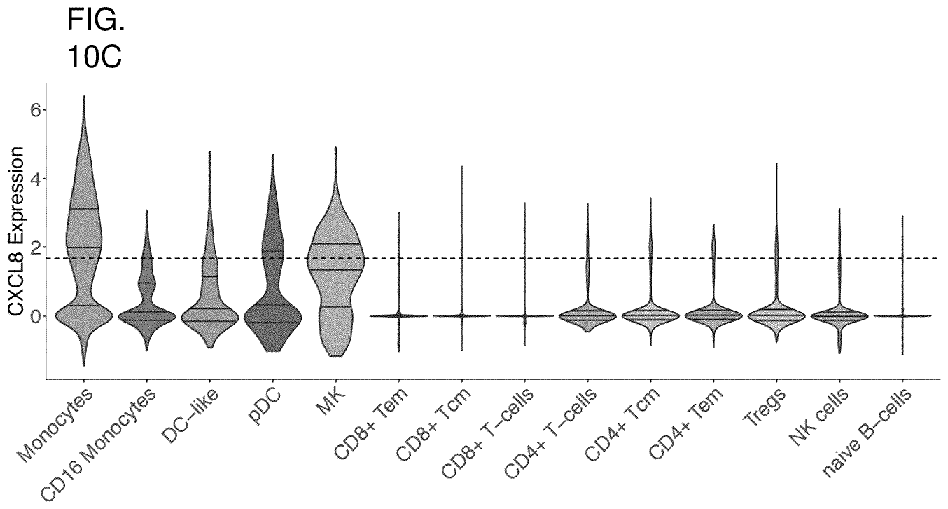

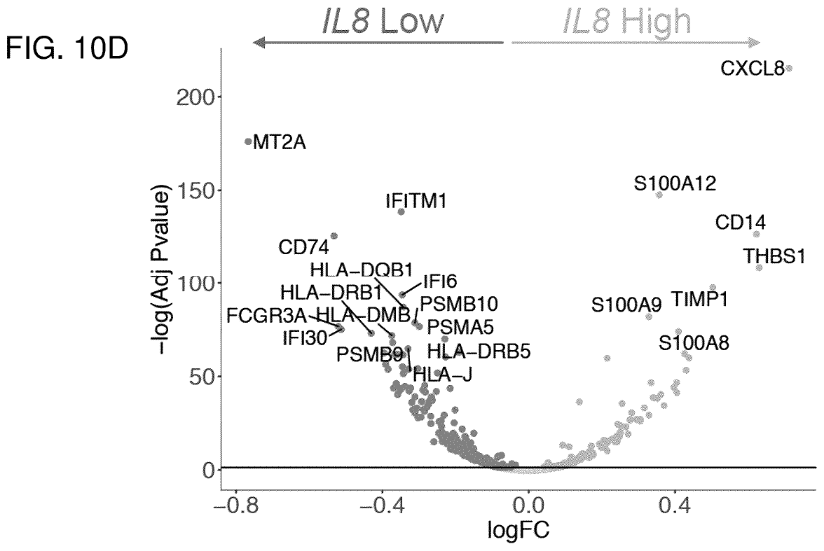

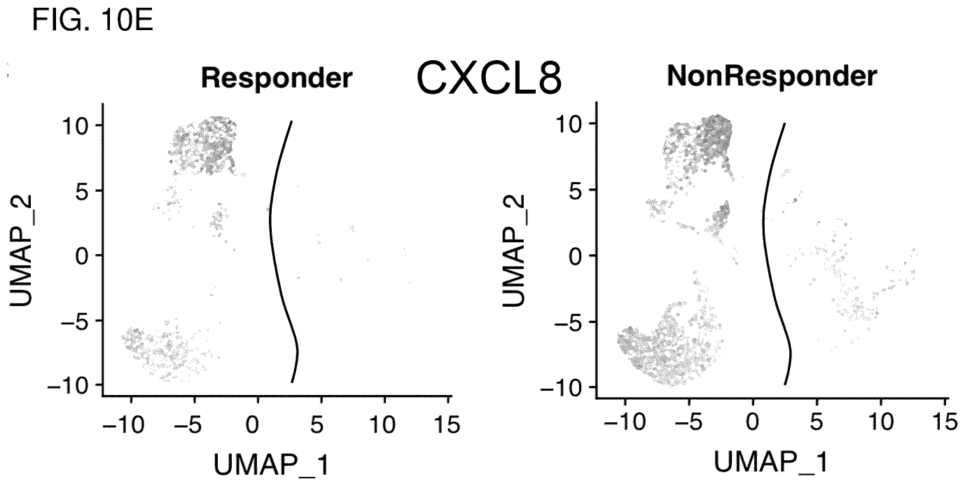

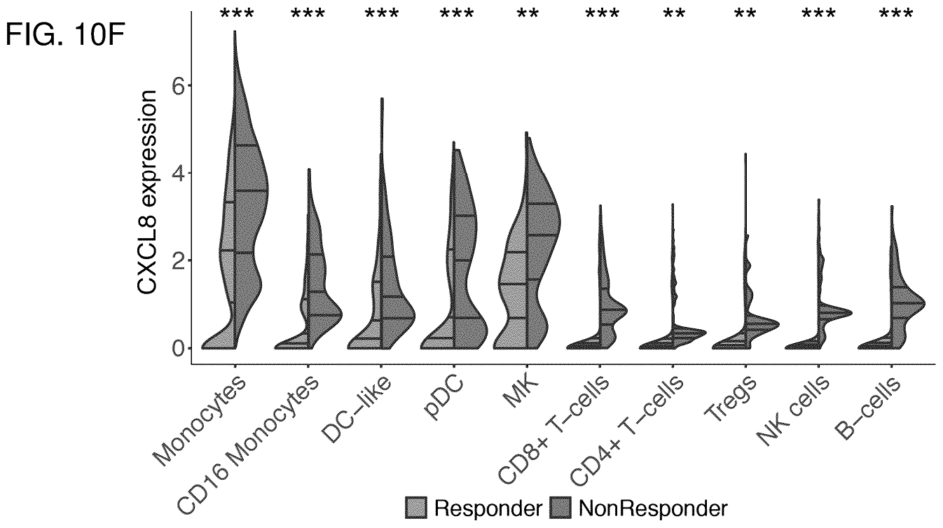

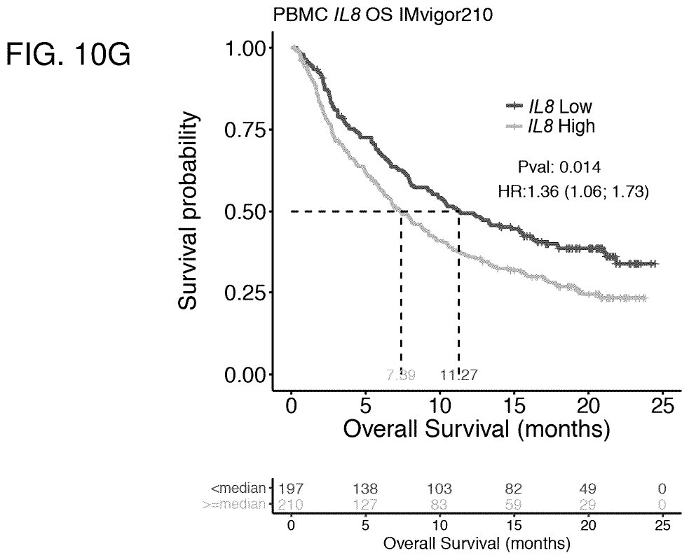

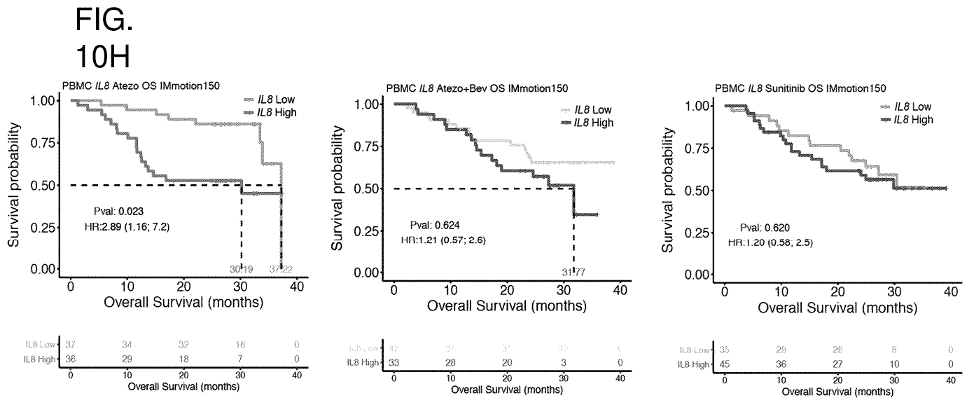

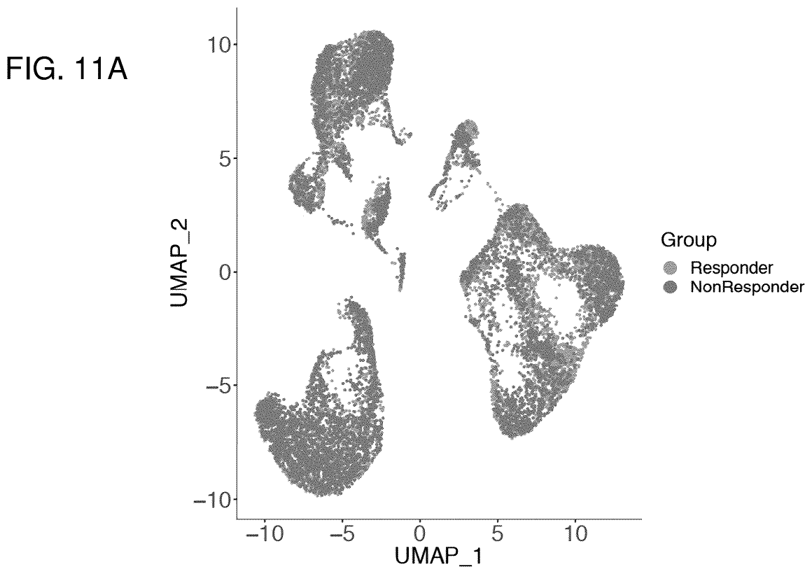

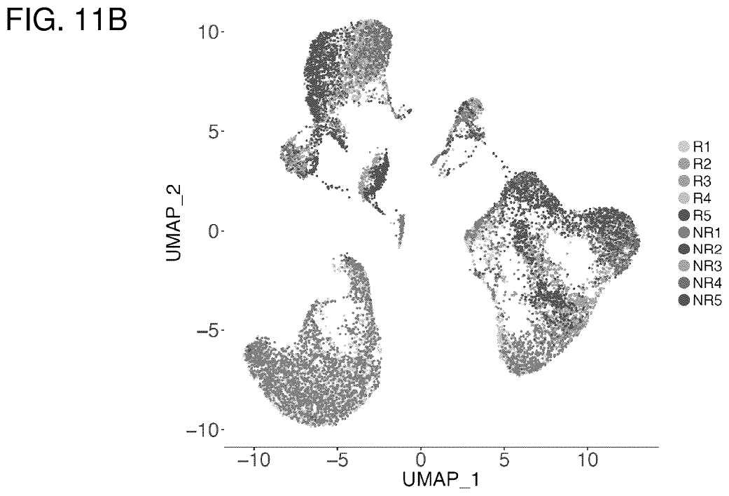

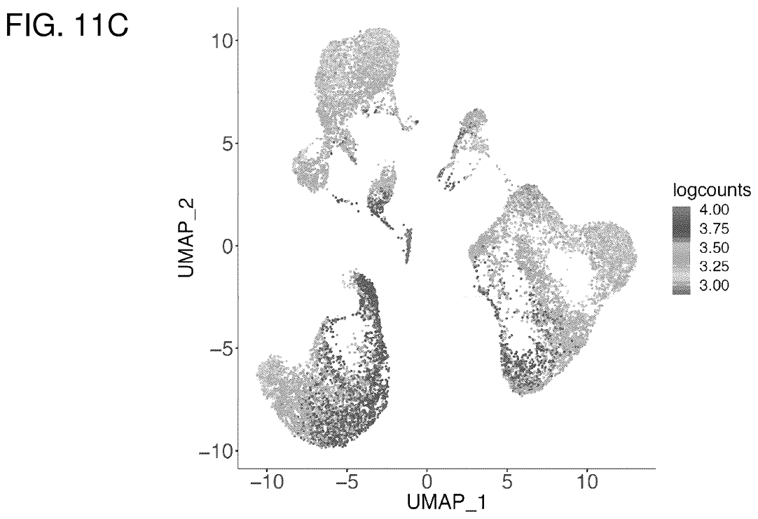

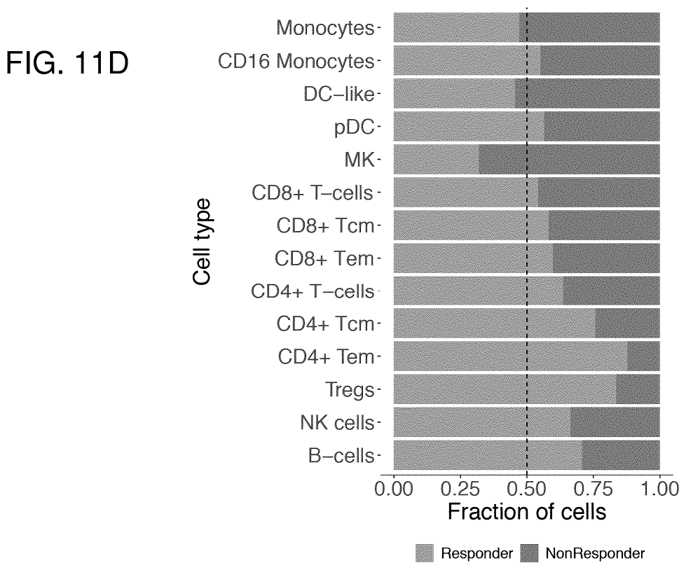

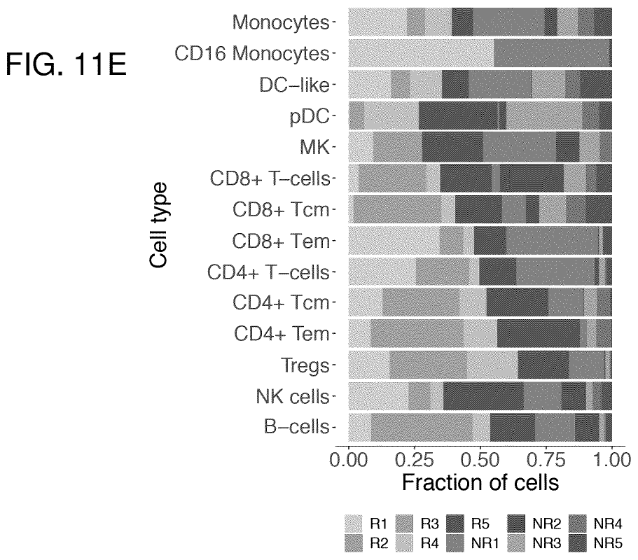

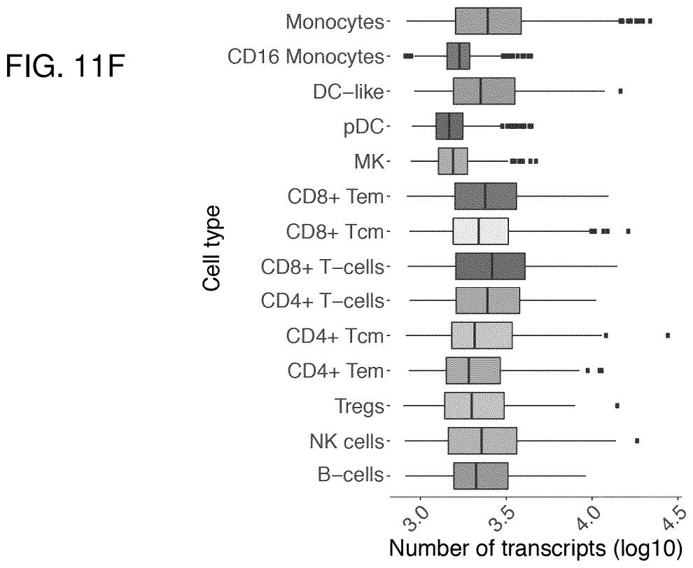

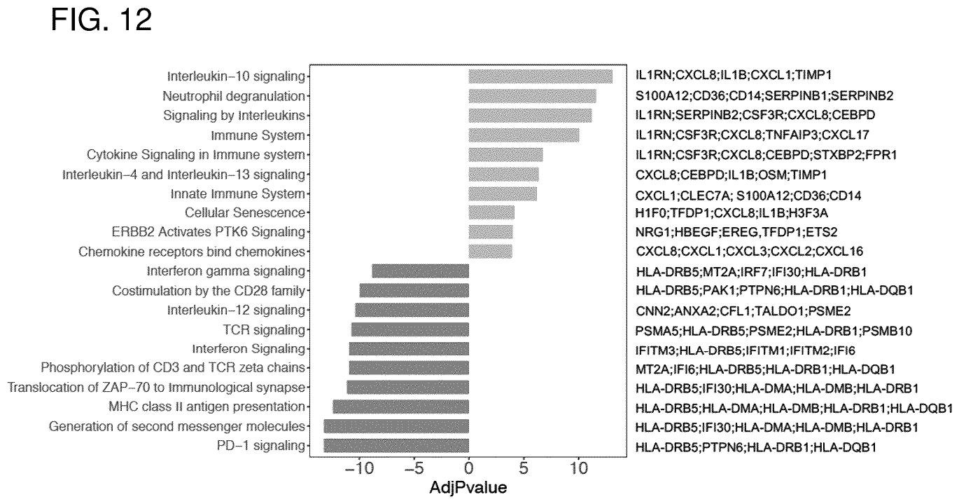

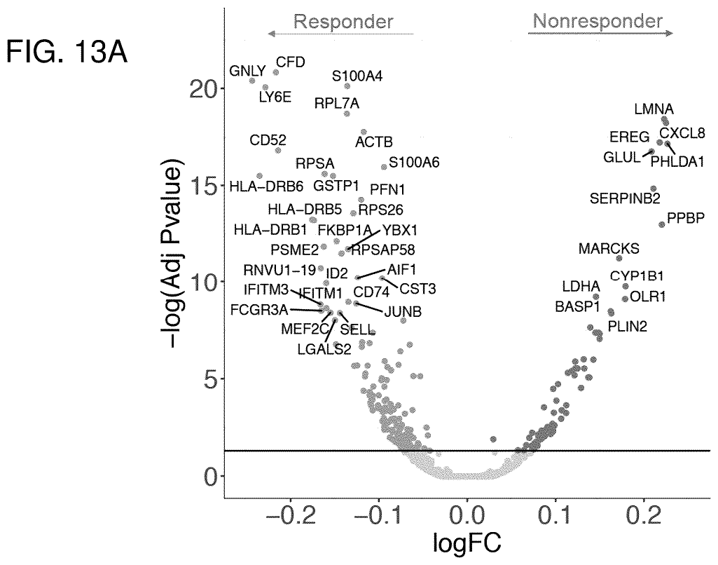

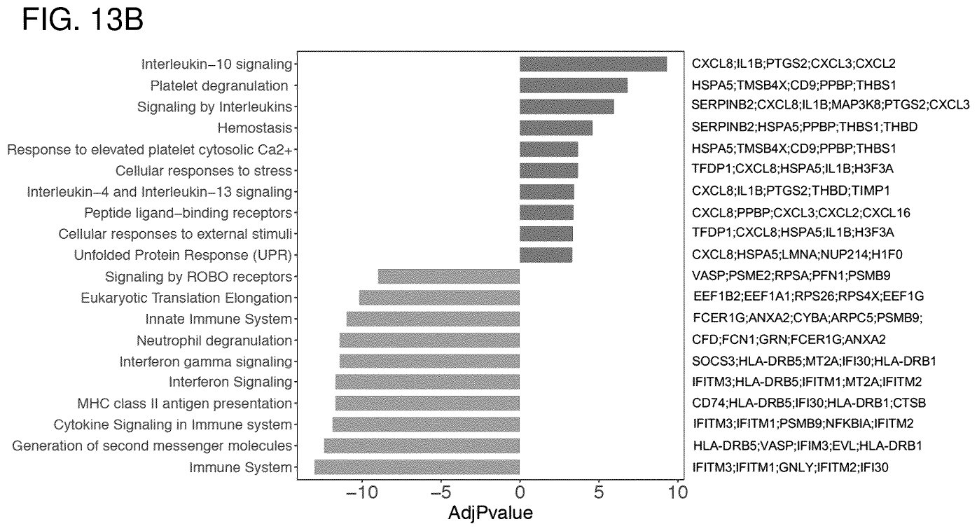

We evaluated whether IL8 expression on a specific subset of circulating peripheral blood mononuclear cells (PBMCs) was associated with efficacy. We performed single-cell RNA-sequencing (scRNA-seq) of PBMCs from five responders and five non-responders from the IMvigor210 study (FIGS. 10A and 11A-11F). Uniform Manifold Approximation and Projection (UMAP) identified myeloid and lymphoid cells through lineage specific marker genes (FIG. 10B). Compared to lymphoid clusters, IL8 expression was high in monocyte-like (clusters 0, 1, 3, 4, and 13) and megakaryocytes (MK) clusters (cluster 14), with the highest expression in cluster 1 (FIG. 10C). Differential gene expression analysis between IL8 high versus IL8 low myeloid cells revealed enrichment of myeloid inflammatory genes in the IL8 high cells (FIGS. 10D and 12). Concomitantly, there was a downregulation of genes associated with antigen presentation machinery, such as human leukocyte antigen (HLA) genes and interferon gamma (IFNg)-induced genes in IL8 high cells compared with IL8 low cells (FIGS. 10D, 12, 13A, and 13B).

The proportion of IL8-producing myeloid cells as well as the relative expression of IL8 on myeloid cells was higher in non-responders compared to responders (FIGS. 10E and 10F). We extended our analysis of IL8 gene expression in PBMCs to entire cohorts of patients in IMvigor210 and IMmotion150. Indeed, gene expression analysis by a NanoString platform confirmed that high IL8 gene expression in PBMCs was significantly associated with worse OS (HR: 1.36, 95% CI: 1.07, 1.73, P=0.013) (FIG. 10G) in mUC patients in Mvigor210. Similarly, high PBMC IL8 gene expression in mRCC patients was also associated with worse PFS in atezolizumab monotherapy arm (HR 2.52; 95% CI, 1.29, 4.90, p=0.007) but not in atezolizumab+bevacizumab or sunitinib arms. (FIG. 10H). Genes that are upregulated in IL8 high PBMCs in bladder cancer (e.g., locally advanced or metastatic urothelial carcinoma) are shown in Table 4. Genes that are downregulated in IL8 high PBMCs in bladder cancer (e.g., locally advanced or metastatic urothelial carcinoma) are shown in Table 7. The expression level of one or more of the genes set forth in Table 4 or Table 7 can be used as proxies for IL8 expression.

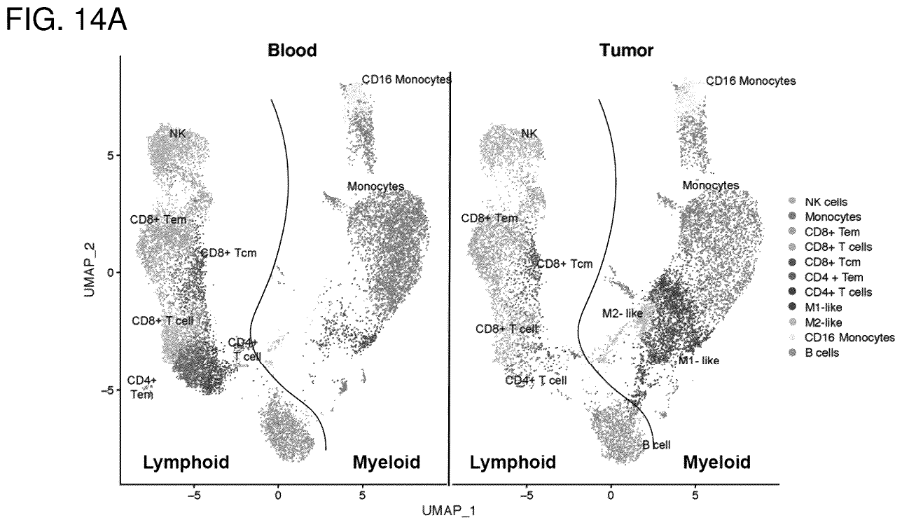

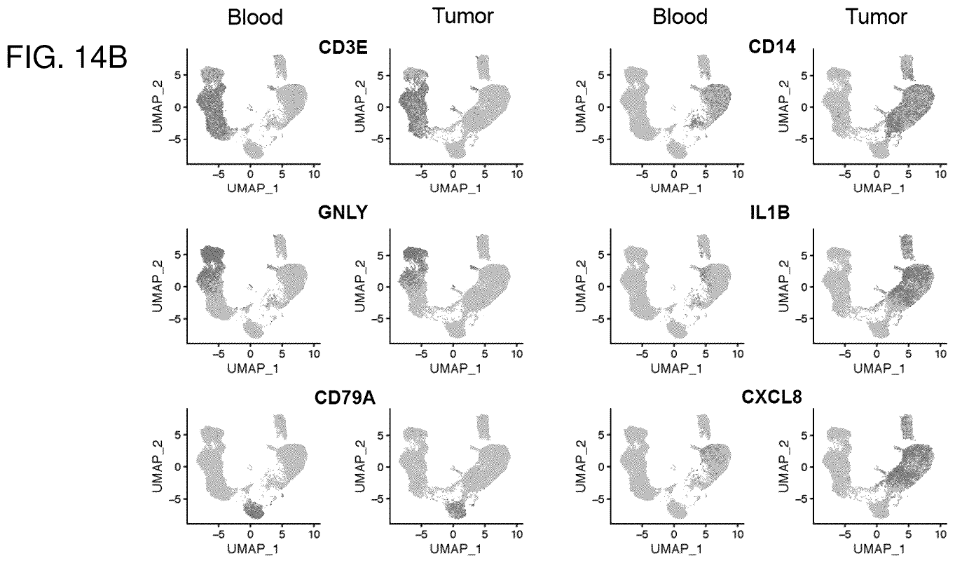

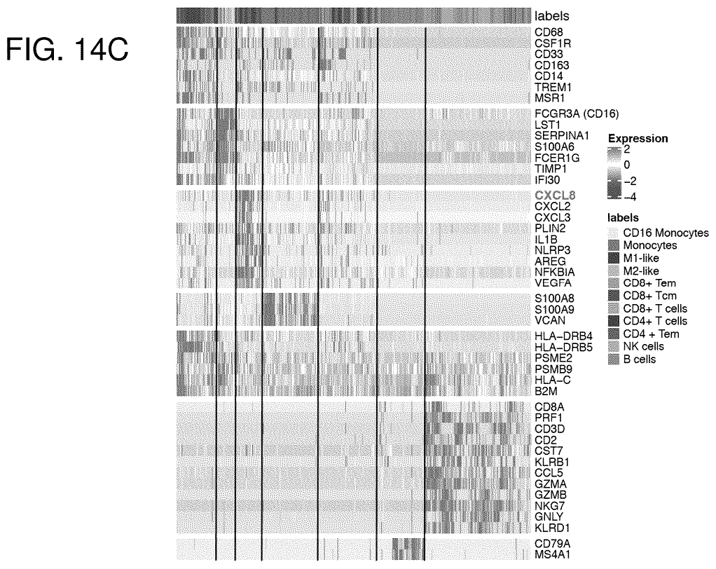

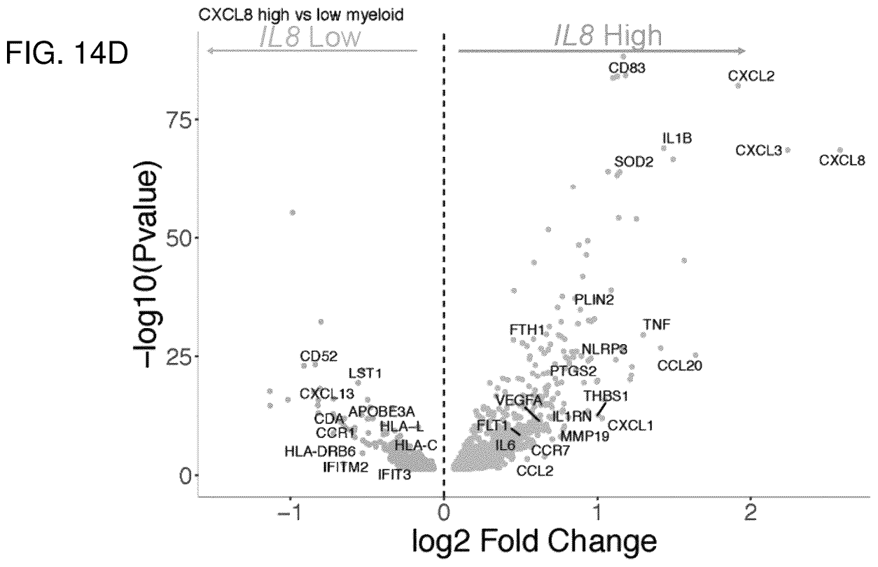

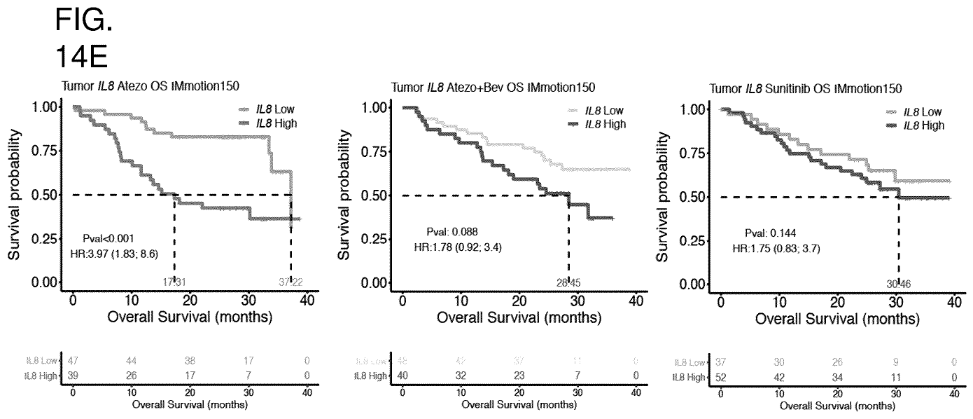

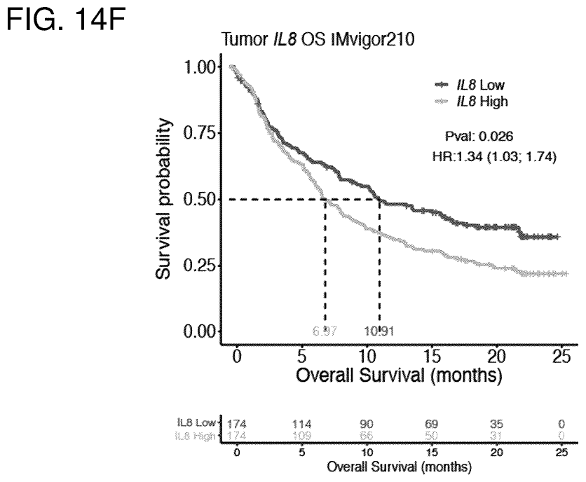

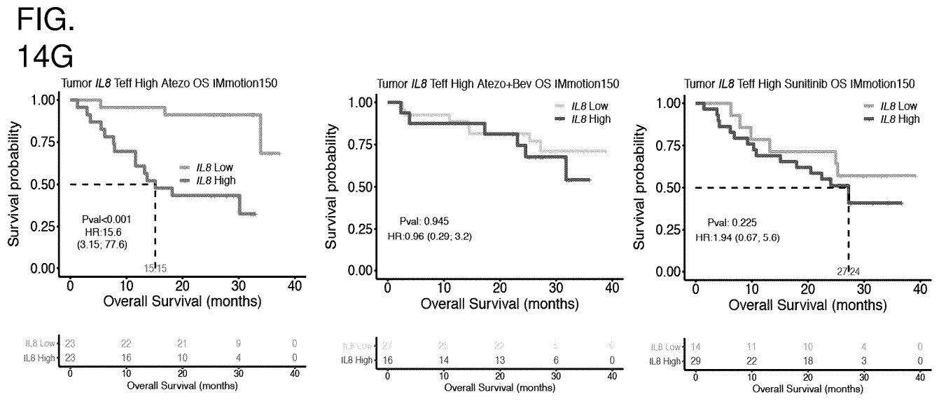

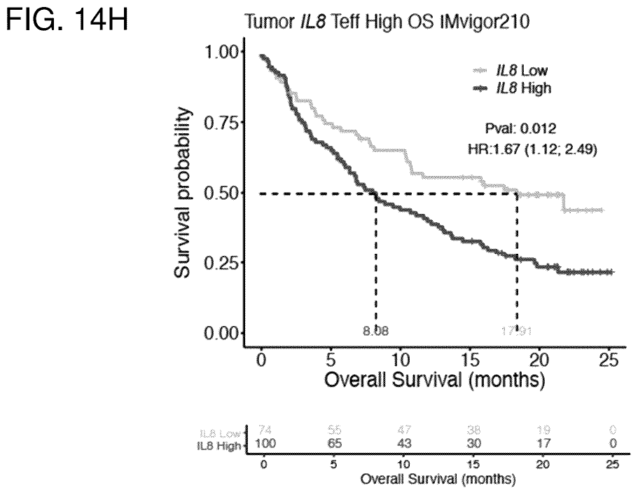

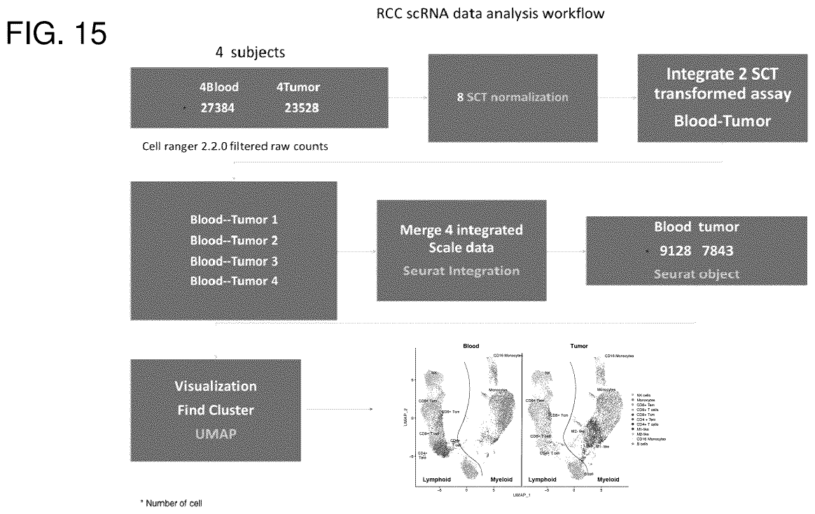

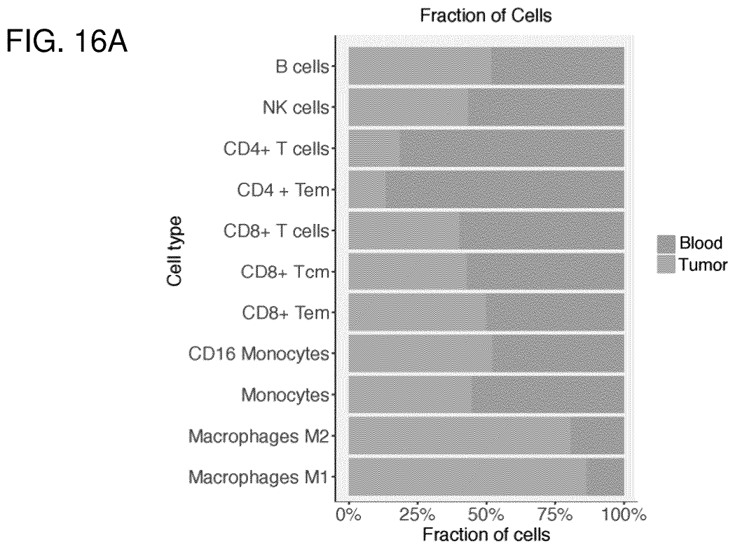

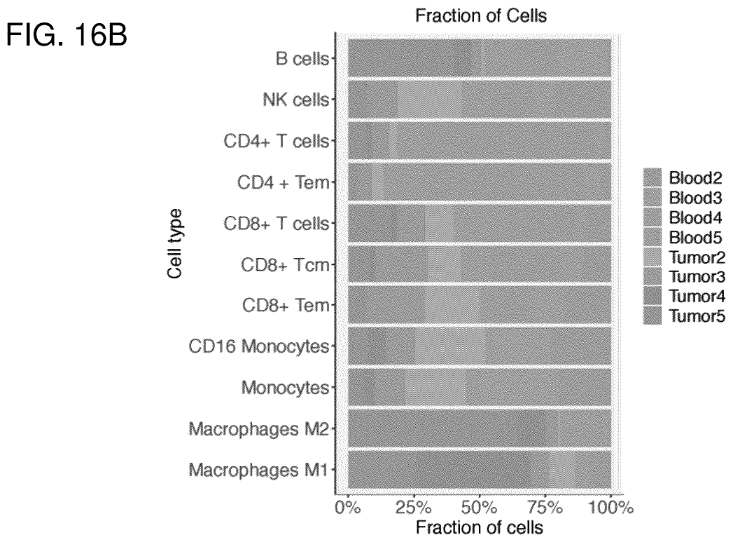

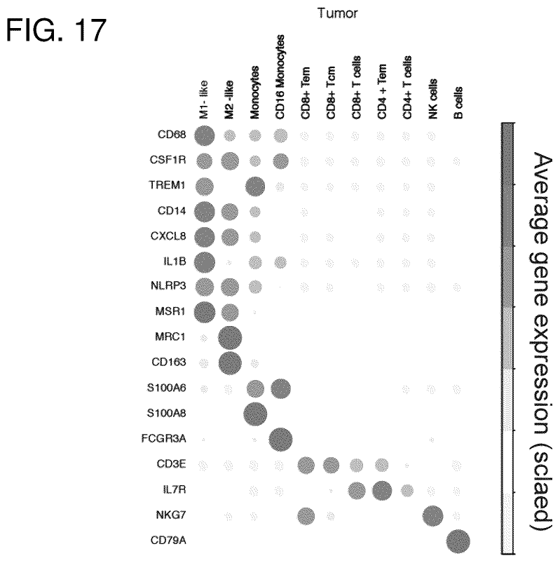

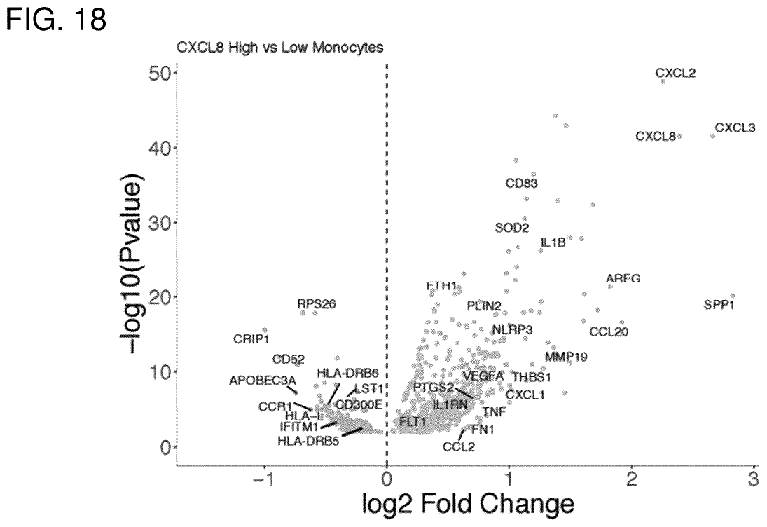

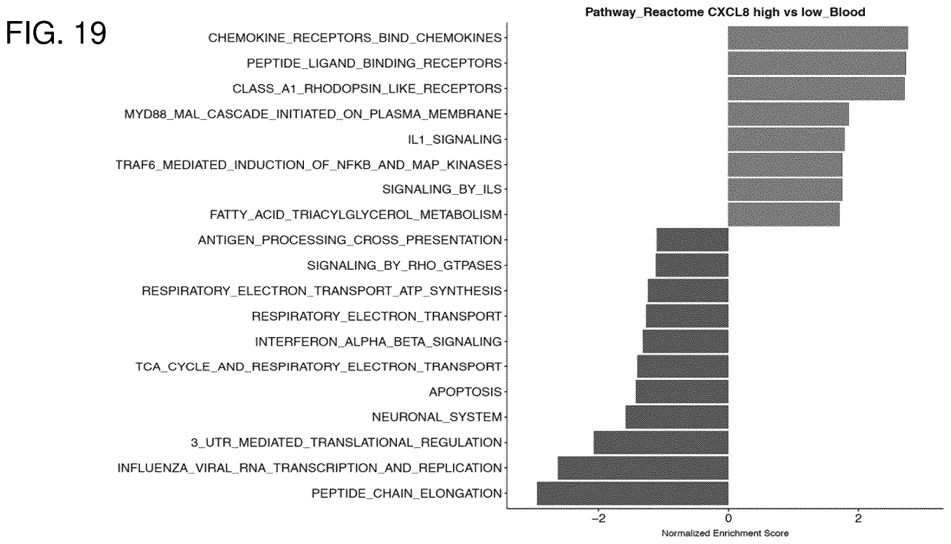

Myeloid cells can acquire different phenotypes within the tumor microenvironment (TME). We therefore procured fresh tumors and peripheral blood from 4 RCC patients and evaluated gene expression profiles by scRNA-seq of intratumoral and matched PBMCs. UMAP visualization showed that lymphoid and myeloid cells clustered separately into 13 subsets (FIGS. 14A, 15, 16A, and 16B). Relative to blood, tumors were enriched in myeloid cells, with concurrent reductions in B cell and T cell subsets (FIG. 14B). Similar to our observation in mUC PBMCs, IL8 expression was specific to myeloid cells and its expression was higher in tumor infiltrating myeloid cells compared to peripheral blood myeloid cells, especially in cluster 7, a tumor specific myeloid cell subset (FIGS. 14B and 17). Differential gene expression analysis of IL8 high versus IL8 low intratumoral myeloid cells revealed higher expression of inflammatory genes (e.g., IL1B, PTGS2, IL1RN, and NLRP3) and among other genes, reduced expression of genes involved in antigen processing and presentation (e.g., HLA-DR5, HLA-DRB6, HLA-C, and B2M) in IL8 high cells (FIGS. 14C and 18). scRNA-seq analysis of IL8 high versus low myeloid populations in blood from RCC patients also showed a similar trend (FIGS. 14D and 19), which was consistent with our observations from PBMC analysis in mUC patients. Genes that are upregulated in IL8 high tumors in kidney cancer (e.g., RCC) are shown in Table 3. Genes that are downregulated in IL8 high tumors in kidney cancers (e.g., RCC) are shown in Table 6. The expression level of one or more of the genes set forth in Table 3 or Table 6 can be used as proxies for IL8 expression.

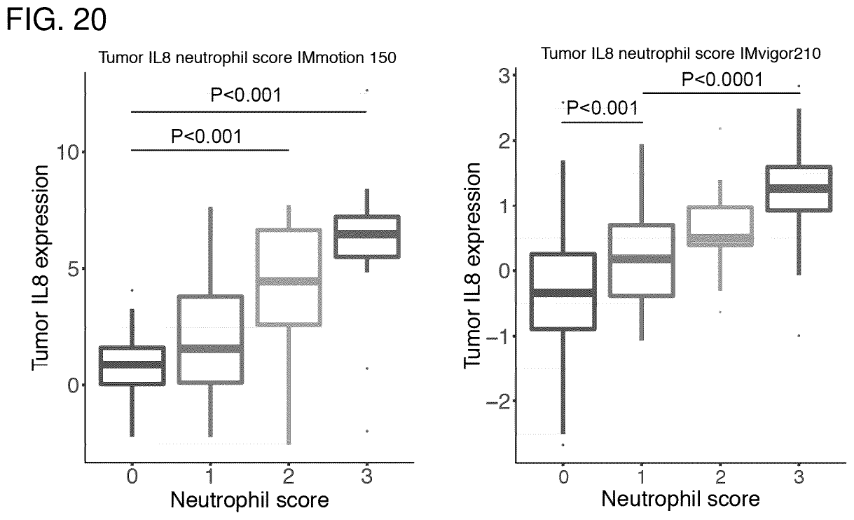

We further interrogated IL8 gene expression in mUC (IMvigor 210) and mRCC (IMmotion 150) tumors and found that elevated IL8 gene expression correlated with higher neutrophil presence by histological assessment (FIG. 20). High IL8 gene expression in tumor associated with worse PFS in mRCC (HR: 2.73, 95% CI: 1.49, 5.0, p=0.0001, FIG. 14E) and worse OS in mUC patients (HR: 1.34, 95% CI: 1.03, 1.74, P=0.026, FIG. 14F) treated with atezolizumab. We also evaluated the effect of IL8 expression on clinical outcomes in Ter high subsets and found that high tumor IL8 expression remained associated with worse PFS even in these immune infiltrated tumors in mRCC (HR: 4.33, 95% CI: 1.75, 10.7, P=0.001, FIG. 14G) and worse OS in mUC (HR: 1.67, 95% CI: 1.12, 2.49, P=0.011) (FIG. 14H) in patients treated with atezolizumab, but not in mRCC patients treated with atezolizumab+bevacizumab or sunitinib (FIG. 14G).

In summary, our data indicate that peripheral and intratumoral IL8 and associated myeloid inflammation may confer resistance to checkpoint blockade. Moreover, expression of IL8 can be used as a predictive biomarker for treatment response to CPI therapy, including anti-cancer therapy that includes a PD-L1 axis binding antagonist. In particular, patients with low IL8 expression are likely to respond to treatment with an anti-cancer therapy that includes a PD-L1 axis binding antagonist, while patients with high IL8 expression may benefit from treatment from anti-cancer therapy other than or in addition to a PD-L1 axis binding antagonist (e.g., a combination therapy that includes a PD-L1 axis binding antagonist and one or more additional anti-cancer therapeutic agents).

B. Materials and Methods

1. Clinical Sample Collection

Samples for this analysis were collected from IMvigor210, a single arm phase 2 study investigating atezolizumab in metastatic urothelial carcinoma patients (NCT02951767, NCT02108652), Phase 3 mUC trial IMvigor211 (NCT02302807) in which patients were treated with either chemotherapy (taxane or vinflunine) or atezolizumab as a second-line or higher treatment. Tumor tissues were taken from all patients 2 years prior to study entry. RECIST v1.1 was used to assess response to therapy.

2. mRCC Sample Acquisition

In our study, a total of fourteen samples from 5 patients diagnosed with clear cell renal carcinoma (ccRCC), five tumors and four matched adjacent normal tissues and whole blood were included in scRNA analysis. Fresh remnant tumor and adjacent normal tissues were collected at the time of elective curative resection.

3. Tumor Tissue Dissociation and Single Cell RNA Sequencing

Surgical resections from treatment-naïve patients with tumors classified as RCC were procured (Discovery Life Sciences, iSpecimen Inc, Avaden BioSciences and TriMetis Life Sciences) and shipped overnight to our institution. A total of eight fresh samples (four tumors and four matched whole blood) from four patients diagnosed with clear cell renal carcinoma (ccRCC) were included in single cell RNA analysis. Upon arrival, samples were rinsed with PBS until no traces of blood were visually detected. Subsequently, samples were digested with a combination of Collagenase D (0.5 mg/ml) and DNAse (0.1 mg/ml) from 15 min at 37° C. with gentle shaking. Subsequently, the samples were subjected to a gentleMACS™ dissociator (Miltenyi Biotec) followed by an additional 10 min incubation at 37° C. Within 24 hours, tissues were processed to single-cell suspensions using the human tumor dissociation kit and gentleMACS™ protocols. Following enzymatic dissociation of tissues, cells were stained with anti-CD45 (Biolegend, San Diego, CA), and CD45+ cells were purified by fluorescence-activated cell sorting (FACS®) on a Becton Dickinson FACSARIA™ cell sorter equipped with four lasers (405 nm, 488 nm, 561 nm, 638 nm). A 70 μm nozzle running at 70 psi and 90 kHz was used as the setup for each sort session. FACS® gates were drawn to include only live single cells based on Calcein Blue AM+ and Propidium Iodide− (Thermo Fisher Scientific, Waltham, MA).

Viable CD45+ cells isolated from RCC blood and tumor were loaded into wells of a 10× CHROMIUM™ single cell capture chip targeting a cell recovery rate of 2000-4000 cells. Single-cell gel beads in emulsion (GEMs) were created on a CHROMIUM™ Single Cell Controller and scRNA-seq libraries were prepared using the CHROMIUM™ Single Cell 5′ Library and Gel Bead kit according to the manufacturer's protocol (10× Genomics). Sequencing libraries were loaded at 1.3 μM on an ILLUMINA® NextSeq500 with High Output 150 cycle kit (Illumina) for paired-end sequencing using the following read length: 26 bp Read1, 8 bp i7 Index, 0 bp i5 Index, and 98 bp Read2.

4. Pre-Enrichment and FACS® Sorting

Following enzymatic dissociation of tissues, single-cell suspensions were subjected to one round of live cell or CD45+ enrichment using magnetic bead separation followed by antibody staining with a cocktail of anti-CD45 and anti-CD3 for identification of T cells, anti-EPCAM for exclusion of epithelial cells, and anti-CD56, anti-CD14, anti-CD16, anti-CD11 b, and anti-CD19 for exclusion of non-T cells from the sorting gate. Cells were purified by fluorescence-activated cell sorting (FACS®) on a Becton Dickinson FACSAria™ cell sorter equipped with 4 lasers (405 nm, 488 nm, 561 nm, 638 nm). A 70-micron nozzle running at 70 psi and 90 kHz was used as the setup for each sort session. FACS® gates were drawn to include only live single cells based on Calcein Blue AM+ and propidium iodide- (Thermo Fisher Scientific). Further gates were drawn to arrive at CD3+CD45+EpCAM− or CD45+EpCAM− cells.

5. Preparation of Single Cell Suspension—CHROMIUM™ Single-Cell 5′RNA-Seq Sequencing Library Construction Using the 10× Genomics CHROMIUM™ Platform

Cellular suspensions were loaded on a CHROMIUM™ Single Cell Controller instrument (10× Genomics) to generate single-cell gel beads in emulsion (GEMs). Single-cell RNA-Seq libraries were prepared using the Chromium Single Cell 5′ Library & Gel Bead Kit (part number (P/N) 1000006 and P/N 220112, 10× Genomics). GEM-RT was performed in a C1000 TOUCH™ thermal cycler with 96-Deep Well Reaction Module (Bio-Rad; P/N 1851197): 53° C. for 45 min, 85° C. for 5 min; held at 4° C. and stored at −20° C. The GEMs were shipped to 10× Genomics on dry ice, then broken and the single-strand cDNA was cleaned up with DYNABEADS® MyOne™ Silane Beads (Thermo Fisher Scientific; P/N 2000048). Barcoded, full-length cDNA was amplified using the C1000 TOUCH™ thermal cycler with 96-Deep Well Reaction Module: 98° C. for 45 s; cycled 133: 98° C. for 20 s, 67° C. for 30 s, and 72° C. for 1 min; 72° C. for 1 min; held at 4° C. Amplified cDNA product was cleaned up with the SPRIselect® reagent Kit (0.63SPRI; Beckman Coulter; P/N B23318).

5′ gene expression was constructed using the reagents in the CHROMIUM™ Single Cell 30/50 Library Construction kit (P/N 1000020). For 5′ gene expression library construction, these steps were followed: (1) fragmentation, end repair and A-tailing; (2) post-fragmentation, end repair and A-tailing cleanup with SPRIselect® reagent; (3) adaptor ligation; (4) post-ligation cleanup with SPRIselect® reagent; (5) sample index PCR double-sided cleanup with SPRIselect® reagent; and (5) post library construction quality control (QC).

For the enriched library construction, these steps were followed: (1) fragmentation, end repair and A-tailing; (2) adaptor ligation; (3) post-ligation cleanup with SPRIselect® reagent; and (4) sample index PCR and cleanup. The barcode sequencing libraries were quantified by quantitative PCR (KAPA Biosystems Library Quantification Kit for ILLUMINA® platforms, P/N KK4824).

6. Software Cell Ranger 2.2.0

The Cell Ranger Single Cell Software Suite v.2.2.1 was used to perform sample de-multiplexing, alignment, filtering, and universal molecular identifier (UMI) counting. The data for each respective subpopulation were aggregated for direct comparison of single cell transcriptomes. A total of 52,671 single cells from the 4 paired tumor samples were captured, with the number of cells recovered per channel ranging from 3695 to 9809. The mean reads per cell varied from 91,346 and 33,789 with median UMIs of 1408 to 4187 per cell. Low-quality cells were discarded if the cell number with expressed genes was smaller than 200. Cells were also removed if their proportions of mitochondrial gene expression were larger than 40%. The final cell number from the 4 paired patients was 25,459. The variation caused by tissue location, that is, adjacent normal and tumor, were removed utilizing the “SCTtransform” function with Seurat.

7. Single Cell Analysis of the Technical Data

Seurat (Butler et al. Nature Biotechnol. 36:411-420, 2018) (version 3.0) was used to analyze the gene expression (GEX) data in FIGS. 10A-10H and 14A-14G. Genes with detected expression in at least 5 cells, and cells with at least 10 genes detected were used. Variable genes were identified with x.low.cutoff=0.05 and y.cutoff=0.1. The first 20 principal components were used for clustering (resolution=0.6) and for UMAP visualization. SingleR (version 1.0.1) was used to annotate immune cells type (Aran et al., Nat. Immunol. 20, 163-172, 2019). SingleR calculates correlations using the variable genes in the reference dataset to assign cellular identity for single cell transcriptomes by comparison to reference data sets of pure cell types sequenced by RNA-sequencing (RNA-seq). Here, we used SingleR to identify monocytes, macrophages and lymphoid cells for RCC tumor cells. In addition, we applied Seurat clustering approach based on SingleR marker correlations to subcluster M1 and M2 macrophages. Immunophenotyping of PBMCs and RCC tumors was inferred from the annotation of cluster-specific genes; CD3 T cells (CD3D, CD3E), CD8 T cells (CD3E, CD8A), B cells (CD79A), CD14 monocytes (CD14, S100A8), and natural killer (NK) cells (NKG7 and CD3E negative).

8. Single Cell Analysis of the RCC Tumor

Seurat (Butler et al. id.) (version 3.0) was used to perform basic quality control on the raw 50 GEX matrices output from Cell Ranger. Prior to the analysis of Seurat, cells with less than 30 genes or more than 3000 genes were filtered out, and genes expressed in less than 5 cells were removed. Next, gene dispersion analysis implemented in Seurat was used to select highly variable genes, preserving genes with logarithmic mean expression between 0.0 and 3.0 and with logarithmic dispersion less than 0.5.

For unbiased visualization of the 5′ GEX data, Principal Component Analysis (PCA), UMAP implemented in Seurat was used: the first 20 principal components were selected for all samples and no major differences were seen when using larger numbers of principal components. Given the 2D visualization by UMAP, feature plots were generated by displaying cells that express CXCL8 gene and specific genes (CD3 T (CD3D, CD3E), CD8 T (CD3E, CD8A), B cells (CD79A), CD14 Monocytes (CD14) and NK cells (NKG7+ and CD3E negative). Next, each cell visualized in the UMAP plots were annotated as one of M (macrophage) cell, NK cell, or B cell, based on its expression. Individual cells were classified by the enrichment of cell type markers: myeloid subsets (CD14, S100A8, IL1B, CXCL8), NK (NKG7), B cell (CD79A), or T cell (CD3D, CD8A).

To classify a cell as a certain type, it was required that the total UMI counts for all the positive markers sum to at least 4 (with the exception of macrophages which requires 8 UMIs as more markers are used).

9. Reagents and Antibodies

Antibodies used in FACS sorting are listed below:
CD45: Biolegend, 2D1, B237418, 368512, APC or 368516 APC/CY®7
CD3: Biolegend, HIT3a, B256208, 300308, PE.
EpCAM: Biolegend, 9C4, B255542, 324222, PE/CY®7.
CD56: Biolegend, 5.1H11, B263355, 362546, FITC.
CD14: Biolegend, 63D3, B257234, 367116, FITC.
CD11b: Biolegend, ICRF44, B218669, 301330, FITC.
CD16: Biolegend, B73.1, B238206, 360716, FITC.
CD19: Biolegend, HIB19, B2448329, 302206, FITC.

10. Software Versions

Computational analysis was performed using Cell Ranger software (10× Genomics) version 2.2.0, Perl version 5.18.4, R version 3.6.0, and the following packages and versions in R for analysis: Seurat, 3.0.0 [2]; edgeR, 3.26.0; cluster, 2.0.8; dynamicTreeCut, 1.63-1; UMAP, WGCNA, 1.66 [53]; and survival, 2.42-6.

11. PBMC Isolation

Whole blood drawn into acid citrate dextrose (ACD) blood collection tubes were shipped overnight and then diluted >3× with Dulbecco's Phosphate-Buffered Saline (DPBS) without calcium or magnesium (Lonza). Diluted cell suspensions were carefully underlayered with FICOLL-PAQUE™ PLUS (GE Healthcare) in 50 ml or 15 ml conical tubes. The tubes were then centrifuged for 30 min at 900×g at room temperature (RT), with the brake off. The interphase containing PBMCs was harvested and washed with PBS and subsequently centrifuged for 10 minutes at 500×g at RT before further processing of the cell pellet.

12. PBMC RNA Extraction

PBMC were isolated from patients in ACD vacutainer tubes (Becton, Dickinson and Company). PBMC cell pellets were then stored in RNAlater® (Qiagen) and stored at 4° C. for 1 to 5 days, until frozen storage at −20° C. until extraction. Extractions were performed using the PureLink® RNA Mini Kit (Thermo Fisher Scientific) and quantified using a NanoDrop™ spectrophotometer (Thermo Fisher Scientific).

13. PBMC NanoString Gene Expression Analysis

Patients were divided into IL8 high-versus low-expression categories using median mRNA expression levels as cutoffs as measured by a NanoString immune panel, and with P values determined by t-test. PBMC NanoString gene expression data were processed using the R/Bioconductor package NanoStringQCPro. Raw counts were adjusted by positive control counts before probe- and lane-specific background was calculated based on both negative controls and blank measurements. After background correction, counts were log 2-transformed and normalized by housekeeping gene expression (TMEM55B, VPS33B, TBP and TUBB).

14. Plasma IL8 Assay

Ethylenediaminetetraacetic acid (EDTA) plasma samples (baseline and cycle 3 day 1 (C3D1) on treatment) were collected from patients before and after treatment and stored at −80° C. IL8 levels were monitored by previously qualified immunoassays on the SIMPLE PLEX® Ella multi-analyte platform. The samples were diluted 2-fold in sample diluent and loaded onto the cartridge for data acquisition.

15. RNA-Seq Gene Expression Profiling

Whole transcriptome profiles for all 298 samples were generated using TruSeq® RNA Access technology. RNA reads were first aligned with the ribosomal RNA sequences to remove ribosomal reads. The remaining reads were aligned to the human reference Genome (GRCh38) using GSNAP aligner allowing maximum of two mismatches with the following parameters: -M 2 -n 10 -B 2 -i 1 -N 1 -w 200000 -E 1 --pairmax-rna=200000 --clip-overlap. To quantify gene expression levels, the number of reads mapped to the exons of each RefSeq gene was calculated in a strand-specific manner using the functionality provided by the R/Bioconductor package GenomicAlignments.

16. Gene Expression Analyses

To calculate scores for each of these signatures, counts were first normalized using edgeR's normalization factors (Robinson et al. Bioinformatics 26(1):139-140, 2010), followed by filtering out genes with low coverage (i.e., not reaching 0.25 CPM (counts per million) in at least one-tenth of available samples) and log 2-transformation using limma's voom (Ritchie et al. Nucl. Acids Res. 43(7):e47, 2015). Gene expression score for each PPI was calculated as described (Mariathasan et al. Nature 554(7693):544-548, 2018). A principal component analysis was performed on the z-score transformed expression of each gene, and principal component 1 was extracted to serve as gene signature scores.

17. Pathway Analysis

Individual REACOME pathways were assessed for significant enrichment by assessing whether the number (S) of significantly differentially expressed genes within a pathway is more than expected by chance given the total number (N) of genes. The p-value (P) was determined using a hypergeometric test and was then corrected for tests over multiple pathways using the method of Benjamini and Hochberg (1995) to yield an adjusted p-value (P adj.).

18. Statistical Analyses

Time-to-event outcomes were estimated using the Kaplan-Meier method, which was used to estimate the probability of OS and to estimate the median OS for the IMvigor210 and IMvigor211 cohorts or PFS for IMmotion150 cohorts, and Kaplan-Meier curves were produced. The OS or PFS were compared by the log-rank test. For OS and PFS analysis, data for patients who were alive were censored at the time of the last contact. The hazard ratios and 95% confidence intervals for OS and PFS were estimated by a Cox regression model. Cox proportional hazards and linear regression model was performed to identify prognostic factors in univariate and multivariate analysis.

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### SEQUENCE LISTING

Table 9 shows sequences that are used throughout the application.

TABLE 9
Sequence Listing

| SEQ ID NO: | SEQUENCE |
|---|---|
| SEQ ID NO: 1 | QVQLVESGGGVVQPGRSLRLDCKASGITFSNSGMHWVRQAPGKGLEWVAVIWYDG |
| | SKRYYADSVKGRFTISRDNSKNTLFLQMNSLRAEDTAVYYCATNDDYWGQGTLVTVS |
| | SASTKGPSVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPPCPAPE |
| | FLGGPSVFLFPPKPKDTLMISRTPEVTCVVVDVSQEDPEVQFNWYVDGVEVHNAKTK |
| | PREEQFNSTYRVVSVLTVLHQDWLNGKEYKCKVSNKGLPSSIEKTISKAKGQPREPQ |
| | VYTLPPSQEEMTKNQVSLTCLVKGFYPSDIAVEWESNGQPENNYKTTPPVLDSDGSF |
| | FLYSRLTVDKSRWQEGNVFSCSVMHEALHNHYTQKSLSLSLGK |
| SEQ ID NO: 2 | EIVLTQSPATLSLSPGERATLSCRASQSVSSYLAWYQQKPGQAPRLLIYDASNRATGI |
| | PARFSGSGSGTDFTLTISSLEPEDFAVYYCQQSSNWPRTFGQGTKVEIKRTVAAPSV |
| | FIFPPSDEQLKSGTASVVCLLNNFYPREAKVQWKVDNALQSGNSQESVTEQDSKDST |
| | YSLSSTLTLSKADYEKHKVYACEVTHQGLSSPVTKSFNRGEC |
| SEQ ID NO: 3 | EVQLVESGGGLVQPGGSLRLSCAASGFTFSDSWIHWVRQAPGKGLEWVAWISPYG |
| | GSTYYADSVKGRFTISADTSKNTAYLQMNSLRAEDTAVYYCARRHWPGGFDYWGQ |
| | GTLVTVSA |
| SEQ ID NO: 4 | DIQMTQSPSSLSASVGDRVTITCRASQDVSTAVAWYQQKPGKAPKLLIYSASFLYSGV |
| | PSRFSGSGSGTDFTLTISSLQPEDFATYYCQQYLYHPATFGQGTKVEIKR |
| SEQ ID NO: 5 | GFTFSX₁SWIH, wherein X₁ is D or G |
| SEQ ID NO: 6 | AWIX₂PYGGSX₃YYADSVKG, wherein X₂ is S or L and X₃ is T or S |
| SEQ ID NO: 7 | RHWPGGFDY |
| SEQ ID NO: 8 | EVQLVESGGGLVQPGGSLRLSCAAS |
| SEQ ID NO: 9 | WVRQAPGKGLEWV |
| SEQ ID NO: 10 | RFTISADTSKNTAYLQMNSLRAEDTAVYYCAR |
| SEQ ID NO: 11 | WGQGTLVTVSA |
| SEQ ID NO: 12 | RASQX₄X₅X₆TX₇X₈A, wherein X₄ is D or V, X₅ is V or I, X₆ is S or N, |
| | X₇ is A or F, and X₈ is V or L |
| SEQ ID NO: 13 | SASX₉LX₁₀S, wherein X₉ is F or T and X₁₀ is Y or A |
| SEQ ID NO: 14 | QQX₁₁X₁₂X₁₃X₁₄PX₁₅T, wherein X₁₁ is Y, G, F, or S, X₁₂ is L, Y, F or W, |
| | X₁₃ is Y, N, A, T, G, F or I, X₁₄ is H, V, P, T or I, and X₁₅ is A, W, |
| | R, P or T |
| SEQ ID NO: 15 | DIQMTQSPSSLSASVGDRVTITC |
| SEQ ID NO: 16 | WYQQKPGKAPKLLIY |
| SEQ ID NO: 17 | GVPSRFSGSGSGTDFTLTISSLQPEDFATYYC |
| SEQ ID NO: 18 | FGQGTKVEIKR |
| SEQ ID NO: 19 | GFTFSDSWIH |
| SEQ ID NO: 20 | AWISPYGGSTYYADSVKG |
| SEQ ID NO: 21 | RHWPGGFDY |
| SEQ ID NO: 22 | RASQDVSTAVA |
| SEQ ID NO: 23 | SASFLYS |
| SEQ ID NO: 24 | QQYLYHPAT |
| SEQ ID NO: 25 | EVQLVESGGGLVQPGGSLRLSCAASGFTFSDSWIHWVRQAPGKGLEWVAWISPYG |
| | GSTYYADSVKGRFTISADTSKNTAYLQMNSLRAEDTAVYYCARRHWPGGFDYWGQ |
| | GTLVTVSS |
| SEQ ID NO: 26 | EVQLVESGGGLVQPGGSLRLSCAASGFTFSDSWIHWVRQAPGKGLEWVAWISPYG |
| | GSTYYADSVKGRFTISADTSKNTAYLQMNSLRAEDTAVYYCARRHWPGGFDYWGQ |
| | GTLVTVSSASTK |
| SEQ ID NO: 27 | WGQGTLVTVSS |
| SEQ ID NO: 28 | FGQGTKVEIK |
| SEQ ID NO: 29 | EVQLVESGGGLVQPGGSLRLSCAASGFTFS |
| SEQ ID NO: 30 | WVRQAPGKGLEWVA |
| SEQ ID NO: 31 | WGQGTLVTVSSASTK |
| SEQ ID NO: 32 | EVQLVESGGGLVQPGGSLRLSCAASGFTFSDSWIHWVRQAPGKGLEWVAWISPYG |
| | GSTYYADSVKGRFTISADTSKNTAYLQMNSLRAEDTAVYYCARRHWPGGFDYWGQ |
| | GTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKKVEPKSCDKTH |
| | TCPPCPAPELLGGPSVFLFPPKPKDTLMISRTPEVTCVVVDVSHEDPEVKFNWYVDG |
| | VEVHNAKTKPREEQYASTYRVVSVLTVLHQDWLNGKEYKCKVSNKALPAPIEKTISKA |
| | KGQPREPQVYTLPPSREEMTKNQVSLTCLVKGFYPSDIAVEWESNGQPENNYKTTP |
| | PVLDSDGSFFLYSKLTVDKSRWQQGNVFSCSVMHEALHNHYTQKSLSLSPG |
| SEQ ID NO: 33 | DIQMTQSPSSLSASVGDRVTITCRASQDVSTAVAWYQQKPGKAPKLLIYSASFLYSGV |
| | PSRFSGSGSGTDFTLTISSLQPEDFATYYCQQYLYHPATFGQGTKVEIKRTVAAPSVFI |
| | FPPSDEQLKSGTASVVCLLNNFYPREAKVQWKVDNALQSGNSQESVTEQDSKDSTY |
| | SLSSTLTLSKADYEKHKVYACEVTHQGLSSPVTKSFNRGEC |

1-146. (canceled)

147. A method of treating human individual having a cancer, the method comprising:
(a) determining the expression level of IL8 in a sample from the individual, wherein an expression level of IL8 in the sample that is below a reference level of IL8 identifies the individual as likely to respond to treatment with an anti-cancer therapy comprising an anti-PD-L1 antibody, wherein the response is indicated by overall survival (OS) or objective response rate (ORR); and
(b) administering an effective amount of an anti-cancer therapy comprising the anti-PD-L1 antibody to the individual based on the expression level of IL8 determined in step (a), wherein the anti-PD-L1 antibody comprises the following hypervariable regions (HVRs):
(i) an HVR-H1 sequence of GFTFSDSWIH (SEQ ID NO: 19);
(ii) an HVR-H2 sequence of AWISPYGGSTYYADSVKG (SEQ ID NO: 20);
(iii) an HVR-H3 sequence of RHWPGGFDY (SEQ ID NO: 21);
(iv) an HVR-L1 sequence of RASQDVSTAVA (SEQ ID NO: 22);
(v) an HVR-L2 sequence of SASFLYS (SEQ ID NO: 23); and
(vi) an HVR-L3 sequence of QQYLYHPAT (SEQ ID NO: 24).

148. The method of claim 147, wherein the sample is a tumor tissue sample, a plasma sample, a whole blood sample, or a cell sample.

149. The method of claim 148, wherein:
(a) the tumor tissue sample comprises tumor cells, tumor-infiltrating immune cells, stromal cells, or a combination thereof; or
(b) the whole blood sample or the cell sample comprises peripheral blood mononuclear cells (PBMCs).

150. The method of claim 149, wherein the tumor-infiltrating immune cells comprise tumor-infiltrating myeloid cells.

151. The method of claim 147, wherein the sample is a formalin-fixed and paraffin-embedded (FFPE) sample, a fresh sample, or a frozen sample.

152. The method of claim 147, wherein the sample is obtained from the individual at a time point prior to or concurrently with administration of the anti-PD-L1 antibody.

153. The method of claim 147, wherein the cancer is a bladder cancer or a kidney cancer.

154. The method of claim 153, wherein:
(a) the bladder cancer is a urothelial carcinoma (UC); or
(b) the kidney cancer is a renal cell carcinoma (RCC).

155. The method of claim 154, wherein:
(a) the UC is a locally advanced or metastatic UC; or
(b) the RCC is a metastatic RCC (mRCC).

156. The method of claim 155, wherein:
(a) the individual having a locally advanced or metastatic UC has received a prior platinum-based chemotherapy or is previously untreated for the locally advanced or metastatic UC; or
(b) the individual having an mRCC is previously untreated for the mRCC.

157. The method of claim 156, wherein the individual who is previously untreated for the locally advanced or metastatic UC is cisplatin-ineligible.

158. The method of claim 147, wherein the method further comprises administering bevacizumab to the individual.

159. The method of claim 147, wherein the expression level is:
(a) an mRNA expression level; or
(b) a protein expression level.

160. The method of claim 159, wherein:
(a) the mRNA expression level is determined by RNA sequencing (RNA-seq), Nanostring, in situ hybridization (ISH), or a combination thereof; or
(b) the protein expression level is determined by enzyme-linked immunosorbent assay (ELISA).

161. The method of claim 159, wherein the mRNA expression level is a normalized mRNA expression level.

162. The method of claim 161, wherein the normalized mRNA expression level is normalized against the mRNA expression levels of TMEM55B, VPS33B, TBP and TUBB.

163. The method of claim 147, wherein the reference level of IL8 is a median expression level of IL8 determined in a population of patients having the cancer.

164. The method of claim 147, wherein the individual has an expression level of a T effector (T_{eff}) signature in a tumor sample that is at or above a reference level for the T_{eff} signature.

165. The method of claim 164, wherein the T_{eff} signature comprises CD8A, GZMA, GZMB, and PRF1.

166. The method of claim 147, wherein the anti-PD-L1 antibody comprises:
(a) a VH domain comprising the amino acid sequence of SEQ ID NO: 25; and
(b) a VL domain comprising the amino acid sequence of SEQ ID NO: 4.

167. The method of claim 147, wherein the anti-PD-L1 antibody is atezolizumab.


SEQUENCE LISTING (33 sequences)

SEQ ID NO: 1 - protein, 440 aa, synthetic construct
  source 1..440: note = Synthetic Polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, synthetic construct
  source 1..214: note = Synthetic Polypetide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 118 aa, synthetic construct
  source 1..118: note = Synthetic Polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 4 - protein, 108 aa, synthetic construct
  source 1..108: note = Synthetic Polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 5 - protein, 10 aa, synthetic construct
  source 1..10: note = Synthetic Polypeptide
  VARIANT 6: note = MOD_RES - Xaa is Asp or Gly
GFTFSXSWIH

SEQ ID NO: 6 - protein, 18 aa, synthetic construct
  source 1..18: note = Synthetic Polypeptide
  VARIANT 4: note = MOD_RES - Xaa is Ser or Leu
  VARIANT 10: note = MOD_RES - Xaa is Thr or Ser
AWIXPYGGSXYYADSVKG

SEQ ID NO: 7 - protein, 9 aa, synthetic construct
  source 1..9: note = Synthetic Polypeptide
RHWPGGFDY

SEQ ID NO: 8 - protein, 25 aa, synthetic construct [sequence omitted]

SEQ ID NO: 9 - protein, 13 aa, synthetic construct
  source 1..13: note = Synthetic Polypeptide
WVRQAPGKGLEWV

SEQ ID NO: 10 - protein, 32 aa, synthetic construct
  source 1..32: note = Synthetic Polypeptide
RFTISADTSKNTAYLQMNSLRAEDTAVYYCAR

SEQ ID NO: 11 - protein, 11 aa, synthetic construct
  source 1..11: note = Synthetic Polypeptide
WGQGTLVTVSA

SEQ ID NO: 12 - protein, 11 aa, synthetic construct
  source 1..11: note = Synthetic Polypeptide
  VARIANT 5: note = MOD_RES - Xaa is Asp or Val
  VARIANT 6: note = MOD_RES - Xaa is Val or Ile
  VARIANT 7: note = MOD_RES - Xaa is Ser or Asn
  VARIANT 9: note = MOD_RES - Xaa is Ala or Phe
  VARIANT 10: note = MOD_RES - Xaa is Val or Leu
RASQXXXTXXA

SEQ ID NO: 13 - protein, 7 aa, synthetic construct
  source 1..7: note = Synthetic Polypeptide
  VARIANT 4: note = MOD_RES - Xaa is Phe or Thr
  VARIANT 6: note = MOD_RES - Xaa is Tyr or Ala
SASXLXS

SEQ ID NO: 14 - protein, 9 aa, synthetic construct
  source 1..9: note = Synthetic Polypeptide
  VARIANT 3: note = MOD_RES - Xaa is Tyr, Gly, Phe, or Ser
  VARIANT 4: note = MOD_RES - Xaa is Leu, Tyr, Phe, or Trp
  VARIANT 5: note = MOD_RES - Xaa is Tyr, Asn, Ala, Thr, Gly, Phe, or Ile
  VARIANT 6: note = MOD_RES - Xaa is His, Val, Pro, Thr, or Ile
  VARIANT 8: note = MOD_RES - Xaa is Ala, Trp, Arg, Pro, or Thr
QQXXXXPXT

SEQ ID NO: 15 - protein, 23 aa, synthetic construct
  source 1..23: note = Synthetic Polypeptide
DIQMTQSPSSLSASVGDRVTITC

SEQ ID NO: 16 - protein, 15 aa, synthetic construct
  source 1..15: note = Synthetic Polypeptide
WYQQKPGKAPKLLIY

SEQ ID NO: 17 - protein, 32 aa, synthetic construct
  source 1..32: note = Synthetic Polypeptide
GVPSRFSGSGSGTDFTLTISSLQPEDFATYYC

SEQ ID NO: 18 - protein, 11 aa, synthetic construct
  source 1..11: note = Synthetic Polypeptide
FGQGTKVEIKR

SEQ ID NO: 19 - protein, 10 aa, synthetic construct
  source 1..10: note = Synthetic Polypeptide
GFTFSDSWIH

SEQ ID NO: 20 - protein, 18 aa, synthetic construct
  source 1..18: note = Synthetic Polypeptide
AWISPYGGSTYYADSVKG

SEQ ID NO: 21 - protein, 9 aa, synthetic construct
  source 1..9: note = Synthetic Polypeptide
RHWPGGFDY

SEQ ID NO: 22 - protein, 11 aa, synthetic construct
  source 1..11: note = Synthetic Polypeptide
RASQDVSTAVA

SEQ ID NO: 23 - protein, 7 aa, synthetic construct
  source 1..7: note = Synthetic Polypeptide
SASFLYS

SEQ ID NO: 24 - protein, 9 aa, synthetic construct
  source 1..9: note = Synthetic Polypeptide
QQYLYHPAT

SEQ ID NO: 25 - protein, 118 aa, synthetic construct [sequence omitted]

SEQ ID NO: 26 - protein, 122 aa, synthetic construct
  source 1..122: note = Synthetic Polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTK

SEQ ID NO: 27 - protein, 11 aa, synthetic construct
  source 1..11: note = Synthetic Polypeptide
WGQGTLVTVSS

SEQ ID NO: 28 - protein, 10 aa, synthetic construct
  source 1..10: note = Synthetic Polypeptide
FGQGTKVEIK

SEQ ID NO: 29 - protein, 30 aa, synthetic construct
  source 1..30: note = Synthetic Polypeptide
EVQLVESGGGLVQPGGSLRLSCAASGFTFS

SEQ ID NO: 30 - protein, 14 aa, synthetic construct
  source 1..14: note = Synthetic Polypeptide
WVRQAPGKGLEWVA

SEQ ID NO: 31 - protein, 15 aa, synthetic construct
  source 1..15: note = Synthetic Polypeptide
WGQGTLVTVSSASTK

SEQ ID NO: 32 - protein, 447 aa, synthetic construct
  source 1..447: note = Synthetic Polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 214 aa, synthetic construct
  source 1..214: note = Synthetic Polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC